(19) United States
(12) Patent Application Publication
(10) Pub. No.: US 2026/0325686 A1
(43) Pub. Date: Oct. 22, 2026

(54) STRUCTURALLY-REINFORCED STABLE THERANOSTIC COPPER RADIOPHARMACEUTICALS TARGETING FIBROBLAST ACTIVATION PROTEIN FOR CANCER DIAGNOSIS AND THERAPY

(71) Applicant: The Board of Regents of The University of Texas System, Austin, TX (US)

(72) Inventors: Pawan Thapa, Dallas, TX (US); Sashi Debnath, Dallas, TX (US); Hans Hammers, Dallas, TX (US); Xiankai Sun, Dallas, TX (US)

(21) Appl. No.: 19/479,238

(22) PCT Filed: Apr. 29, 2024
(86) PCT No.: PCT/US2024/026770

§ 371 Date: Oct. 27, 2025

Related U.S. Application Data
(60) Provisional application No. 63/498,896, filed on Apr. 28, 2023.

(51) Int. Cl. A61K 51/04; A61P 35/00

(52) U.S. Cl. CPC A61K 51/0497; A61K 51/0455; A61P 35/00

(57) ABSTRACT

Provided herein are multivalent FAP-targeting radiopharmaceutical compositions built upon a stable and neutral bifunctional chelator for enhanced specific binding and tumor retention of FAP-targeted theranostics. Also provided are method for using these compositions.

### CROSS REFERENCE TO RELATED APPLICATION

This application claims priority to U.S. Provisional Patent Application Ser. No. 63/498,896, filed Apr. 28, 2023, and titled “STABLE THERANOSTIC COPPER RADIOPHARMACEUTICALS,” which is incorporated by reference herein in its entirety.

### BACKGROUND

### 1. Field

The present disclosure relates to structurally stable copper radiopharmaceuticals that target the fibroblast activation protein. In one aspect, the disclosure relates to compositions comprising the radiopharmaceuticals provided herein and methods of using the compositions for cancer diagnosis and treatment.

### 2. Discussion of Related Art

Fibroblast Activation Protein (FAP) is a type II integral membrane serine protease and is a tumor-associated antigen with selective over-expression in more than 90% of human epithelial carcinoma. FAP expression in heterogeneously populated fibroblast tissue called Cancer-associated Fibroblast (CAF) is imperative to tumor progression and is unique to CAF with no or low expression in healthy adult tissues. These features of FAP ensure a key opportunity to navigate solid tumors and stromal components of tumors with imaging and therapeutic radiotracers. FAP-targeting approach has been recognized as a “billion-dollar target for theranostic product” development, thanks to the wide scope of FAP targeting approach including to those in non-malignant conditions such as myocardial infarction, chronic inflammation, fibrosis of the lung, liver and kidney, and rheumatoid arthritis.

Development of selective inhibitors to FAP has been a long-standing goal in the field of drug discovery. In this context, recently discovered quinoline-derived small molecule inhibitors have shown a significant application in FAP targeting studies related to imaging modalities, such as Positron Emission Tomography (PET) and Single Photon Emission Computer Tomography (SPECT), for cancer diagnosis and therapy. One such example is Fibroblast Activation Protein Inhibitor UAMC-1110 emerging as a selective FAP targeting moiety in many radiotracers over the past few years. Recent advances have been made in diagnostic utility using FAPI-04 and other variants including peptide-based radiotracers linked with shorter half-life isotope like ⁶⁸Ga for cancer imaging purposes. Alternatively, longer half-lived radioisotopes and the theranostic pair of ⁶⁴Cu and ⁶⁷Cu [⁶⁷Cu (t_{1/2}=2.58 d; 100% β-; E_{β}-max=562 keV) and ⁶⁴Cu (t_{1/2}=12.7 h; 17.4% β+; E_{β}+max=653 keV)] are favorable for FAPI based theranostic radiopharmaceutical development.

There is therefore a need in the field for copper based theranostics targeting FAP, that exhibit high in-vivo stability.

### SUMMARY

The present disclosure relates to compound of Formula (I), any positional isomer, any stereo isomer, or any pharmaceutically acceptable salt thereof:

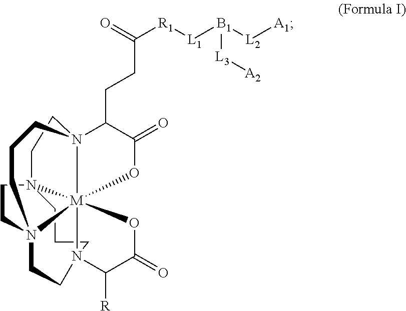

In Formula (I), A₁ is a Fibroblast Activated Protein (FAP) targeting group (FAPI) or an albumin binder (AB) group, wherein the FAPI or the AB group is capable of covalent or non-covalent binding with the FAP or albumin respectively; A₂ is a Fibroblast Activated Protein (FAP) targeting group (FAPI), an albumin binder (AB) group, or absent, wherein the FAPI or the AB group is capable of covalent or non-covalent binding with the FAP or albumin respectively; R₁ is —NH, piperazine, piperidine, an alkylamine, a dicarboxylate amide, or is absent; L₁ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof; L₂ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent; L₃ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent; B₁ is a heterocyclic group, a heterocyclic alkyl, an amidoalkyl, an alkylamide, or is absent; R is hydrogen or Formula (Ia)

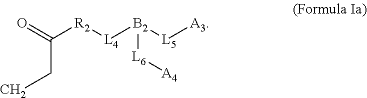

In Formula (Ia), A₃ is a Fibroblast Activated Protein (FAP) targeting group (FAPI) or an albumin binder (AB) group, wherein the FAPI or the AB group is capable of covalent or non-covalent binding with the FAP or albumin respectively; A₄ is a Fibroblast Activated Protein (FAP) targeting group (FAPI) or an albumin binder (AB) group, or is absent, wherein the FAPI or the AB group is capable of covalent or non-covalent binding with the FAP or albumin respectively; R₂ is —NH, piperazine, piperidine, an alkylamine, a dicarboxylate amide, or is absent; L₄ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof; L₅ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent; L₆ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent; B₂ is a heterocyclic group, a heterocyclic alkyl, an amidoalkyl, an alkylamide, or is absent; and M is ⁶⁴Cu, ⁶⁰Cu, ⁶¹Cu, ⁶²Cu or ⁶⁷Cu.

One aspect of the present disclosure relates to the compound of Formula (I), and the FAPI comprises one of the following groups:
targeting group A (TGA):

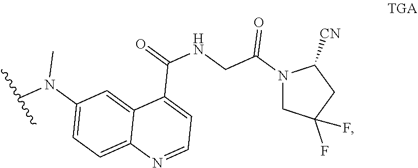 [SMILES: *N(C)c1ccc2nccc(C(=O)NCC(=O)N3CC(F)(F)C[C@H]3C#N)c2c1]

targeting group B (TGB):

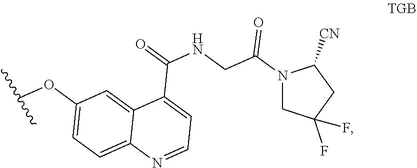 [SMILES: *Oc1ccc2nccc(C(=O)NCC(=O)N3CC(F)(F)C[C@H]3C#N)c2c1]

targeting group C (TGC):

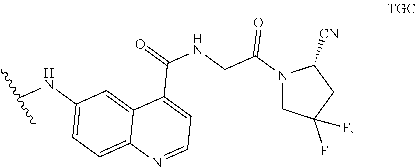 [SMILES: *Nc1ccc2nccc(C(=O)NCC(=O)N3CC(F)(F)C[C@H]3C#N)c2c1]

targeting group D (TGD):

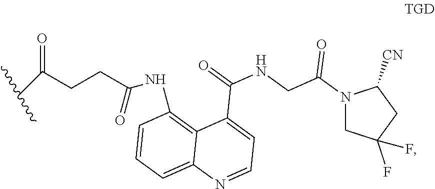 [SMILES: *C(=O)CCC(=O)Nc1cccc2nccc(C(=O)NCC(=O)N3CC(F)(F)C[C@H]3C#N)c12]

targeting group E (TGE):

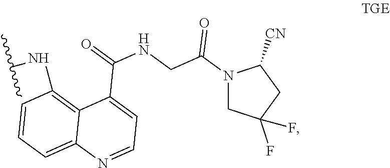 [SMILES: *Nc1cccc2nccc(C(=O)NCC(=O)N3CC(F)(F)C[C@H]3C#N)c12]

targeting group F (TGF):

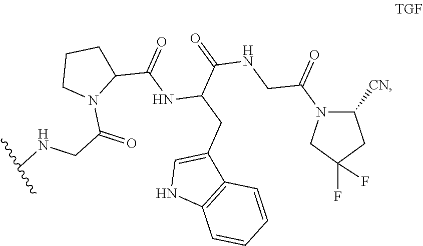 [SMILES: *NCC(=O)N1CCCC1C(=O)NC(Cc1c[nH]c2ccccc12)C(=O)NCC(=O)N1CC(F)(F)C[C@H]1C#N]

targeting group G (TGG):

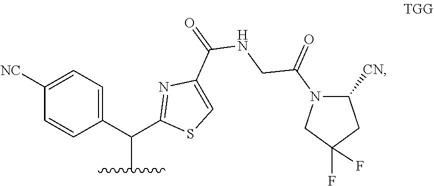 [SMILES: *C(c1ccc(C#N)cc1)c1nc(C(=O)NCC(=O)N2CC(F)(F)C[C@H]2C#N)cs1]

targeting group H (TGH):

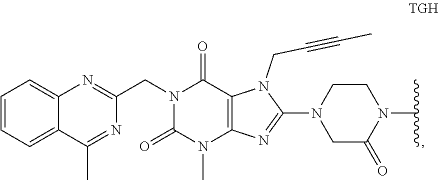 [SMILES: *N1CCN(c2nc3c(c(=O)n(Cc4nc(C)c5ccccc5n4)c(=O)n3C)n2CC#CC)CC1=O]

targeting group I (TGI):

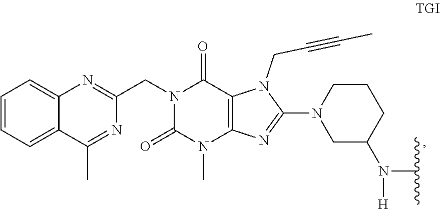 [SMILES: *NC1CCCN(c2nc3c(c(=O)n(Cc4nc(C)c5ccccc5n4)c(=O)n3C)n2CC#CC)C1]

or
targeting group J (TGJ):

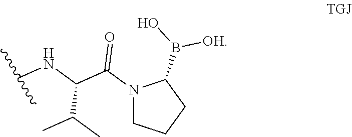 [SMILES: *N[C@H](C(=O)N1CCC[C@H]1B(O)O)C(C)C]

Another aspect of the present disclosure relates to the compound of Formula (I), and the FAPI or the AB group is a tautomer, an enantiomer, a diastereomer, or a derivative thereof.

Another aspect of the present disclosure relates to the compound of Formula (I), and the polyethylene glycol (PEG) and the functionalized polyethylene glycol (functionalized PEG) linker comprise 1 to 12 ethylene glycol repeating units; and the functionalized PEG linker comprises one or more functional groups selected from the group consisting of amine, amide, carbonyl, carboxylate, heterocyclic, amidoalkylcarbonyl, amidoalkylcarboxylate, alkylamine, alkylamide, alkylcarbony, alkylcarboxylate, heterocyclic alkyl, and any combination thereof.

Another aspect of the present disclosure relates to the compound of Formula (I), and the peptidomimetic or peptide linker comprises 1 to 10 amino acid residues. In another aspect, the peptidomimetic or peptide linker comprises a lysine residue.

Another aspect of the present disclosure relates to the compound of Formula (I), and the AB group comprises any one of the following:

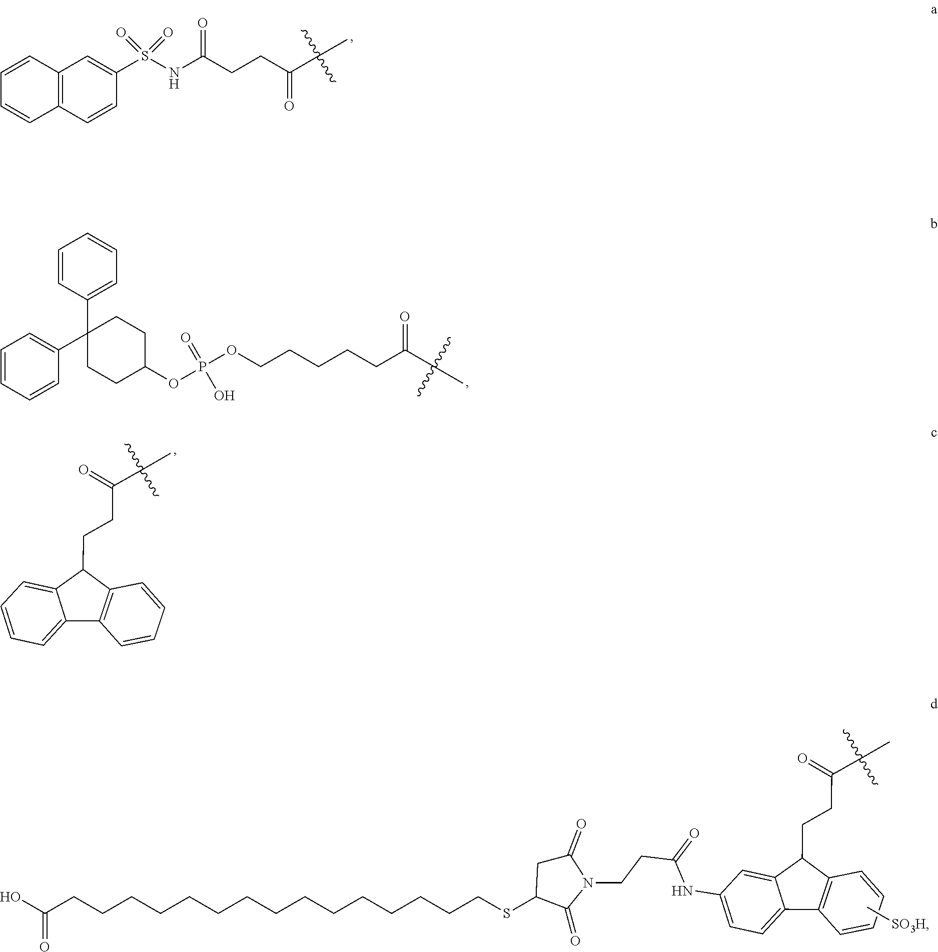

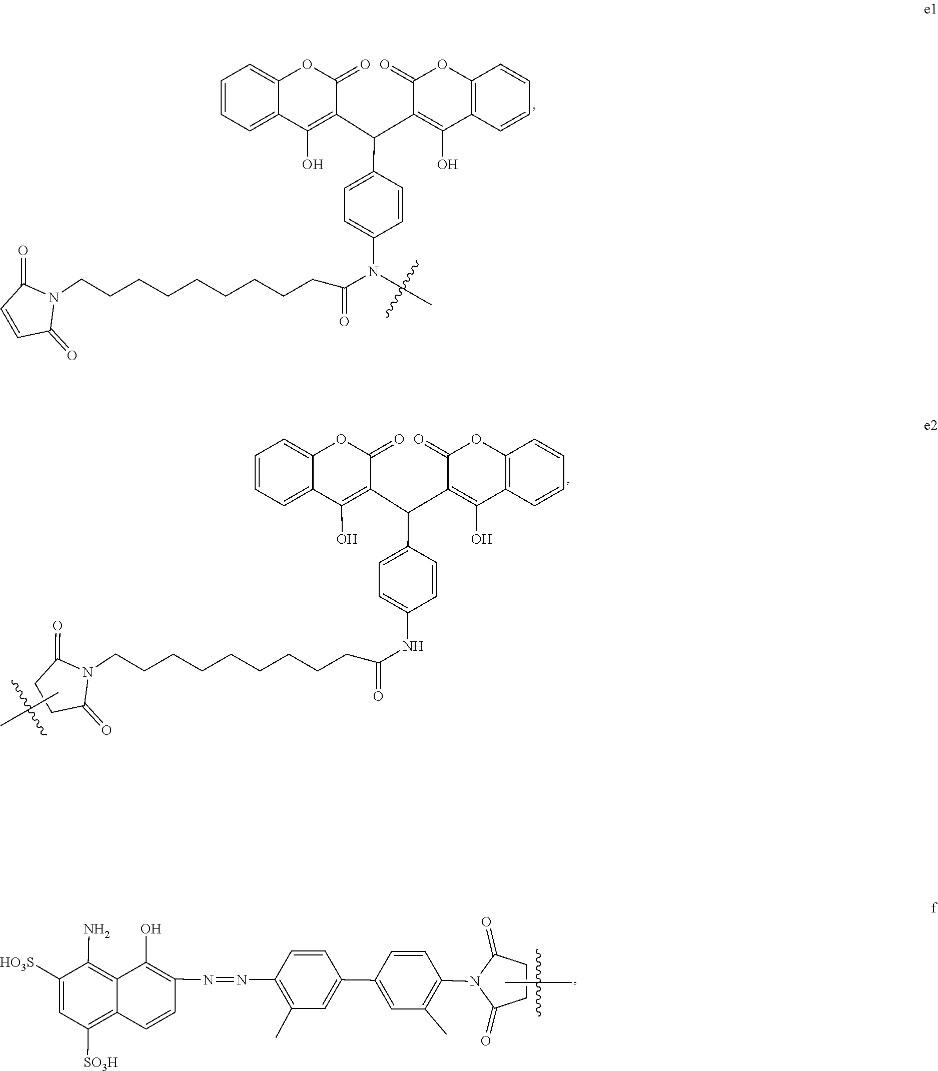

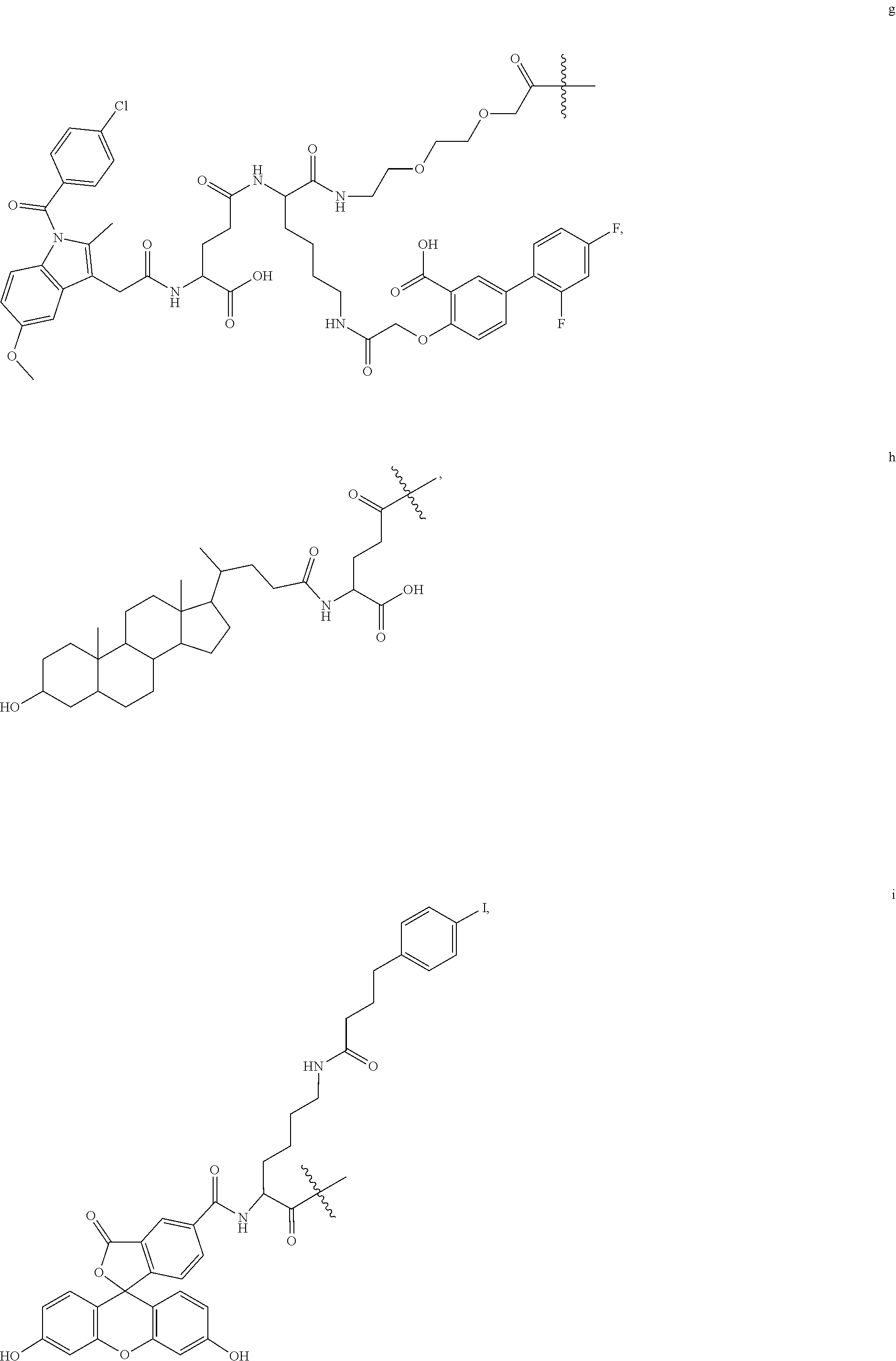 [SMILES: *C(=O)COCCOCCNC(=O)C(CCCCNC(=O)COc1ccc(-c2ccc(F)cc2F)cc1C(=O)O)NC(=O)CCC(NC(=O)Cc1c(C)n(C(=O)c2ccc(Cl)cc2)c2ccc(OC)cc12)C(=O)O ; *C(=O)CCC(NC(=O)CCC(C)C1CCC2C3CCC4CC(O)CCC4(C)C3CCC12C)C(=O)O ; *C(=O)C(CCCCNC(=O)CCCc1ccc(I)cc1)NC(=O)c1ccc2c(c1)C(=O)OC21c2ccc(O)cc2Oc2cc(O)ccc21]

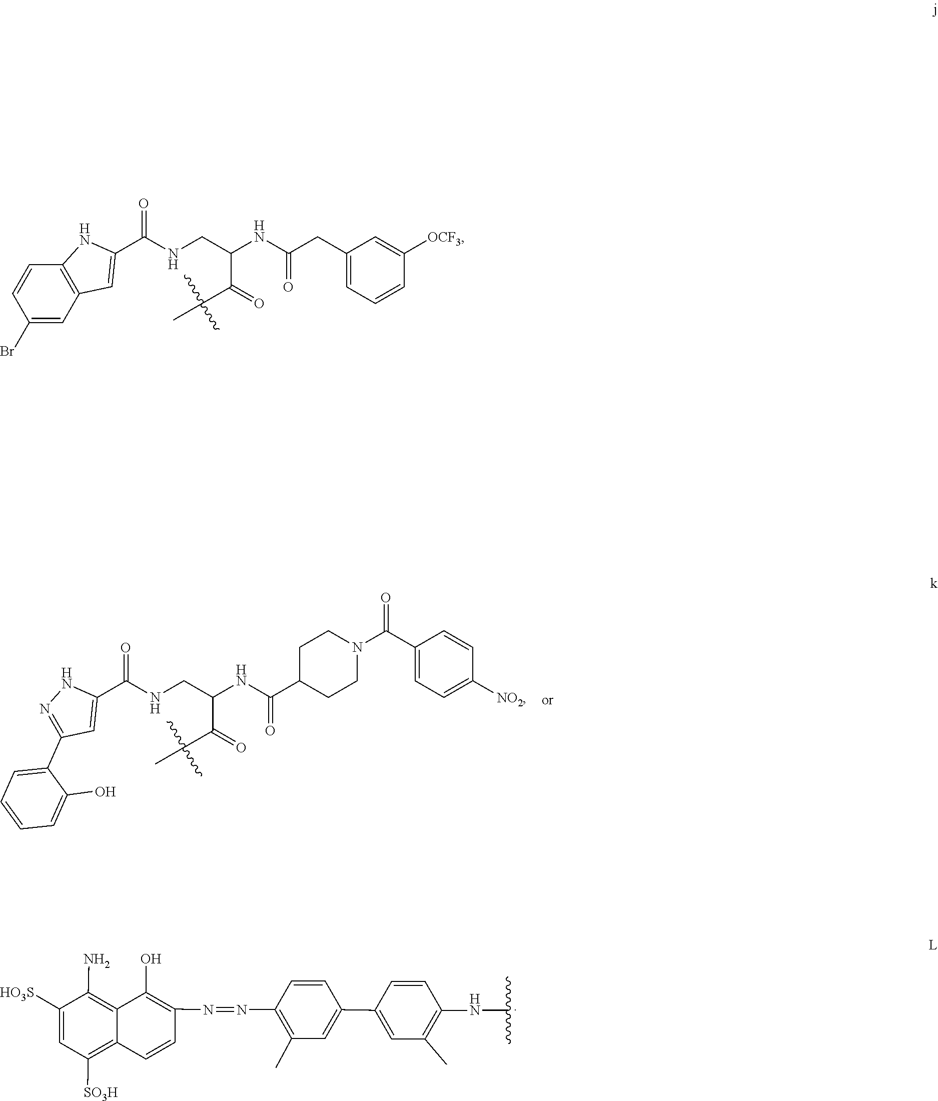 [SMILES: *C(=O)C(CNC(=O)c1cc2cc(Br)ccc2[nH]1)NC(=O)Cc1cccc(OC(F)(F)F)c1 ; *C(=O)C(CNC(=O)c1cc(-c2ccccc2O)n[nH]1)NC(=O)C1CCN(C(=O)c2ccc([N+](=O)[O-])cc2)CC1 ; *Nc1ccc(-c2ccc(N=Nc3ccc4c(S(=O)(=O)O)cc(S(=O)(=O)O)c(N)c4c3O)c(C)c2)cc1C]

Another aspect of the present disclosure relates to the compound of Formula (I), and the heterocyclic group of B₁ and/or B₂ is a triazine, a triazine amine, or a triazine aminoalkyl. Another aspect is the compound of Formula (I) capable of targeting to a Fibroblast Activated Protein (FAP).

Another aspect of the present disclosure relates to the compound of Formula (II):

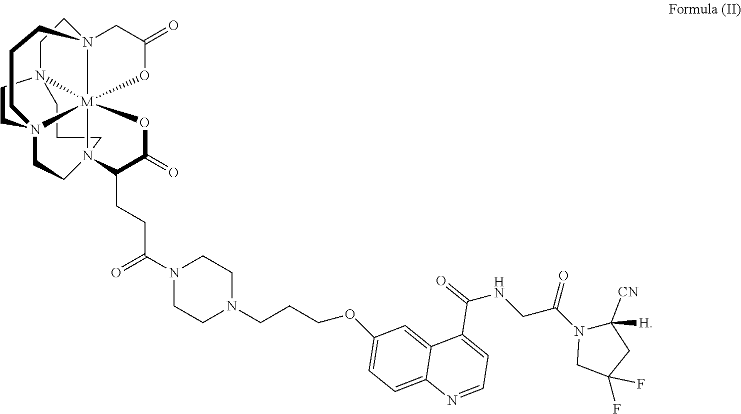

Another aspect of the present disclosure relates to the compound of Formula (III):

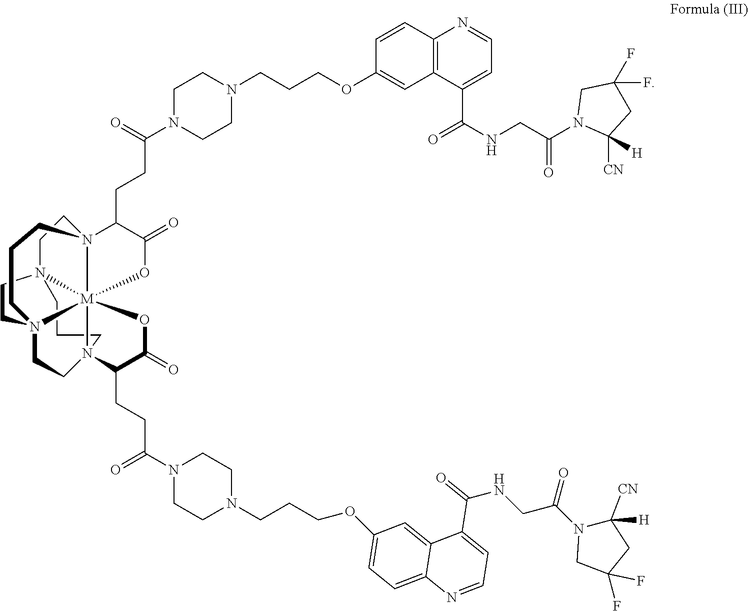

Another aspect of the present disclosure relates to the compound of Formula (I), and the compound is selected from Formula (IV) to (LXIII) shown below, wherein n is 1 to 12 ethylene glycol repeating units or 1 to 6 amino acids; and wherein R₃ comprises —OH, —SH, or —NH2:

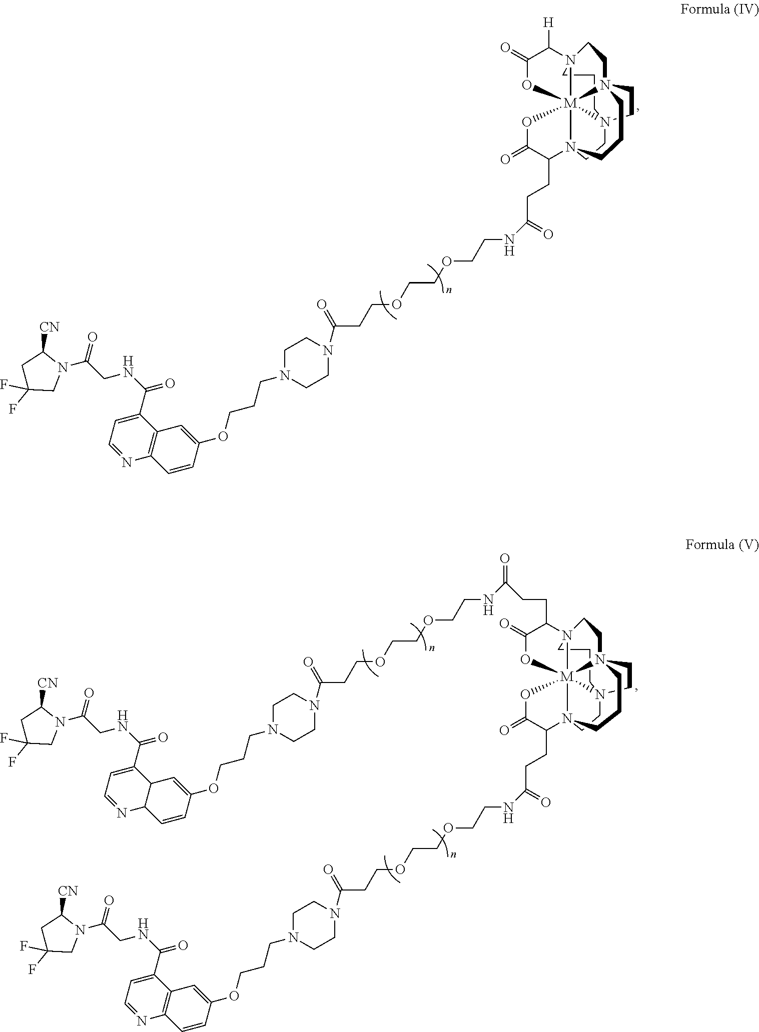

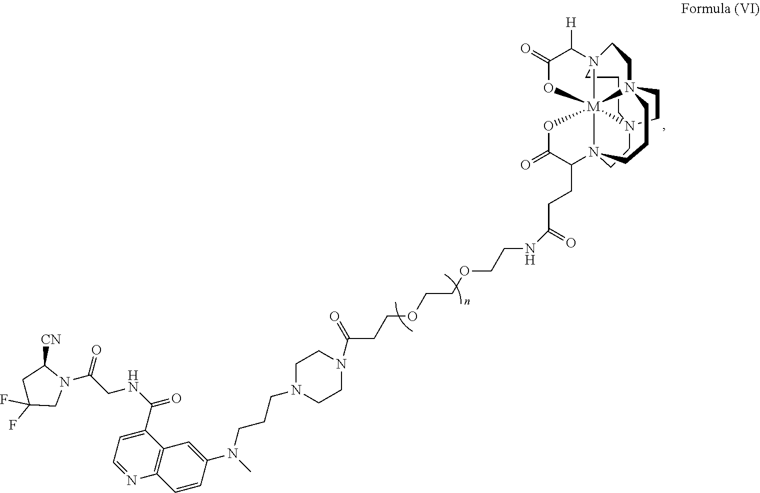

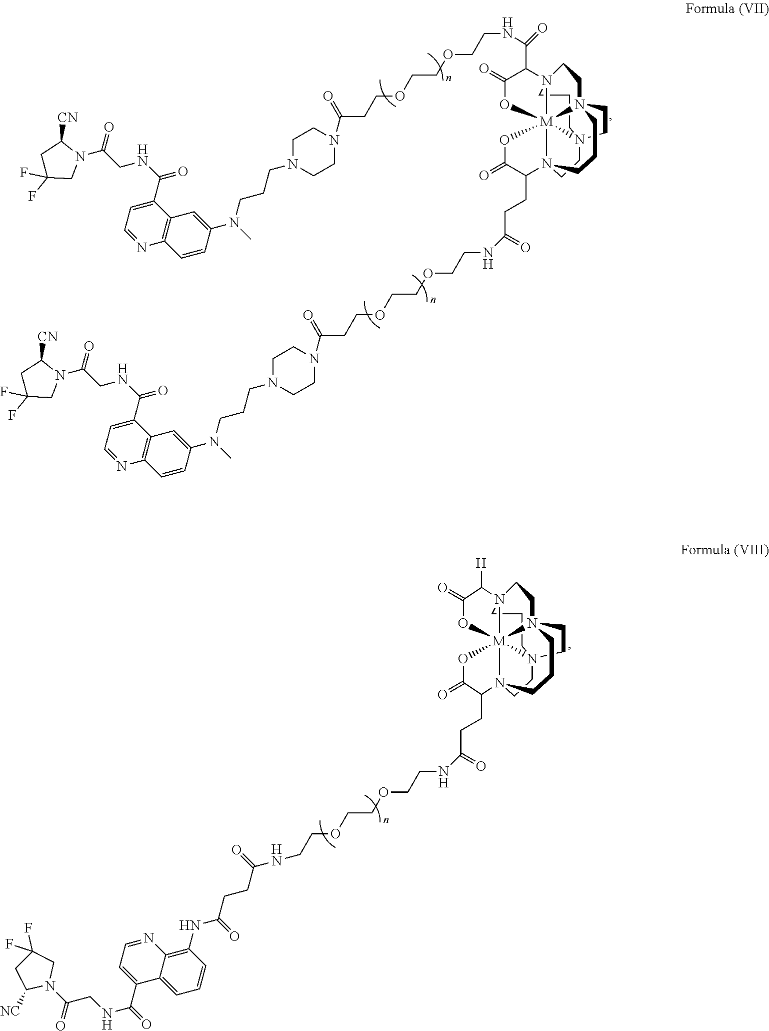

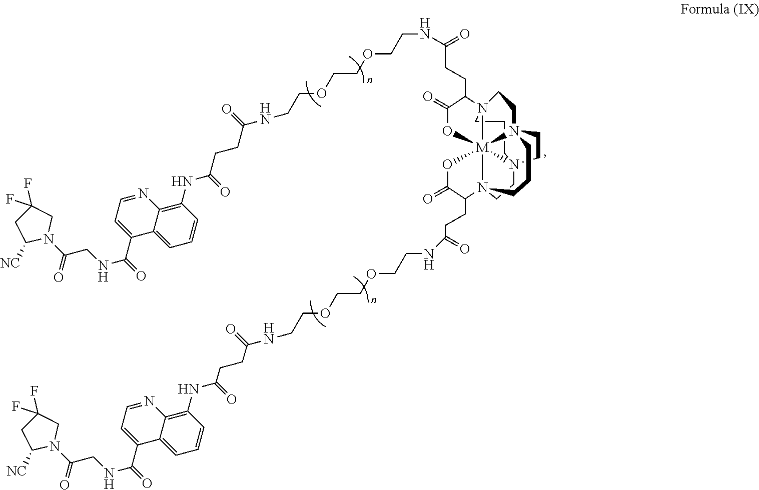

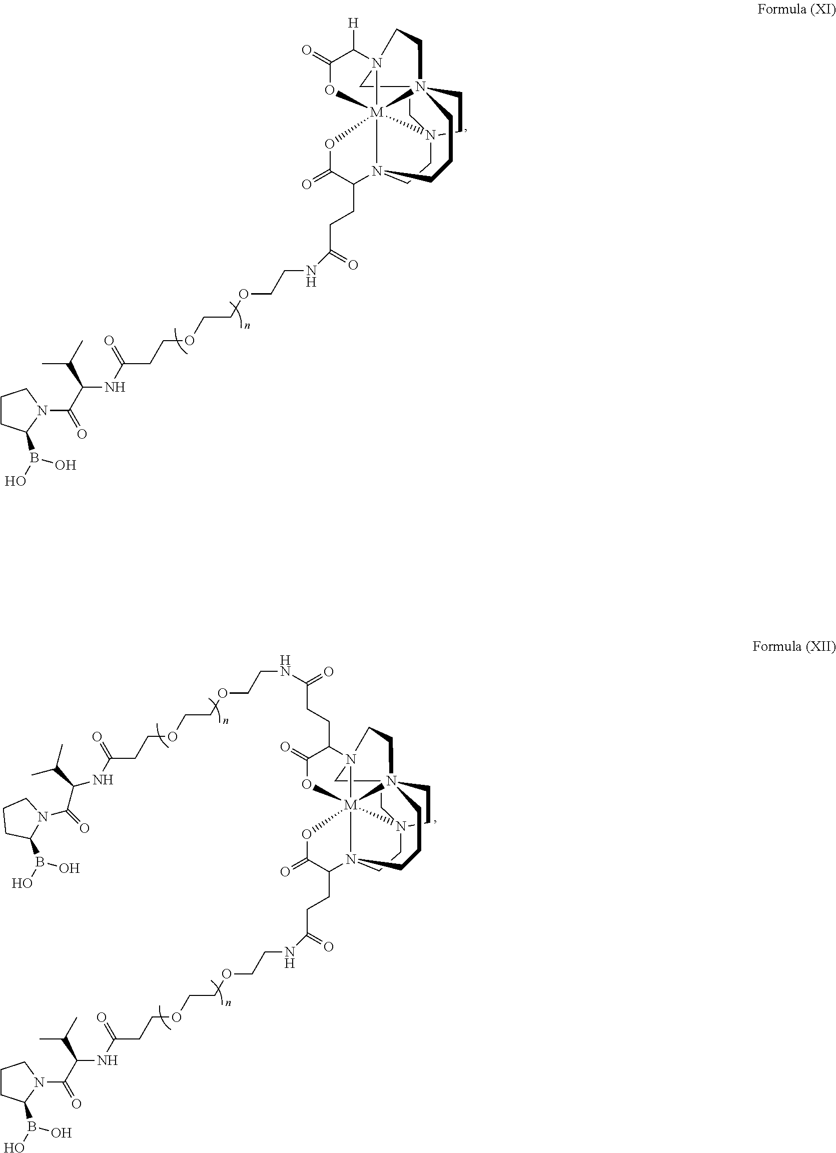

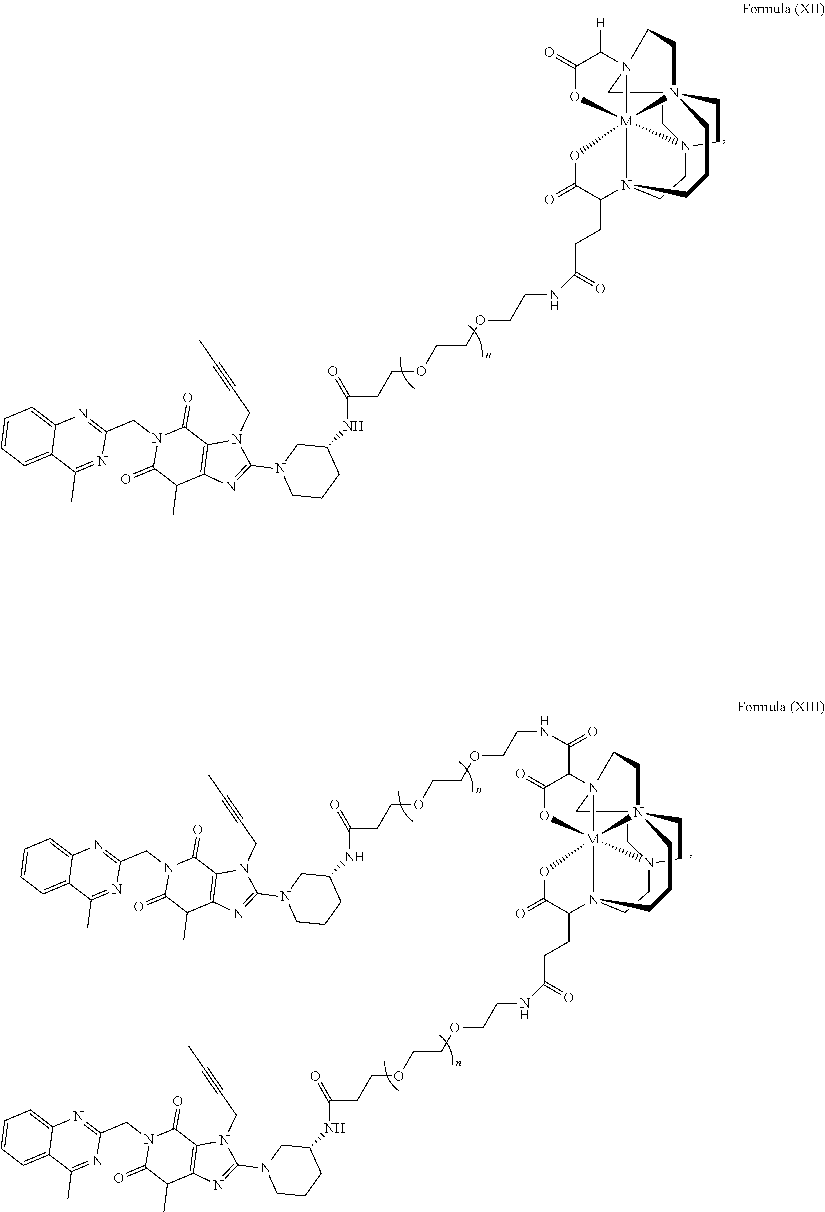

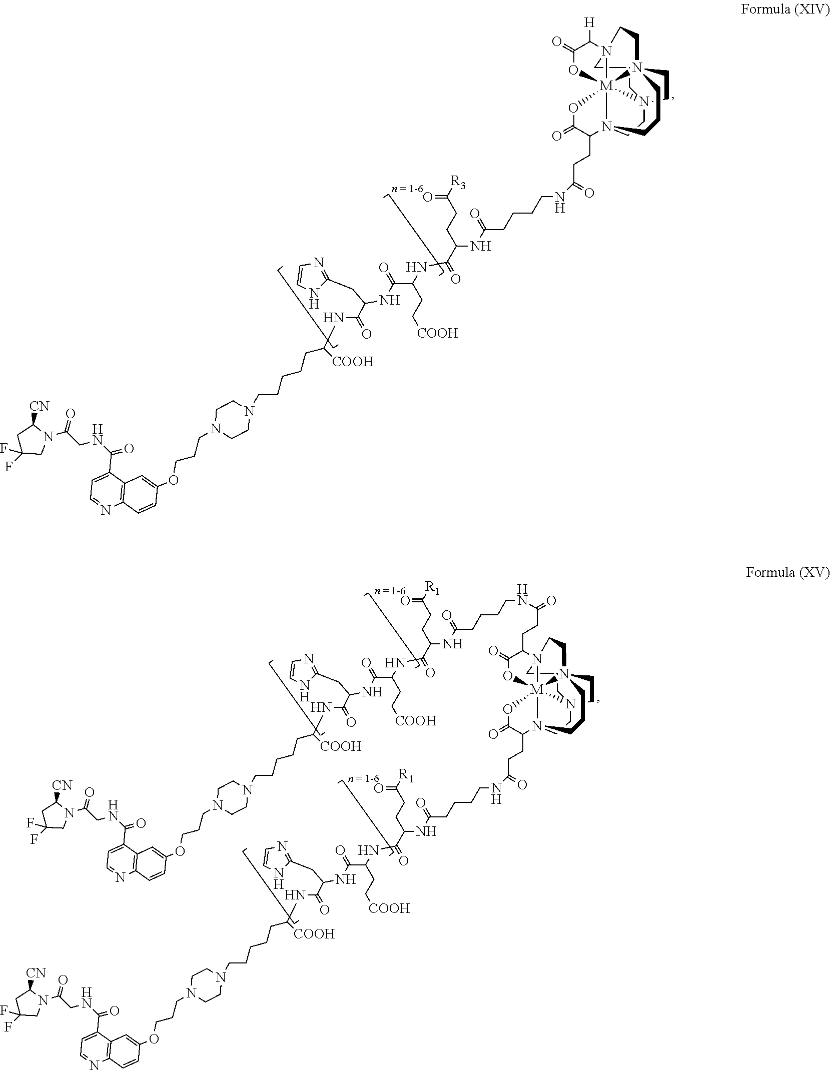

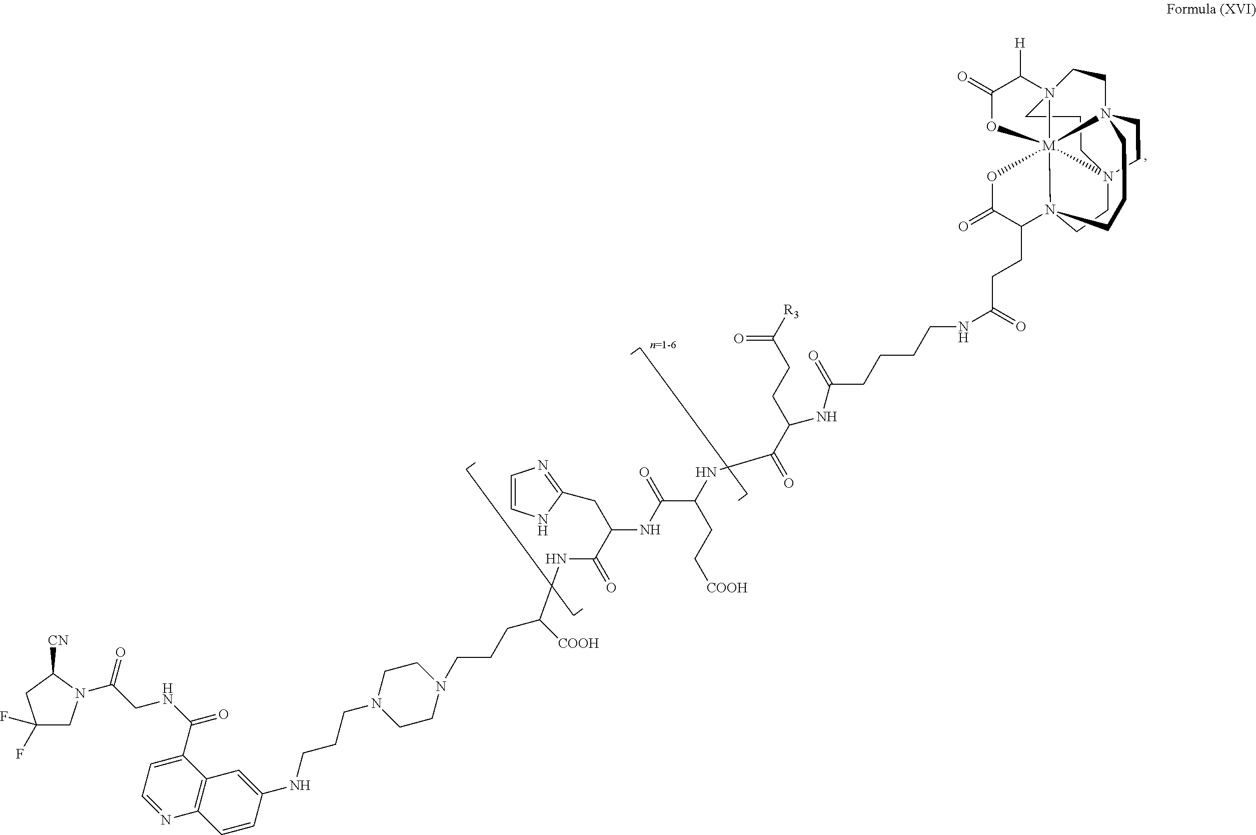

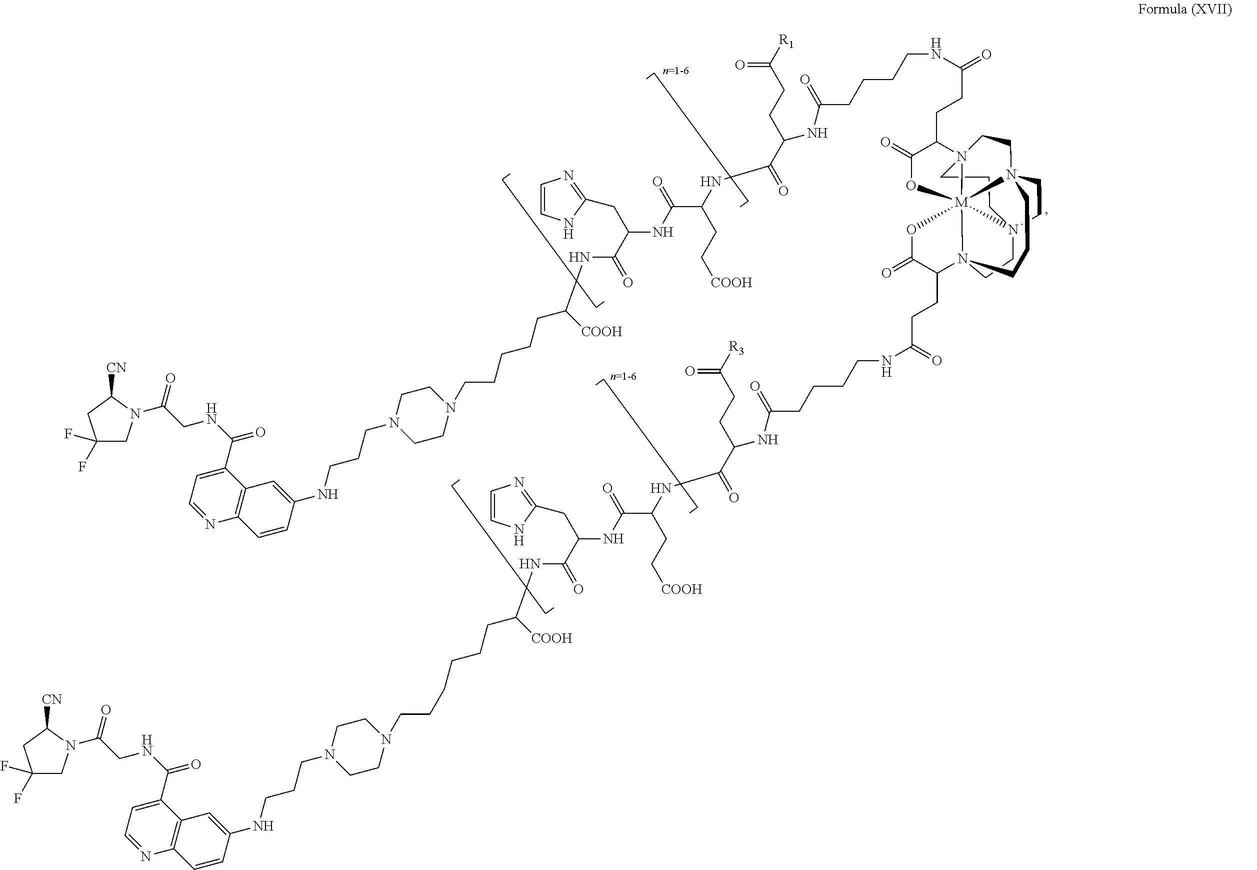

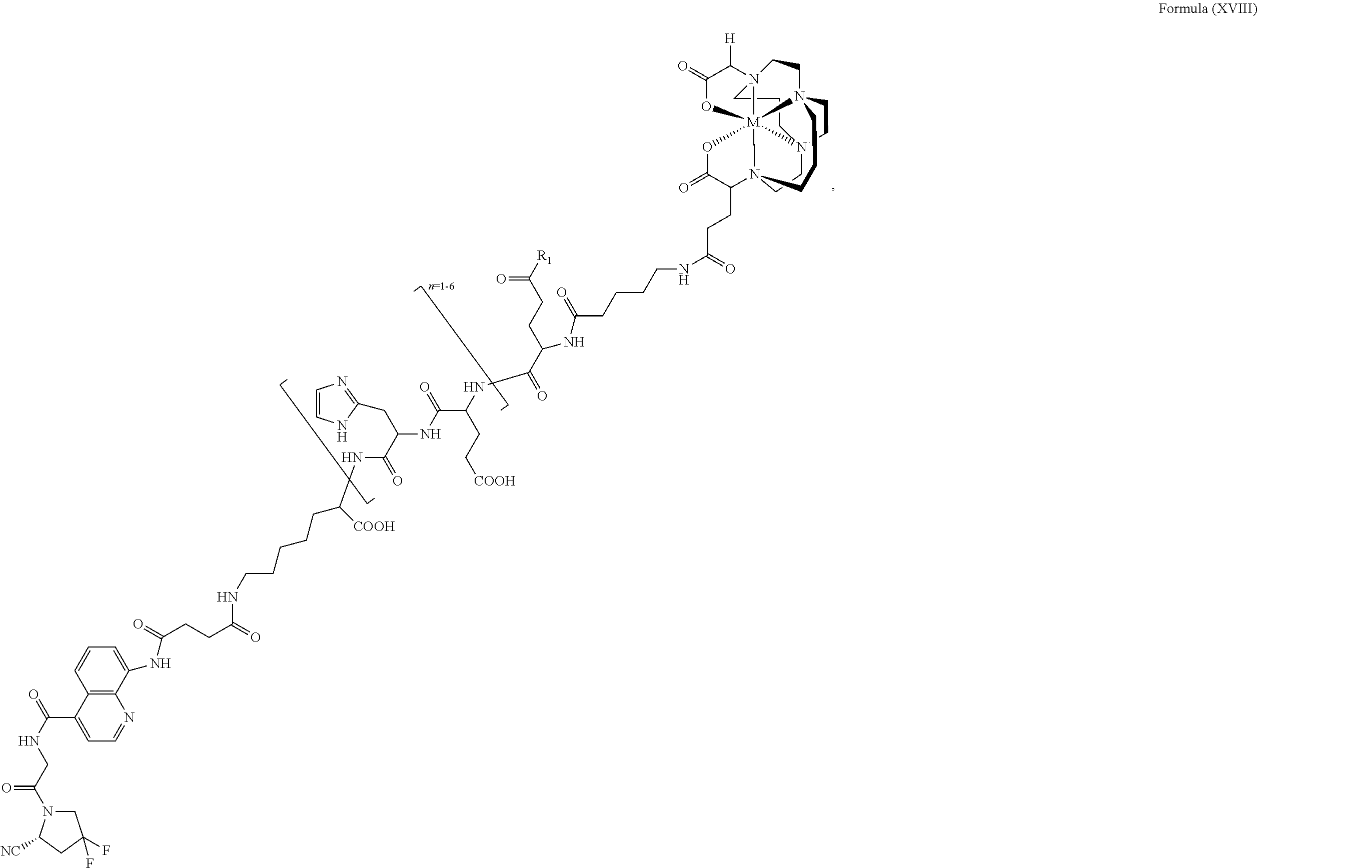

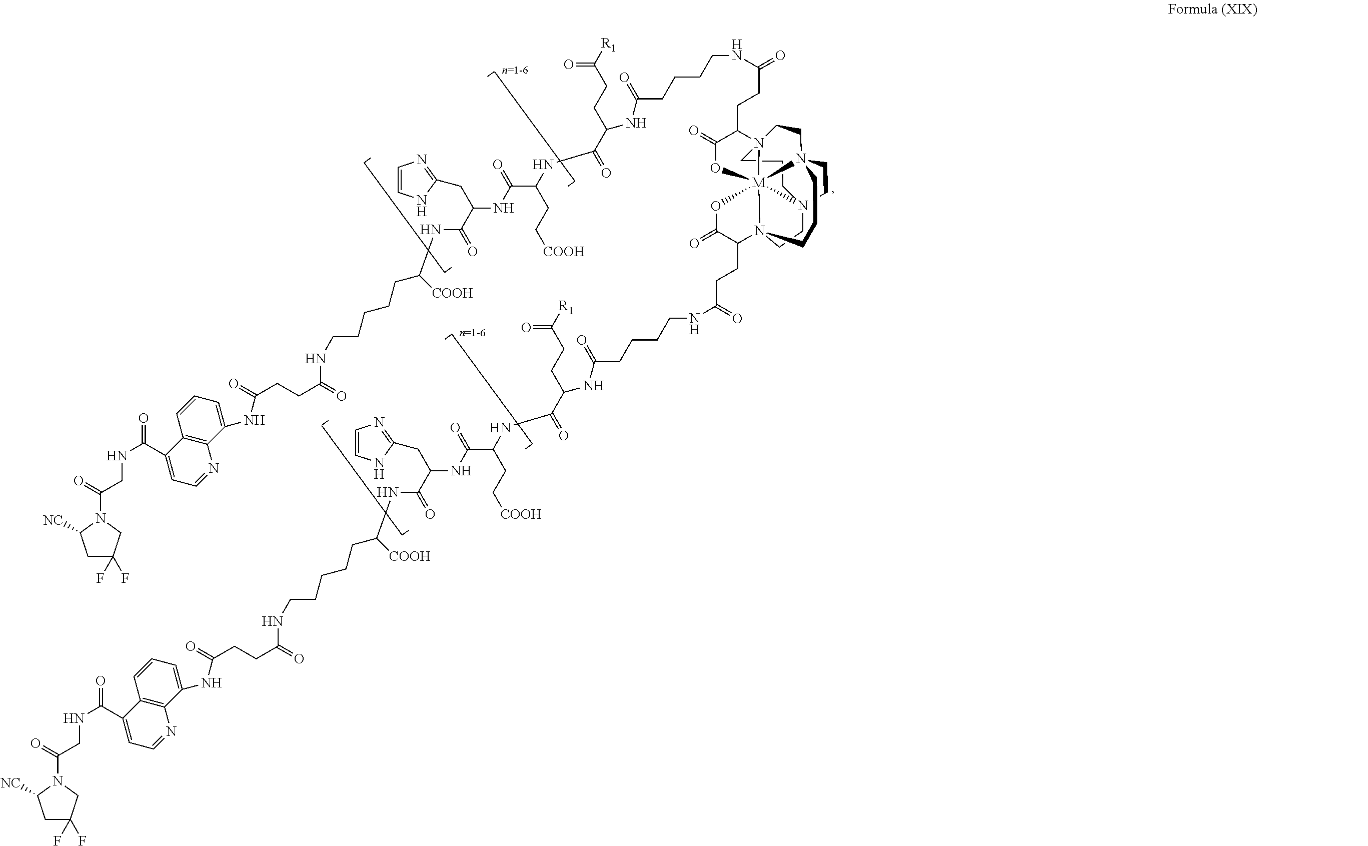

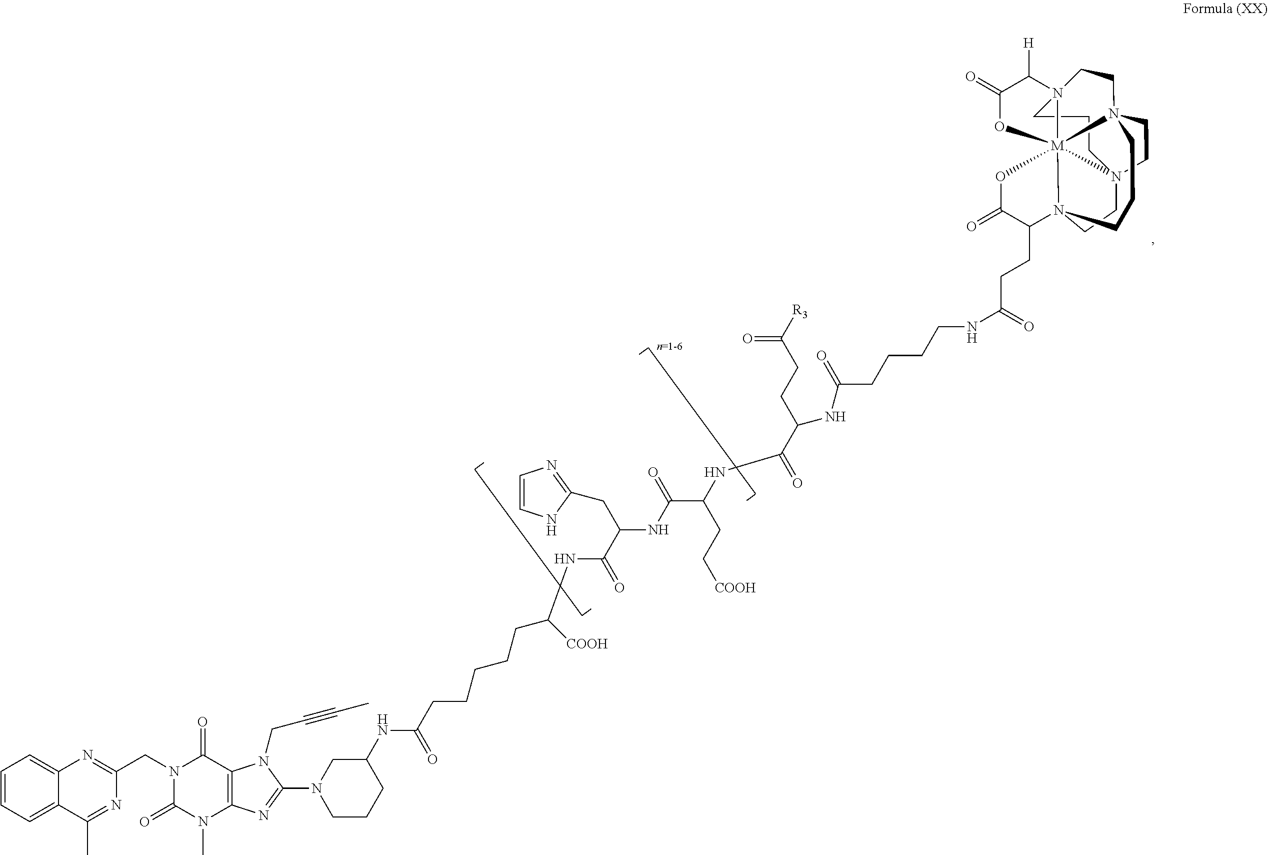

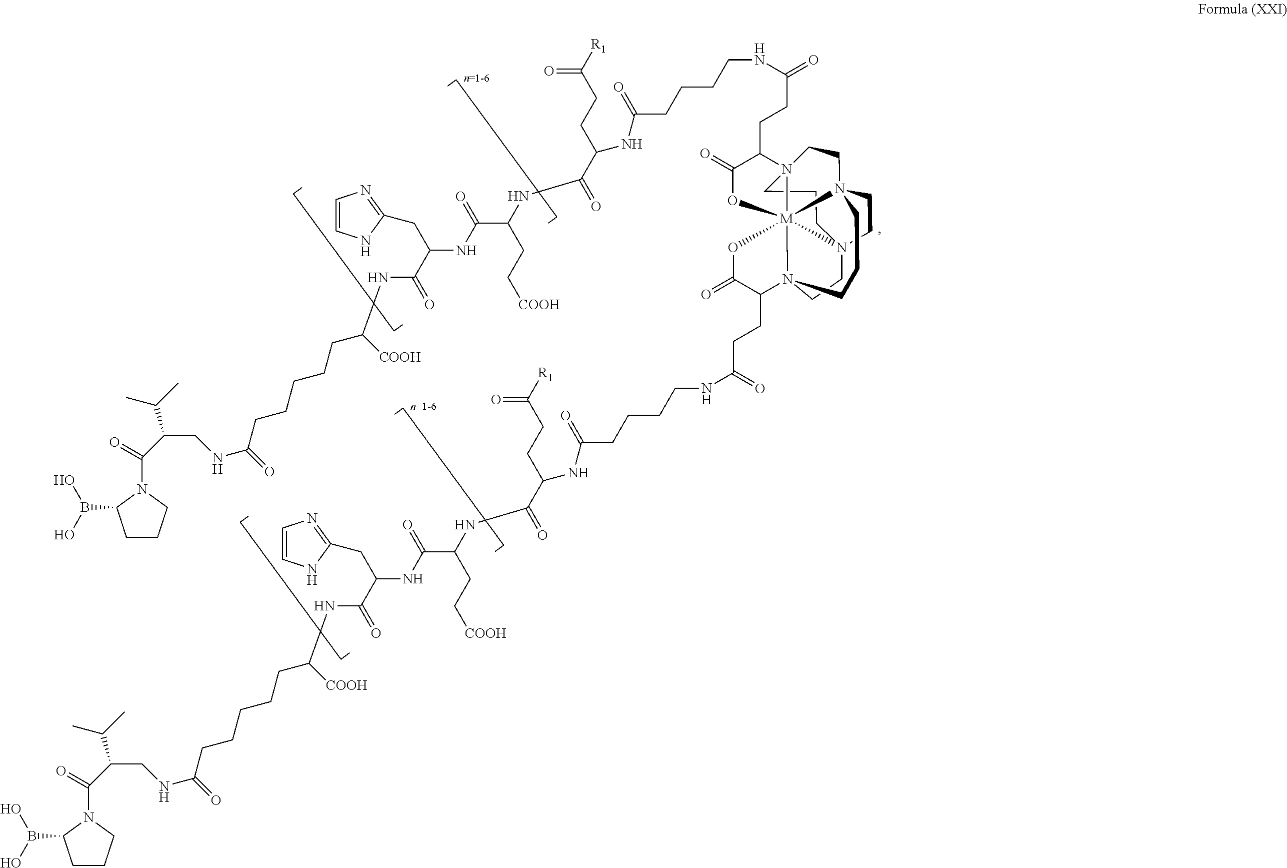

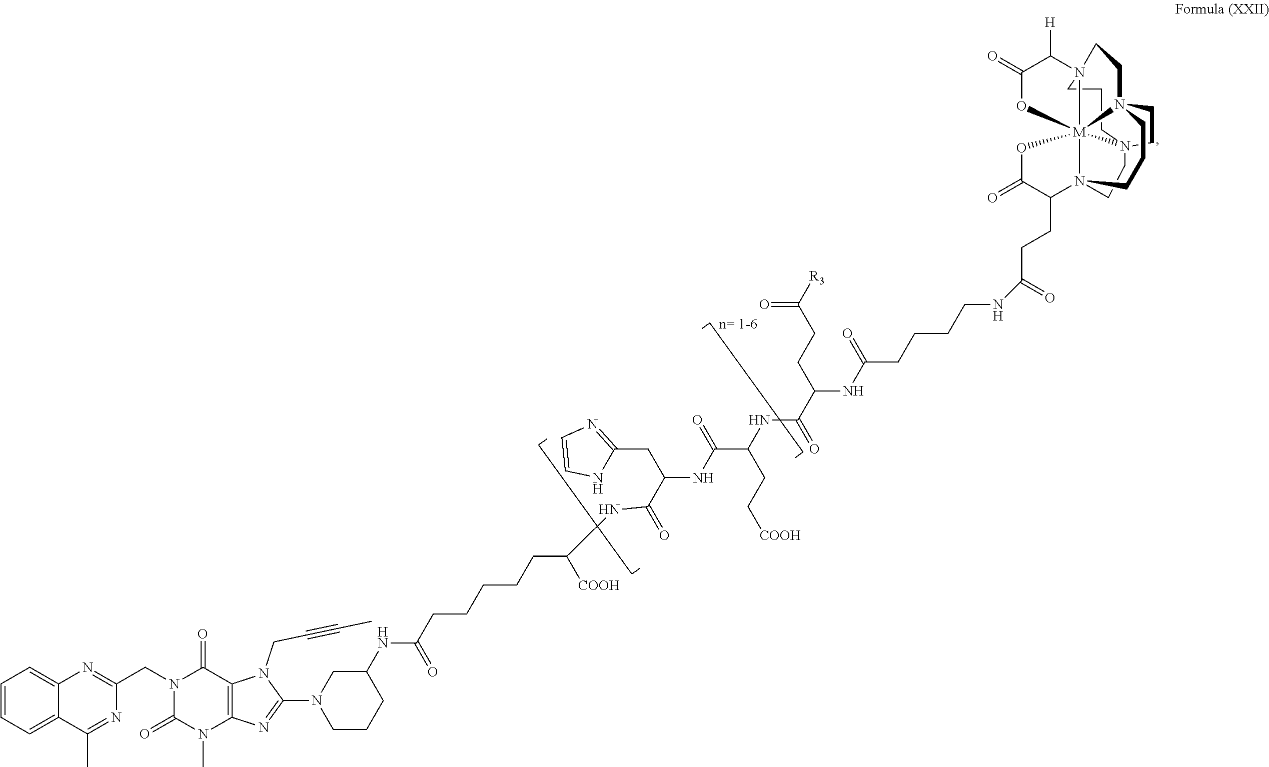

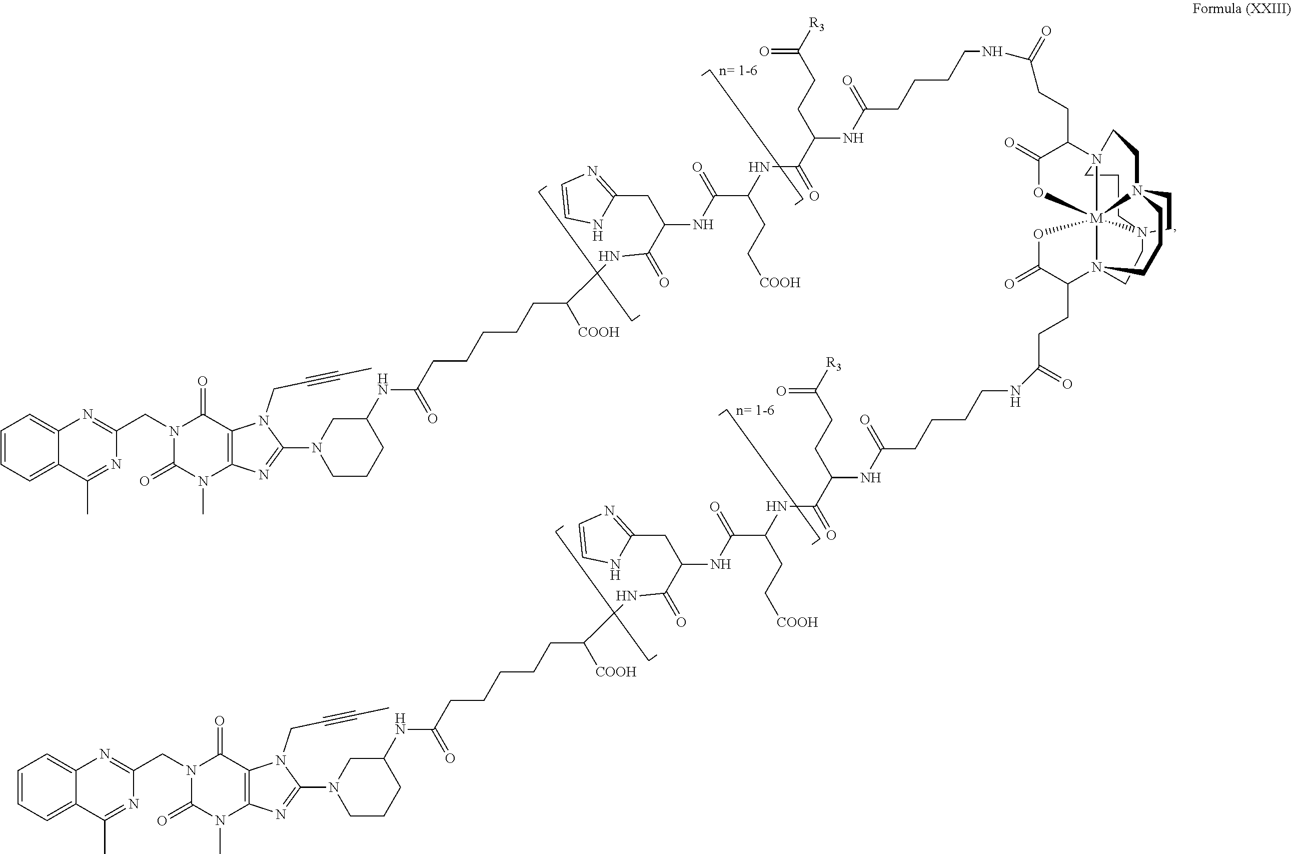

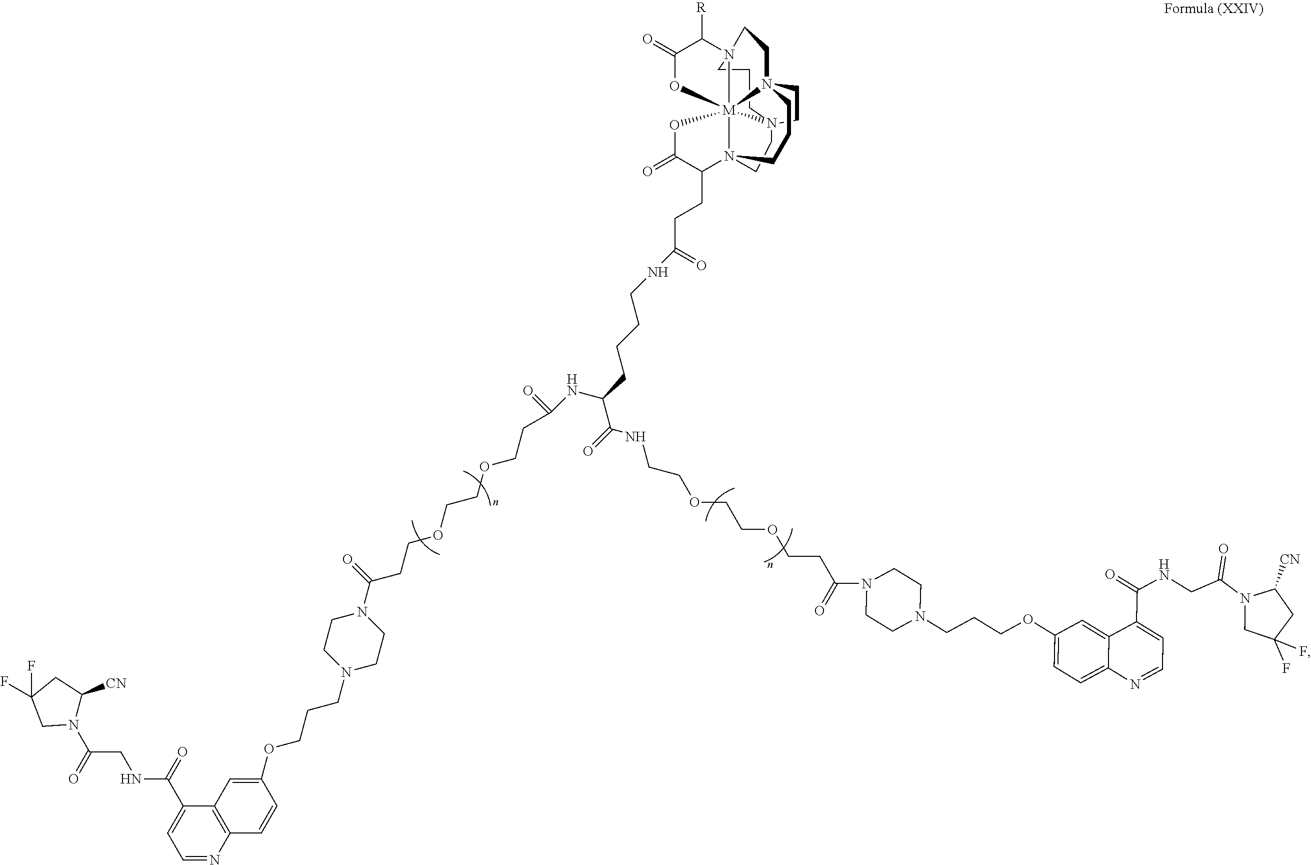

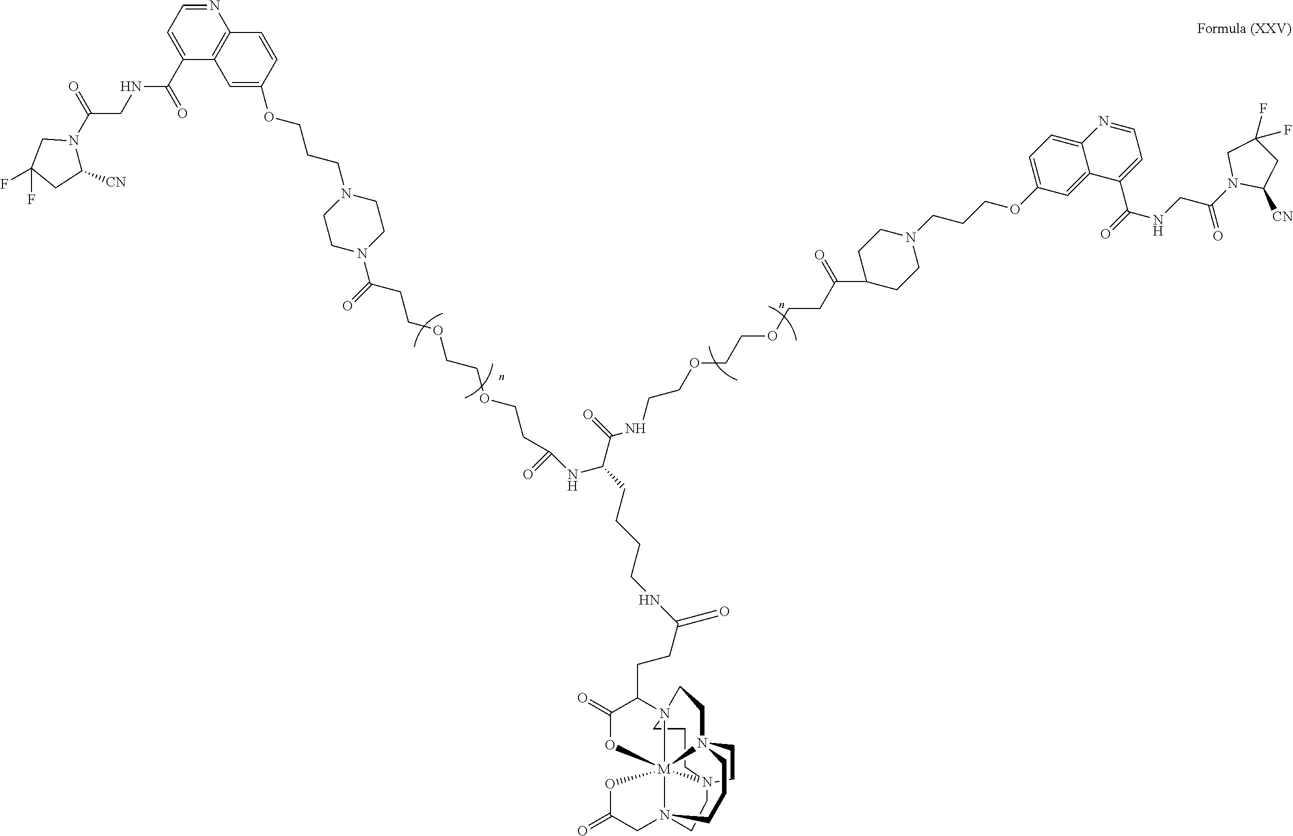

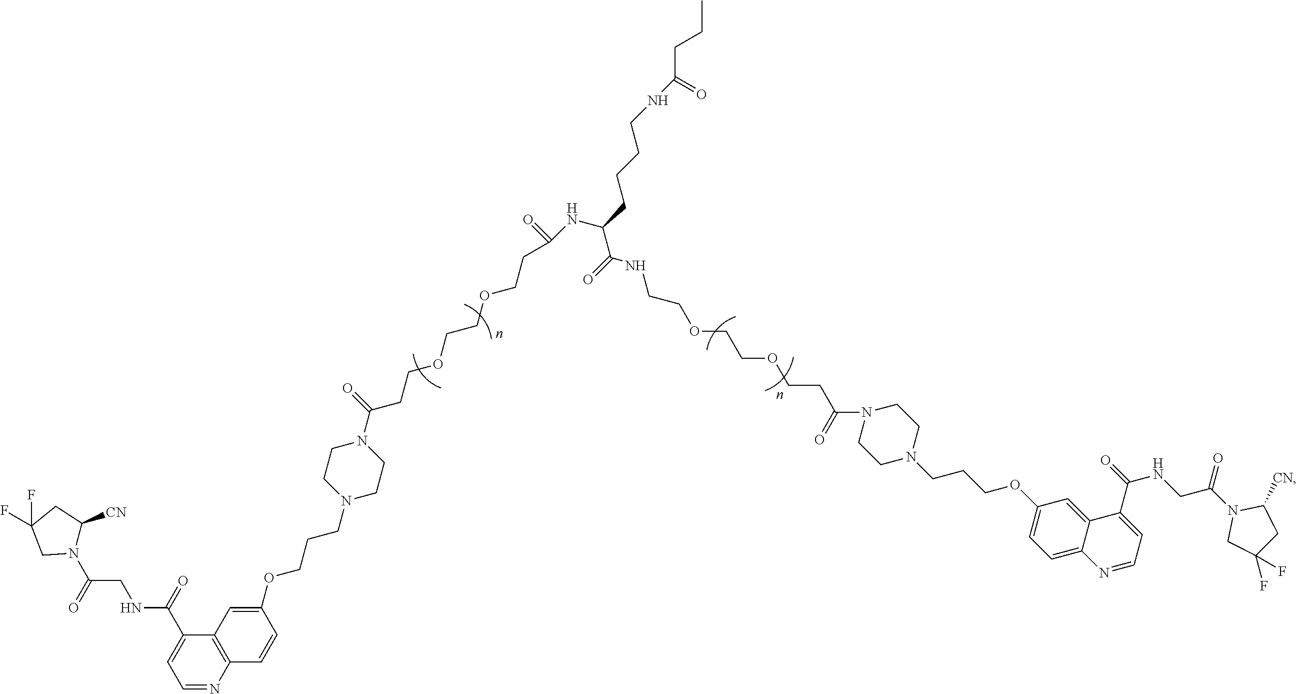

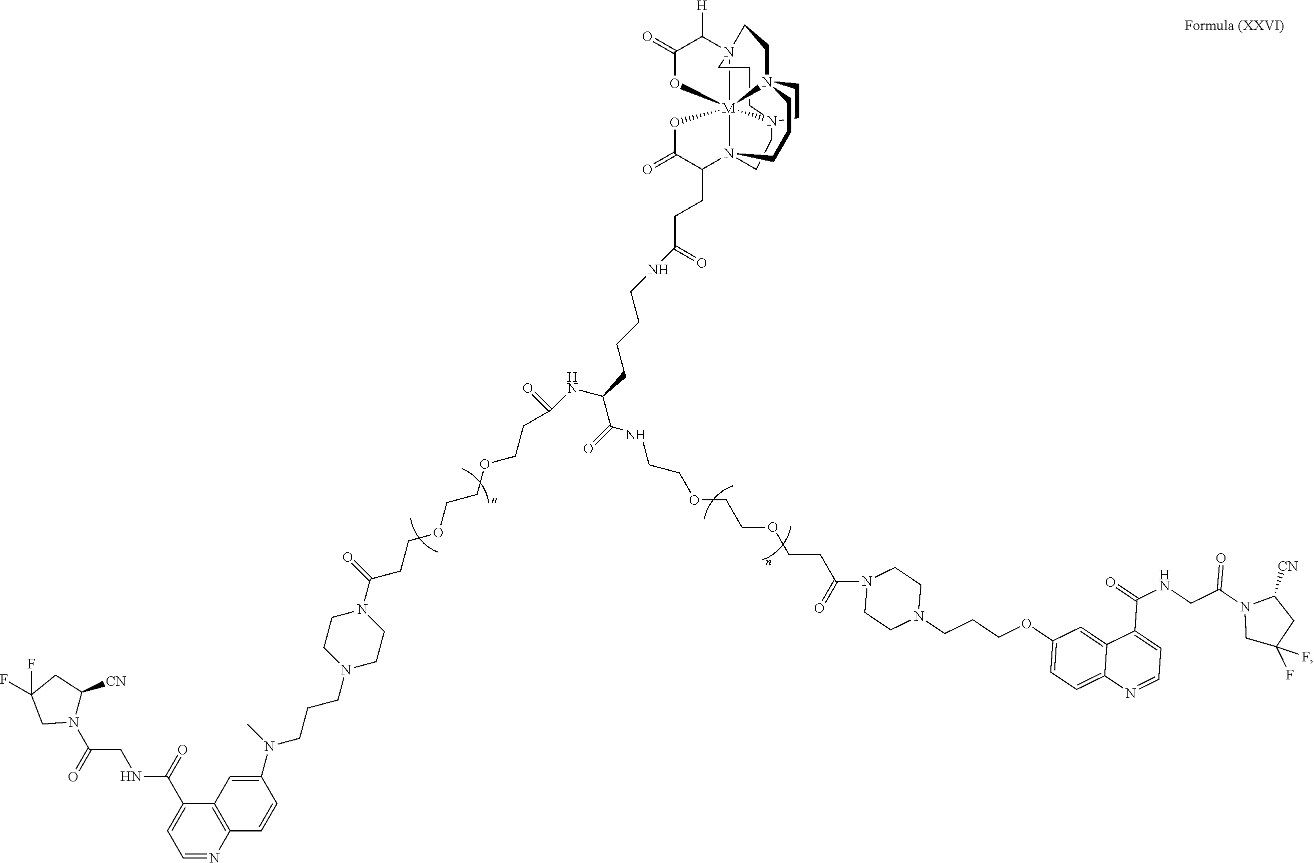

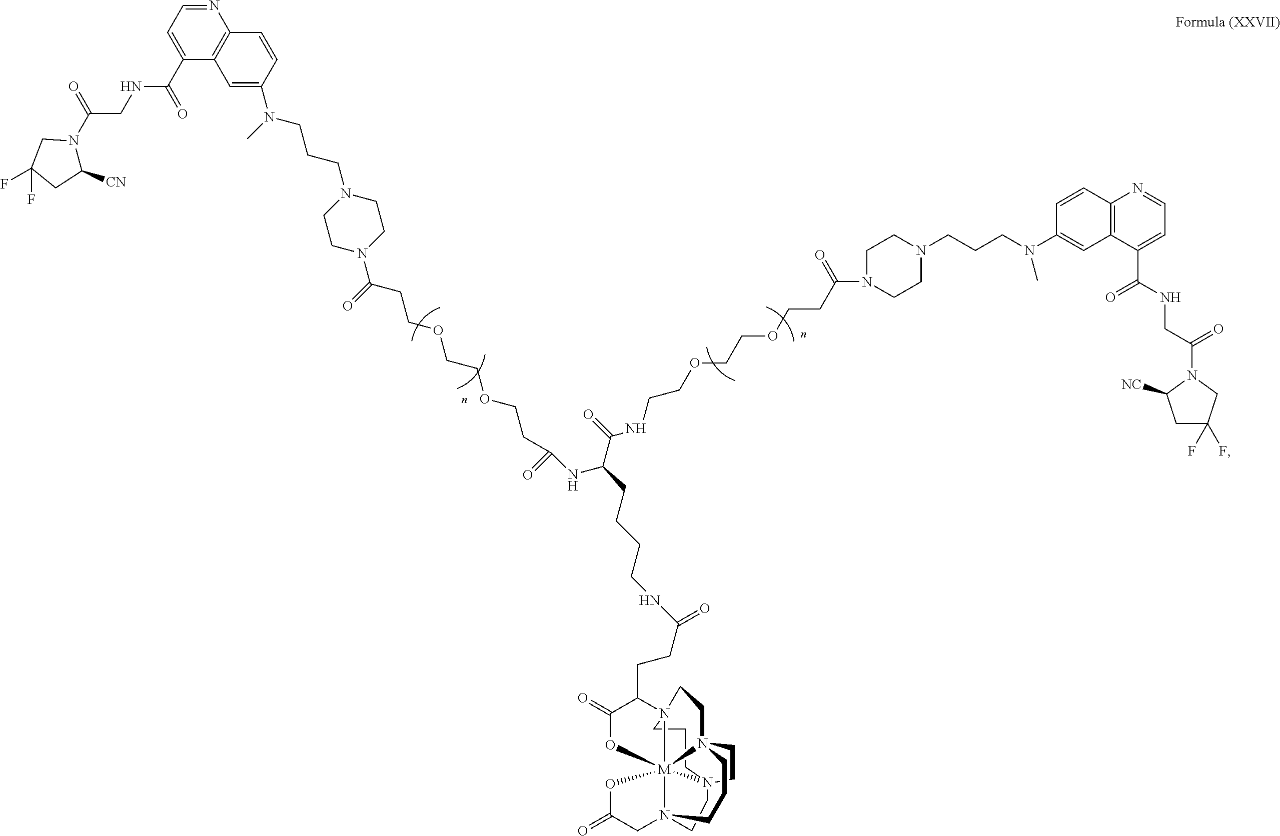

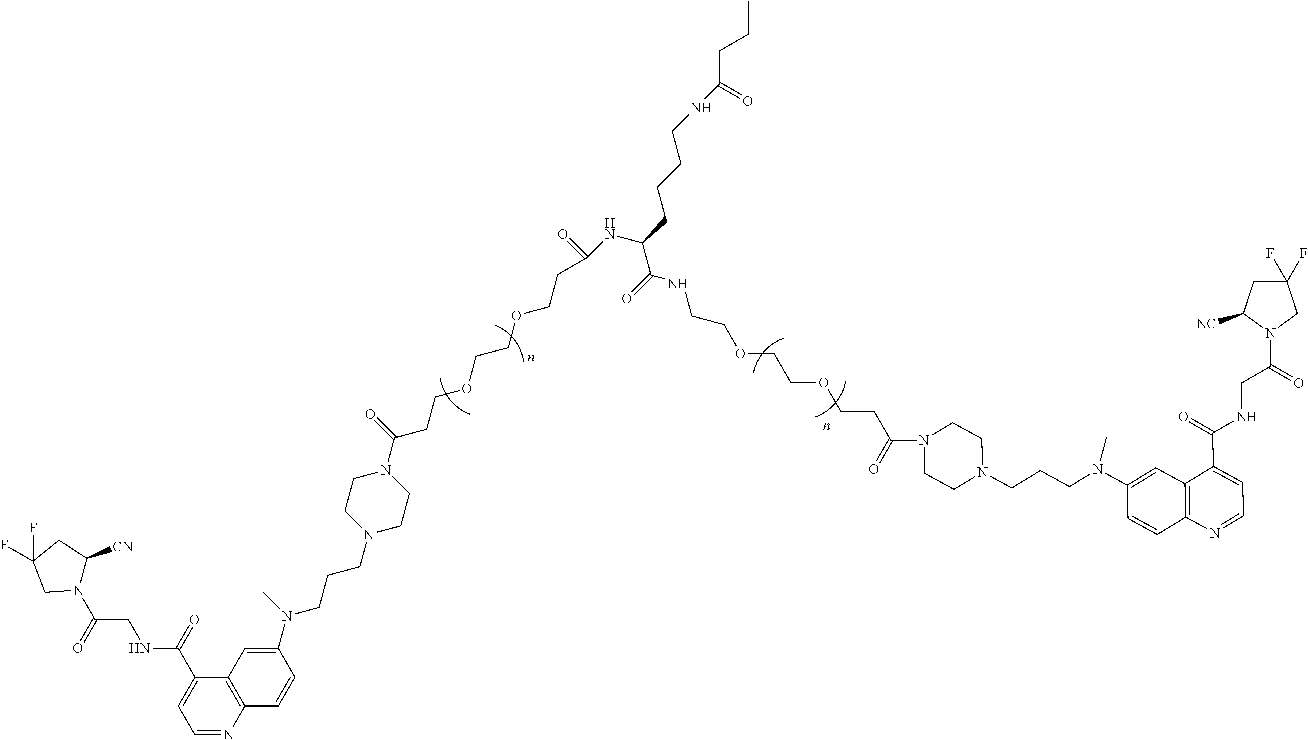

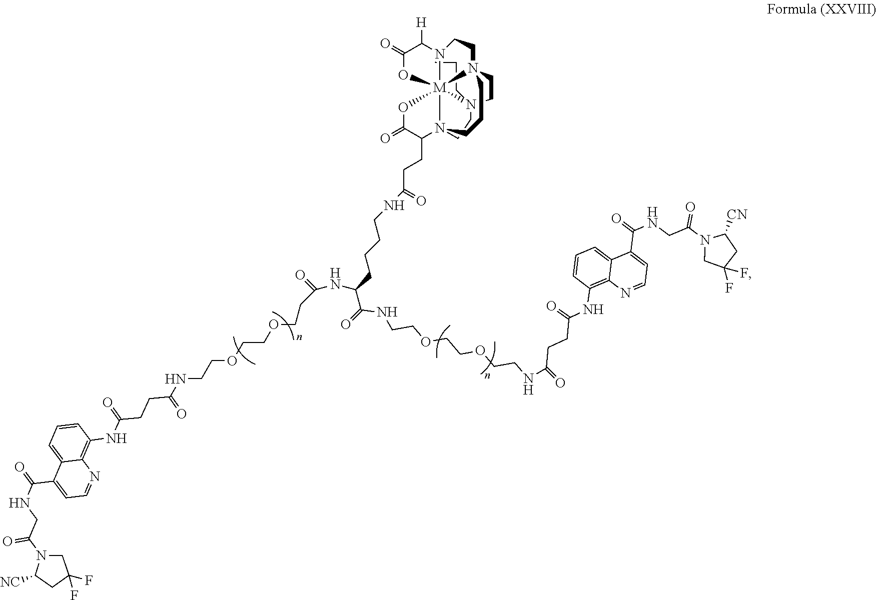

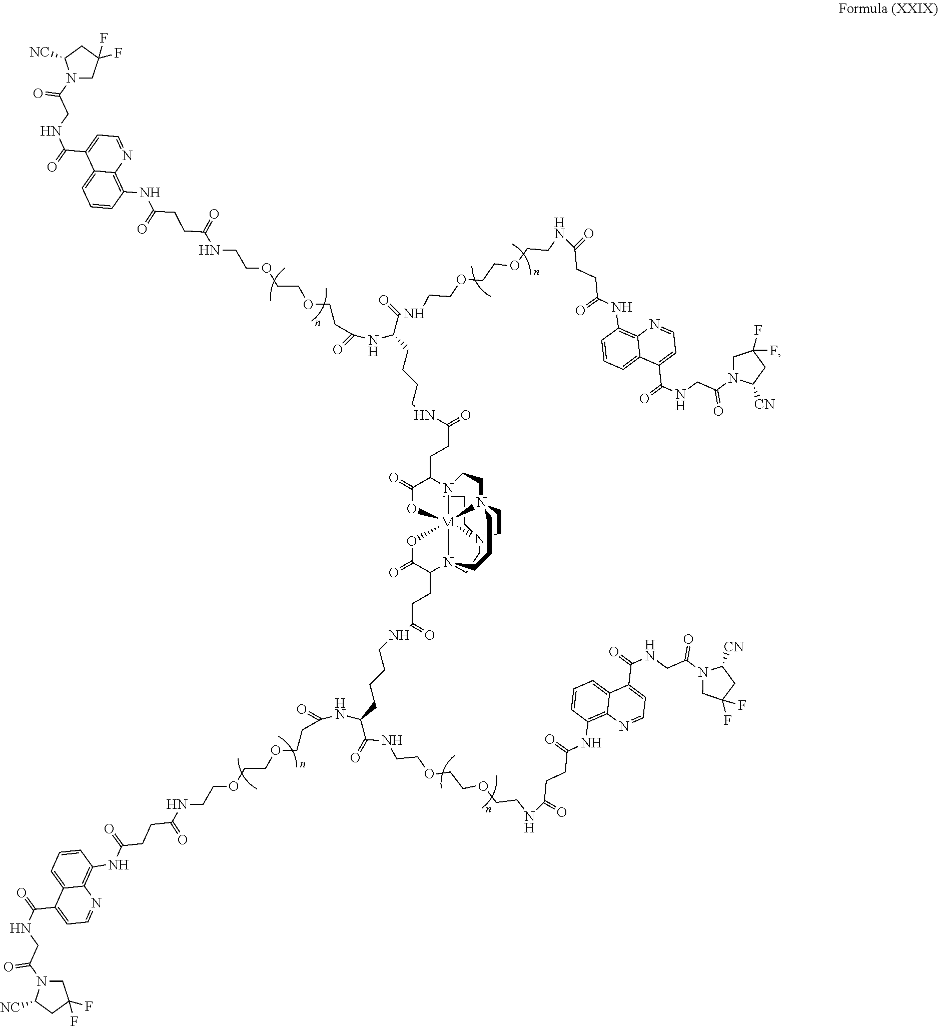

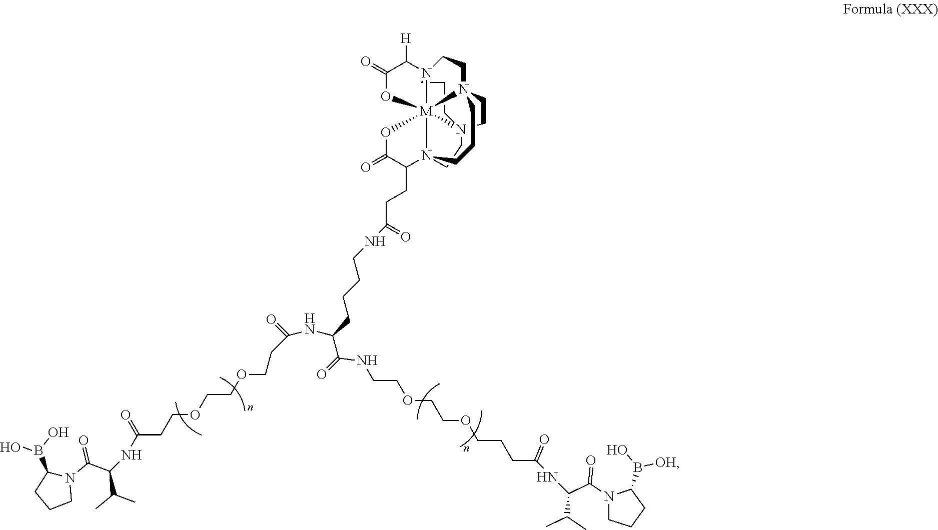

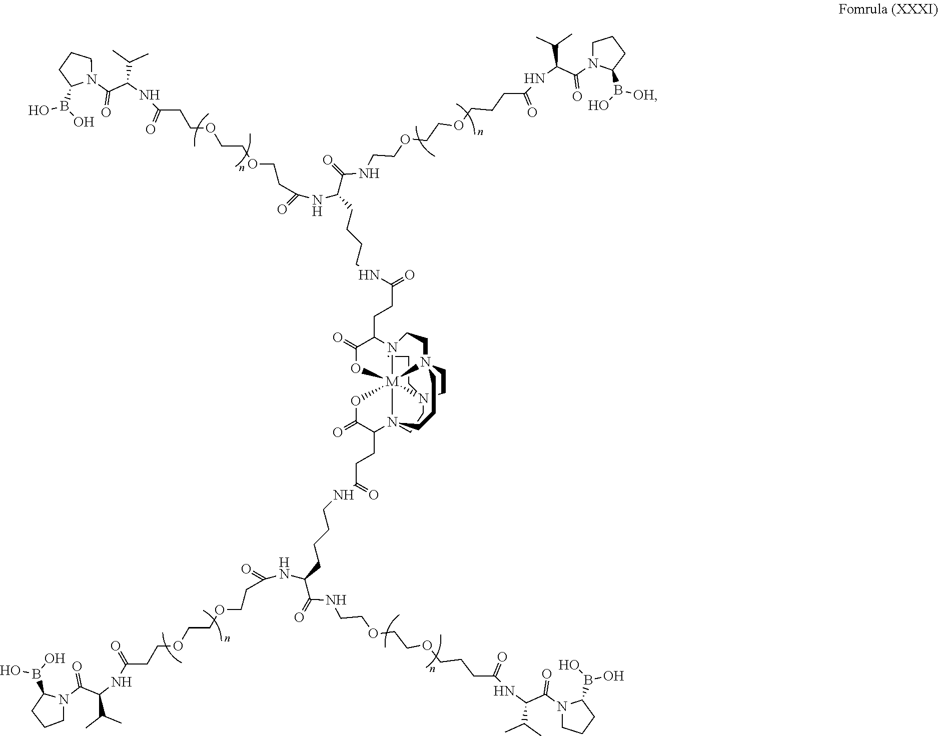

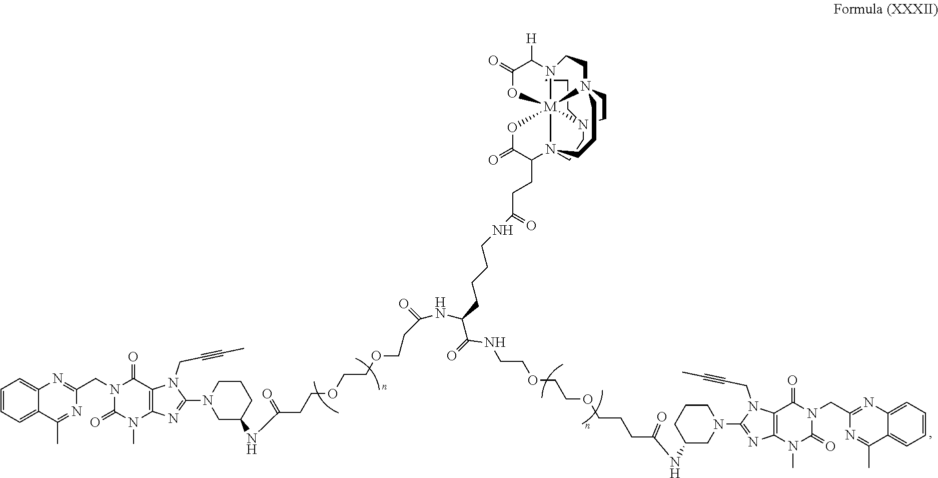

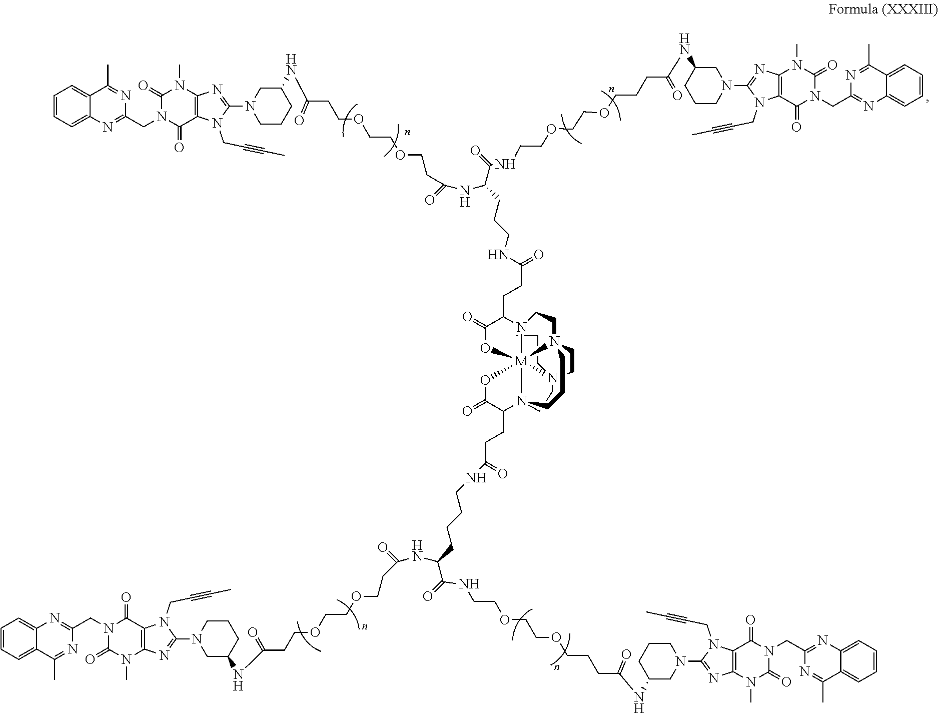

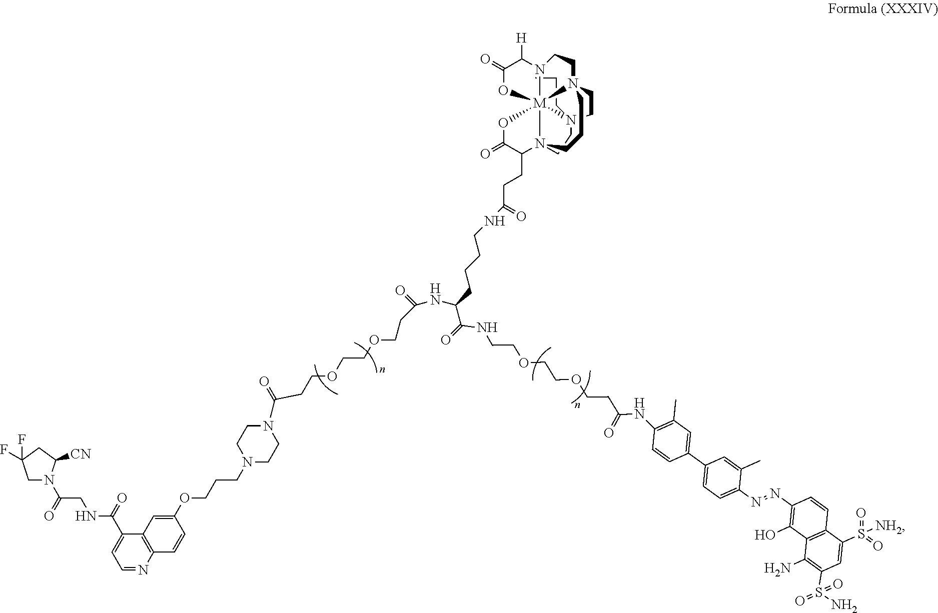

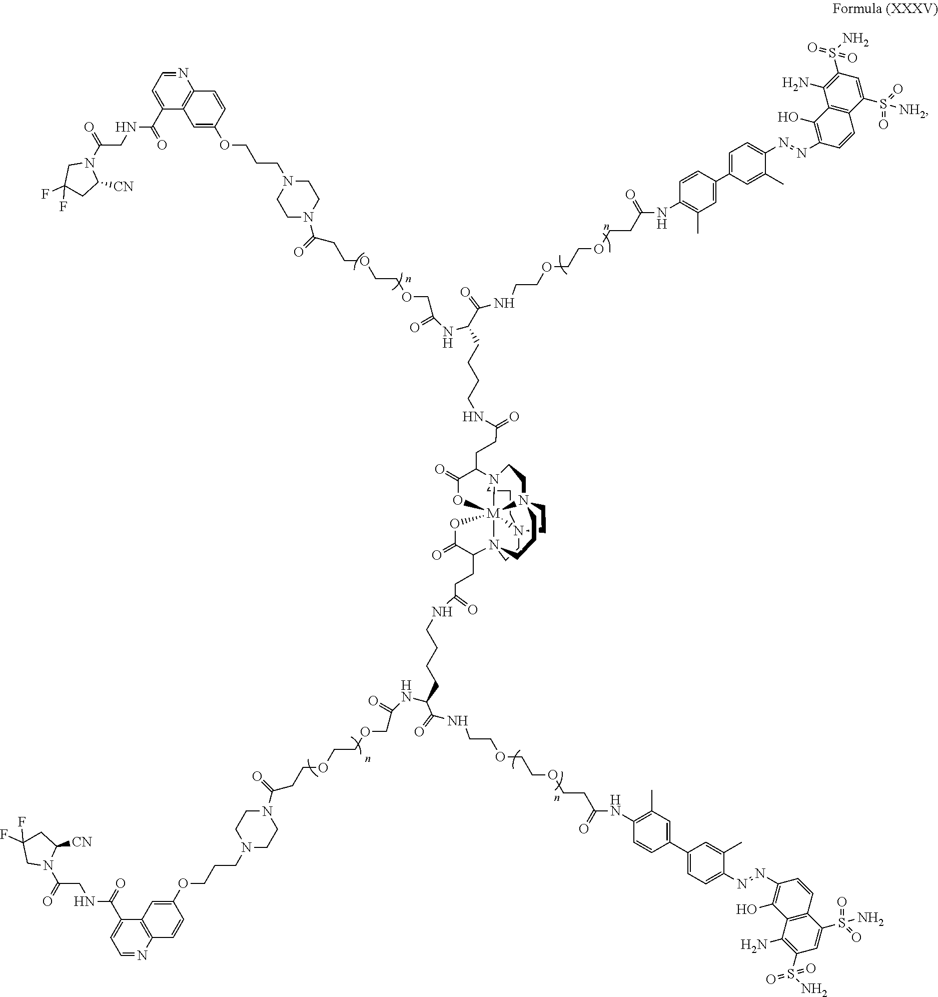

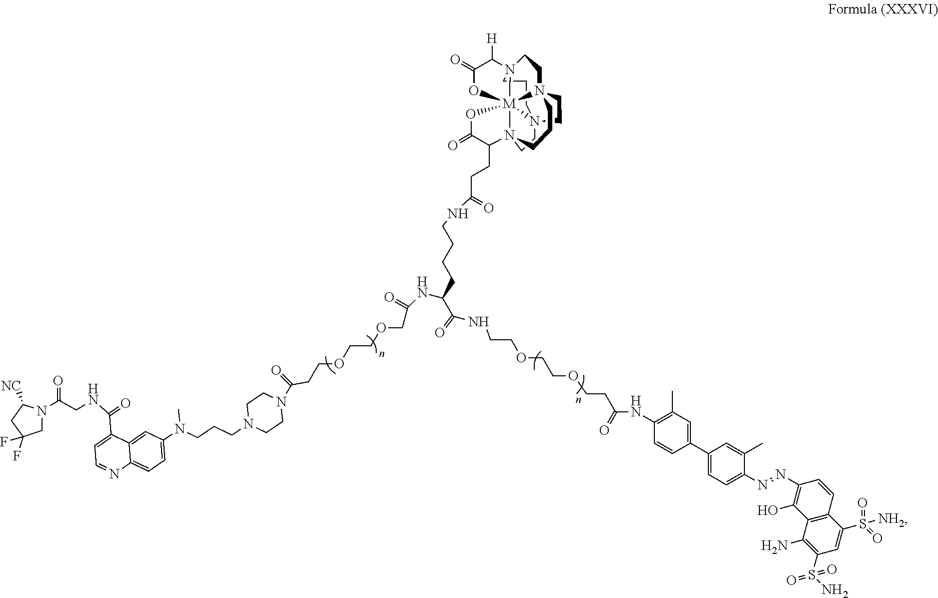

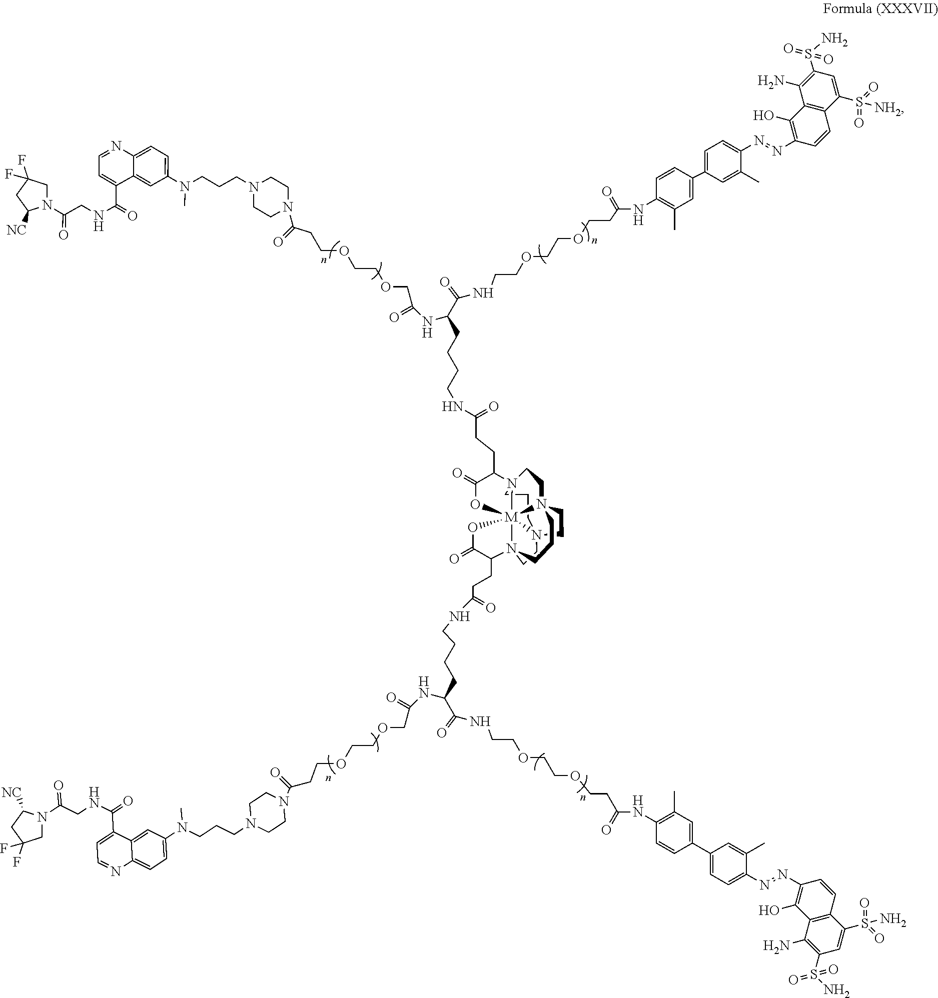

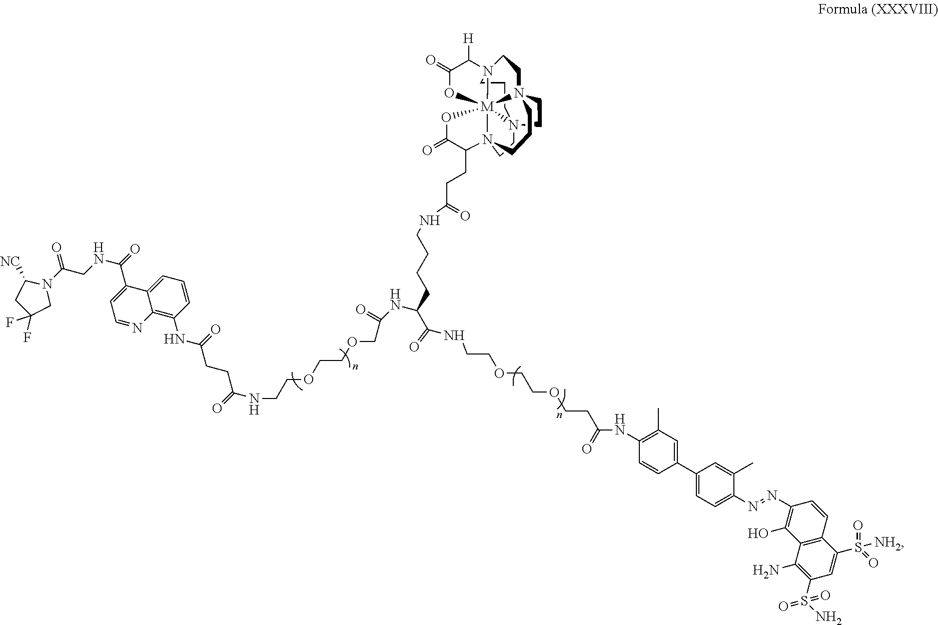

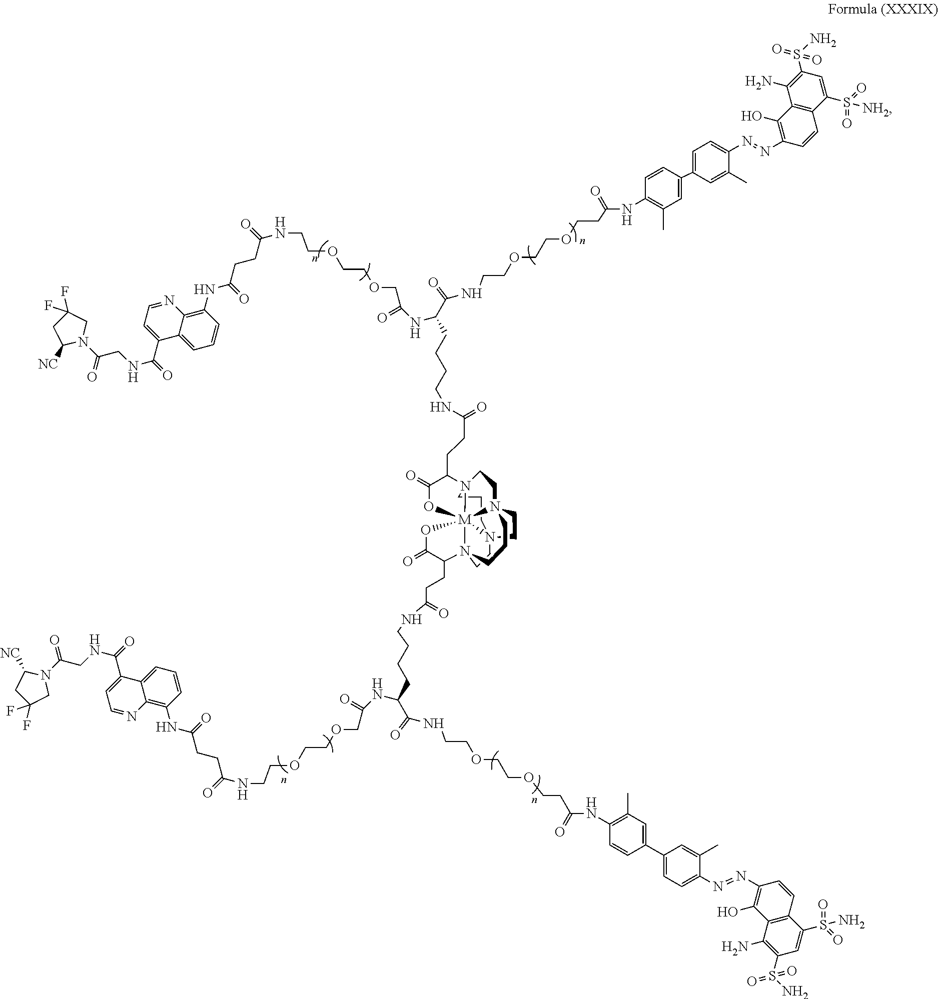

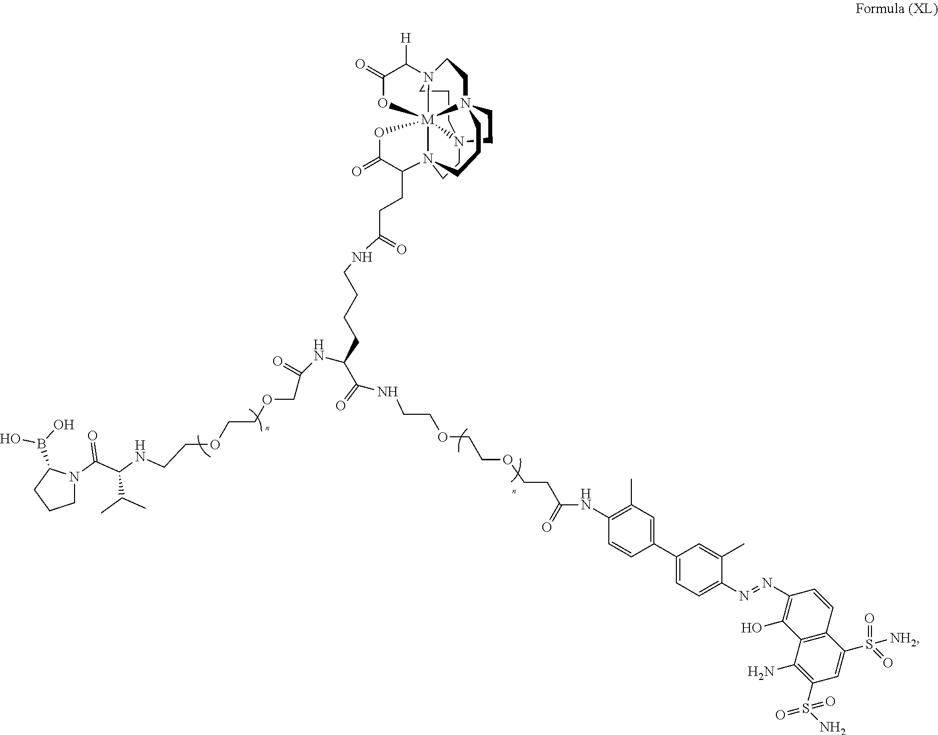

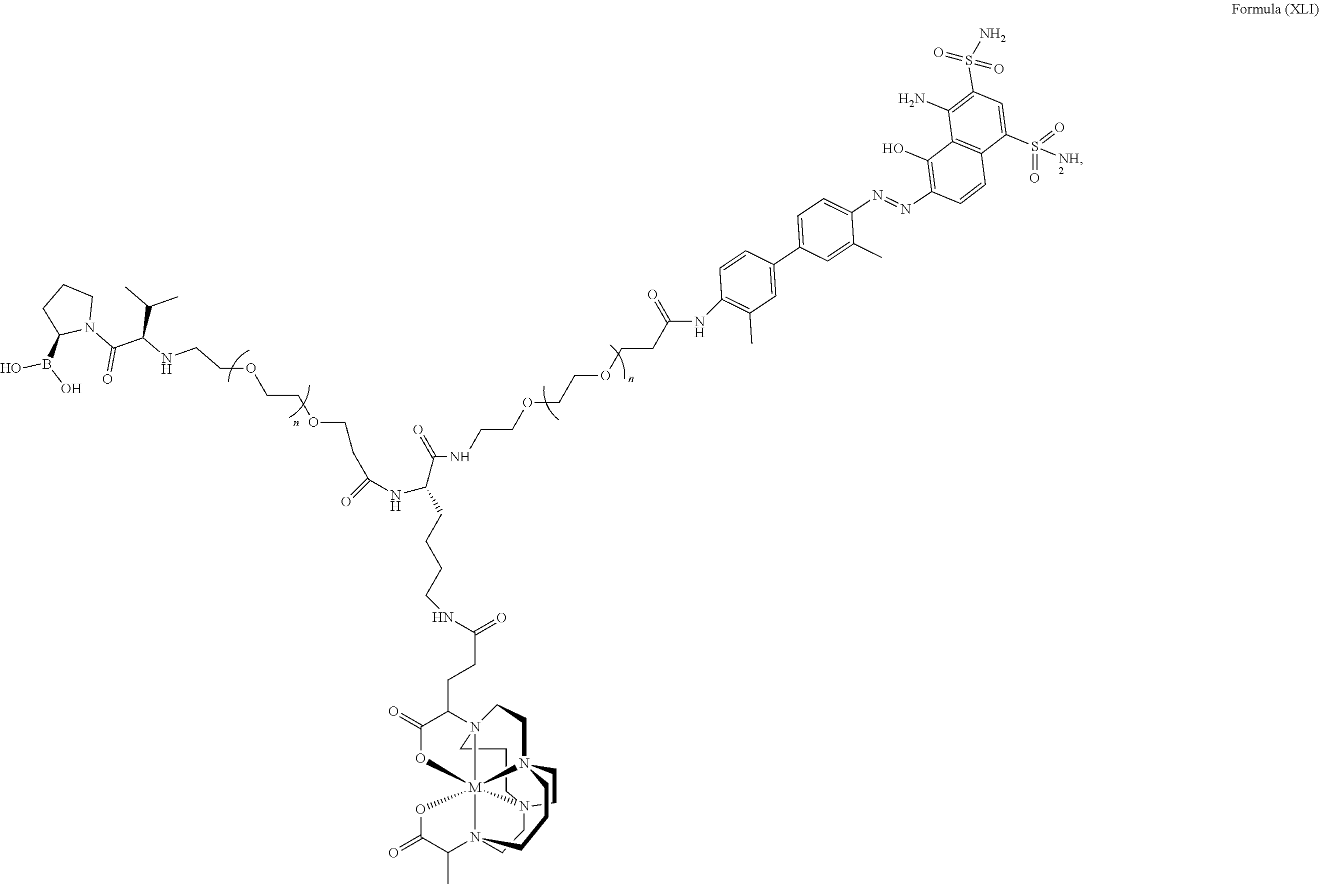

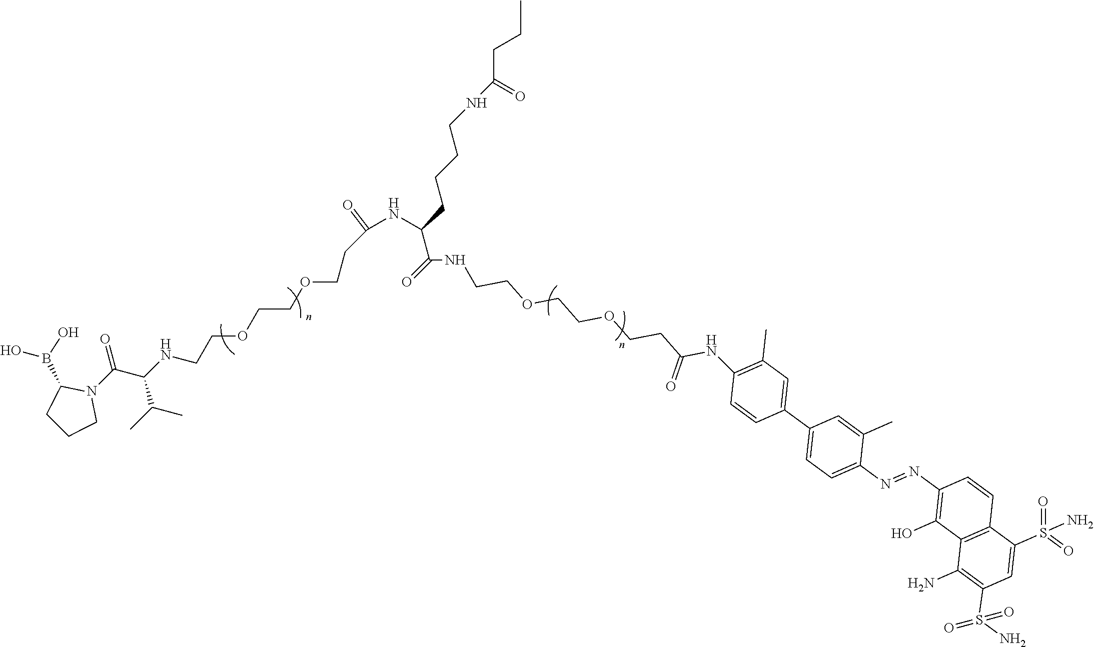

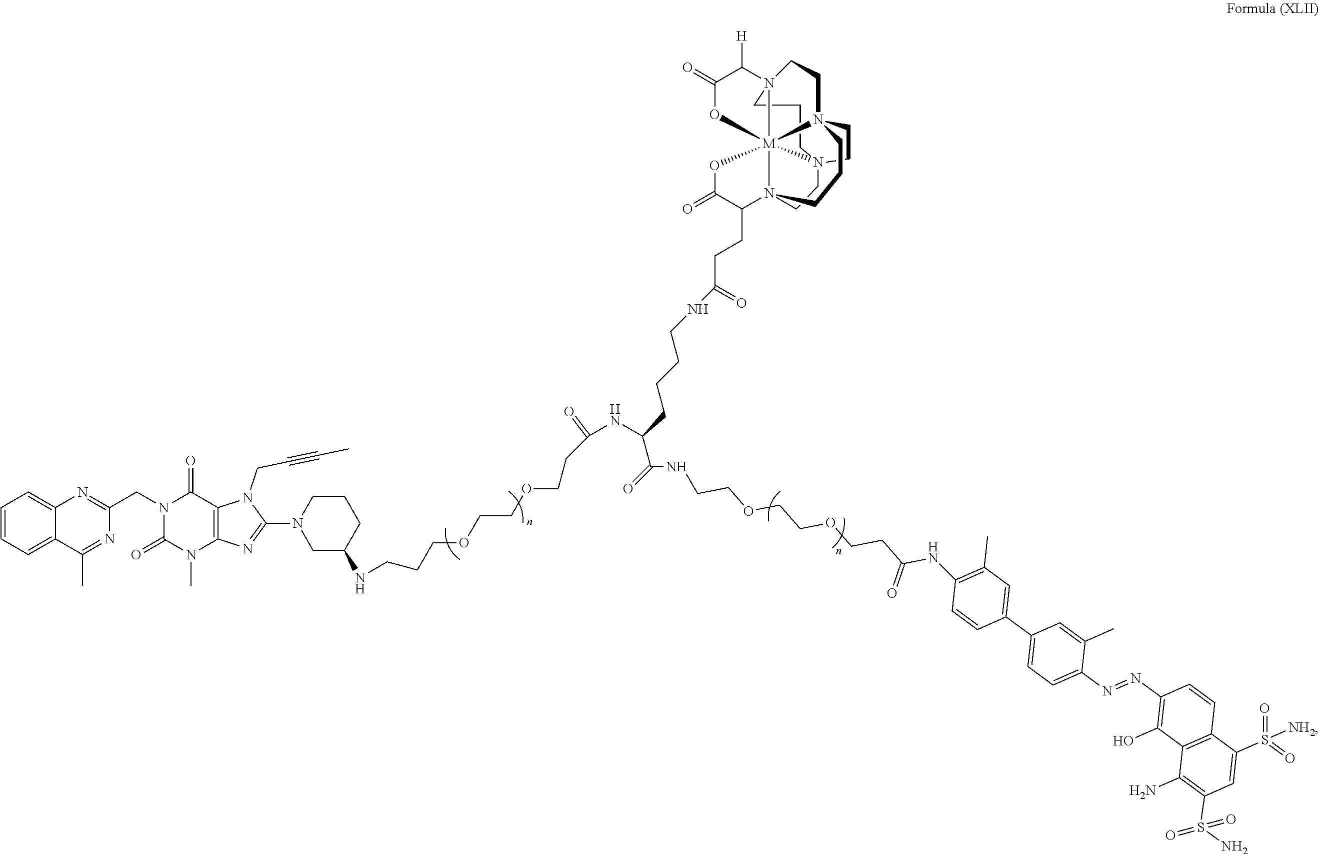

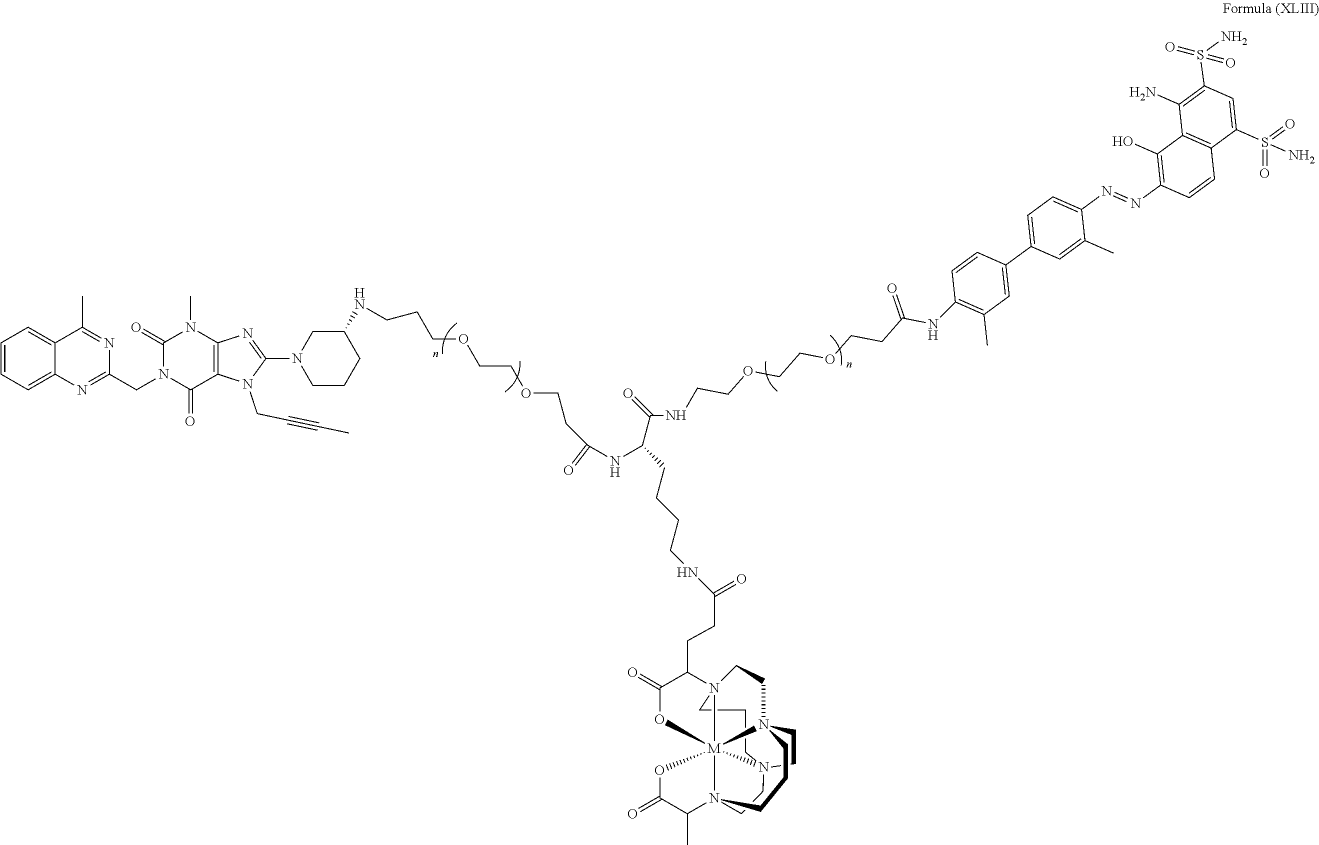

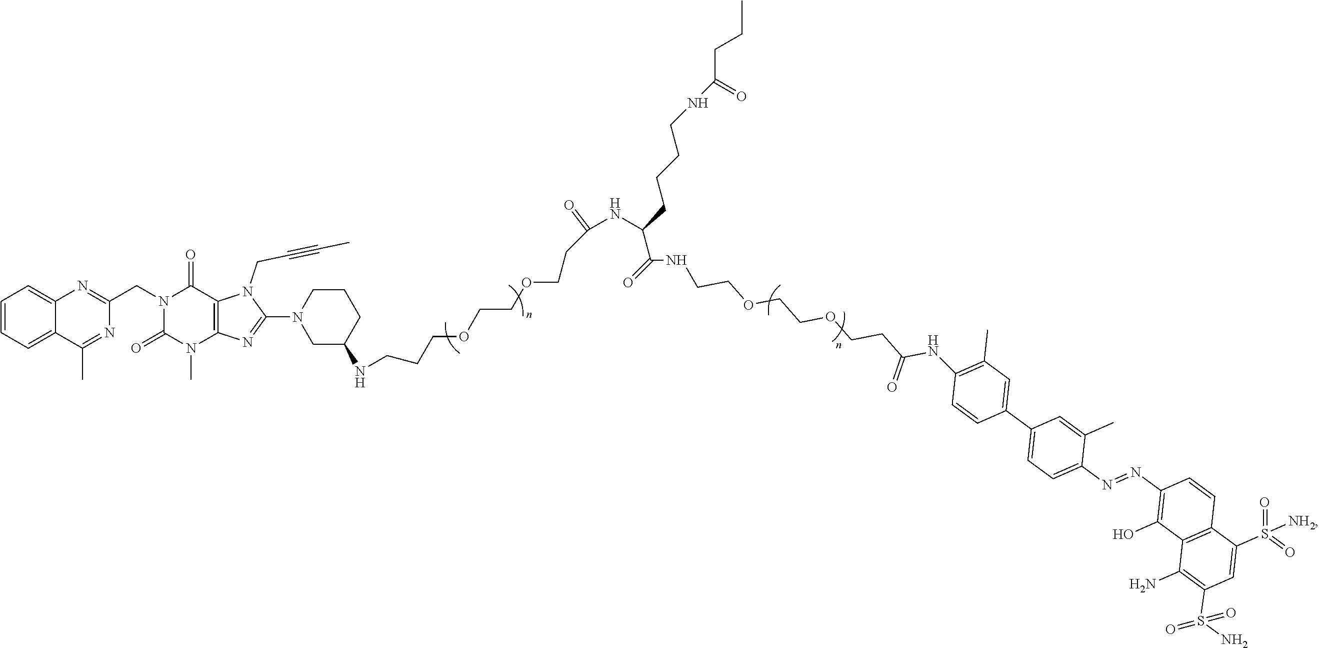

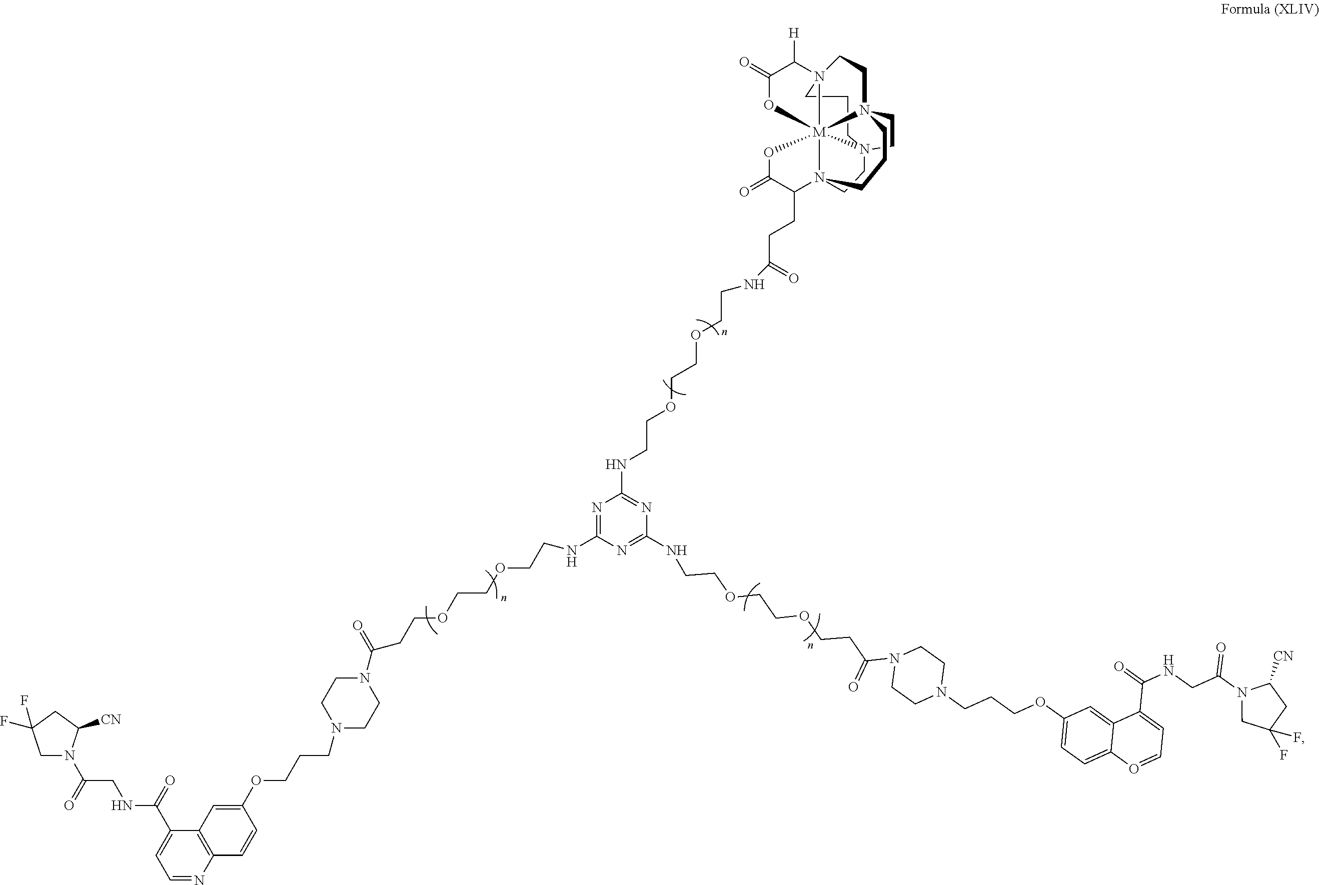

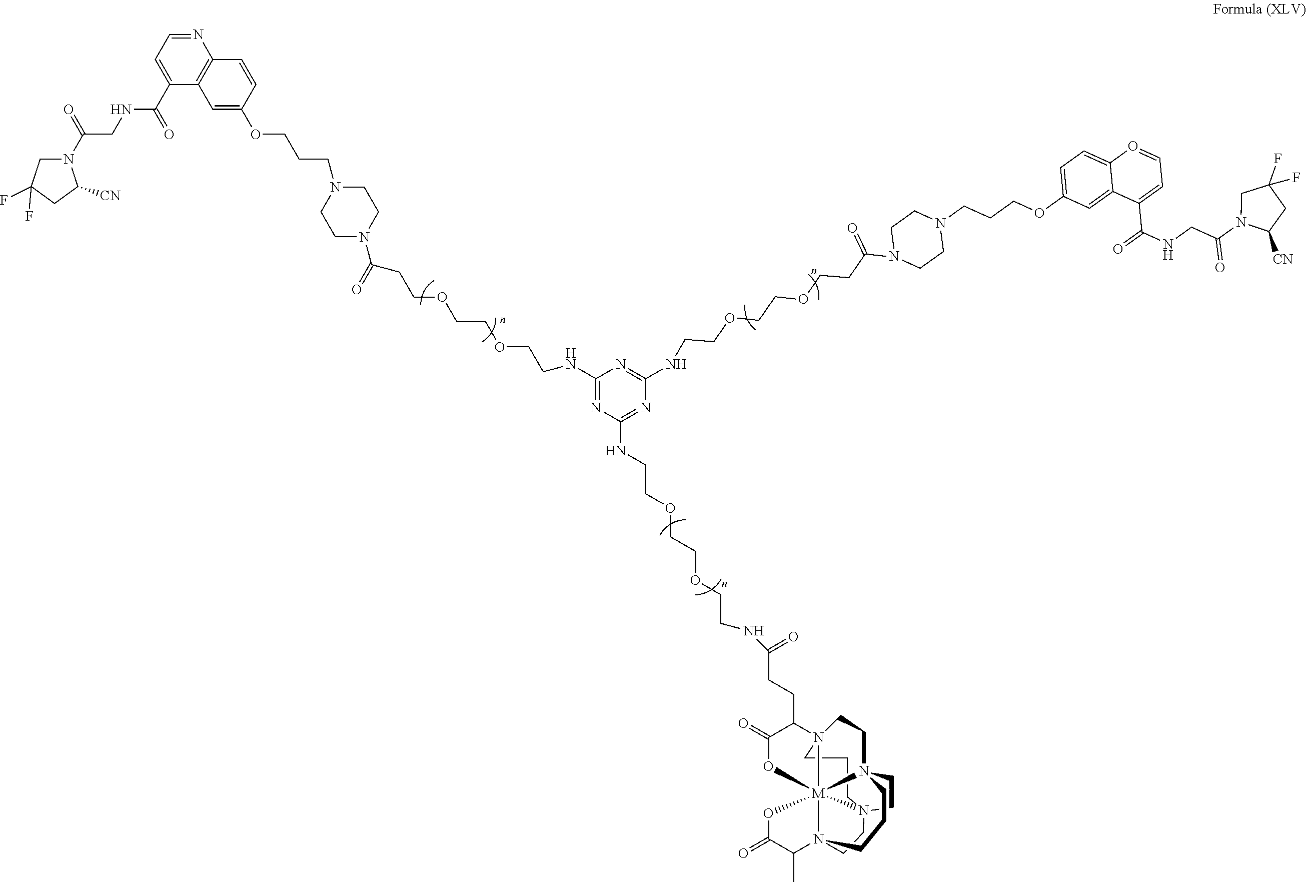

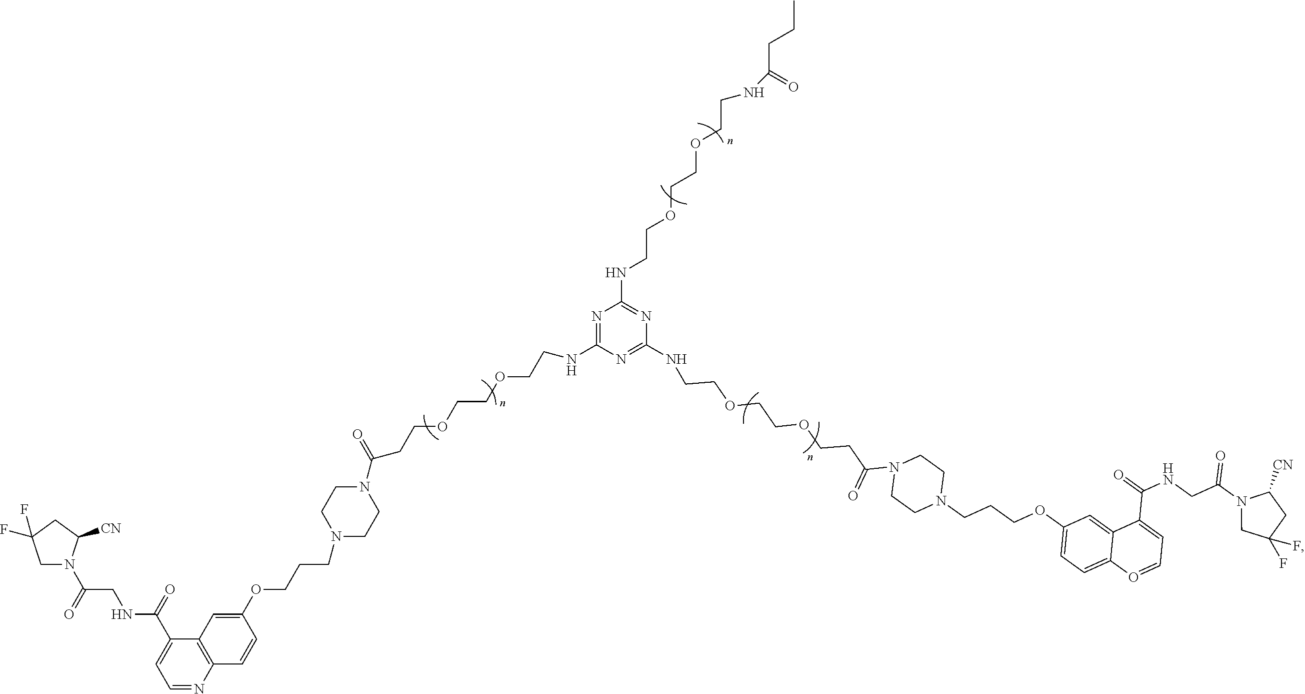

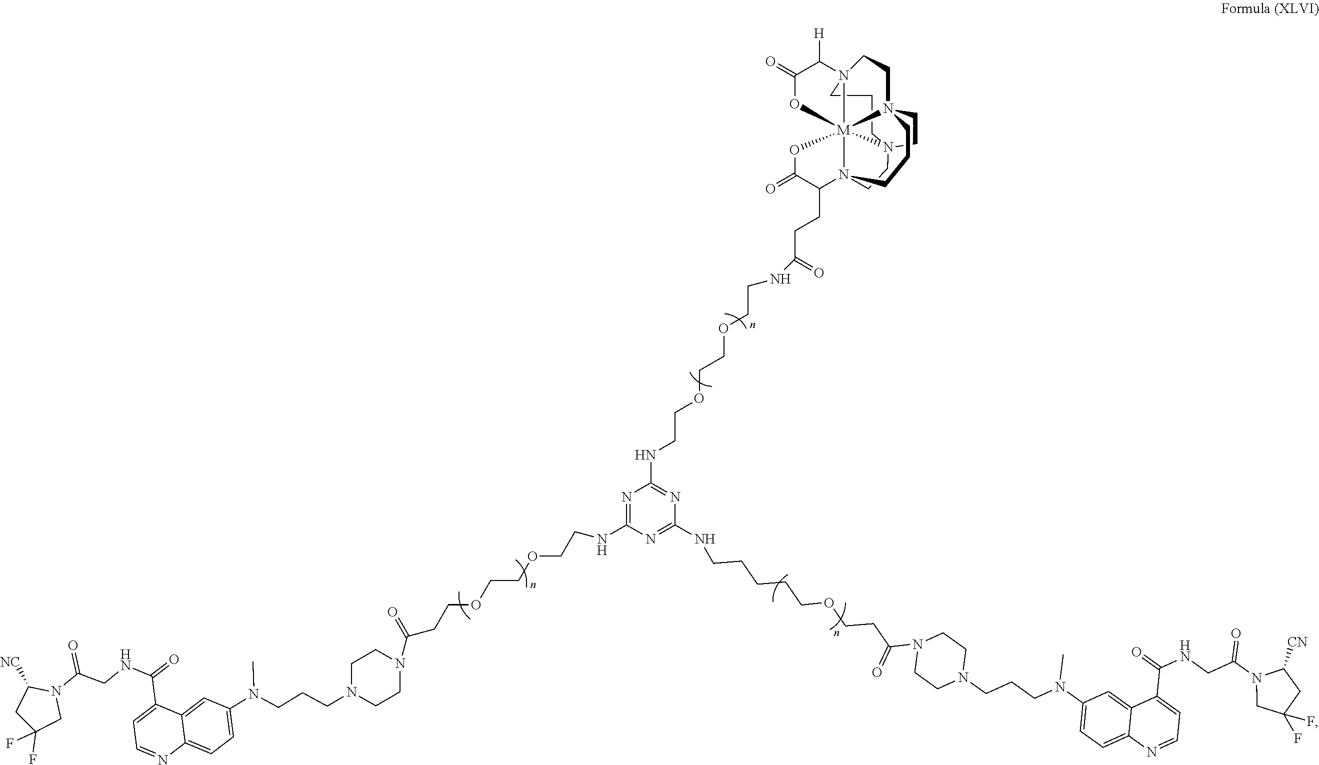

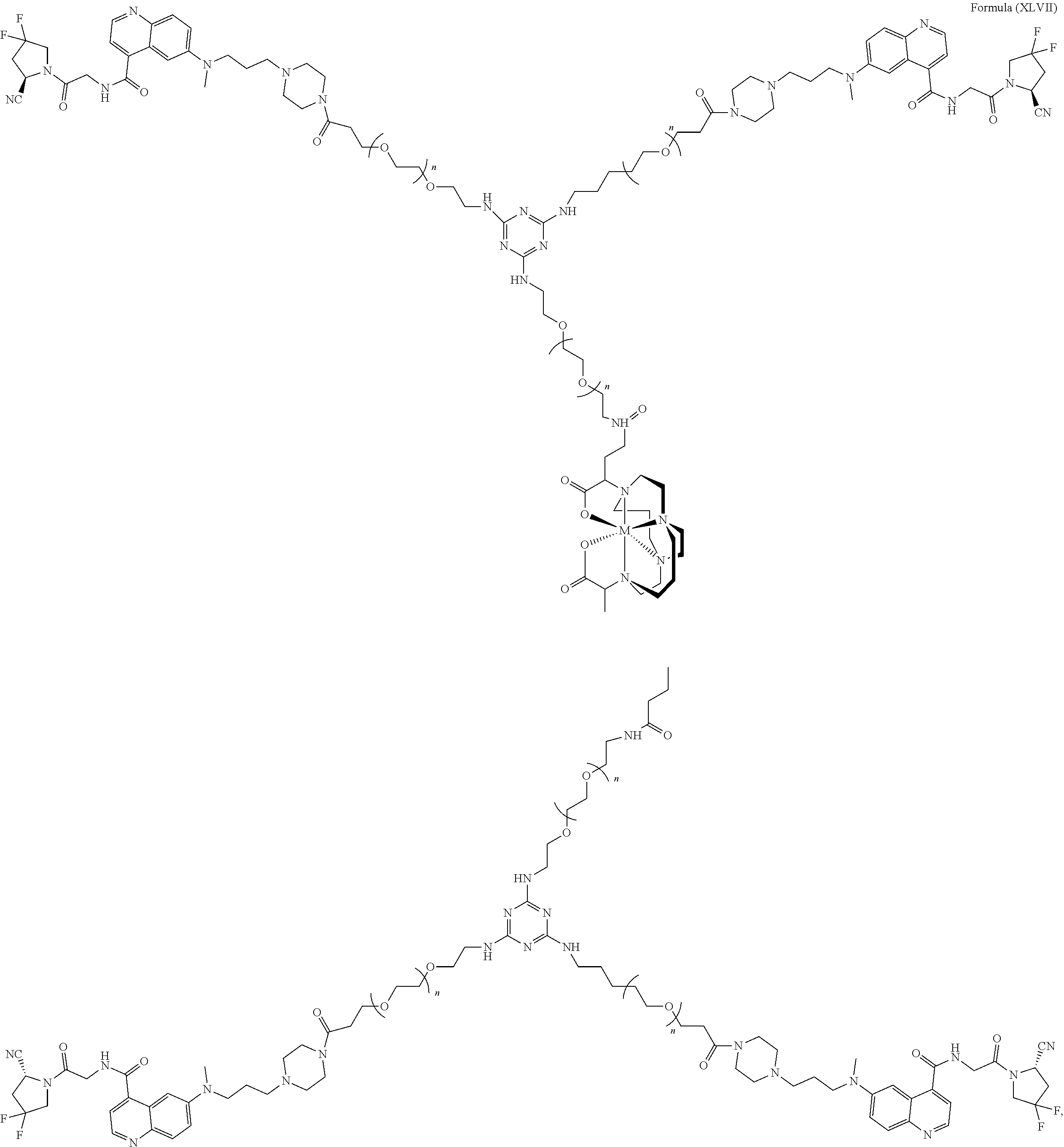

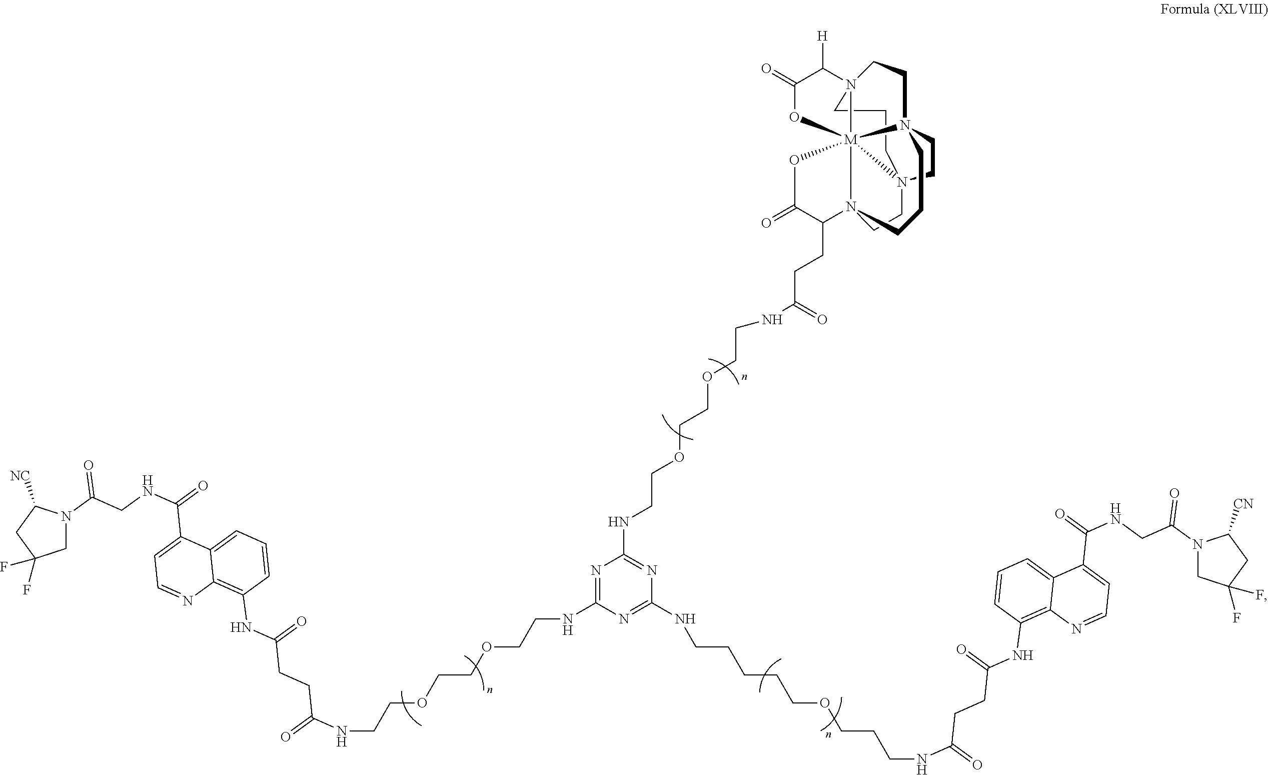

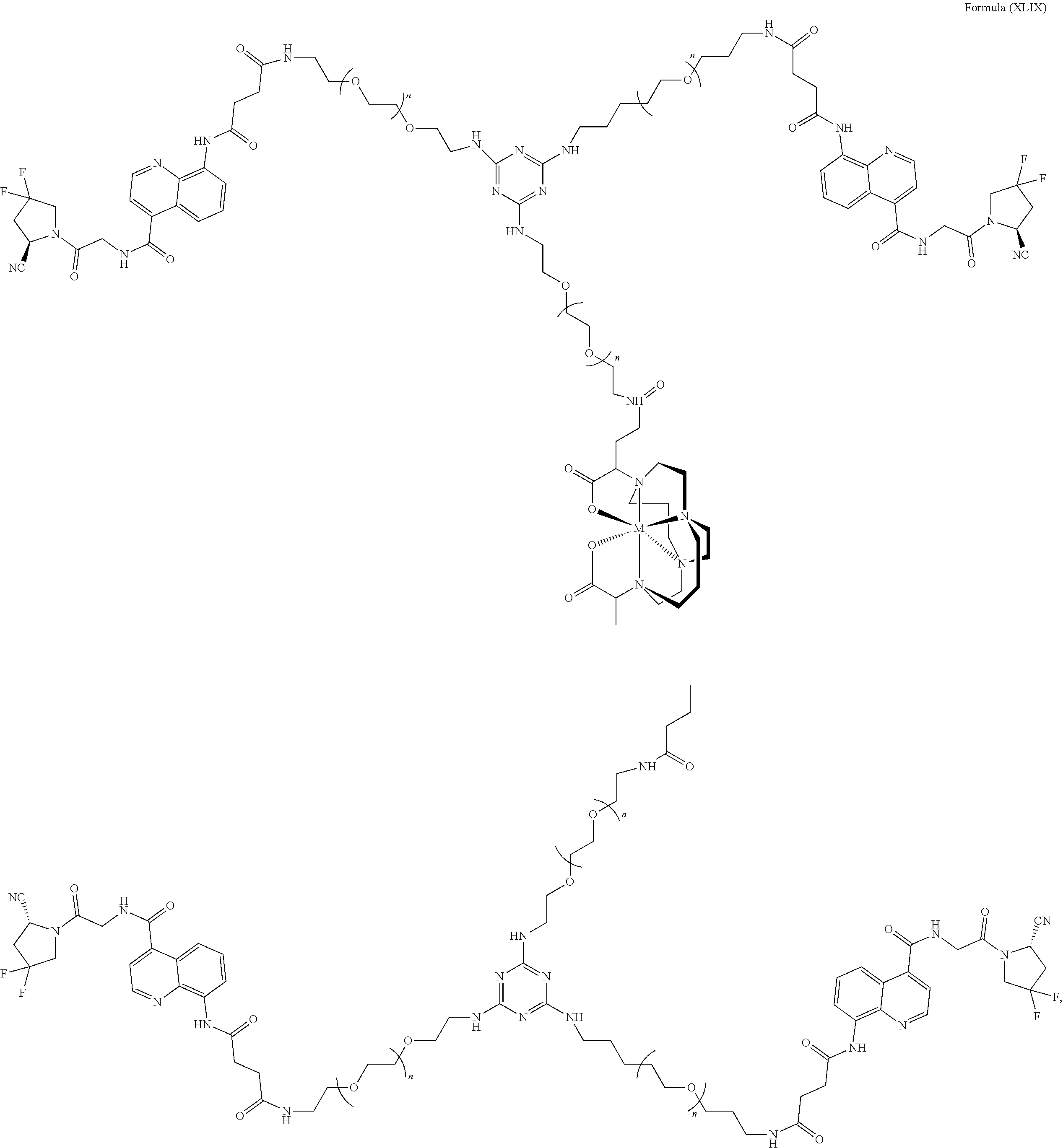

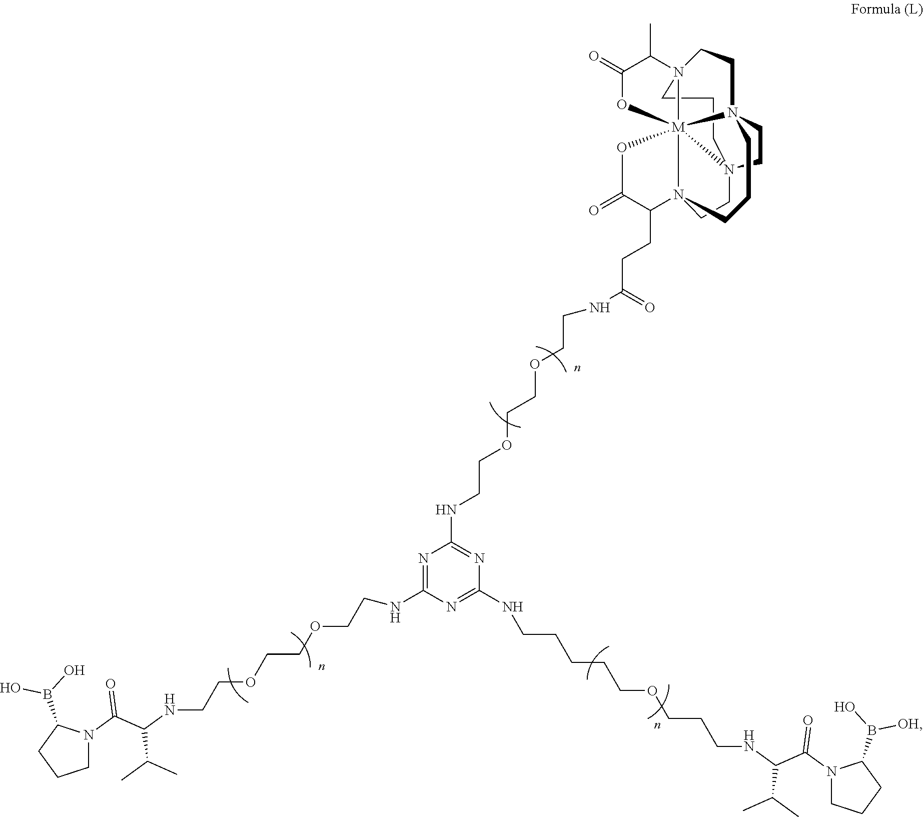

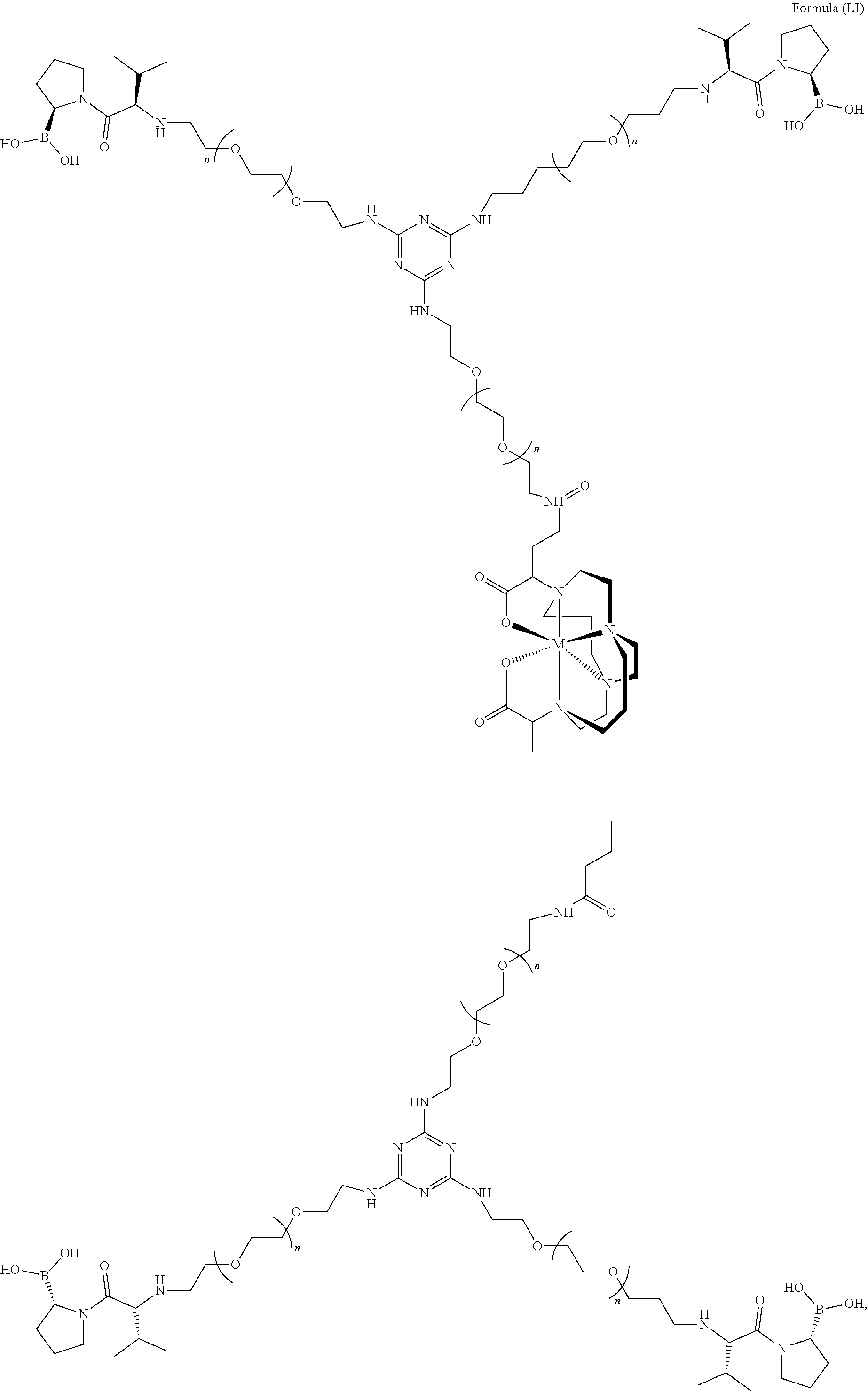

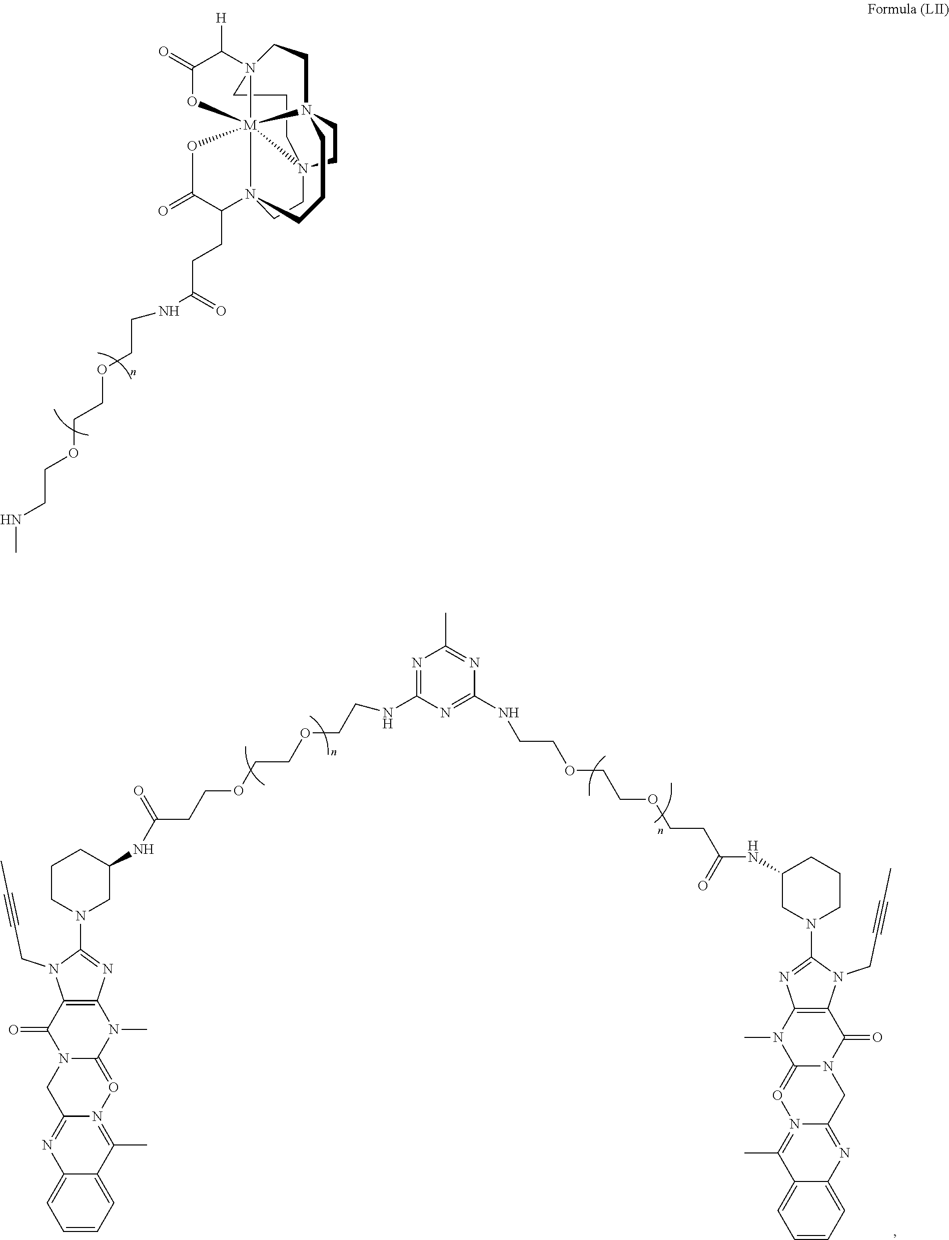

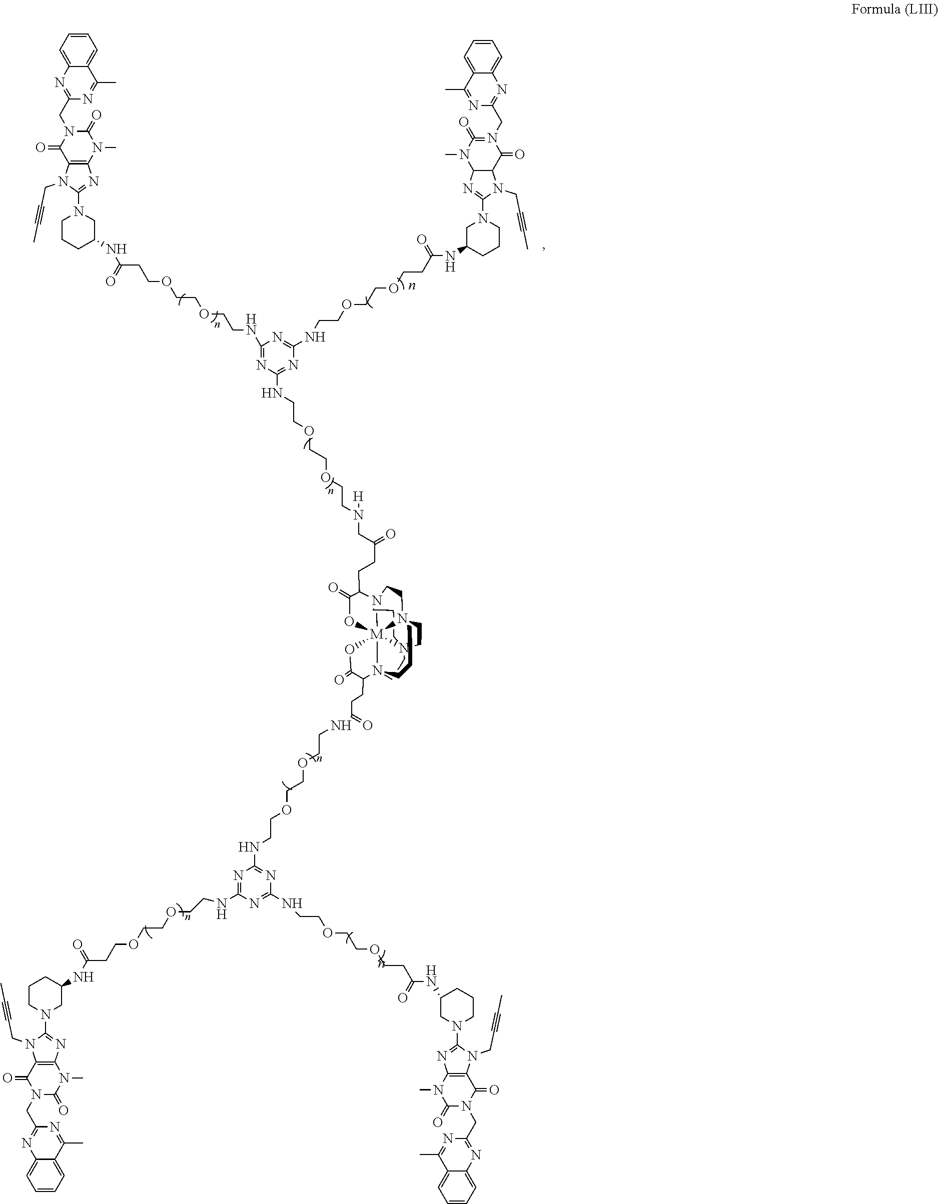

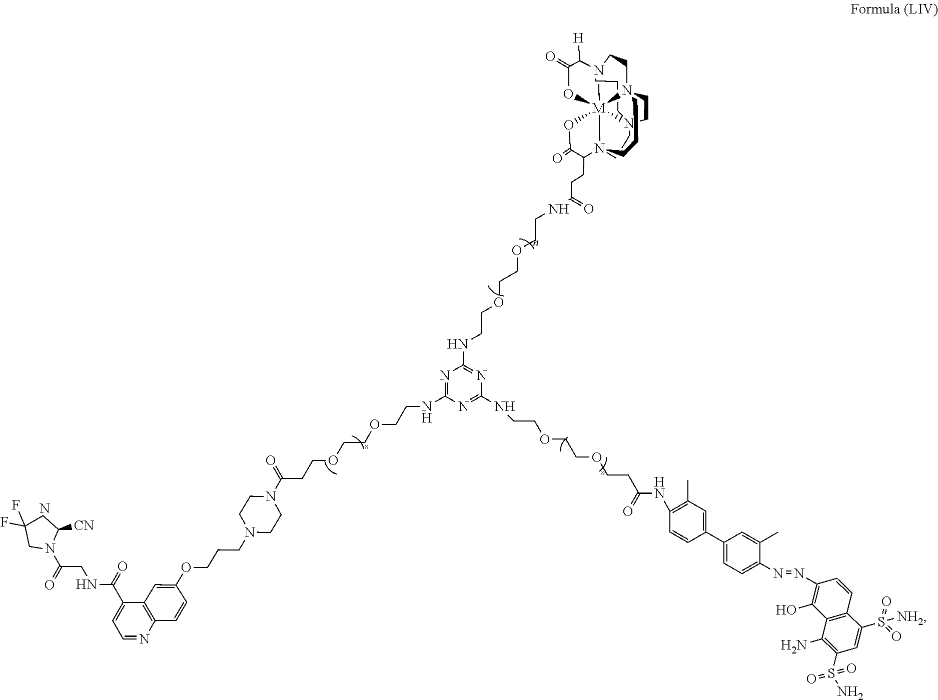

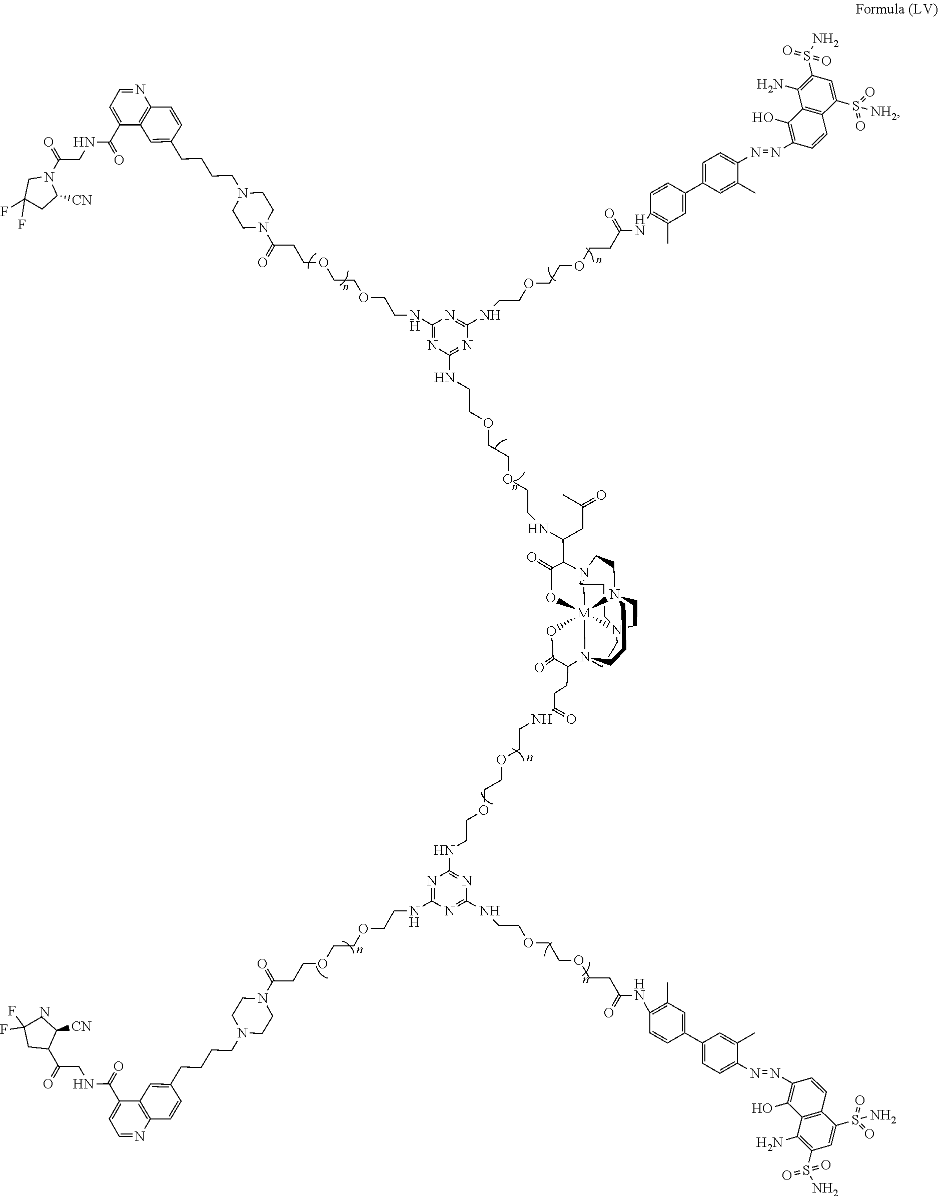

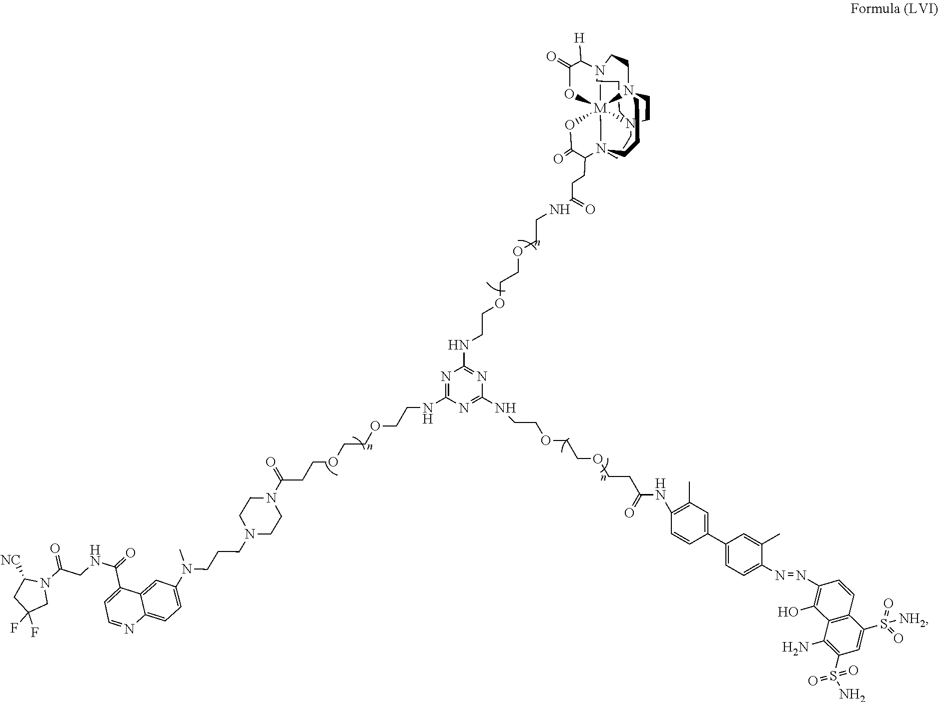

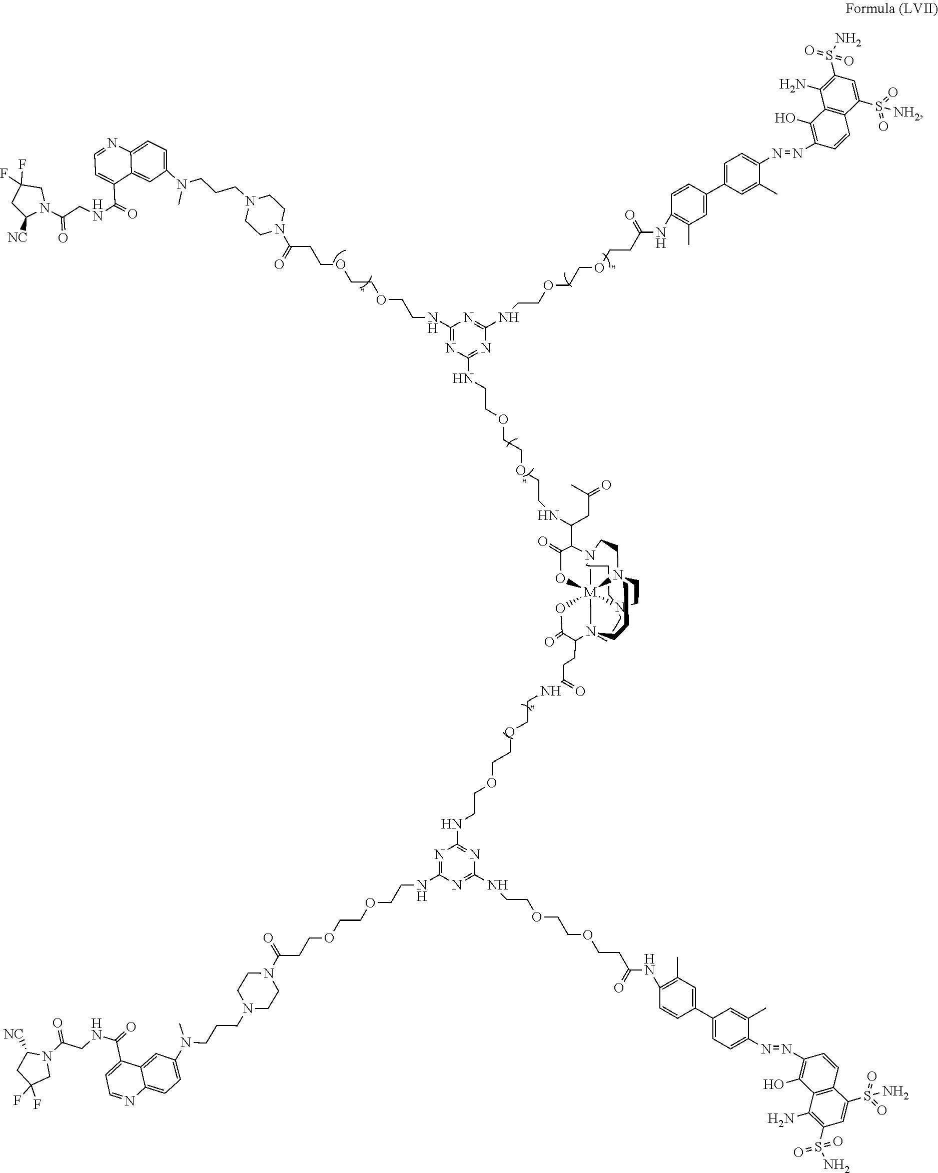

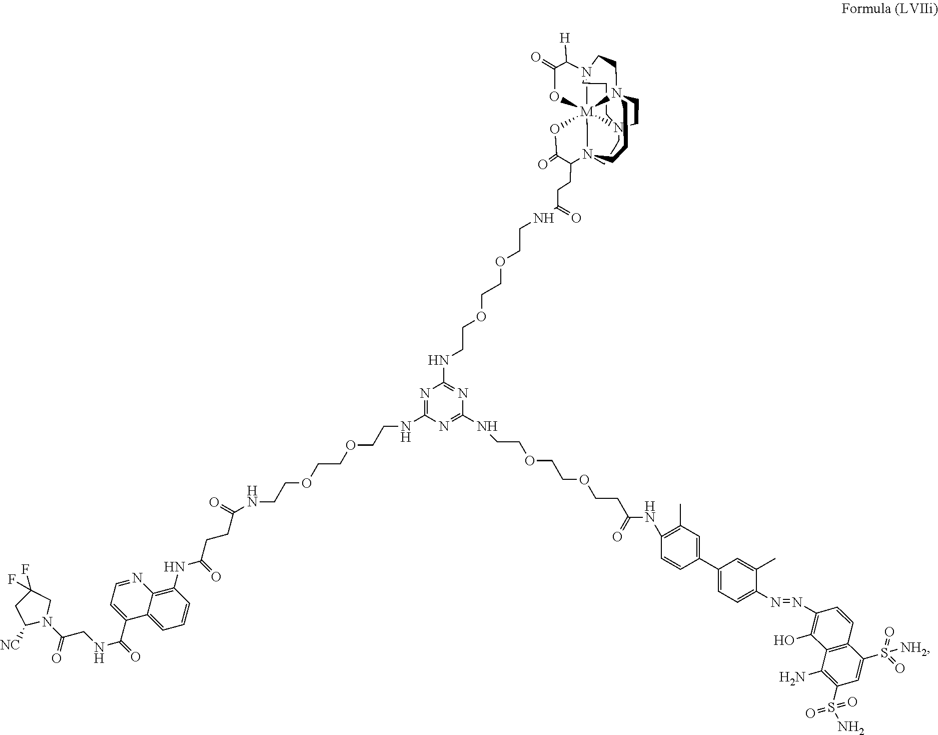

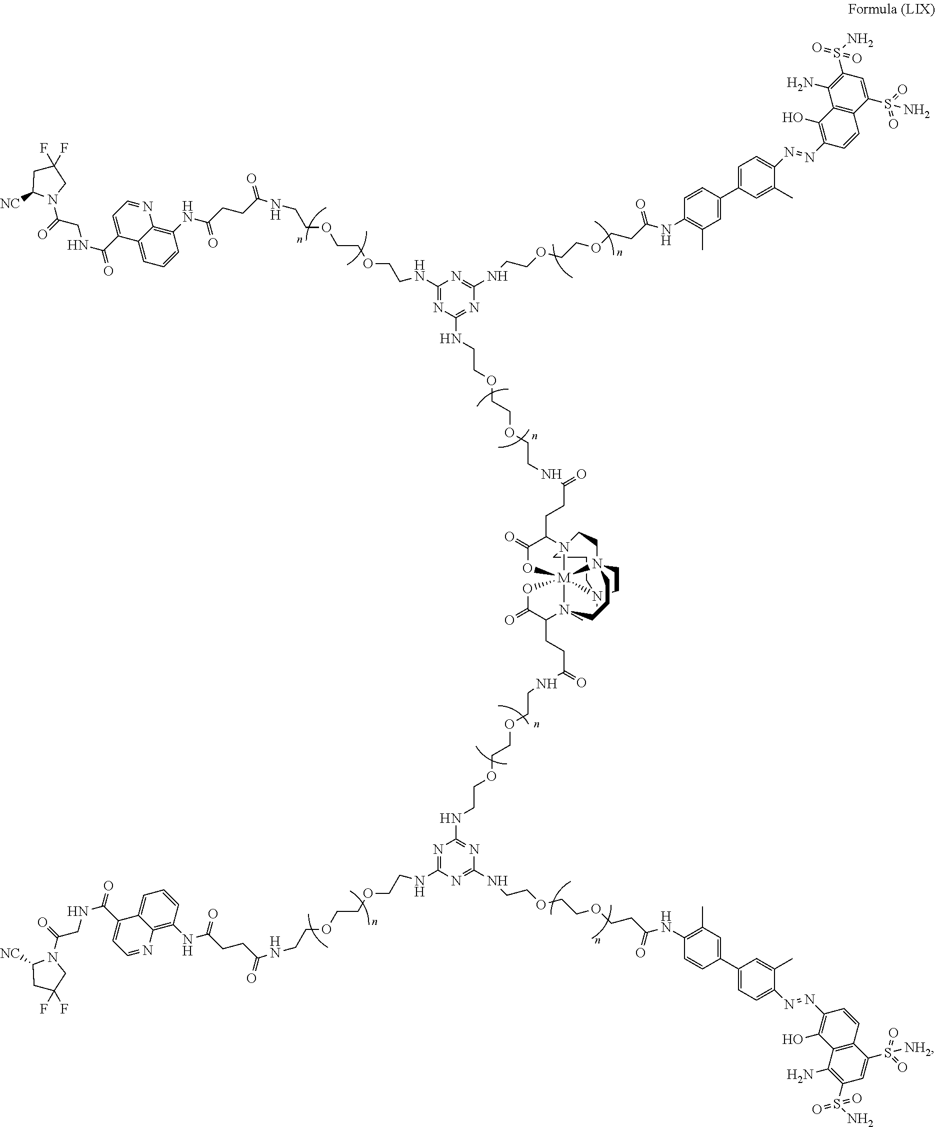

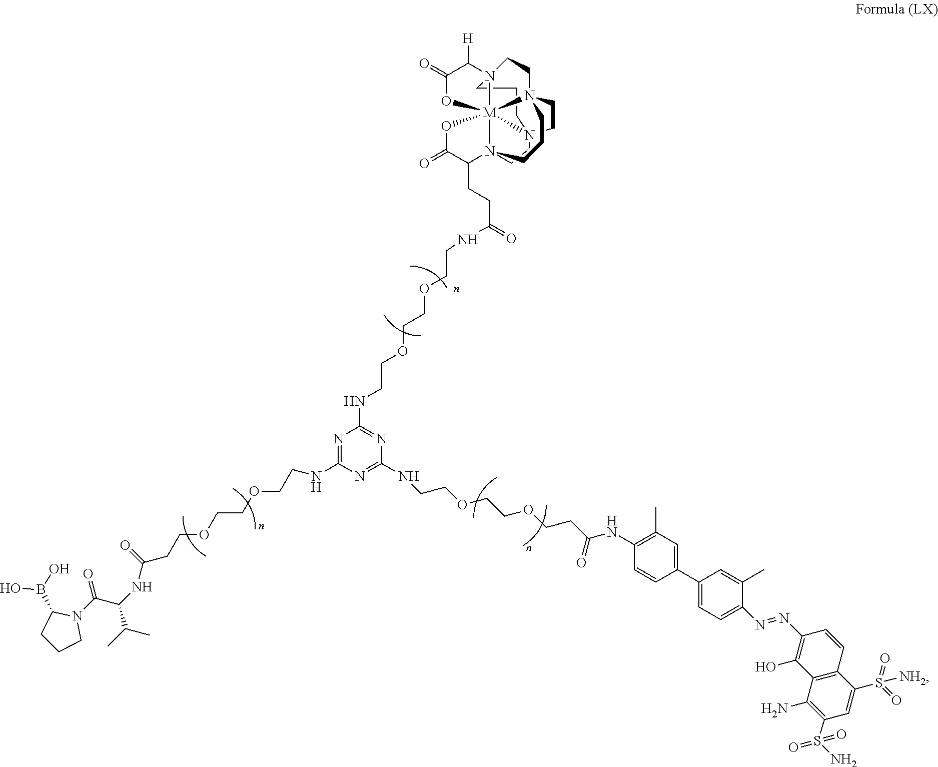

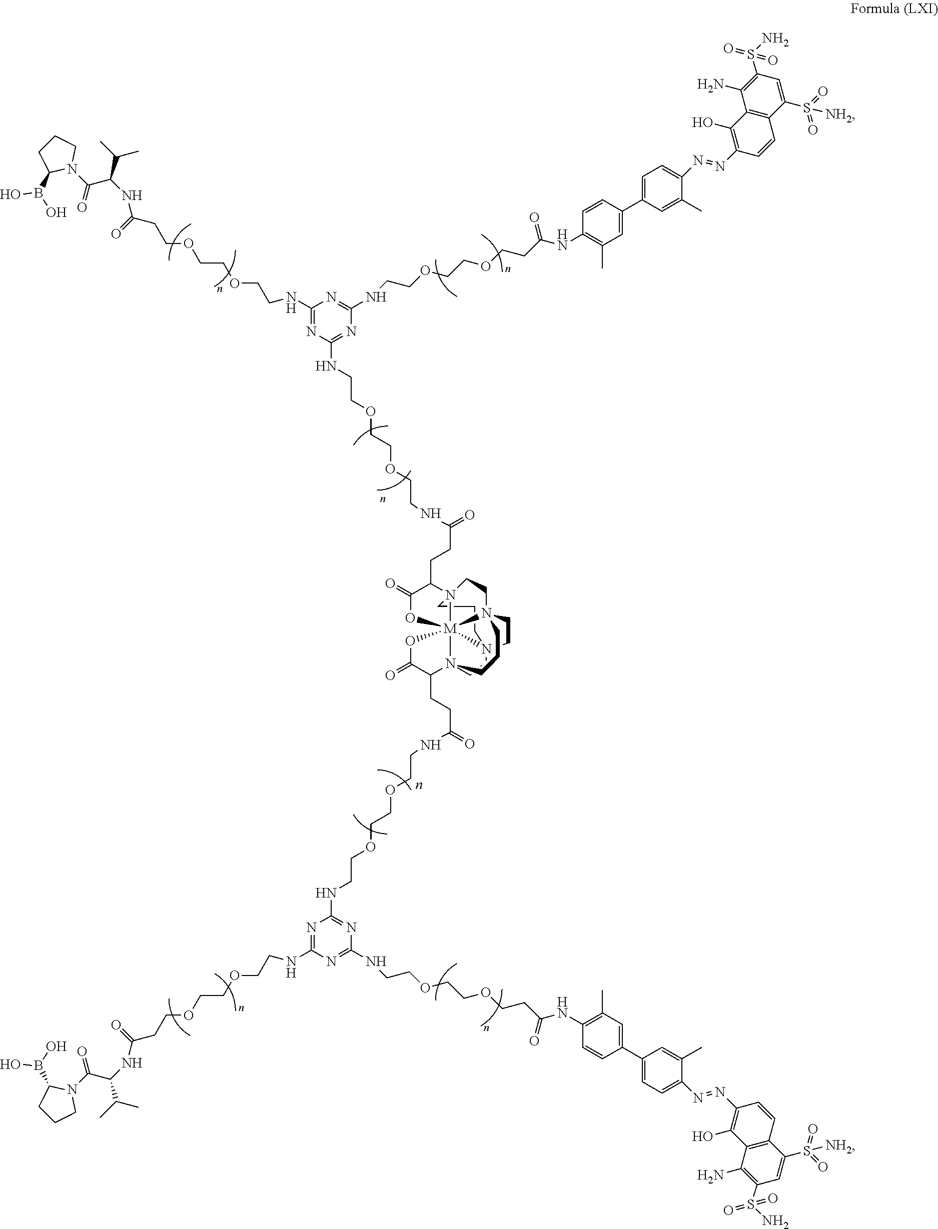

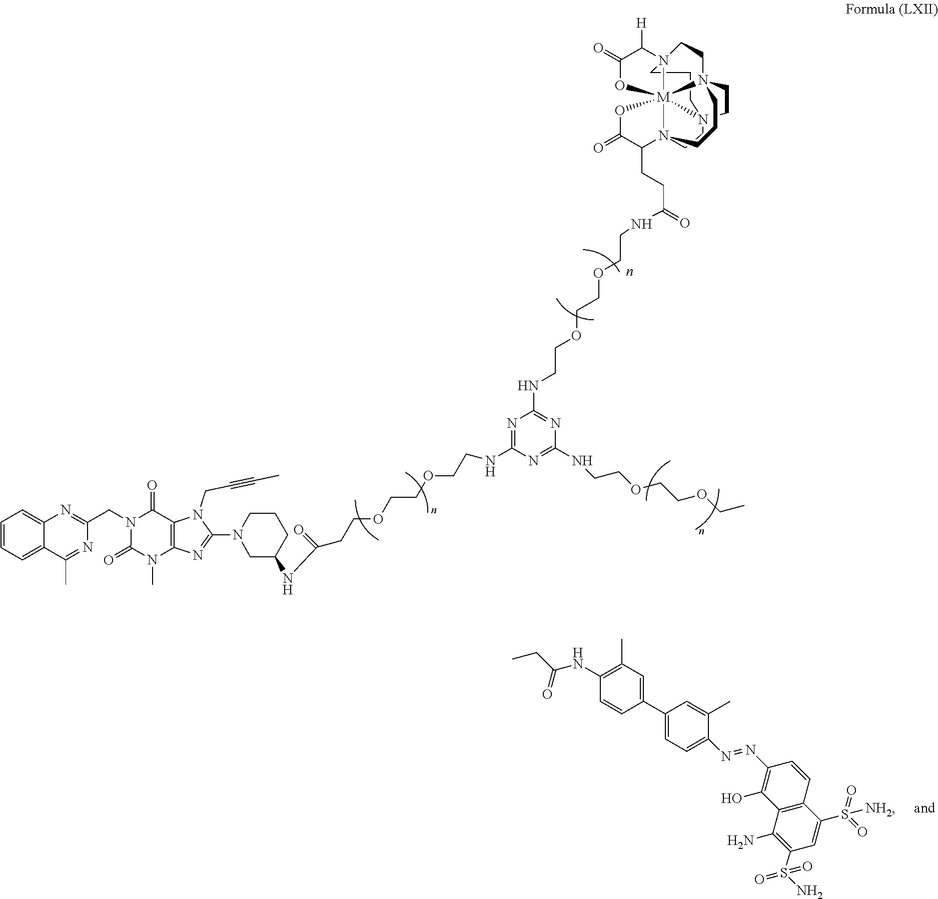

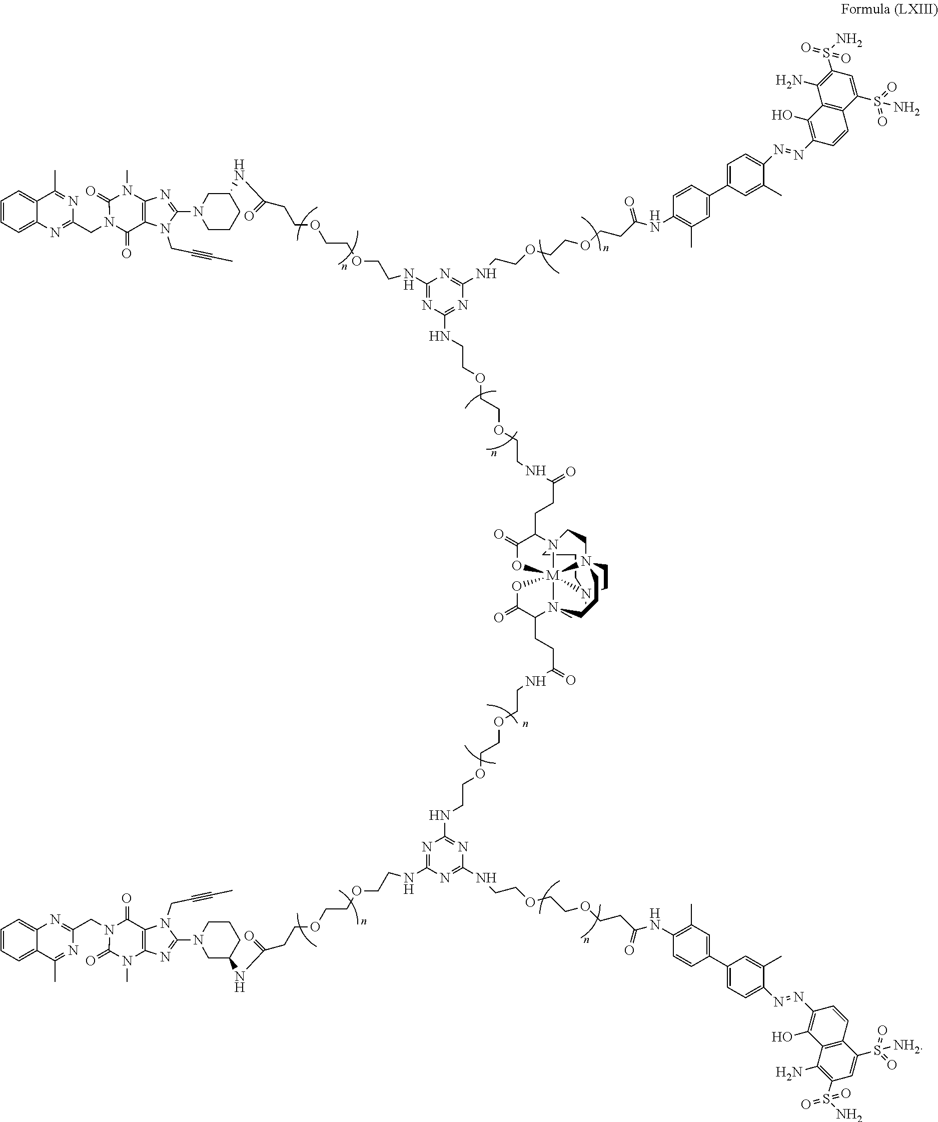

In yet another aspect, the compound is of Formula (a) through Formula (h.b3), any positional isomer, any stereo isomer, or any pharmaceutically acceptable salts thereof:

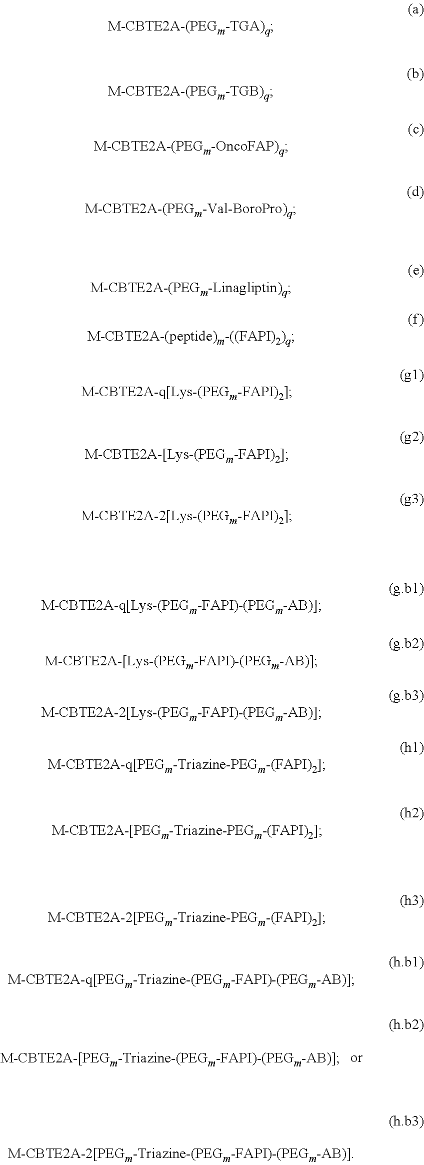

In the above structures, M is a chelating copper comprising ⁶⁰Cu, ⁶¹Cu, ⁶²Cu, ⁶⁴Cu, or ⁶⁷Cu; m is zero, or any integer of 1 to 12; q is 1 or 2; PEG is polyethylene glycol; AB is an albumin binder; Lys is lysine; and CBTE2A represents one of the following two moieties

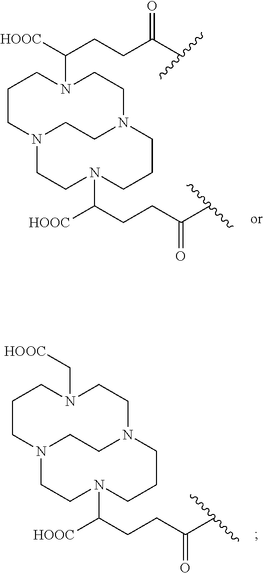 [SMILES: *C(=O)CCC(C(=O)O)N1CCCN2CCN(CCCN(C(CCC(*)=O)C(=O)O)CC2)CC1 ; *C(=O)CCC(C(=O)O)N1CCCN2CCN(CCCN(CC(=O)O)CC2)CC1]

FAPI is a Fibroblast Activation Protein targeting group, comprising TGA, TGB, OncoFAP, Val-BoroPro, or Linagliptin:

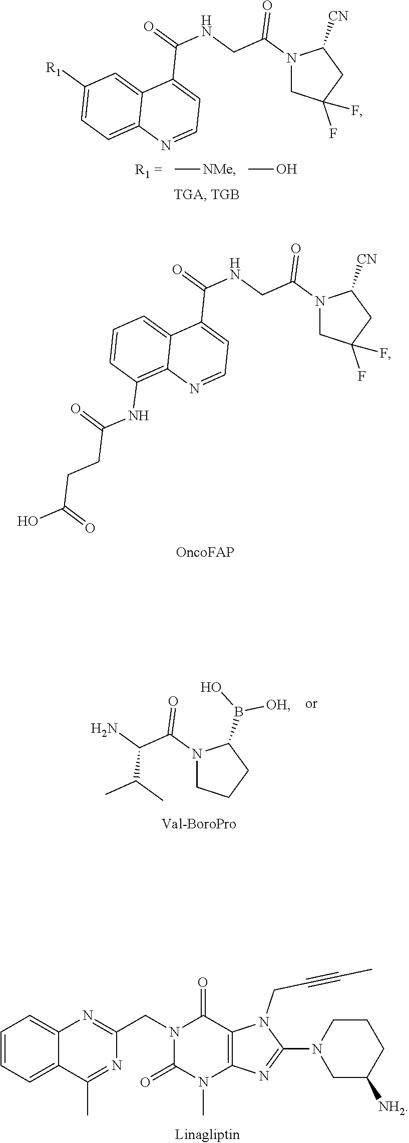

Yet in another aspect, the compound of Formula (a), Formula (b), Formula (c), Formula (d), Formula (e), Formula (f), Formula (g), Formula (g.b), Formula (h), or Formula (h.b) has the following structures:

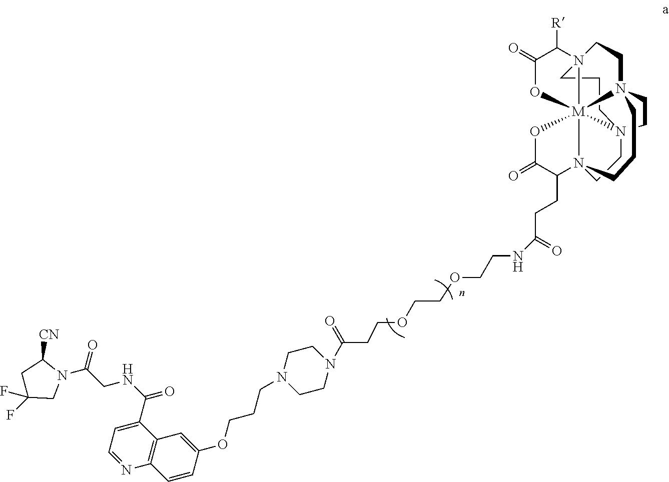

in Formula (a), if R′ is —H, a is M-CBTE2A-PEGₘ-TGB; and if R′ is —CH₂CH₂CO-PEGₘ-TGB, a is M-CBTE2A-(PEGₘ-TGB)₂;

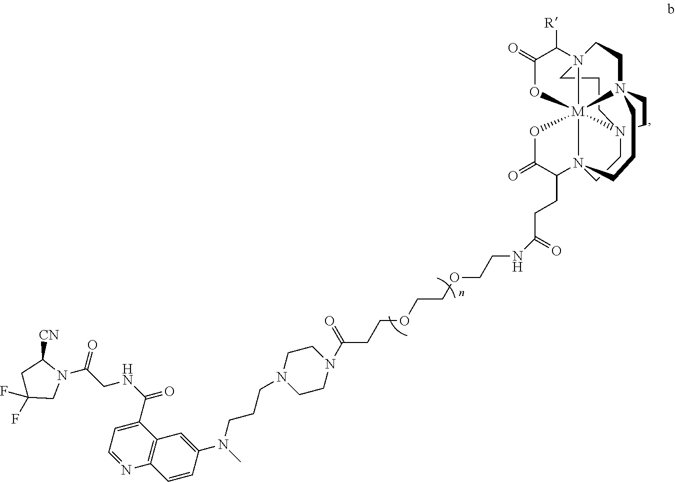

in Formula (b), if R′ is —H, b is M-CBTE2A-PEGₘ-TGA; and if R′ is —CH₂CH₂CO-PEGₘ-TGA, bis M-CBTE2A-(PEGₘ-TGA)₂;

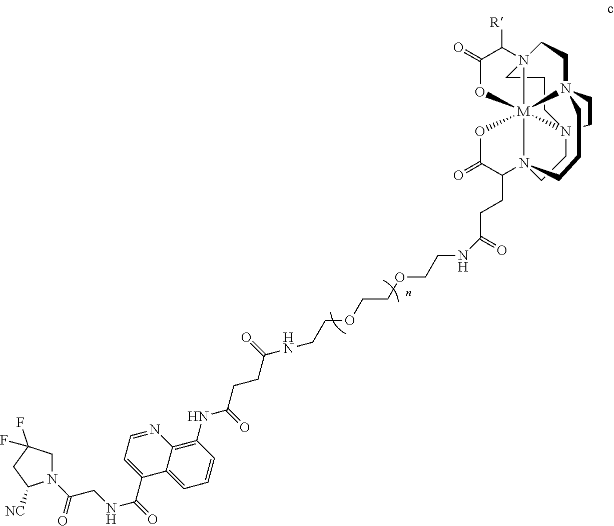

in Formula (c), if R′ is —H, c is M-CBTE2A-PEGₘ-OncoFAP; and if R′ is —CH₂CH₂CO-PEGₘ-OncoFAP, cis M-CBTE2A-(PEGₘ,OncoFAP)₂;

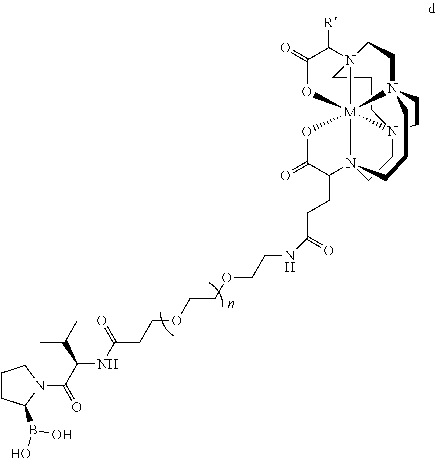

in Formula (d), if R′ is —H, d is M-CBTE2A-PEGₘ-ValBoro-Pro; and if R′ is —CH₂CH₂CO—PEGₘ-ValBoro-Pro, dis M-CBTE2A-(PEGₘ-ValBoro-Pro)₂;

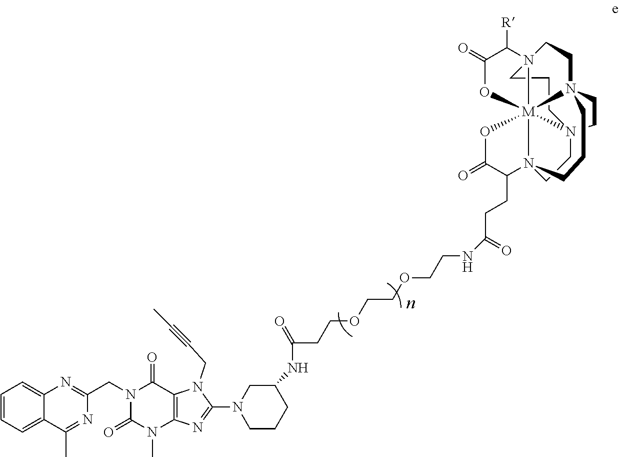

in Formula (e), if R′ is —H, e is M-CBTE2A-PEGₘ-Linagliptin, and if R′ is —CH₂CH₂CO—PEGₘ-Linagliptin, e is M-CBTE2A-(PEGₘ-Linagliptin)₂;

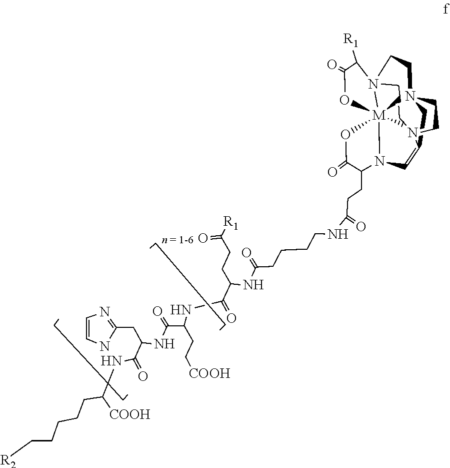

in Formula (f), if R′ is —H, f is M-CBTE2A-Peptide-FAPI; and if R′ is —CH₂CH₂CO—Peptide-FAPI, f is -CBTE2A-(Peptide-FAPI)₂, R₁′ is —OH or —NH₂, and R₂′ is a chemical moiety of i, ii, iii, iv, or v:

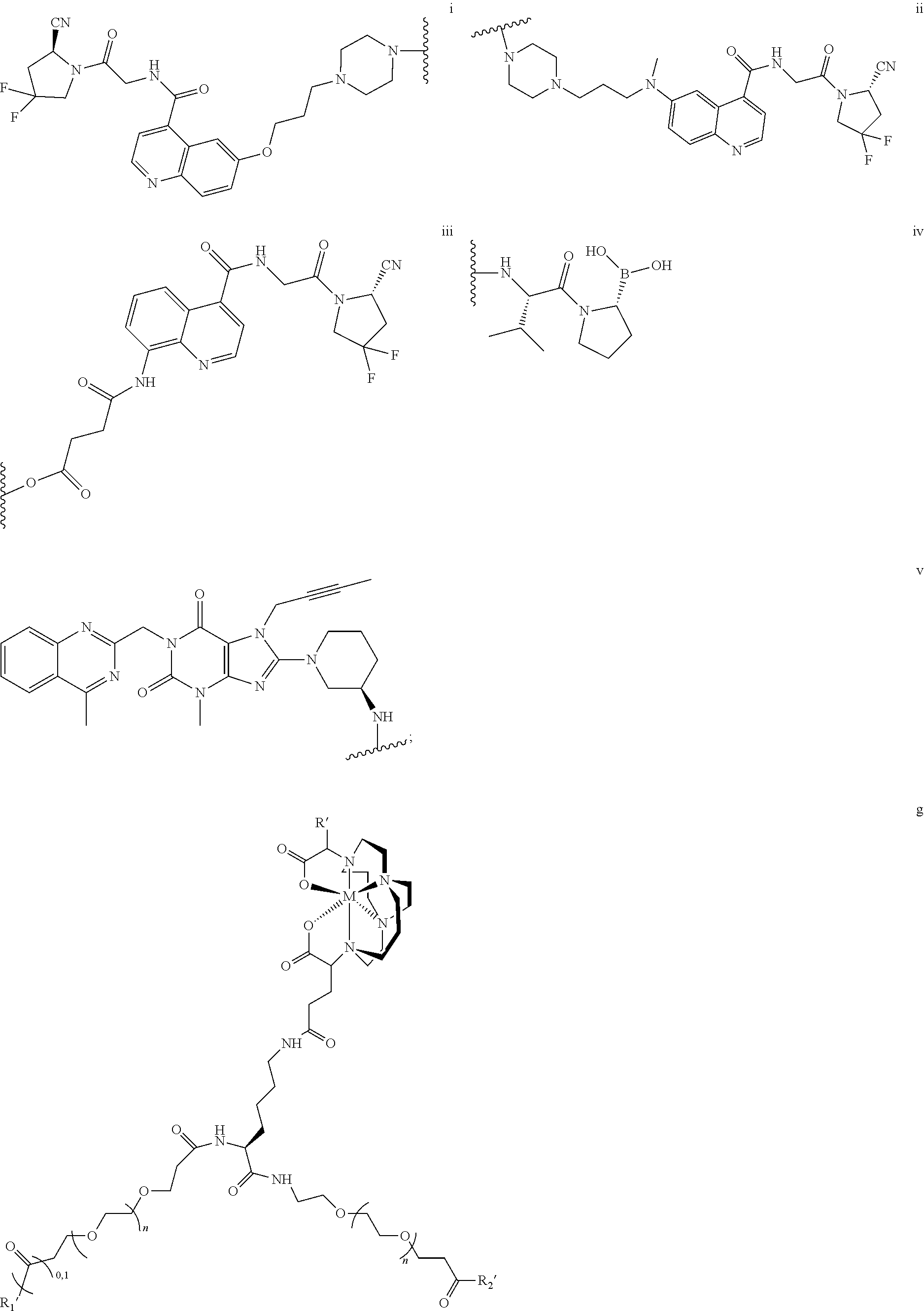

in Formula (g), if R′ is —H, g is M-CBTE2A-[Lys-PEGₘ-(FAPI)₂]; and if R′ is —CH₂CH₂CO-[Lys-PEGₘ-(FAPI)₂], g is M-CBTE2A-[Lys-PEGₘ-(FAPI)₂]; R₁′ and R₂′ each is independently selected from the group consisting of i, ii, iiia, iv, and v:

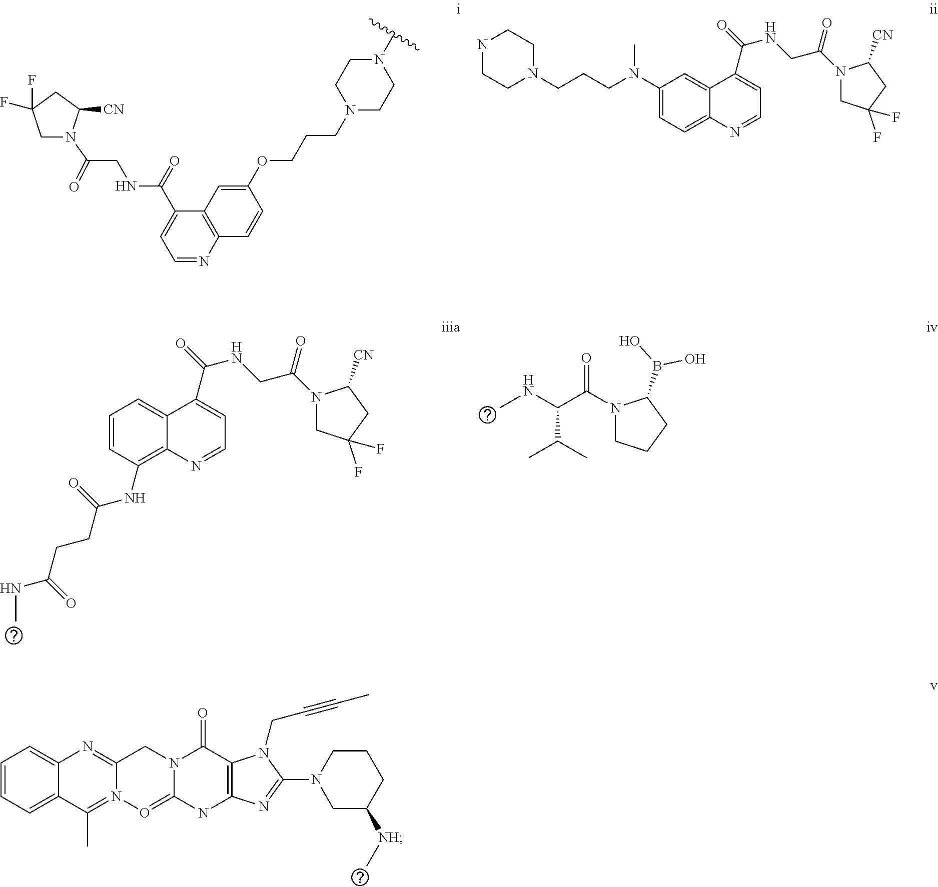

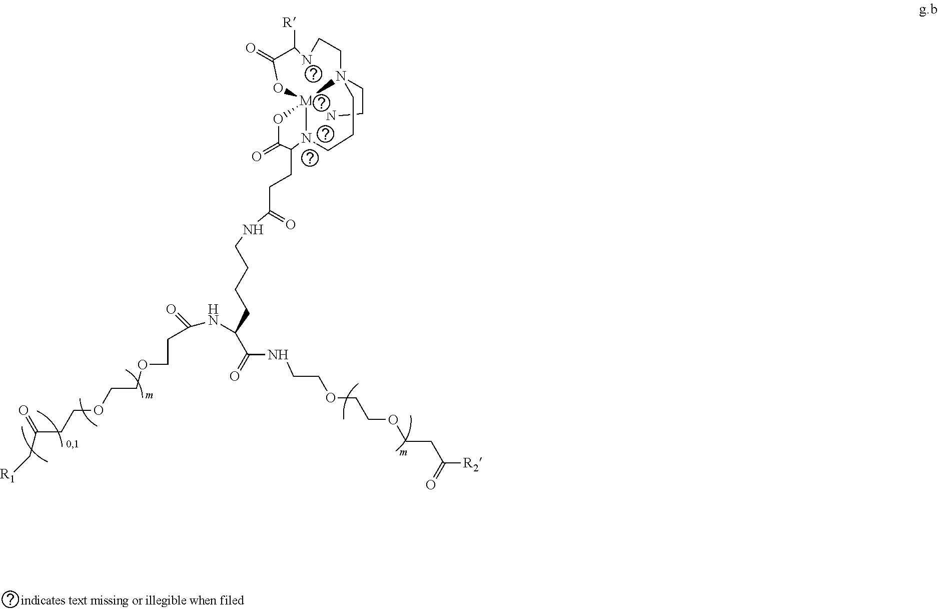

in Formula (g.b), if R′ is —H, (g.b) is M-CBTE2A-[Lys-(PEGₘ-FAPI)-(PEGₘ-AB)], and if R′ is —CH₂CH₂CO-[Lys-(PEGₘ-FAPI)-(PEGₘ-AB)], (g.b) is M-CBTE2A-2[Lys-(PEGₘ-FAPI)-(PEGₘ-AB)]; and R₁′ or R₂′ each is independently selected from the group consisting of i, ii, iiia, iv, v, and vi:

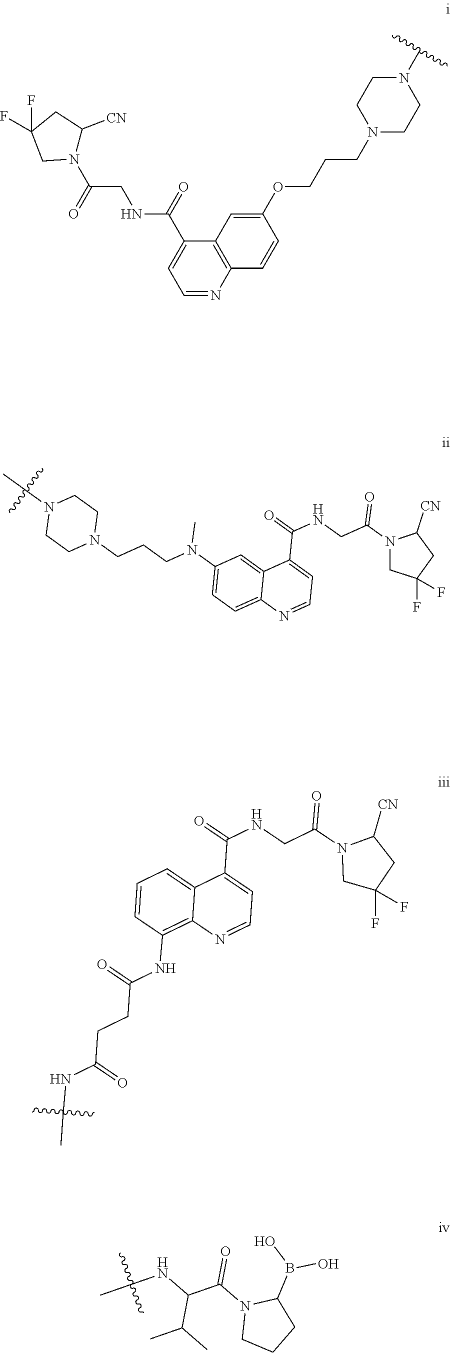 [SMILES: *N1CCN(CCCOc2ccc3nccc(C(=O)NCC(=O)N4CC(F)(F)CC4C#N)c3c2)CC1 ; *N1CCN(CCCN(C)c2ccc3nccc(C(=O)NCC(=O)N4CC(F)(F)CC4C#N)c3c2)CC1 ; *NC(=O)CCC(=O)Nc1cccc2c(C(=O)NCC(=O)N3CC(F)(F)CC3C#N)ccnc12 ; *NC(C(=O)N1CCCC1B(O)O)C(C)C]

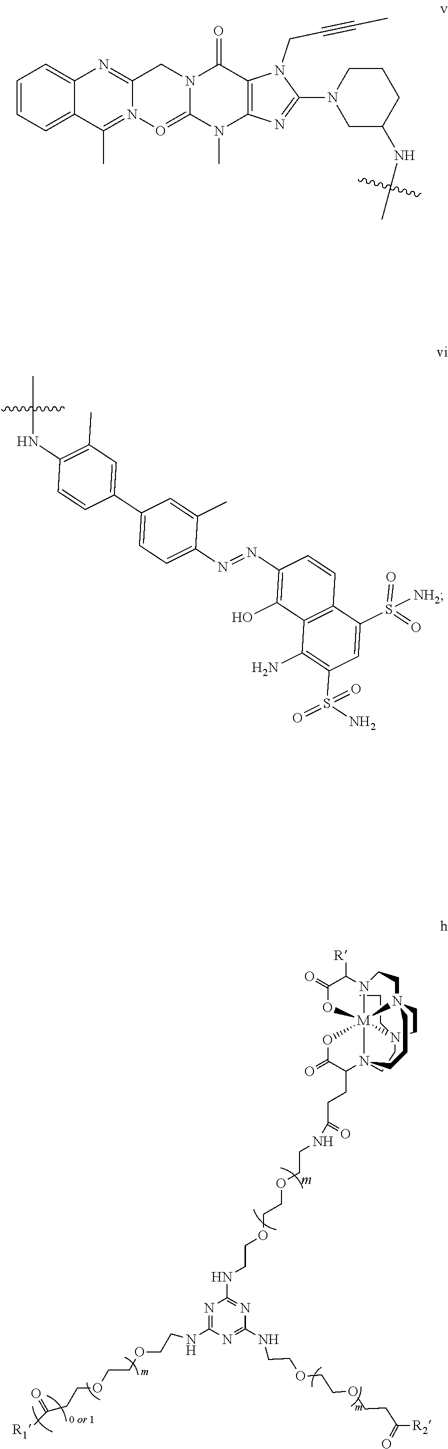

in Formula (h), if R′ is —H, h is M-CBTE2A-[PEGₘ-Triazine-(PEGₘ-FAPI)₂], and if R′ is —CH₂CH₂CO-[PEGₘ-Triazine-(PEGₘ-FAPI)₂], h is M-CBTE2A-2[PEGₘ-Triazine-(PEGₘ-FAPI)₂]; R₁′ and R₂′ each is independently selected from the group consisting of i, ii, iiia, iv, and v.

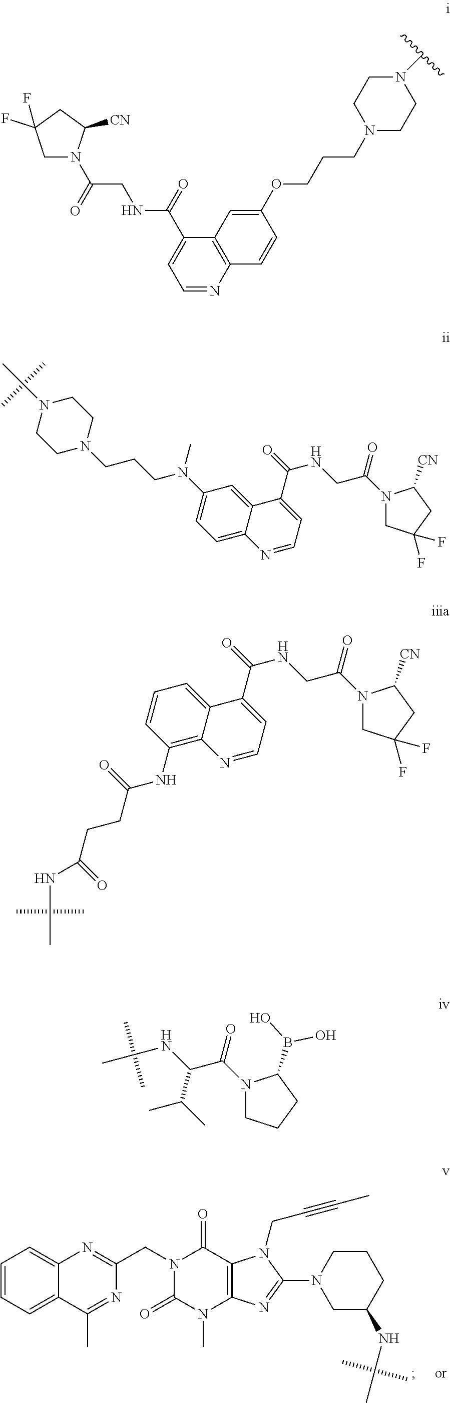

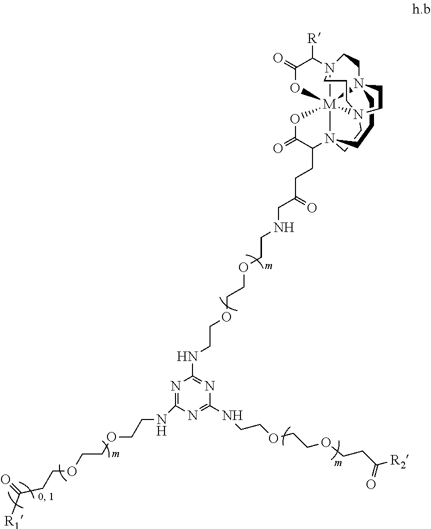

in Formula (h.b), if R′ is —H, (h.b) is M-CBTE2A-[PEGₘ-Triazine-(PEGₘ-FAPI)-(PEGₘ-AB)], and if R′ is —CH₂CH₂CO-[PEGₘ-Triazine-(PEGₘ-FAPI-(PEGₘ-AB)], (h.b) is M-CBTE2A-2[PEGₘ-Triazine-(PEGₘ-FAPI)-(PEGₘ-AB)]; R₁′ or R₂′ each is independently selected from the group consisting of i, ii, iiia, iv, v, and vi:

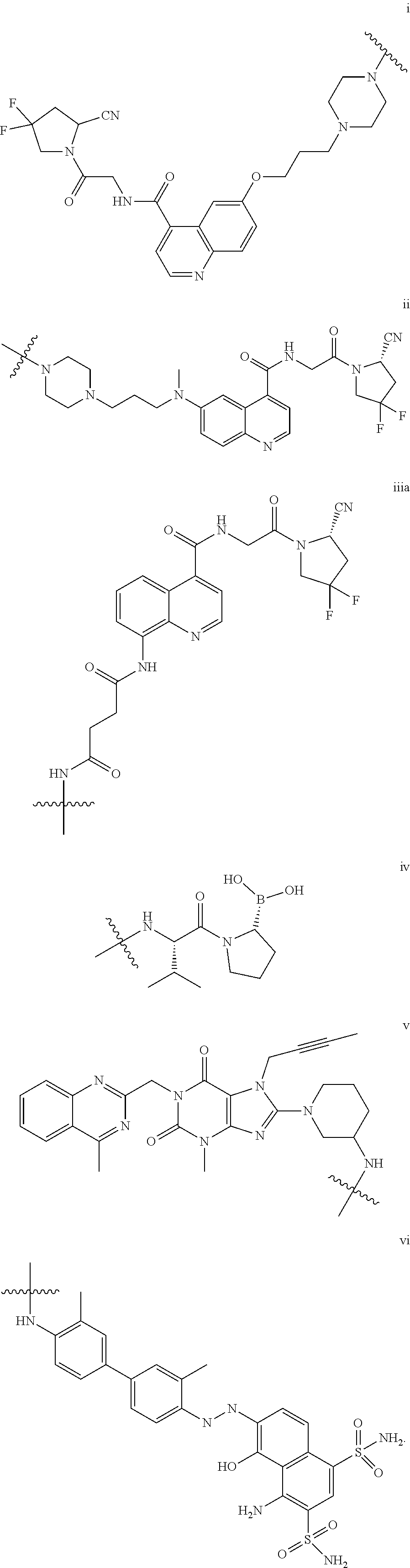

In yet another aspect, the compound comprises Formula (IIa), Formula (IIIa), or a tautomer, an enantiomer, a diastereomer, or a derivative thereof.

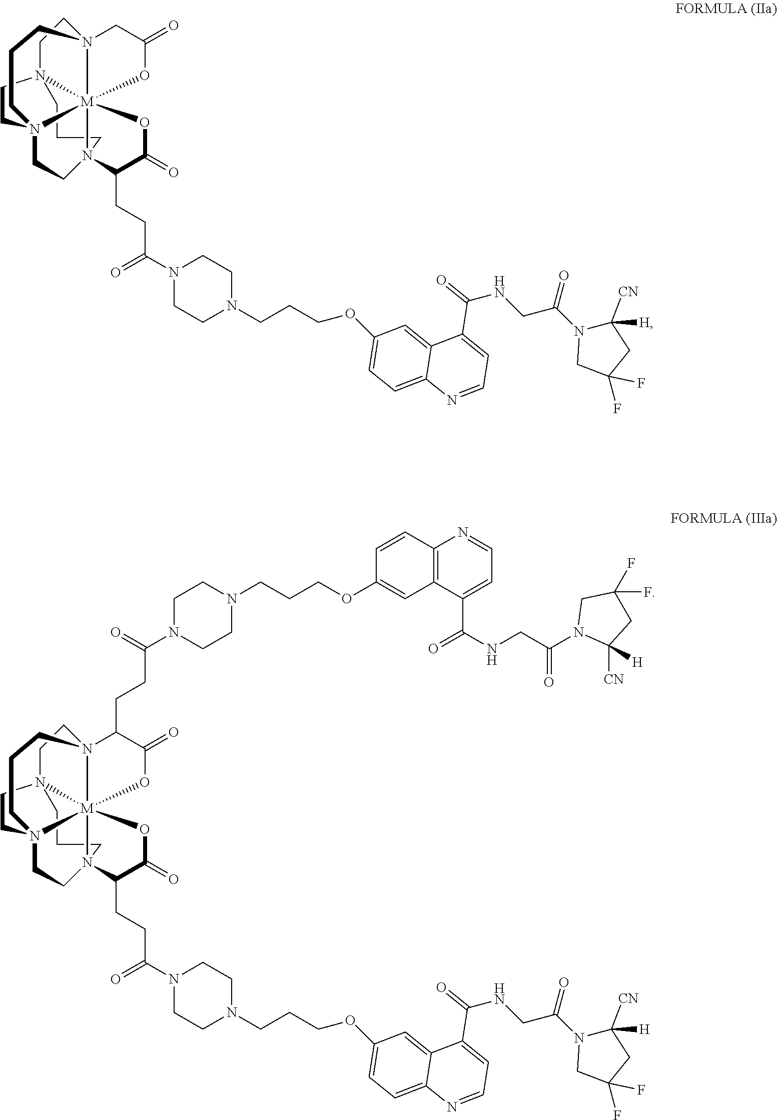

Another aspect of the present disclosure relates to a pharmaceutical composition comprising the compound disclosed above and at least one pharmaceutically acceptable excipient or carrier.

In yet another aspect, the present disclosure relates to a method of preparing the compound of Formula (I) through following steps:

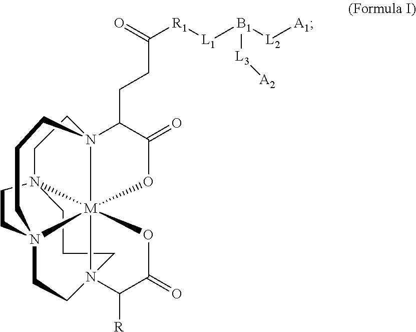

(a) contacting the compound of Formula (LXIV):

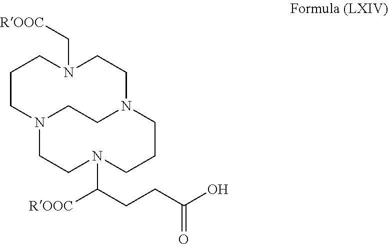

with the compound of Formula (LXV):

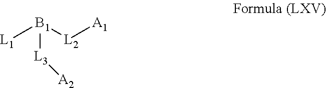

in the presence of an acyl coupling reagent and proton acceptor to produce the compound of Formula (LXVI):

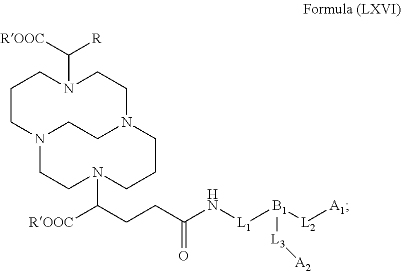

(b) contacting the produced compound of Formula (LXVI) with a proton acceptor or a proton donor to produce the compound of Formula (LXVII).

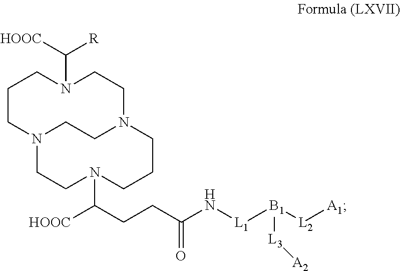

(c) contact the produced compound of Formula (LXVII) with a M-salt in the presence of a buffer to form the compound of Formula (I).

In Formula (I), A₁ is a Fibroblast Activated Protein (FAP) targeting group (FAPI) or an albumin binder (AB) group, wherein the FAPI or the AB group is capable of covalent or non-covalent binding with the FAP or albumin respectively; A₂ is a Fibroblast Activated Protein (FAP) targeting group (FAPI), an albumin binder (AB) group, or absent, wherein the FAPI or the AB group is capable of covalent or non-covalent binding with the FAP or albumin respectively; R₁ is —NH, piperazine, piperidine, an alkylamine, a dicarboxylate amide, or is absent; L₁ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof; L₂ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent; L₃ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent; B₁ is a heterocyclic group, a heterocyclic alkyl, an amidoalkyl, an alkylamide, or is absent; and R is a H hydrogen or Formula (Ia):

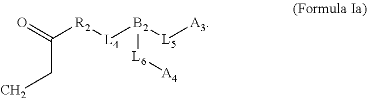

In Formula (Ia), A₃ is a Fibroblast Activated Protein (FAP) targeting group (FAPI) or an albumin binder (AB) group, wherein the FAPI or the AB group is capable of covalent or non-covalent binding with the FAP or albumin respectively; A₄ is a Fibroblast Activated Protein (FAP) targeting group (FAPI), an albumin binder (AB) group, or absent, wherein the FAP targeting group (FAPI) or the AB group is capable of covalent or non-covalent binding with the FAP or albumin respectively; R₂ is —NH, piperazine, piperidine, an alkylamine, a dicarboxylate amide, or is absent; L₄ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof; L₅ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent; L₆ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent; B₂ is a heterocyclic group, a heterocyclic alkyl, an amidoalkyl, an alkylamide, or is absent; M is ⁶⁴Cu, ⁶⁰Cu, ⁶¹Cu, ⁶²Cu or ⁶⁷Cu. In one aspect, the above method yields the compound of Formula (I) in a yield greater than 60%. In another aspect, the above method yields the compound of Formula (I) in greater than 95% radiochemical purity.

In another aspect, the present disclosure relates to a method of treating and/or diagnosing of a disease characterized by overexpression of fibroblast activation protein (FAP), the method comprising administering an effective amount of a compound according to the above compounds or a pharmaceutical composition comprising such compound to a subject in need thereof. In another aspect, the method is a method of treating a disease and M is ⁶⁷Cu. In yet another aspect, the method is a method of diagnosing a disease and M is ⁶⁴Cu, ⁶⁰Cu, ⁶¹Cu, ⁶²Cu or ⁶⁷Cu. In yet another aspect, the method relates to treat or diagnose a disease characterized by overexpression of fibroblast activation protein (FAP) selected from cancer, chronic inflammation, atherosclerosis, fibrosis, tissue remodeling and keloid disorder, breast cancer, renal cell carcinoma, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer. In yet another aspect, the method relates to treat or diagnose renal cell carcinoma, and the method comprises imaging evaluation of the subject. In one aspect, such imaging evaluation comprises one or more of a positron emission tomography (PET), or single photon emission computer tomography (SPECT). In yet another aspect, the method further comprises administering additional treatments including but not restricted to chemotherapy, radiation therapy and/or drugs before, simultaneously or after the pharmaceutical composition. In one aspect, the subject is a human. In yet another aspect, the subject is diagnosed with or is suspected of having a disease characterized by overexpression of fibroblast activation protein (FAP). In yet another aspect, the method is for a subject being diagnosed with or suspected of having any one or more of a cancer, chronic inflammation, atherosclerosis, fibrosis, tissue remodeling and keloid disorder, breast cancer, renal cell carcinoma, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.

In one aspect, the present disclosure relates to use one or more of the above compounds as a radiopharmaceutical active or a radiotheranostic active. In another aspect, the present disclosure relates to use one or more of the above compounds in the manufacture of a medicament for radiopharmaceutical therapy or radiotheranostic therapy.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered to be limiting of its scope. Further, the application contains at least one drawing executed in color. The color drawings are needed to fully convey to the reader the subject matter sought to be patented. Copies of this patent application publication with color drawing(s) will be provided by the Office upon request and payment of the necessary fee. Aspects of the present inventive concept are illustrated by way of example in which like reference numerals indicate similar elements and in which:

FIG. 1 shows the structure of FAPI and the molecular design of copper-based radiopharmaceuticals targeting FAP.

FIG. 2A provides structures of synthesized FAP targeting monovalent and divalent precursor conjugates: CB-TE2A-FAPI and CB-TE2A-(FAPI)₂.

FIG. 2B provides the synthetic route for monovalent and divalent cross-bridged cyclam and FAPI conjugates. Reagents and reaction condition for monovalent conjugate include FAPI (1.2 equiv.), CB-TE2A-(tBu)₂-COOH (1 equiv.), HBTU (1 equiv.), DIPEA (5 equiv.), TFA reaction for 6 hours. Reagents and reaction condition for divalent conjugate include FAPI (3.5 equiv.), CB-TE2A-(tBu)₂-(COOH)₂ (1 equiv.), HBTU (2 equiv.), DIPEA (10 equiv.), TFA reaction for 3 hours.

FIG. 3 provides the synthetic route to Boc-FAPI.

FIG. 4A shows a Western blot analysis of FAP on XP185.

FIG. 4B shows an immunohistochemistry staining of XP-185 tumor xenograft using FAP-α-antibody.

FIG. 5 shows the radiolabeling reaction of monovalent and divalent CB-TE2A FAPI conjugates with ⁶⁴CuCl₂

FIG. 6 provides data for in vitro serum stability of ⁶⁴Cu-CB-TE2A-(FAPI)₂.

FIG. 7A provides microPET-CT images of XP-185 tumor bearing mice (n=4) at 0-10 min, 40-50 min, 4-hr, and 24-hr after intravenous injection of divalent conjugate ⁶⁴Cu-CB-TE2A-(FAPI)₂ (left four images in left panel) and Images obtained with co-injection of 10 μg/kg of FAPI are shown for 1-hr and 4-hr time points (right two images from left panel).

FIG. 7B provides Micro PET-CT images of XP-185 tumor bearing mice (n=3) at 1-hr and 24-hr after intravenous injection of monovalent conjugate ⁶⁴Cu-CB-TE2A-(FAPI) (two images in right panel). Arrows indicate tumors.

FIG. 8A shows bar graphs with quantitative biodistribution of ⁶⁴Cu-CB-TE2A-(FAPI)₂ at different time points after intravenous administration in XP-185 tumor bearing mice (n=4).

FIG. 8B is a bar graph showing the tumor uptake of ⁶⁴Cu-CB-TE2A-(FAPI)₂ after 4-hr post injection compared to blockade experiment with FAPI.

FIG. 8C shows quantitative biodistribution of ⁶⁴Cu-CB-TE2A-(FAPI) at different time points after intravenous administration in XP-185 tumor bearing mice (n=4).

FIG. 8D shows quantitative biodistribution of ⁶⁴Cu-CBTE2A-(FAPI)₂ at different time points after intravenous administration in XP-185 bearing mice (n=4) in the presence of excess of FAPI.

FIG. 9A shows design of copper-based radiopharmaceuticals targeting FAP.

FIG. 9B depicts MicroPET-CT images of XP-185 tumor graft in mice (n=4) after injected with ⁶⁴Cu-CB-TE2A-(FAPI04)₂ (the blockade group was co-injected with 100-fold cold FAPI04).

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The following detailed description references the accompanying drawings that illustrate various aspects of the present inventive concept. The drawings and description are intended to describe aspects and aspects of the present inventive concept in sufficient detail to enable those skilled in the art to practice the present inventive concept. Other components can be utilized, and changes can be made without departing from the scope of the present inventive concept. The following description is, therefore, not to be taken in a limiting sense.

### I. TERMINOLOGY

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to aspects of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising”, “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or C”; “A and B”; “A and C”; “B and C”; “A, B and C.” An exception to this definition will occur only when a combination of elements, functions, steps, or acts are in some way inherently mutually exclusive.

In the following definitions of the terms: alkyl, heteroalkyl, cycloalkyl, heterocycloalkyl, aryl, aralkyl, heteroaryl, heteroaralkyl, alkenyl and alkynyl are provided. These terms will in each instance of its use in the remainder of the specification have the respectively defined meaning and preferred meanings.

The term “alkyl” refers to a saturated straight or branched carbon chain. Preferably, the chain comprises from 1 to 10 carbon atoms, i.e., 1, 2, 3, 4, 5, 6, 7, 8, 9 or 10 e.g., methyl, ethyl methyl, ethyl, propyl, iso-propyl, butyl, iso-butyl, tert-butyl, pentyl, isopentyl, tert-pentyl, neopentyl, hexyl, or octyl. Alkyl groups are optionally substituted.

The term “heteroalkyl” refers to a saturated straight or branched carbon chain. Preferably, the chain comprises from 1 to 9 carbon atoms, i.e. 1, 2, 3, 4, 5, 6, 7, 8, 9 e.g. methyl, ethyl, propyl, iso-propyl, butyl, iso-butyl, sec-butyl, tert-butyl, pentyl, hexyl, pentyl, octyl, which is interrupted one or more times, e.g. 1, 2, 3, 4, 5, with the same or different heteroatoms. Preferably the heteroatoms are selected from O, S, and N, e.g. —CH₃, —S—CH₃, —CH₂—O—CH₃, —CH₂—O—C₂H₅, —CH₂—S—CH₃, —CH₂—S—C₂H₅, —C₂H₄—O—CH₃, —C₂H₄—O—C₂H₅, —C₂H₄—S—CH₃, —C₂H₄—S—C₂H₅ etc. Heteroalkyl groups are optionally substituted.

The terms “heterocyclic” or “cycloalkyl” and “heterocycloalkyl”, by themselves or in combination with other terms, represent, unless otherwise stated, cyclic versions of “alkyl” and “heteroalkyl”, respectively, with preferably 3, 4, 5, 6, 7, 8, 9 or 10 atoms forming a ring, e.g., cyclopropyl, cyclobutyl, cyclopentyl, cyclohexyl, cycloheptyl, cyclooctyl, etc. The terms “cycloalkyl” and “heterocycloalkyl” are also meant to include bicyclic, tricyclic, and polycyclic versions thereof. The term “heterocycloalkyl” preferably refers to a saturated ring having five of which at least one member is a N, O or S atom and which optionally contains one additional O or one additional N; a saturated ring having six members of which at least one member is a N, O or S atom and which optionally contains one additional 0 or one additional N or two additional N atoms; or a saturated bicyclic ring having nine or ten members of which at least one member is a N, O or S atom and which optionally contains one, two or three additional N atoms. “Cycloalkyl” and “heterocycloalkyl” groups are optionally substituted. Additionally, for heterocycloalkyl, a heteroatom can occupy the position at which the heterocycle is attached to the remainder of the molecule. Examples of cycloalkyl include cyclopropyl, cyclobutyl, cyclopentyl, cyclohexyl, 1-cyclohexenyl, 3-cyclohexenyl, cycloheptyl, spiro[3,3]heptyl, spiro[3,4]octyl, spiro[4,3]octyl, spiro[3,5]nonyl, spiro[5.3]nonyl, spiro[3,6]decyl, spiro[6,3]decyl, spiro[4,5]decyl, spiro[5,4]decyl, bicyclo[2.2.1]heptyl, bicyclo[2.2.2]octyl, adamantyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, 1.8 diazo-spiro-[4,5]decyl, 1,7 diazo-spiro-[4,5]decyl, 1,6 diazo-spiro-[4.5]decyl, 2,8 diazo-spiro[4,5]decyl, 2,7 diazo-spiro[4,5]decyl, 2,6 diazo-spiro[4,5]decyl, 1,8 diazo-spiro-[5,4]decyl, 1,7 diazo-spiro-[5,4]decyl, 2,8 diazo-spiro-[5,4]decyl, 2,7 diazo-spiro[5,4]decyl, 3,8 diazo-spiro[5,4]decyl, 3,7 diazo-spiro[5,4]decyl, 1-azo-7,11-dioxo-spiro[5,5]undecyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” preferably refers to an aromatic monocyclic ring containing 6 carbon atoms, an aromatic bicyclic ring system containing 10 carbon atoms, or an aromatic tricyclic ring system containing 14 carbon atoms. Examples are phenyl, naphthyl or anthracenyl. The aryl group is optionally substituted.

The term “aralkyl” refers to an alkyl moiety, which is substituted by aryl, wherein alkyl and aryl have the meaning as outlined above. An example is the benzyl radical. Preferably, in this context the alkyl chain comprises from 1 to 8 carbon atoms, i.e., 1, 2, 3, 4, 5, 6, 7, or 8, e.g., methyl, ethyl methyl, ethyl, propyl, iso-propyl, butyl, iso-butyl, sec-butenyl, tert-butyl, pentyl, hexyl, isopentyl, tert-pentyl, neopentyl, octyl. The aralkyl group is optionally substituted at the alkyl and/or aryl part of the group.

The term “heteroaryl” preferably refers to a five or six-membered aromatic monocyclic ring wherein at least one of the carbon atoms are replaced by 1, 2, 3, or 4 (for the five membered ring) or 1, 2, 3, 4, or 5 (for the six membered ring) of the same or different heteroatoms, preferably selected from O, N and S; an aromatic bicyclic ring system wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system wherein 1, 2, 3, 4, 5, or 6 carbon atoms of the 13, 14, 15, or 16 carbon atoms have been replaced with the same or different heteroatoms, preferably selected from O, N and S. Examples are oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, quinoxalinyl, quinazolinyl, quinolinyl, 1,2,3-benzotriazinyl, or 1,2,4-benzotriazinyl.

The term “heteroaralkyl” refers to an alkyl moiety, which is substituted by heteroaryl, wherein alkyl and heteroaryl have the meaning as outlined above. An example is the 2-alklypyridinyl, 3-alkylpyridinyl, or 2-methylpyridinyl. Preferably, in this context the alkyl chain comprises from 1 to 8 carbon atoms, i.e., 1, 2, 3, 4, 5, 6, 7, or 8, e.g., methyl, ethyl methyl, ethyl, propyl, iso-propyl, butyl, iso-butyl, sec-butenyl, tert-butyl, pentyl, hexyl, pentyl, octyl. The heteroaralkyl group is optionally substituted at the alkyl and/or heteroaryl part of the group.

The terms “alkenyl” and “cycloalkenyl” refer to olefinic unsaturated carbon atoms containing chains or rings with one or more double bonds. Examples are propenyl and cyclohexenyl. Preferably, the alkenyl chain comprises from 2 to 8 carbon atoms, i.e., 2, 3, 4, 5, 6, 7, or 8, e.g., ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, pentenyl, octenyl. Preferably the cycloalkenyl ring comprises from 3 to 8 carbon atoms, i.e., 3, 4, 5, 6, 7, or 8, e.g., 1-cyclopropenyl, 2-cyclopropenyl, 1-cyclobutenyl, 2-cylcobutenyl, 1-cyclopentenyl, 2-cyclopentenyl, 3-cyclopentenyl, cyclohexenyl, cyclopentenyl, cyclooctenyl.

The term “alkynyl” refers to unsaturated carbon atoms containing chains or rings with one or more triple bonds. An example is the propargyl radical. Preferably, the alkynyl chain comprises from 2 to 8 carbon atoms, i.e., 2, 3, 4, 5, 6, 7, or 8, e.g., ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl, octynyl.

In one aspect, carbon atoms or hydrogen atoms in alkyl, heteroalkyl, cycloalkyl, aryl, aralkyl, alkenyl, cycloalkenyl, alkynyl radicals may be substituted independently from each other with one or more elements selected from the group consisting of O, S, N, P or with groups containing one or more elements selected from the group consisting of O, S, N and P.

Aspects include alkoxy, cycloalkoxy, arykoxy, aralkoxy, alkenyloxy, cycloalkenyloxy, alkynyloxy, alkylthio, cycloalkylthio, arylthio, aralkylthio, alkenylthio, cycloalkenylthio, alkynylthio, alkylamino, cycloalkylamino, arylamino, aralkylamino, alkenylamino, cycloalkenylamino, alkynylamino radicals.

Other aspects include hydroxyalkyl, hydroxycycloalkyl, hydroxyaryl, hydroxyaralkyl, hydroxyalkenyl, hydroxycycloalkenyl, hydroxyalinyl, mercaptoalkyl, mercaptocycloalkyk, mercaptoaryl, mercaptoaralkyl, mercaptoalkenyl, mercaptocycloalkenyl, mercaptoalkynyl, aminoalkyl, aminocycloalkyl, aminoaryl, aminoaralkyl, aminoalkenyl, aminocycloalkenyl, aminoalkynyl radicals.

In another aspect, hydrogen atoms in alkyl, heteroalkyl, cycloalkyl, aryl, aralkyl, alkenyl, cycloalkenyl, alkynyl radicals may be substituted independently from each other with one or more halogen atoms. One radical is the trifluoromethyl radical.

If two or more radicals or two or more residues can be selected independently from each other, then the term “independently” means that the radicals or the residues may be the same or may be different.

As used herein a wording defining the limits of a range of length such as, e.g., “from 1 to 6” means any integer from 1 to 6, i.e., 1, 2, 3, 4, 5 and 6. In other words, any range defined by two integers explicitly mentioned is meant to comprise and disclose any integer defining said limits and any integer comprised in said range.

The term “halo” as used herein refers to a halogen residue selected from the group consisting of F, Br, I and Cl. Preferably, the halogen is F. If there is more than one halo atoms, each one is independently selected from F, Br, I and Cl.

The “alkyl” containing moieties, such as termed as alkylamine, alkylamide, alkylcarbonyl, and alkylamidoalkylcarbonyl, each independently refers to a moiety with an alkyl radical connected to the functional groups of amine, amide, carbonyl and amindocarbonyl, respectively, in any positions that are feasible.

The term “PEG” refers to polyethylene glycol with various lengths, such as with repeating units ranging from 1 to 25. One aspect of the disclosure comprises a PEG molecule with repeating units of 1 to 12. A PEG alkyl refers to a PEG chain connected to an alkyl moiety in any position feasible. A triazine-PEG refers to a PEG chain connected to a triazine in either carbon or nitrogen, or both in any position of the chain. The triazine includes any of the three (3) positional isomers, i.e., 1,2,3-triazine, 1,2,4-triazine, and 1,3,5-triazine. A functionalized PEG or a functionalized PEG linker comprises one or more functional groups, such as amine, amide, carbonyl, carboxylate, heterocyclic, arylheterocyclic, amidoalkylcarbonyl, amidoalkylcarboxylate, alkylamine, alkylamide, alkylcarbony, alkylcarboxylate, heterocyclic alkyl, or any combinations thereof.

The term “linker” as used herein refers to any chemically suitable linker. Linker can be cleavable or non-cleavable, Preferably, linker is not or only slowly cleaved under physiological conditions. Thus, it is preferred that the linker does not comprise recognition sequences for proteases or recognition structures for other degrading enzymes. Since it is preferred that the compounds of the disclosure are administered systemically to allow broad access to all compartments of the body and subsequently enrichment of the compounds of the disclosure wherever in the body the tumor is located, it is preferred that the linker is chosen in such that it is not or only slowly cleaved in blood. The cleavage is considered slowly, if less than 50% of the linkers are cleaved 2 h after administration of the compound to a human patient. Suitable linkers, for example, comprises or consists of optionally substituted alkyl, heteroalkyl, cycloalkyl, cycloheteroalkyl, aryl, heteroaryl, aralkyl, heteroaralyl, alkenyl, heteroalkenyl, cycloalkenyl, cycloheteroalkenyl, alkynyl, sulfonyl, amines, ethers, thioethers phosphines, phosphoramidates, carboxamides, esters, imidoesters, amidines, thioesters, sulfonamides, 3-thiopyrrolidine-2,5-dion, carbamates, ureas, guanidines, thioureas, disulfides, oximes, hydrazines, hydrazides, hydrazones, diaza bonds, triazoles, triazolines, tetrazines, platinum complexes and amino acids, or combinations thereof. In some aspects, the linker comprises or consists of 1,4-piperazine, 1,3-propane and a phenolic ether or combinations thereof. In some aspects, the linker comprises an alkyl, a peptide, a functionalized polyethylene glycol (PEG), or any combination thereof.

The expression “optionally substituted” refers to a group in which one, two, three or more hydrogen atoms may have been replaced independently of each other by the respective substituents.

As used herein, the term “amino acid” refers to any organic acid containing one or more amino substituents, e.g., α-, β- or γ-amino, derivatives of aliphatic carboxylic acids. In the polypeptide notation used herein, e.g., Xaa5, i.e., Xaa1Xaa2Xaa3Xaa4Xaa5, wherein Xaa1 to Xaa5 are each and independently selected from amino acids as defined, the left-hand direction is the amino terminal direction and the right-hand direction is the carboxy terminal direction, in accordance with standard usage and convention.

The term “conventional amino acid” refers to the twenty naturally occurring amino acids, and encompasses all stereo-isoforms, i.e., D-, L-, D- and L-amino acids thereof. These conventional amino acids can herein also be referred to by their conventional three-letter or one-letter abbreviations and their abbreviations follow conventional usage (see, for example, Immunology—A Synthesis, 2nd Edition, E. S. Golub and D. R. Gren, Eds., Sinauer Associates, Sunderland Mass. (1991)).

The term “non-conventional amino acid” refers to unnatural amino acids or chemical amino acid analogues, e.g., α,α-disubstituted amino acids, N-alkyl amino acids, homo-amino acids, dehydroamino acids, aromatic amino acids (other than phenylalanine, tyrosine and tryptophan), and ortho-, meta- or para-aminobenzoic acid. Non-conventional amino acids also include compounds which have an amine and carboxyl functional group separated in a 1,3 or larger substitution pattern, such as β-alanine, γ-amino butyric acid, Freidinger lactam, the bicyclic dipeptide (BTD), amino-methyl benzoic acid and others well known in the art. Statin-like isosteres, hydroxyethylene isosteres, reduced amide bond isosteres, thioamide isosteres, urea isosteres, carbamate isosteres, thioether isosteres, vinyl isosteres and other amide bond isosteres known to the art may also be used. The use of analogues or non-conventional amino acids may improve the stability and biological half-life of the added peptide since they are more resistant to breakdown under physiological conditions. The person skilled in the art will be aware of similar types of substitution which may be made.

The term “peptide” refers to a plurality of amino acids. The term peptide refers to peptide, polypeptide, or protein, native or naturally occurring, or biologically active variants or fragments thereof. The term “peptidomimetic” refers to a compound comprising a plurality of amino acid residues joined by a plurality of peptide bonds which are modified by addition, deletion, or modification of one or more linear or branched amino acids. A peptide alkyl or a peptidomimetic alkyl refers to an alkyl moiety connected to the peptide or peptidomimetic chain in any feasible positions.

The term “N-containing aromatic or non-aromatic mono or bicyclic heterocycle” as used herein refers to a cyclic saturated or unsaturated hydrocarbon compound which contains at least one nitrogen atom as constituent of the cyclic chain.

The term “radioactive moiety” as used herein refers to a molecular assembly which carries a radioactive nuclide. The nuclide is bound either by covalent or coordinate bonds which remain stable under physiological conditions.

A “fluorescent isotope” as used herein emits electromagnetic radiation after excitation by electromagnetic radiation of a shorter wavelength.

A “radioisotope” as used herein is a radioactive isotope of an element (included by the term “radionuclide”) emitting α-, β-, and/or γ-radiation.

The term “radioactive drug” is used in the context of the present disclosure to refer to a biologic active compound which is modified by a radioisotope. Especially intercalating substances can be used to deliver the radioactivity to direct proximity of DNA.

The term “chelating agent” or “chelate” are used interchangeably in the context of the present disclosure and refer to a molecule, often an organic one, and often a Lewis base, having two or more unshared electron pairs available for donation to a metal ion. The metal ion is usually coordinated by two or more electron pairs to the chelating agent. The terms, “bidentate chelating agent”, “tridentate chelating agent, and “tetradentate chelating agent” refer to chelating agents having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible.

The term “fluorescent dye” is used in the context of the present disclosure to refer to a compound that emits visible or infrared light after excitation by electromagnetic radiation of a shorter and suitable wavelength. It is understood by the skilled person, that each fluorescent dye has a predetermined excitation wavelength.

The term “contrast agent” is used in the context of the present disclosure to refer to a compound which increases the contrast of structures or fluids in medical imaging. The enhancement is achieved by absorbing electromagnetic radiation or altering electromagnetic fields.

The term “paramagnetic” as used herein refers to paramagnetism induced by unpaired electrons in a medium. A paramagnetic substance induces a magnetic field if an external magnetic field is applied. Unlike diamagnetism the direction of the induced field is the same as the external field and unlike ferromagnetism the field is not maintained in absence of an external field.

The term “nanoparticle” as used herein refers to particles preferably of spheric shape, with diameters of sizes between 1 and 100 nanometers. Depending on the composition, nanoparticles can possess magnetic, optical, or physio-chemical qualities that can be assessed. Additionally surface modification is achievable for many types of nanoparticles.

The term “pharmaceutically acceptable salt” refers to a salt of the compound of the present disclosure. Suitable pharmaceutically acceptable salts of the compound of the present disclosure include acid addition salts which may, for example, be formed by mixing a solution of choline or derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid, or phosphoric acid. Furthermore, where the compound of the disclosure carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counter-anions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide a compound of formula (I). A prodrug is an active or inactive compound that is modified chemically through in vivo physiological action, such as hydrolysis, metabolism, and the like, into a compound of this disclosure following administration of the prodrug to a patient. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme. The suitability and techniques involved in making and using prodrugs are well known by those skilled in the art. For a general discussion of prodrugs involving esters see Svensson and Tunek Drug Metabolism Reviews 16.5 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole, and the like, have been masked with N-acyloxymethyl groups (Bundgaard Design of Prodrugs, Elsevier (1985)). Hydroxyl groups have been masked as esters and ethers. EP 0 039 051 (Sloan and Little, Apr. 11, 1981) discloses Mannich-base hydroxamic acid prodrugs, their preparation and use.

Compounds according to the disclosure can be synthesized according to one or more of the following methods. It should be noted that the general procedures are shown as it relates to preparation of compounds having unspecified stereochemistry. However, such procedures are generally applicable to those compounds of a specific stereochemistry, e.g., where the stereochemistry about a group is (S) or (R), or the compound is (−) or (+). In addition, the compounds having one stereochemistry (e.g., (R)) can often be utilized to produce those having opposite stereochemistry (i.e., (S)) using well-known methods, for example, by inversion.

Certain compounds of the present disclosure can exist in unsolvated forms as well as in solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present disclosure possess asymmetric carbon atoms (optical centers) or double bonds; the racemates, diastereomers, geometric isomers and individual isomers are all intended to be encompassed within the scope of the present disclosure.

The compounds of the present disclosure may also contain unnatural proportions of atomic isotopes or radionuclides at one or more of the atoms that constitute such compounds. For example, the compounds may be radiolabeled with radioactive copper isotopes, such as for example may be ⁶⁰Cu, ⁶¹Cu, ⁶²Cu, ⁶⁴Cu, and ⁶⁷Cu. All isotopic variations of the compounds of the present disclosure, whether radioactive or not, are intended to be encompassed within the scope of the present disclosure.

The term “chelating agent” or “chelate” are used interchangeably in the context of the present disclosure and refer to a molecule, often an organic one, and often a Lewis base, having two or more unshared electron pairs available for donation to a metal ion. The metal ion is usually coordinated by two or more electron pairs to the chelating agent. The terms, “bidentate chelating agent”, “tridentate chelating agent, and “tetradentate chelating agent” refer to chelating agents having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible. In some aspects, the chelating agent in the current disclosure may be bicyclo[6.6.2]tetraamine (CB-TE2A) ligands.

The term “pharmaceutical composition” as used in the present application refers to a substance and/or a combination of substances being used for the identification, prevention or treatment of a tissue status or disease. The pharmaceutical composition is formulated to be suitable for administration to a patient in order to detect, prevent and/or treat disease. Further a pharmaceutical composition refers to the combination of an active agent with a carrier or excipient, inert or active, making the composition suitable for diagnostic and/or therapeutic use. Pharmaceutical compositions can be formulated for parenteral application routes according to their chemical and physical properties. Such parenteral composition may be a liquid composition and may be suitable for either infusion or injection. Such parenteral administration may include sterile solution for injection, sterile suspension for injection, sterile emulsions, and dispersions. Such parenteral composition may comprise a carrier or an excipient, such as one or more chelating agents, a calcium ion, isotonic agents, carriers, buffers, co-solvents, diluents, preservatives, and/or surfactants, or combinations thereof. In certain aspects, the carrier or the excipient is an aqueous carrier. And such aqueous carrier includes, but are not limited to sterile water, water for injection, sodium chloride, Ringer's injection, isotonic dextrose injection, dextrose and lactated Ringer's injection. In some aspect, the parenteral composition comprises water for injection. In some aspect, the water for injection further comprises calcium EDTA or a calcium salt EDTA derivative, or sodium chloride in an amount such that the final solution is isotonic (e.g., 0.1%, 0.25%, 0.45% 0.65%, 0.9% sodium chloride). In some embodiments, the sodium chloride is present in an isotonic amount, such that final concentration of sodium chloride is 0.65%.

“Pharmaceutically acceptable” means approved by a regulatory agency of the Federal or a state government or listed in the U.S. Pharmacopeia or other generally recognized pharmacopeia for use in animals, and more particularly in humans.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable, or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Examples of suitable pharmaceutical carriers are described in “Remington's Pharmaceutical Sciences” by E. W. Martin.

As used herein, the term “detecting” refers to identifying the presence of a cancer. Various characteristics of the cancer may be measured (i.e., detected, determined, etc.). For example, the prevalence, volume, size, location, shape, position, etc., of the cancer may, but need not be, measured (i.e., detected, determined) using a variety of methods standard in the art.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Albumin binder (AB) is capable of binding to albumin either covalently or non-covalent binding with the albumin.

Fibroblast activation protein (FAP) as used herein is also known under the term “seprase”. Both terms can be used interchangeably herein. Fibroblast activation protein is a homodimeric integral protein with dipeptidyl peptidase IV (DPPIV)-like fold, featuring an alpha/beta-hydrolase domain and an eight-bladed beta-propeller domain. FAP overexpression is seen in a number of disease conditions including but not restricted to cancer, chronic inflammation, atherosclerosis, fibrosis, tissue remodeling and keloid disorder. The term “Fibroblast Activated Protein (FAP) targeting group”, or “Fibroblast Activated Protein FAP inhibitor” collectively expressed as “FAPI” and refers to any molecule that specifically binds and/or inhibits FAP. One such example is Fibroblast Activation Protein Inhibitor UAMC-1110 emerging as a selective FAP targeting moiety in many radiotracers over the past few years. FAPI-04 contains the inhibitor UAMC-1110 along with a chelator. Recent advances have been made in diagnostic utility using FAPI-04 and other variants including peptide-based radiotracers linked with shorter half-life isotope like ⁶⁸Ga for cancer imaging purposes. In some aspects, the inhibitors in the current disclosure have several desirable properties like specific uptake by FAP expressing tumors, rapid internalization and successful imaging of tumors. Multiple inhibitors of FAP are available. FAPI may comprise TGA, TGB, OncoFAP, Val-BoroPro, or Linagliptin as shown below:

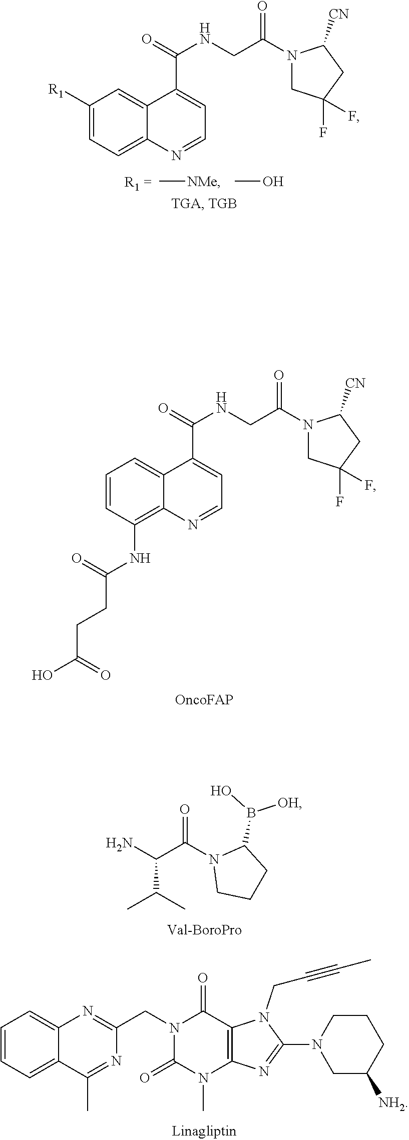

### II. COMPOUNDS AND COMPOSITIONS

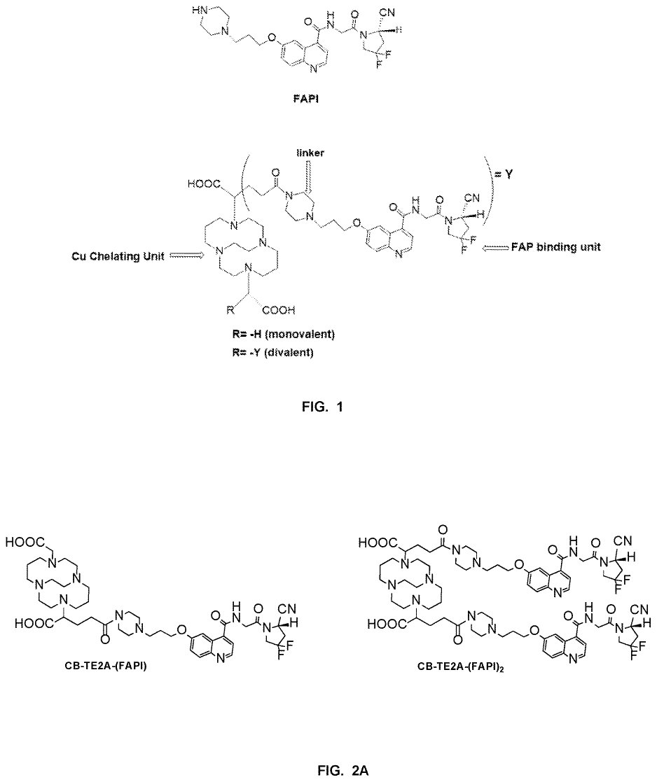

In some aspects, the current disclosure is based on the surprising result that multivalent scaffold with FAP inhibitor appended to stable and neutral bifunctional ^{64/67}Cu-CB-TE2A complex (FIG. 1) can result in enhanced specific binding and tumor retention of FAP-targeted theranostics. FAP-targeted theranostics applications, thus far have suffered from the drawback that existing quinolone-based radiotracers have extremely fast clearance rates from the targeted sites due to numerous Cu-chelating proteins present in vivo. The compositions disclosed herein effectively overcome these draw backs by using bicyclo[6.6.2]tetraamine (CB-TE2A) ligands that provide optimal chelation to ⁶⁴Cu²⁺ with improved in vivo stability. Additionally, these multivalent scaffolds allow for the inclusion of ⁶⁷Cu, making the overall radiotracer viable for endoradiotherapy and SPECT imaging, resulting in a complementary feature to diagnostic utility from ⁶⁴Cu-derived radiotracer. Collectively, the proposed radiotracers have widespread potential applications in cancer diagnostic, therapy and theranostic development.

In an exemplary aspect, the compositions presented herein use a multivalency approach with cross-bridged cyclam ligand to accommodate one or more FAP inhibitor designed to target Fibroblast Activation Protein. In some aspects, the current disclosure encompasses a compound or compositions thereof comprising Formula (I), a positional isomer, a stereo isomer, or a pharmaceutically acceptable salt thereof:

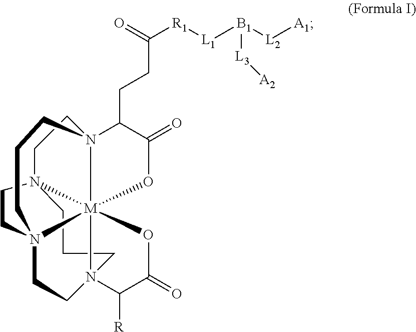

wherein A₁ is a Fibroblast Activated Protein (FAP) targeting group (FAPI) or an albumin binder (AB) group, wherein the FAPI or the AB group is capable of covalent or non-covalent binding with the FAP or albumin respectively;
A₂ is a Fibroblast Activated Protein (FAP) targeting group (FAPI), an albumin binder (AB) group, or absent, wherein the FAPI or the AB group is capable of covalent or non-covalent binding with the FAP or albumin respectively;
R₁ is —NH, piperazine, piperidine, an alkylamine, a dicarboxylate amide, or is absent;
L₁ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof;
L₂ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent;
L₃ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent;
B₁ is a heterocyclic group, a heterocyclic alkyl, an amidoalkyl, an alkylamide, or is absent;
R is a H or Formula (Ia):

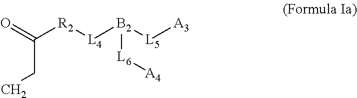

wherein,
   A₃ is a Fibroblast Activated Protein (FAP) targeting group (FAPI) or an albumin binder (AB) group, wherein the FAPI or the AB group is capable of covalent or non-covalent binding with the FAP or albumin respectively;
   A₄ is a Fibroblast Activated Protein (FAP) targeting group (FAPI), an albumin binder (AB) group, or absent, wherein the FAPI or the AB group is capable of covalent or non-covalent binding with the FAP or albumin respectively;
   R₂ is —NH, piperazine, piperidine, an alkylamine, a dicarboxylate amide, or is absent;
   L₄ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof;
   L₅ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent;
   L₆ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent;
   B₂ is a heterocyclic group, a heterocyclic alkyl, an amidoalkyl, an alkylamide, or is absent;
   and
   M is ⁶⁴Cu, ⁶⁰Cu, ⁶¹Cu, ⁶²Cu or ⁶⁷Cu.

In some aspect, the current disclosure encompasses a compound of Formula (I) or compositions thereof, comprising an FAP targeting group for example TGA, TGB, TGC, TGD, TGF, TGE, TGF, TGG, TGH, TGI, and TGJ with structures as provided herein:
targeting group A (TGA):

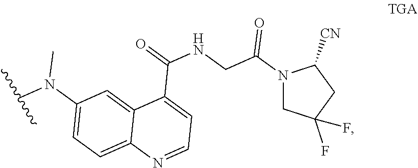 [SMILES: *N(C)c1ccc2nccc(C(=O)NCC(=O)N3CC(F)(F)C[C@H]3C#N)c2c1]

targeting group B (TGB):

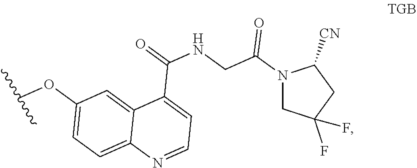 [SMILES: *Oc1ccc2nccc(C(=O)NCC(=O)N3CC(F)(F)C[C@H]3C#N)c2c1]

targeting group C (TGC):

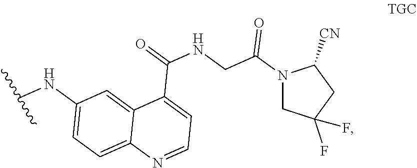 [SMILES: *Nc1ccc2nccc(C(=O)NCC(=O)N3CC(F)(F)C[C@H]3C#N)c2c1]

targeting group D (TGD):

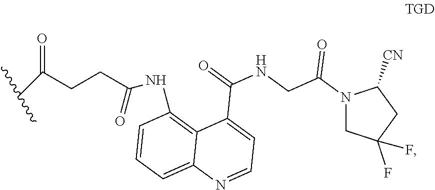 [SMILES: *C(=O)CCC(=O)Nc1cccc2nccc(C(=O)NCC(=O)N3CC(F)(F)C[C@H]3C#N)c12]

targeting group E (TGE):

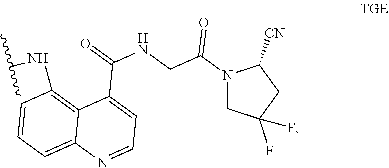 [SMILES: *Nc1cccc2nccc(C(=O)NCC(=O)N3CC(F)(F)C[C@H]3C#N)c12]

targeting group F (TGF):

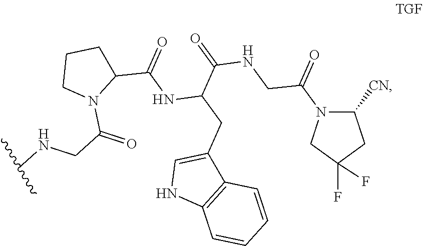 [SMILES: *NCC(=O)N1CCCC1C(=O)NC(Cc1c[nH]c2ccccc12)C(=O)NCC(=O)N1CC(F)(F)C[C@H]1C#N]

targeting group G (TGG):

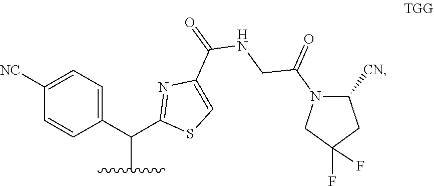 [SMILES: *C(c1ccc(C#N)cc1)c1nc(C(=O)NCC(=O)N2CC(F)(F)C[C@H]2C#N)cs1]

targeting group H (TGH):

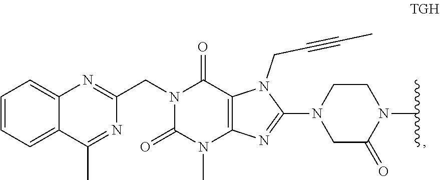 [SMILES: *N1CCN(c2nc3c(c(=O)n(Cc4nc(C)c5ccccc5n4)c(=O)n3C)n2CC#CC)CC1=O]

targeting group I (TGI):

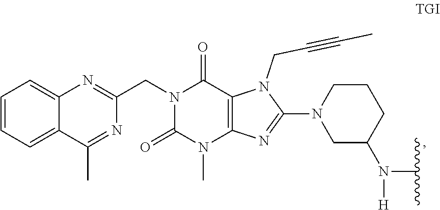 [SMILES: *NC1CCCN(c2nc3c(c(=O)n(Cc4nc(C)c5ccccc5n4)c(=O)n3C)n2CC#CC)C1]

targeting group J (TGJ):

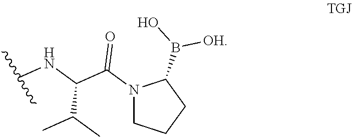 [SMILES: *N[C@H](C(=O)N1CCC[C@H]1B(O)O)C(C)C]

In some aspects, the current disclosure encompasses a compound of Formula (I) or compositions thereof, comprising an AB binding group, wherein the AB binding group may be, for example any of the following moieties:

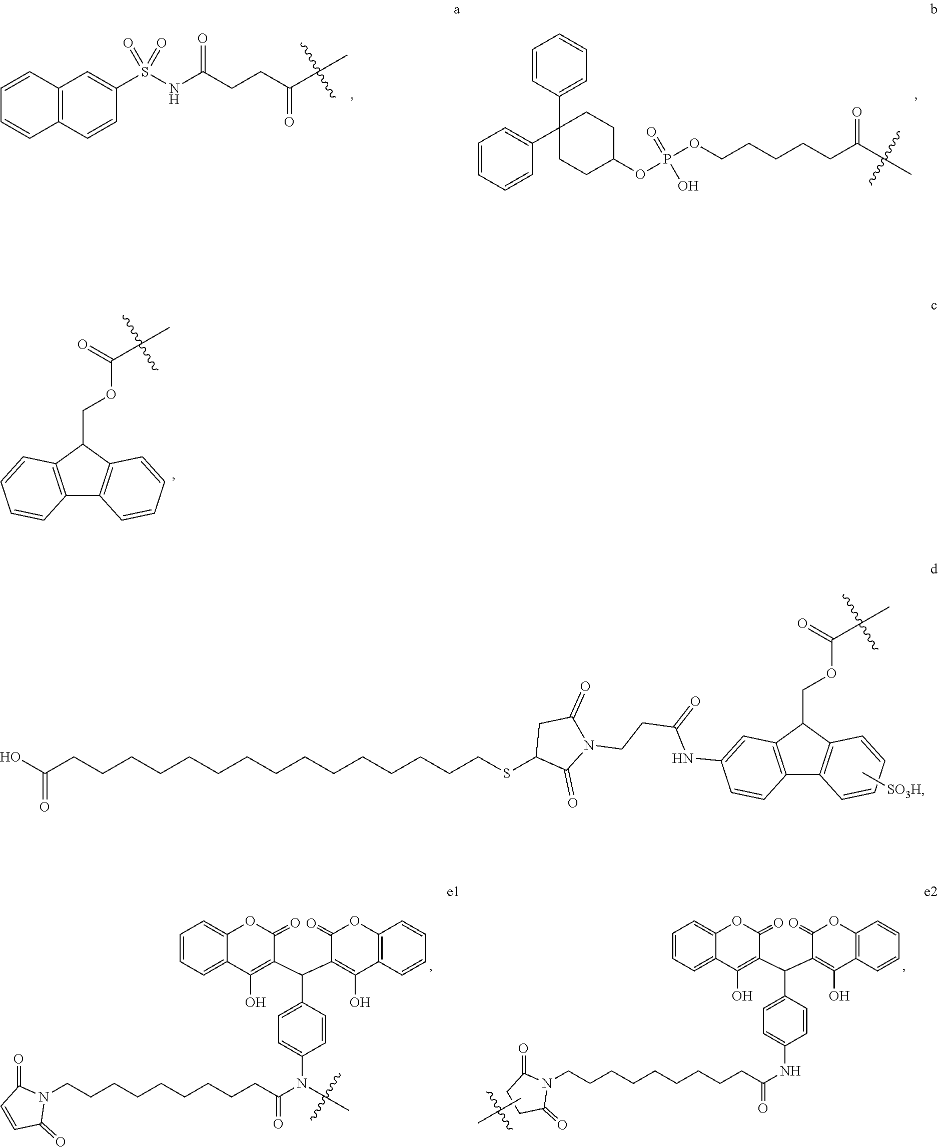

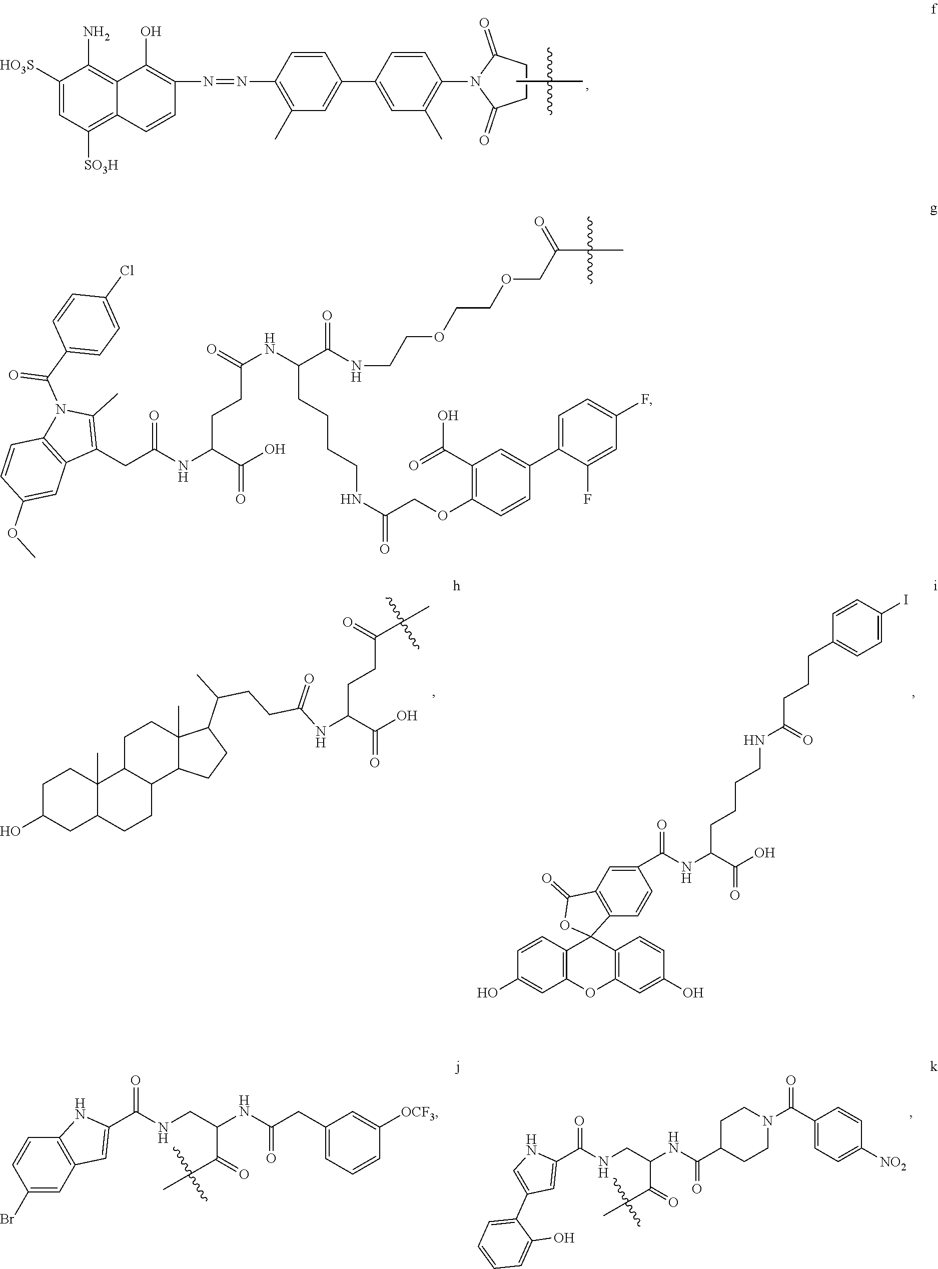

or a variant thereof.

In some aspects, the compounds disclosed herein further comprise one or more linker molecules L₁, L₂, L₃, L₄, L₅, L₆ or more, wherein the linker molecules are independently selected from a group comprising of an alkyl, an alkylamine, an aminocarboxylate, an alkylcarboxylate, an alkylamide, an alkylcarbonyl, an alkylamidoalkylcarbonyl a peptide, a functionalized polyethylene glycol (PEG), a peptidomimetic, a peptidomimetic alkyl, a triazine-PEG, or any combination thereof. In some aspects, the compound comprises at least one alkyl linker, at least one peptide linker, at least one polyethylene glycol (PEG) linker, a disulfide linker, absent, and any combination thereof. In other aspects, L₁, L₂, L₃, L₄, L₅, L₆, are independently selected from a group consisting of at least one peptide linker comprising about 2 to about 10 amino acid residues or a polyethylene glycol linker comprising from 1 to 12 ethylene glycol repeating units or an alkyl linker, or a disulfide linker, an albumin binding entity, absent, and any combination thereof. In some aspects, L₁, is a polyethylene glycol link comprising from 1 to 12 ethylene glycol repeating units, and L₂, and L₃ are absent; L₁ is absent, L₂ is a peptide linker comprising about 2 to about 10 amino acid residues wherein one of the amino acid residues is lysine, and L₃ is a polyethylene glycol linker comprising from 1 to 12 ethylene glycol repeating units; L₁ is absent; L₂ and L₃ are polyethylene glycol linkers comprising from 1 to 12 ethylene glycol repeating units; L₁ is absent, L₂ is a peptide linker comprising 2 to about 10 amino acid residues wherein one of the amino acid residues is lysine and an albumin binding entity and L₃ is a peptide linker comprising 2 to about amino acid residues wherein one of the amino acid residues is lysine; L₁ is an albumin binding entity and an polyethylene glycol linker comprising from 1 to 12 ethylene glycol repeating units and L₂ and L₃ are absent; L₁, L₂, and L₃ are polyethylene glycol linkers comprising from 1 to 12 ethylene glycol repeating units; L₁ is a polyethylene glycol linker comprising from 1 to 12 ethylene glycol repeating unit and a peptide and L₂ and L₃ are absent; L₁ is a polyethylene glycol linker comprising from 1 to 12 ethylene glycol repeating units and a peptide and L₂ and L₃ are absent; L₁ is a polyethylene glycol linker comprising from 1 to 12 ethylene glycol repeating units, L₂ is a polyethylene glycol linker comprising from 1 to 12 ethylene glycol repeating units, and L₃ is a disulfide linker; L₁ is absent, L₂ is a polyethylene glycol linker comprising from 1 to 12 ethylene glycol repeating units and a disulfide group, and L₃ is a polyethylene glycol linker comprising from 1 to 12 ethylene glycol repeating units; or L₁ is absent, L₂ is a disulfide group and a peptide linker comprising 2 to about 10 amino acid residues wherein one of the amino acid residues is lysine or a peptide linker comprising 2 to about 10 amino acid residues wherein one of the amino acid residues is lysine, an albumin binding entity, and a disulfide group, and L₃ is a peptide linker comprising 2 to about 10 amino acid residues wherein one of the amino acid residues is lysine.

In some aspects, the compounds disclosed herein further comprise a heterocyclic group B₁ and/or B₂, wherein the heterocyclic group is selected from a triazine, including 1,2,3-triazine, 1,2,4-triazine, and 1,3,5-triazine.

In some aspects, the compounds disclosed herein comprise a metal chelator bicyclo[6.6.2]tetraamine (CB-TE2A), effective in binding any one of ⁶⁴Cu, ⁶⁰Cu, ⁶¹Cu, ⁶²Cu or ⁶⁷Cu.

In some aspects, the current disclosure also encompasses compounds Formula (II)-Formula (LXIII) as provided herein or a tautomer, an enantiomer, a diastereomer, or a derivative thereof or a composition thereof:

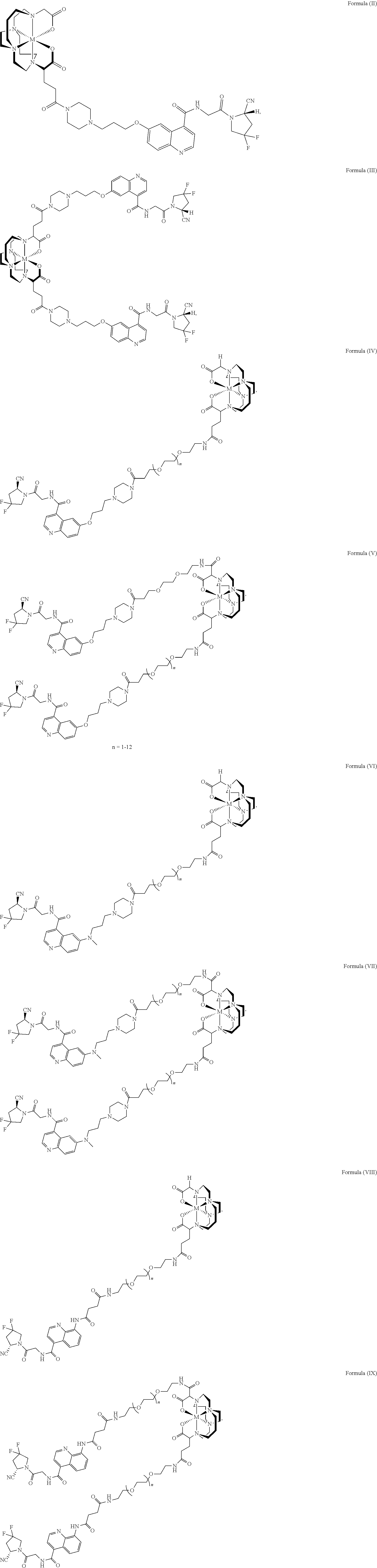

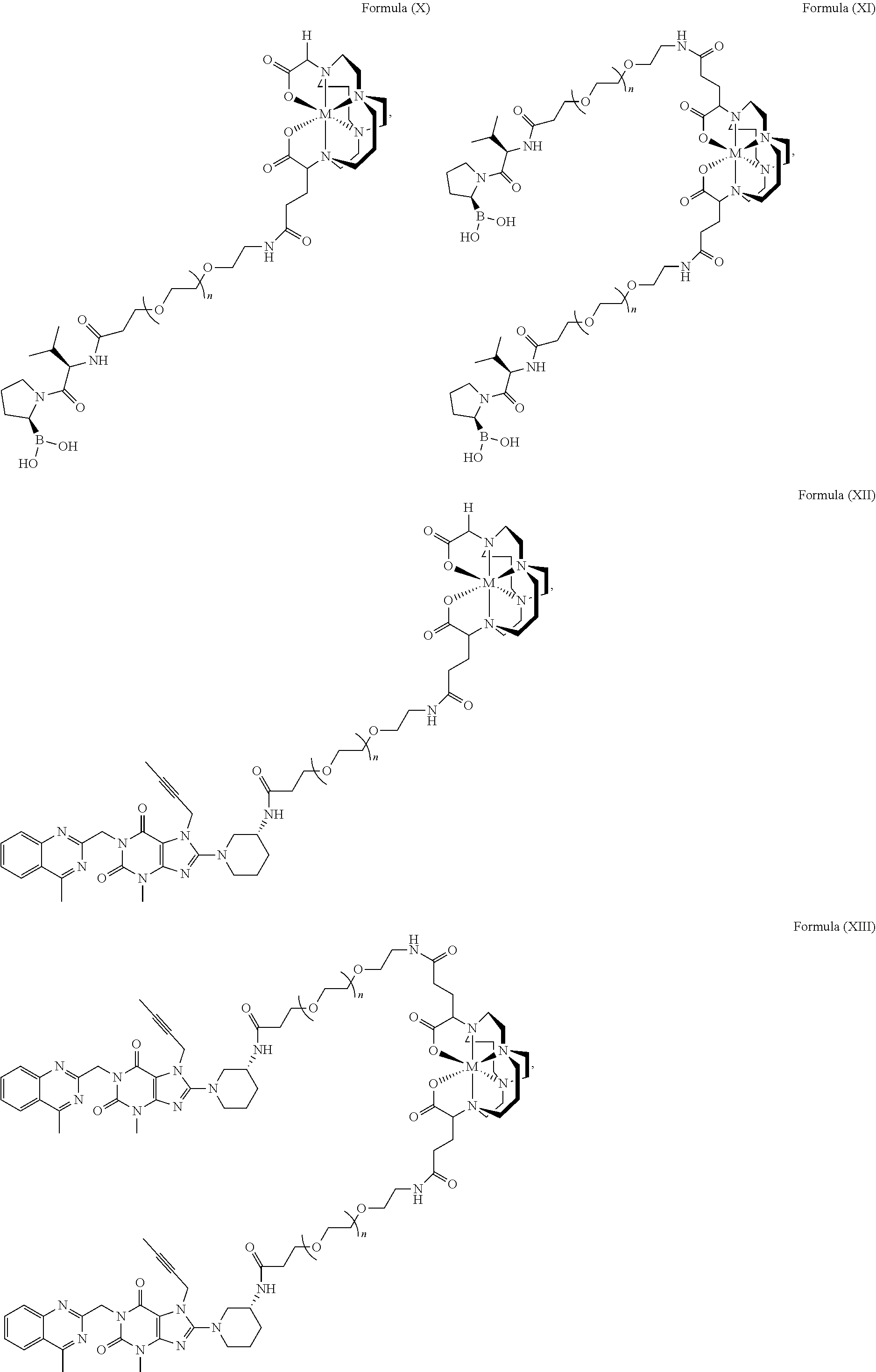

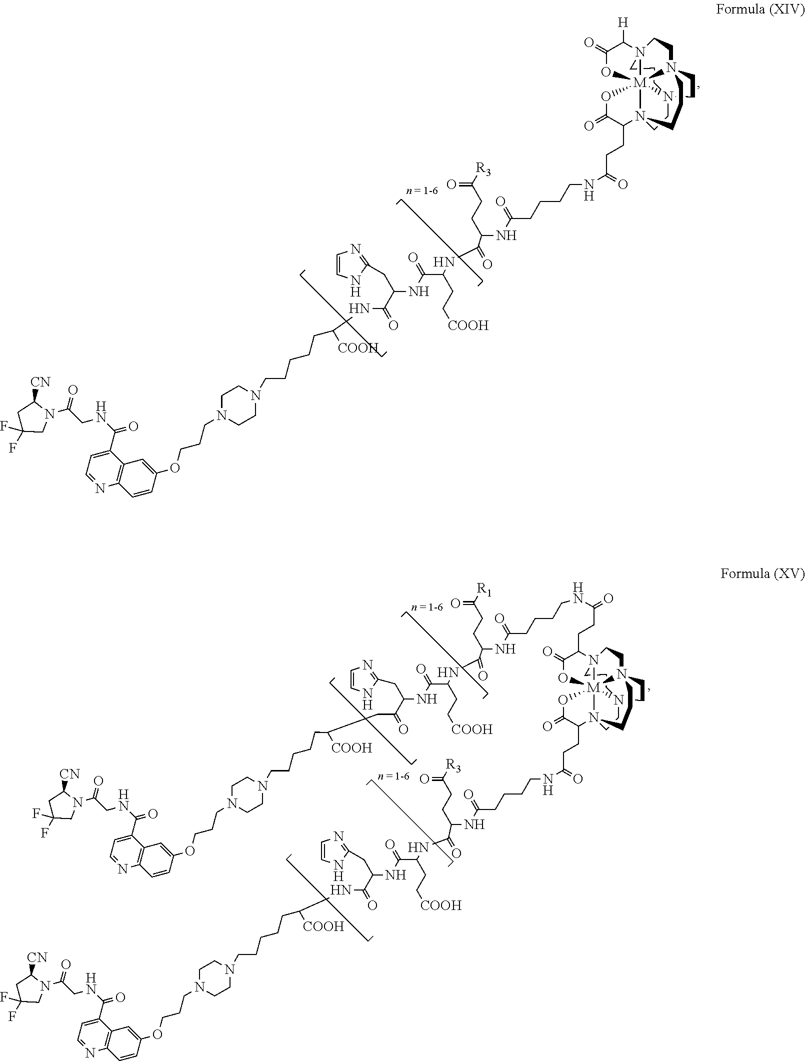

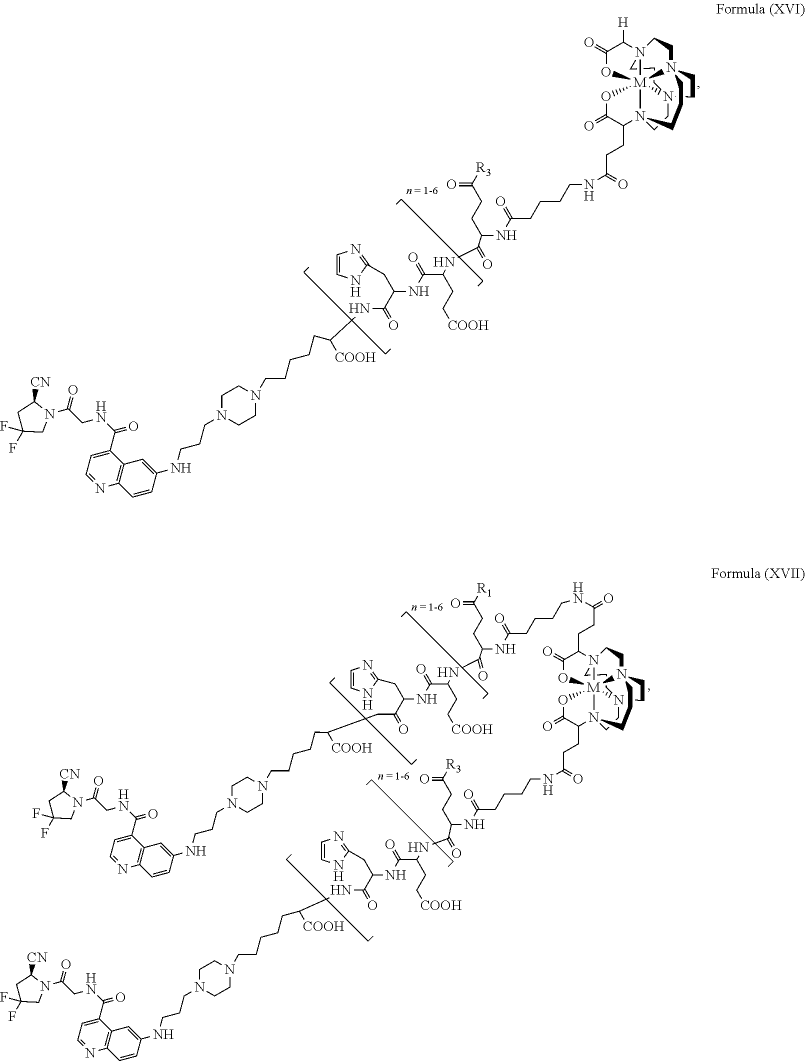

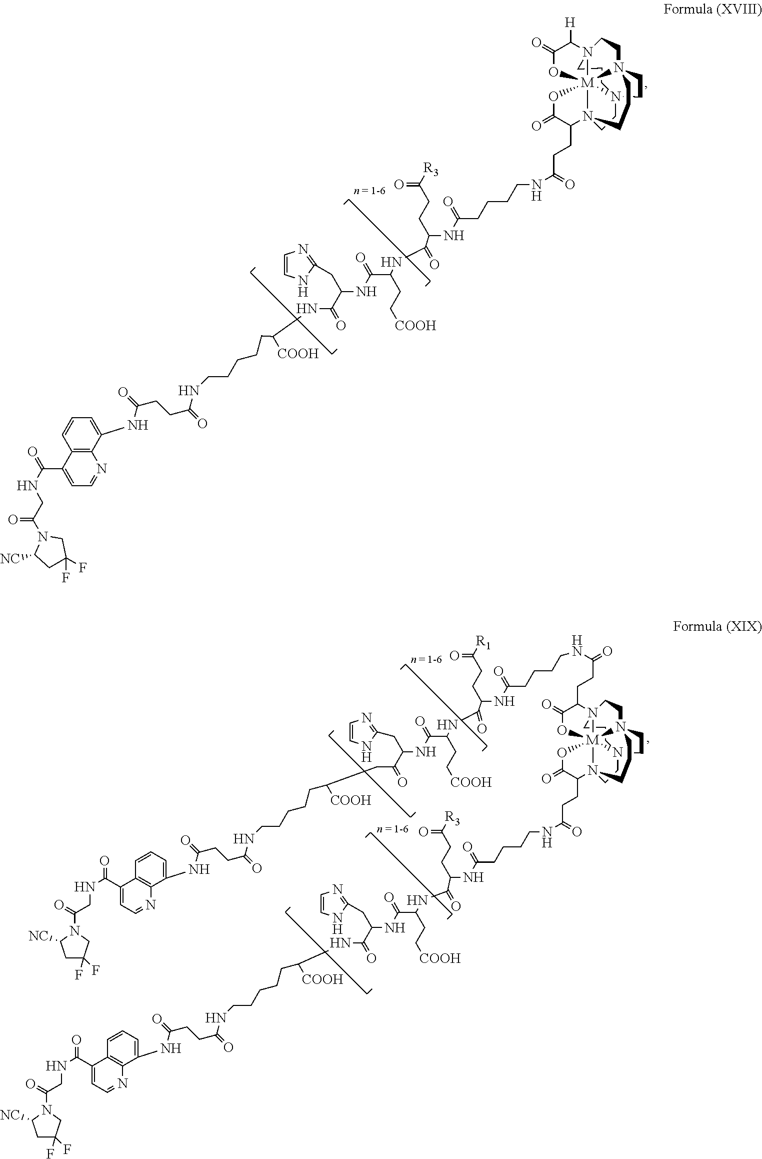

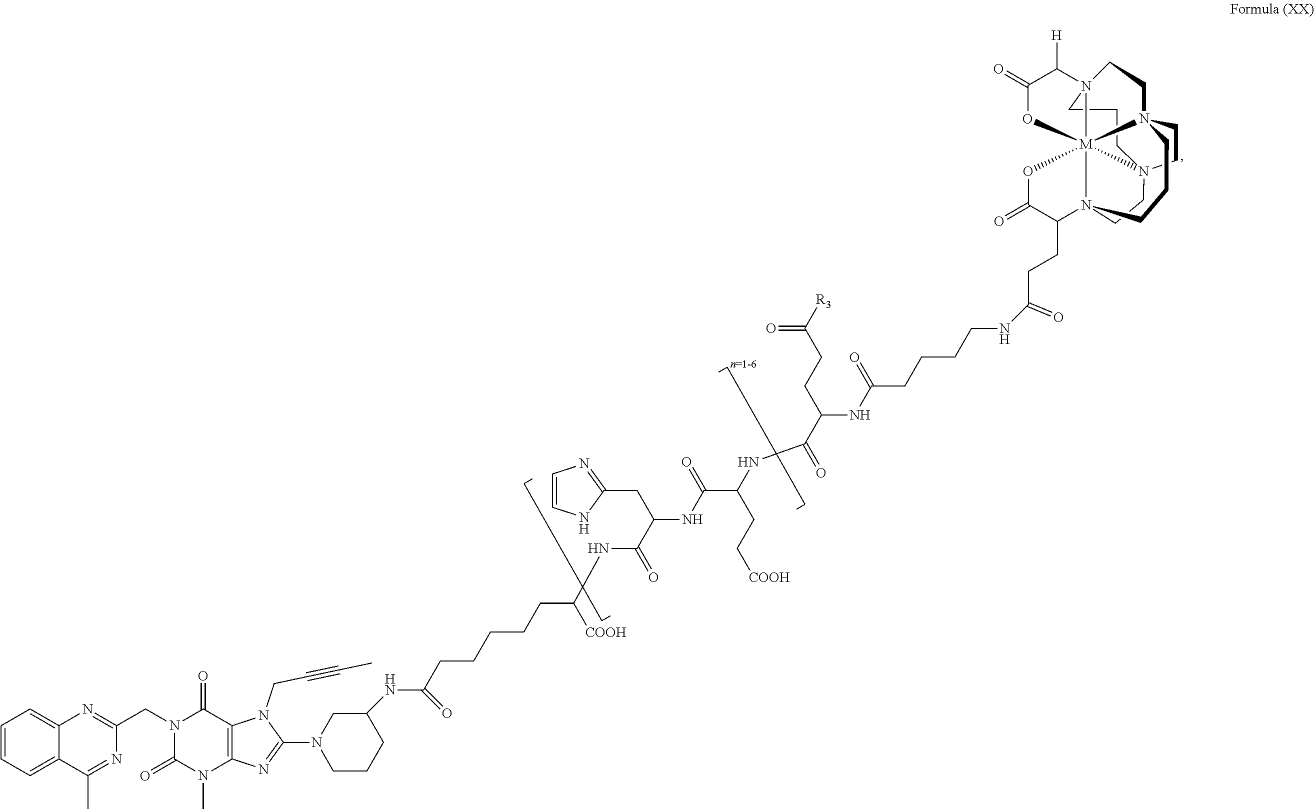

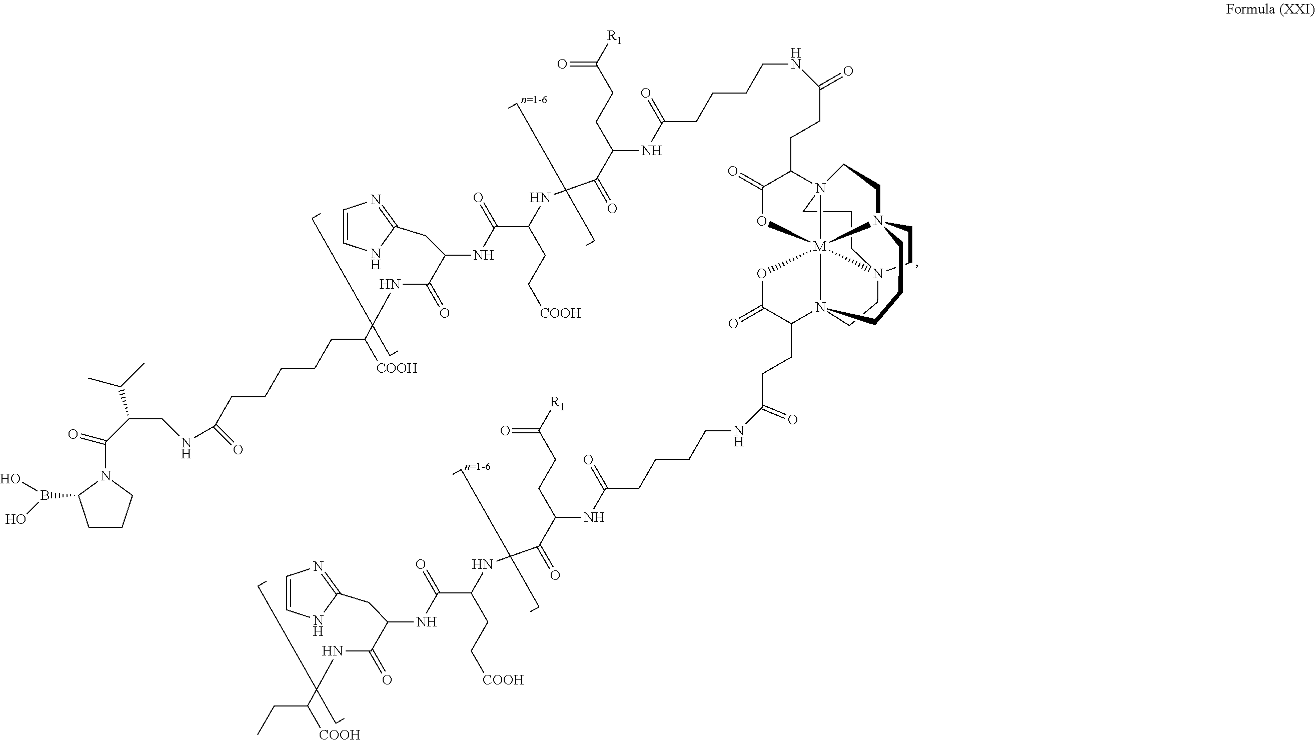

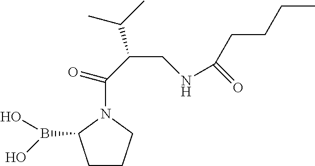 [SMILES: CCCCC(=O)NC[C@H](C(=O)N1CCC[C@@H]1B(O)O)C(C)C]

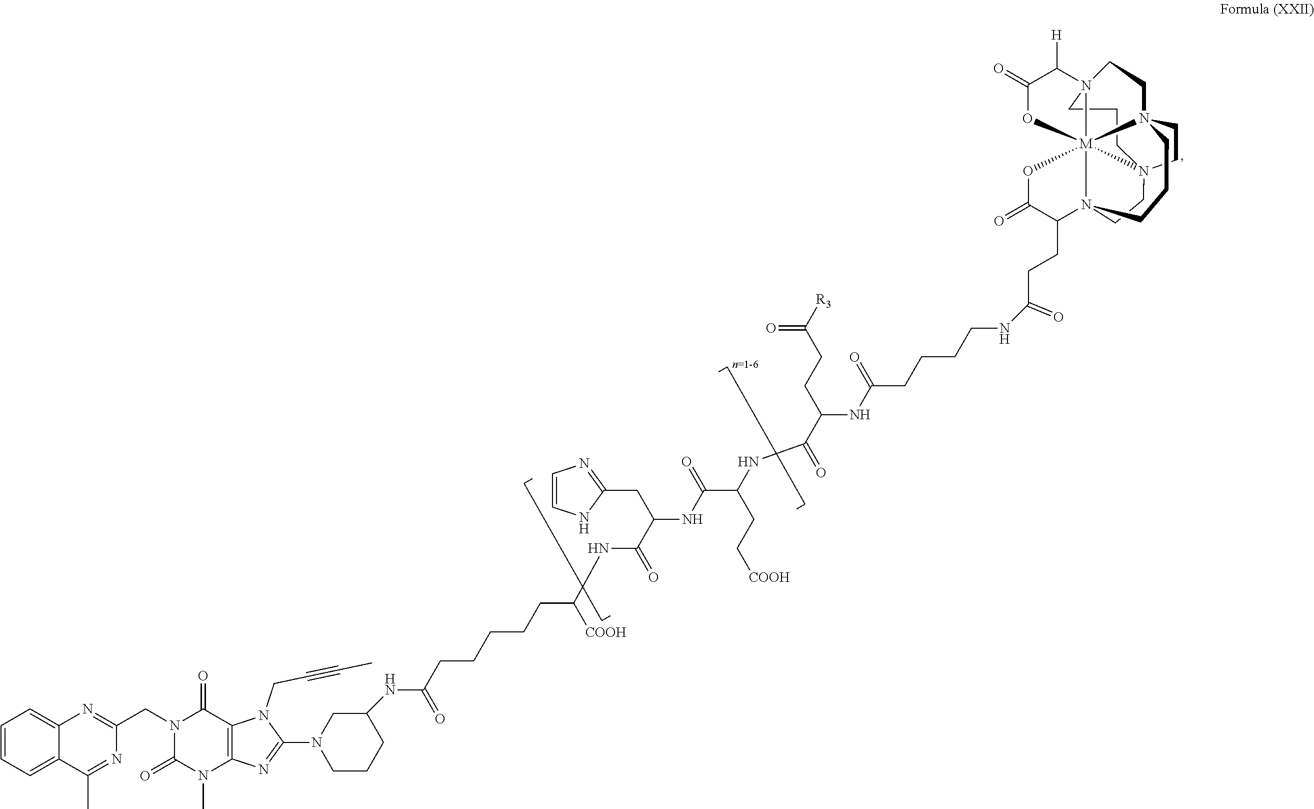

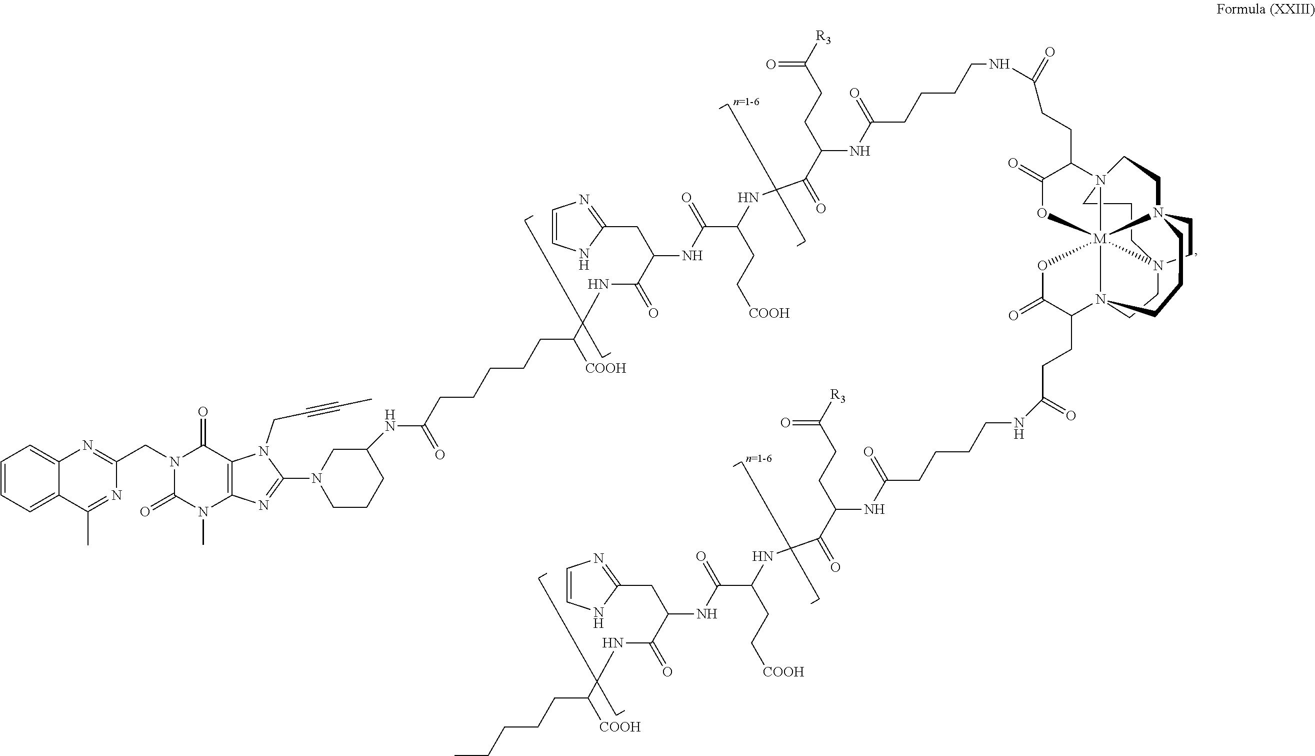

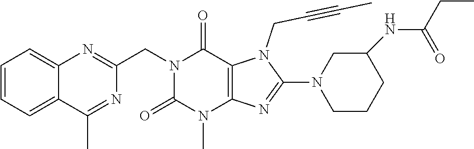 [SMILES: CC#CCn1c(N2CCCC(NC(=O)CC)C2)nc2c1c(=O)n(Cc1nc(C)c3ccccc3n1)c(=O)n2C]

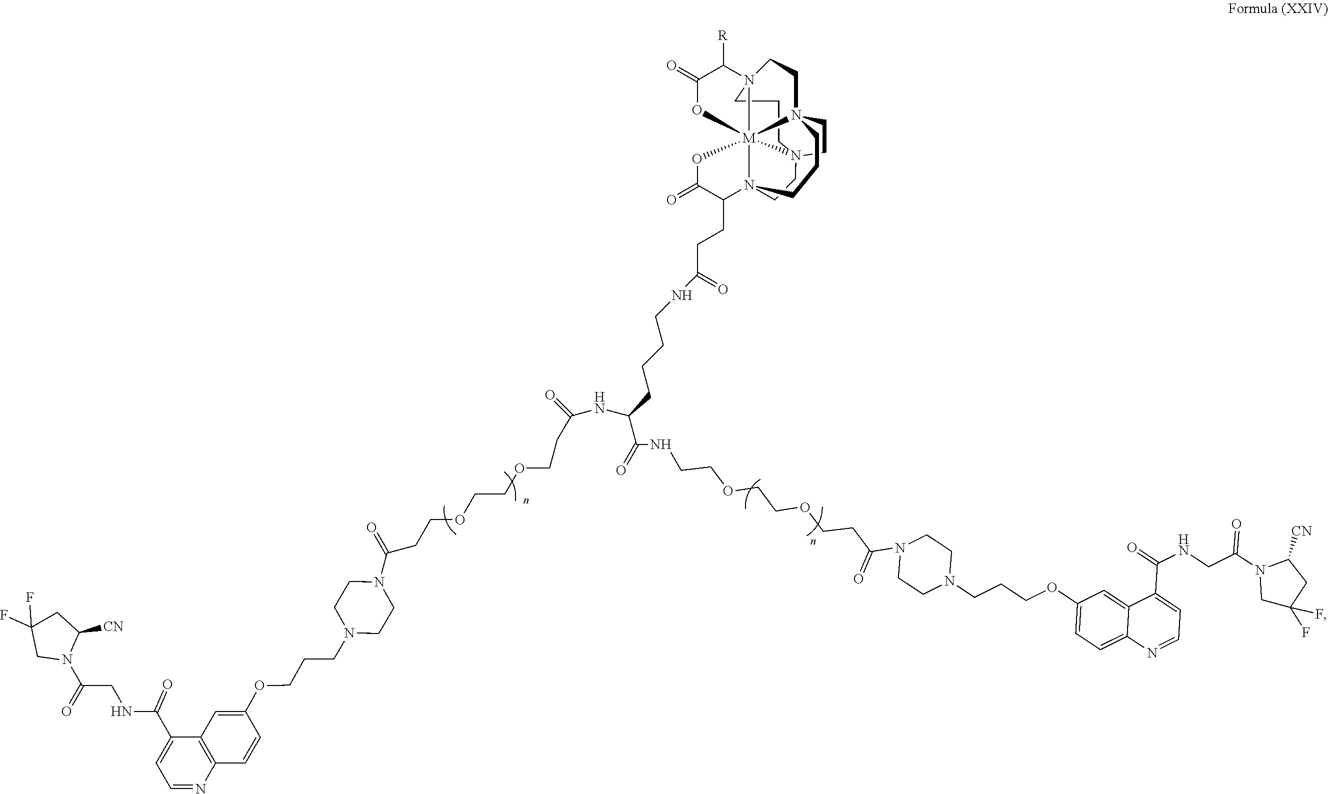

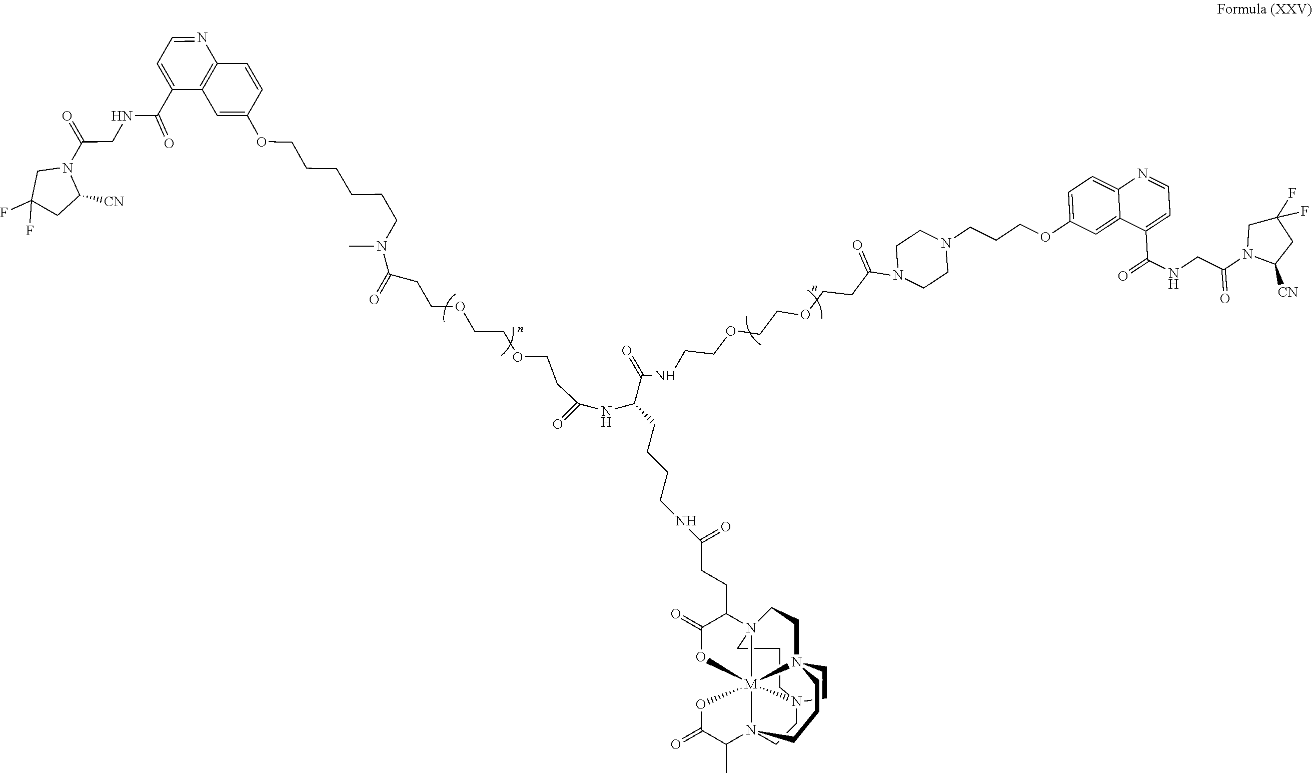

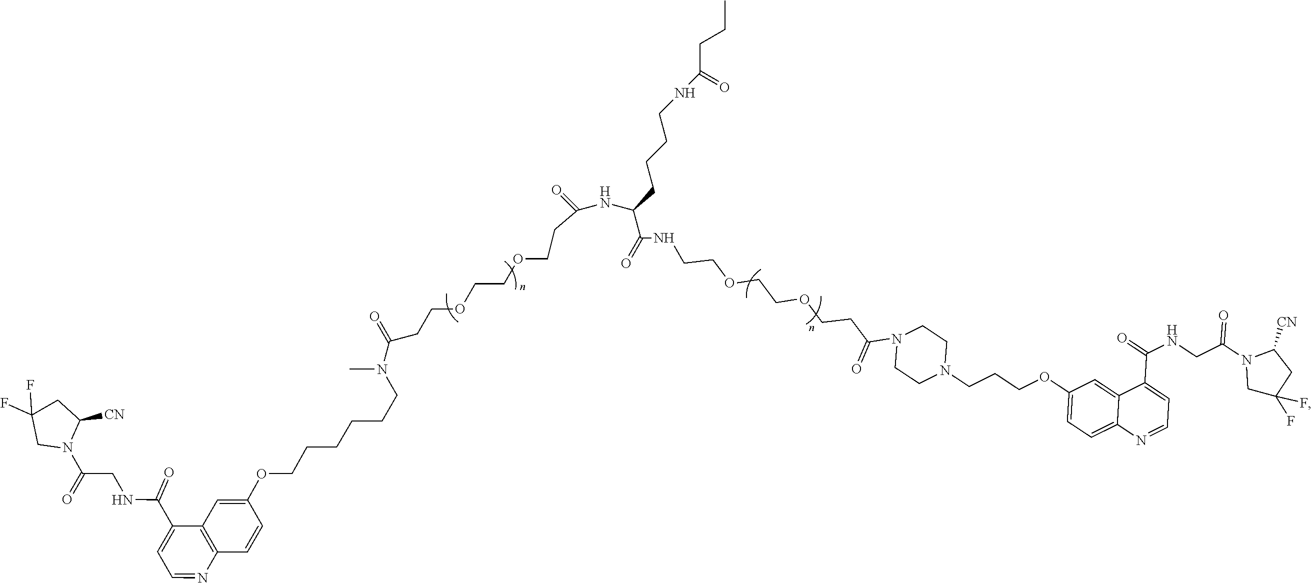

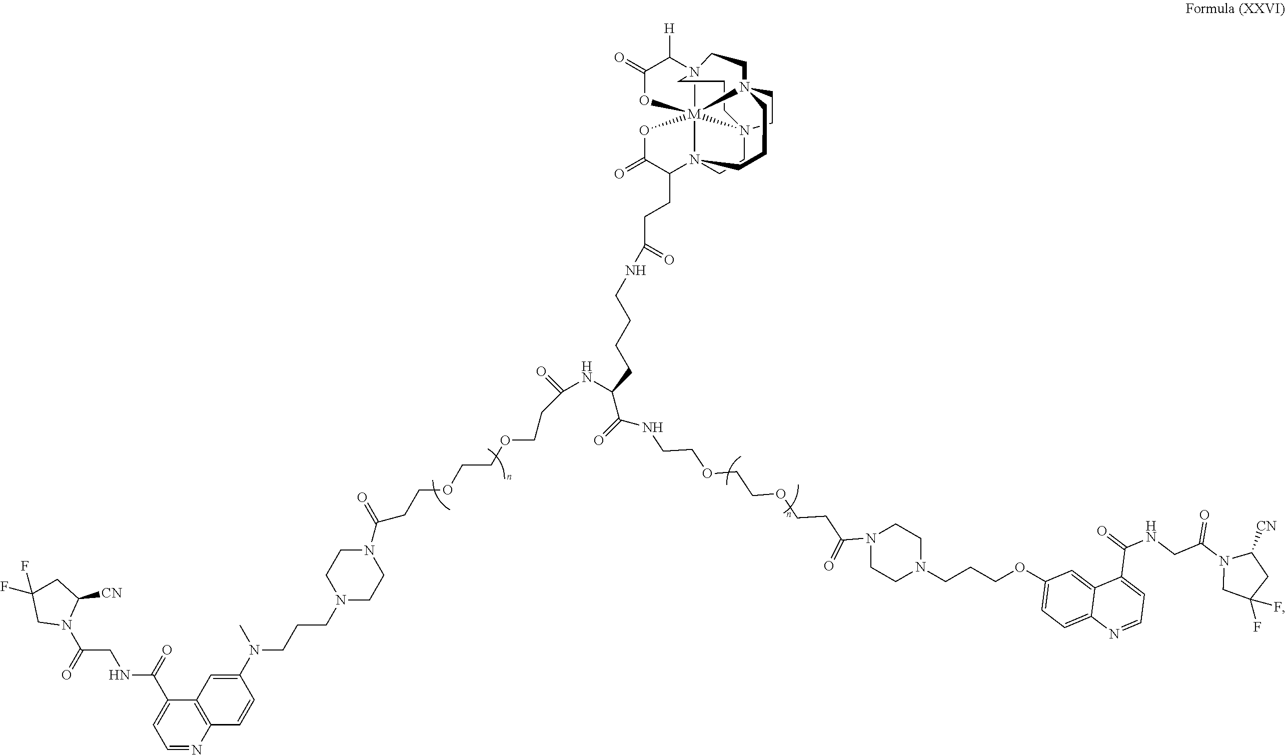

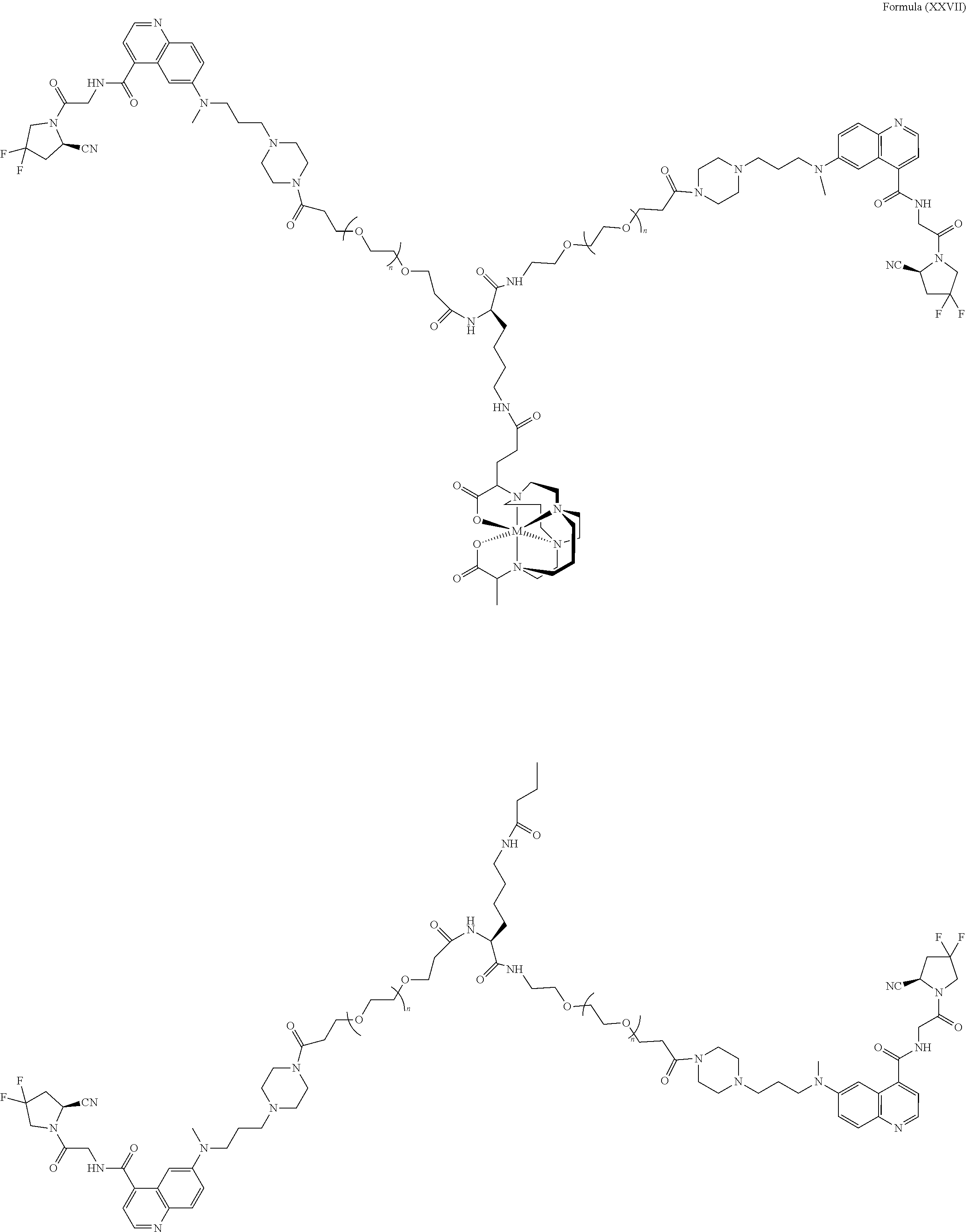

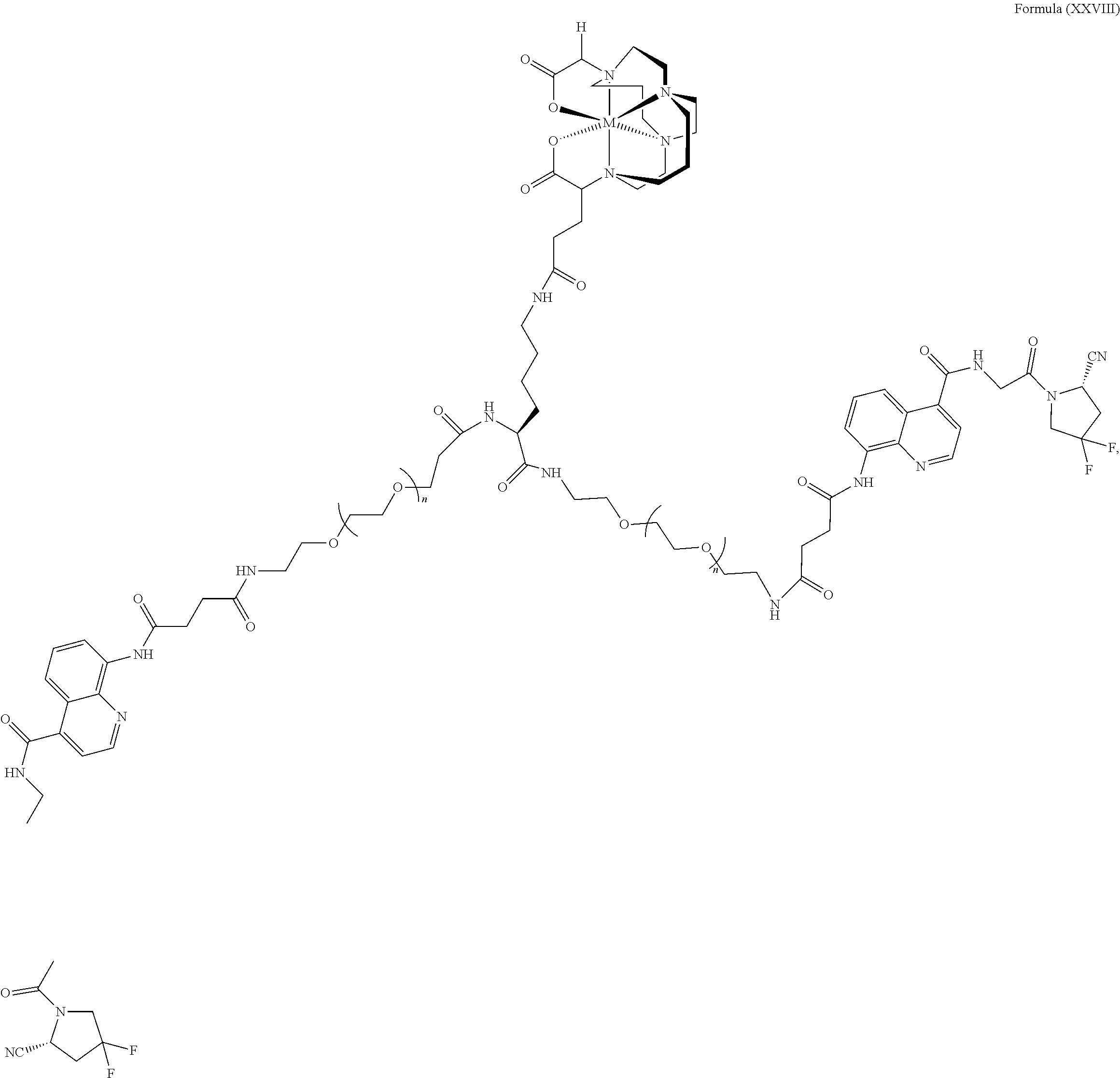

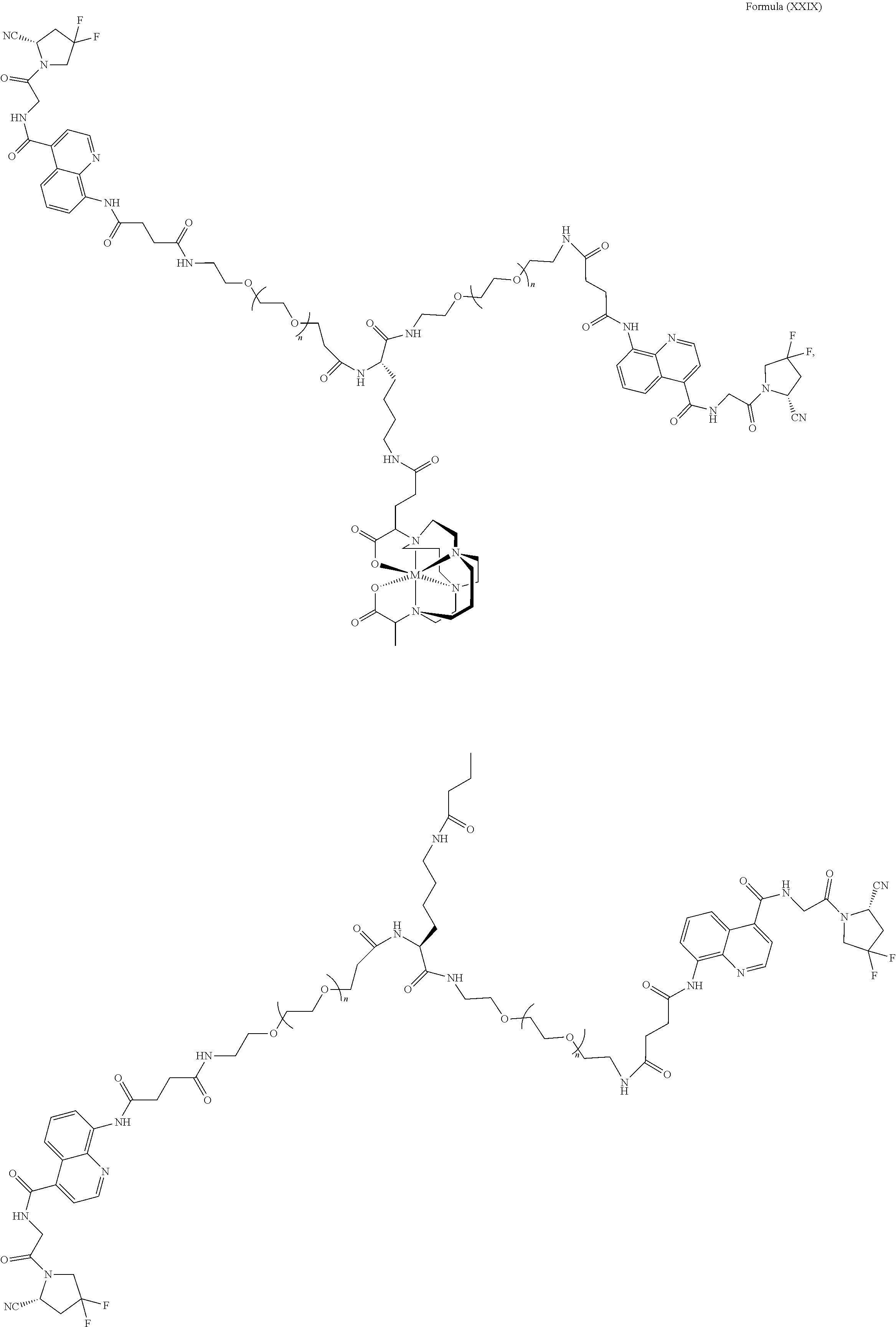

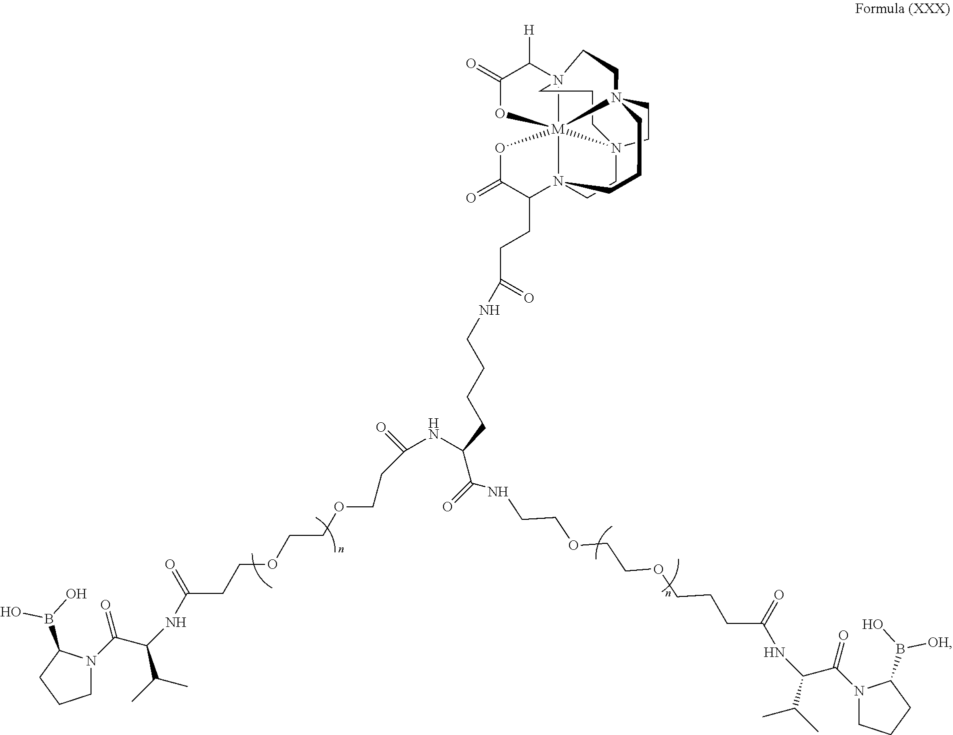

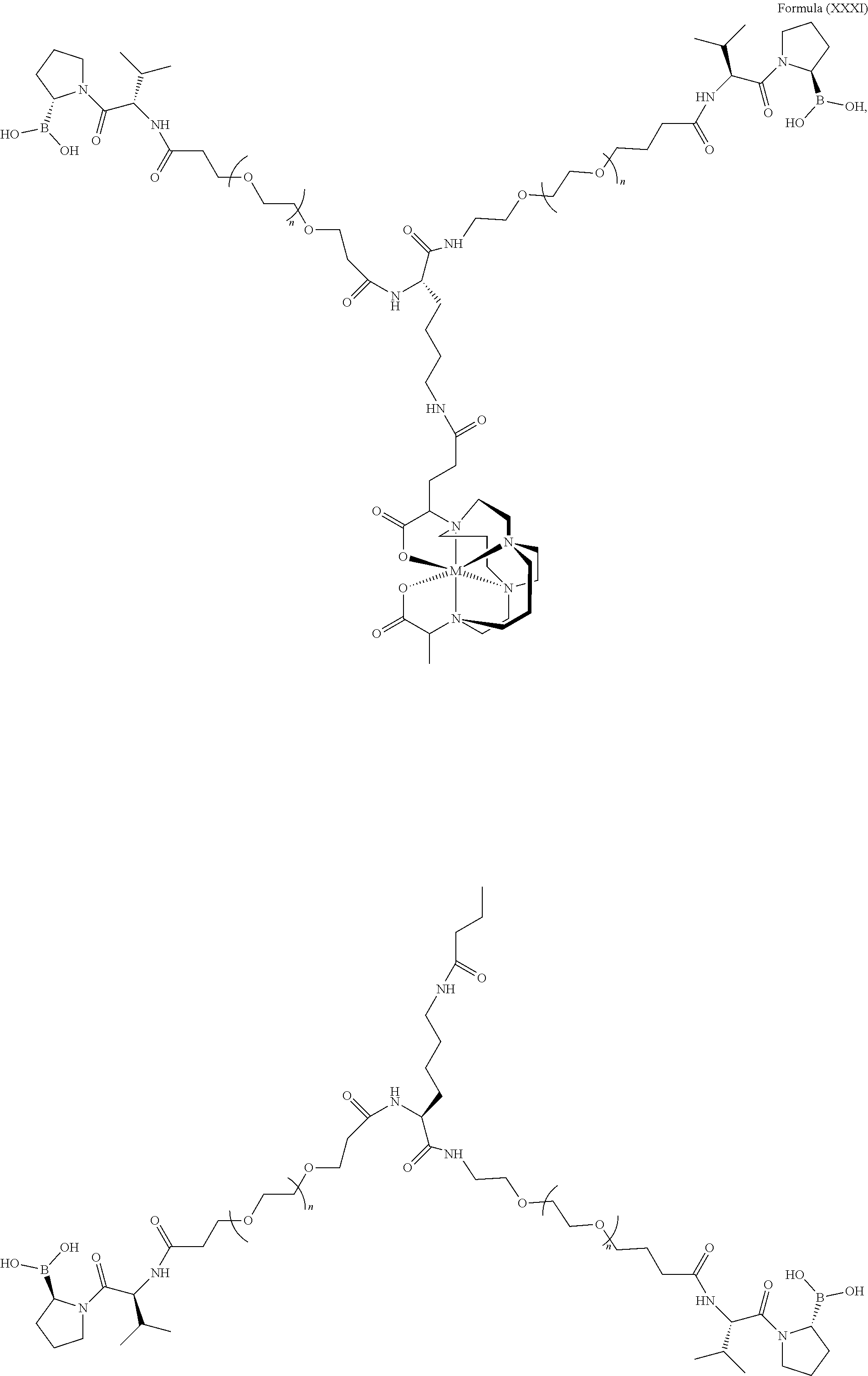

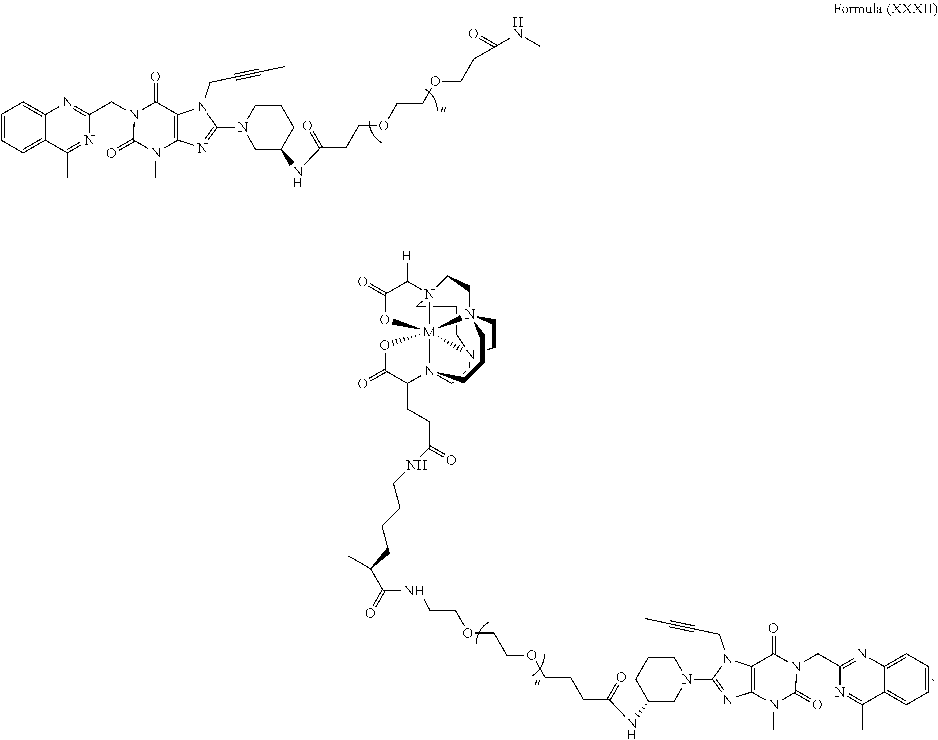

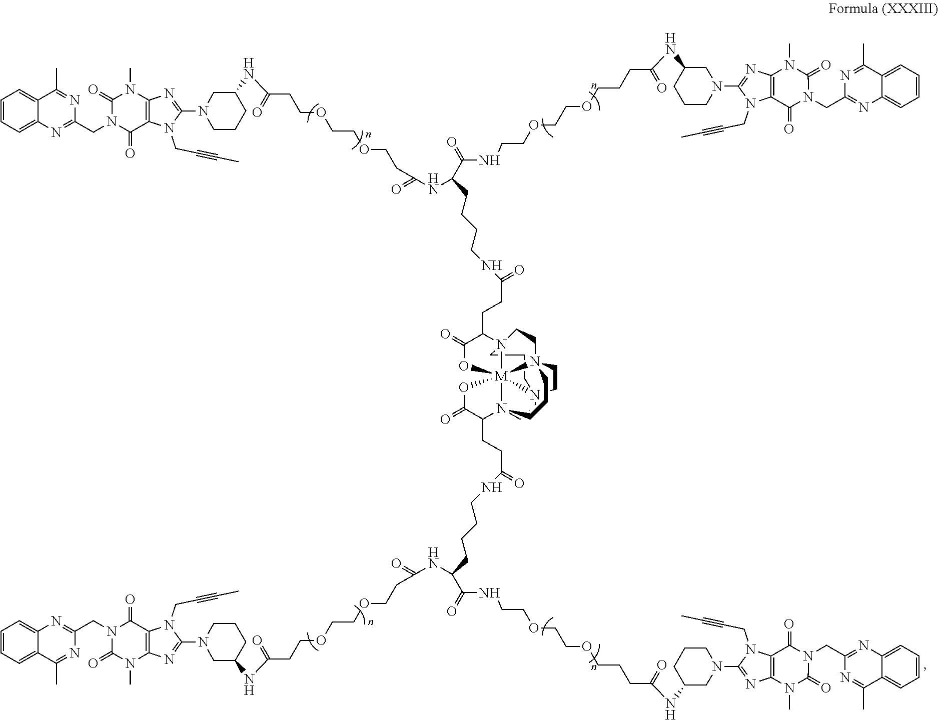

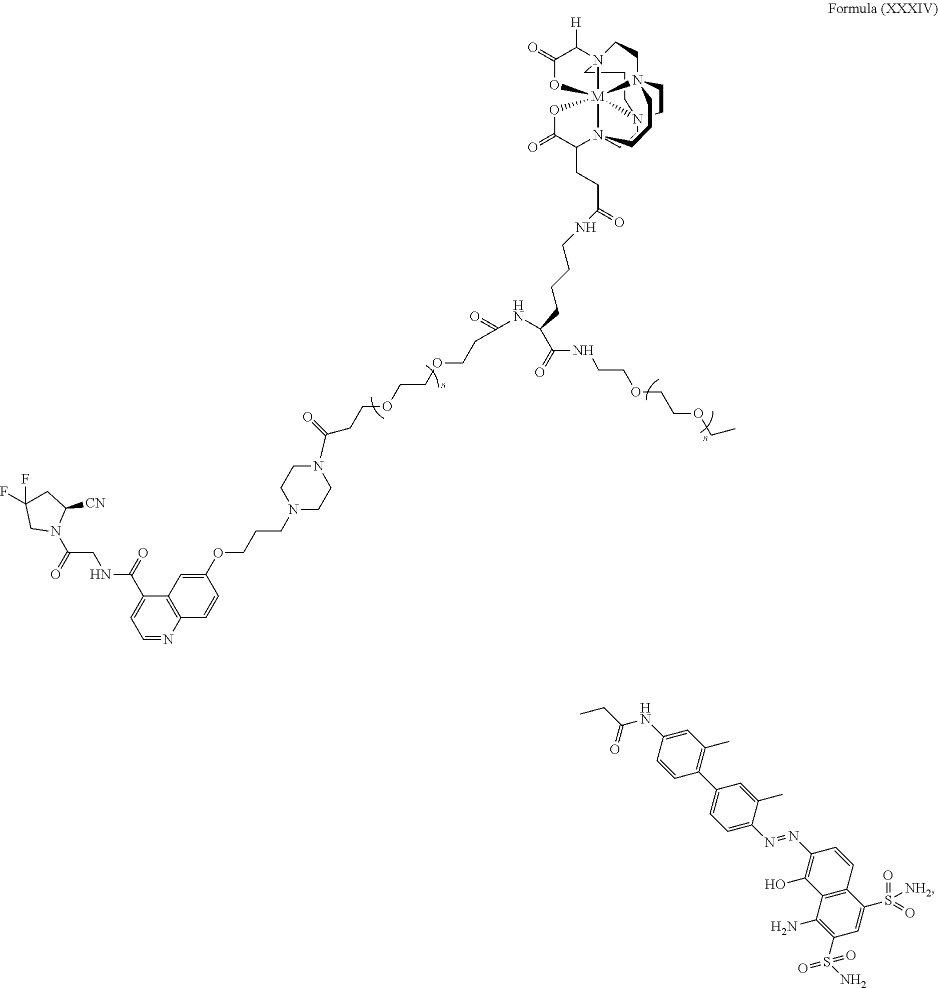

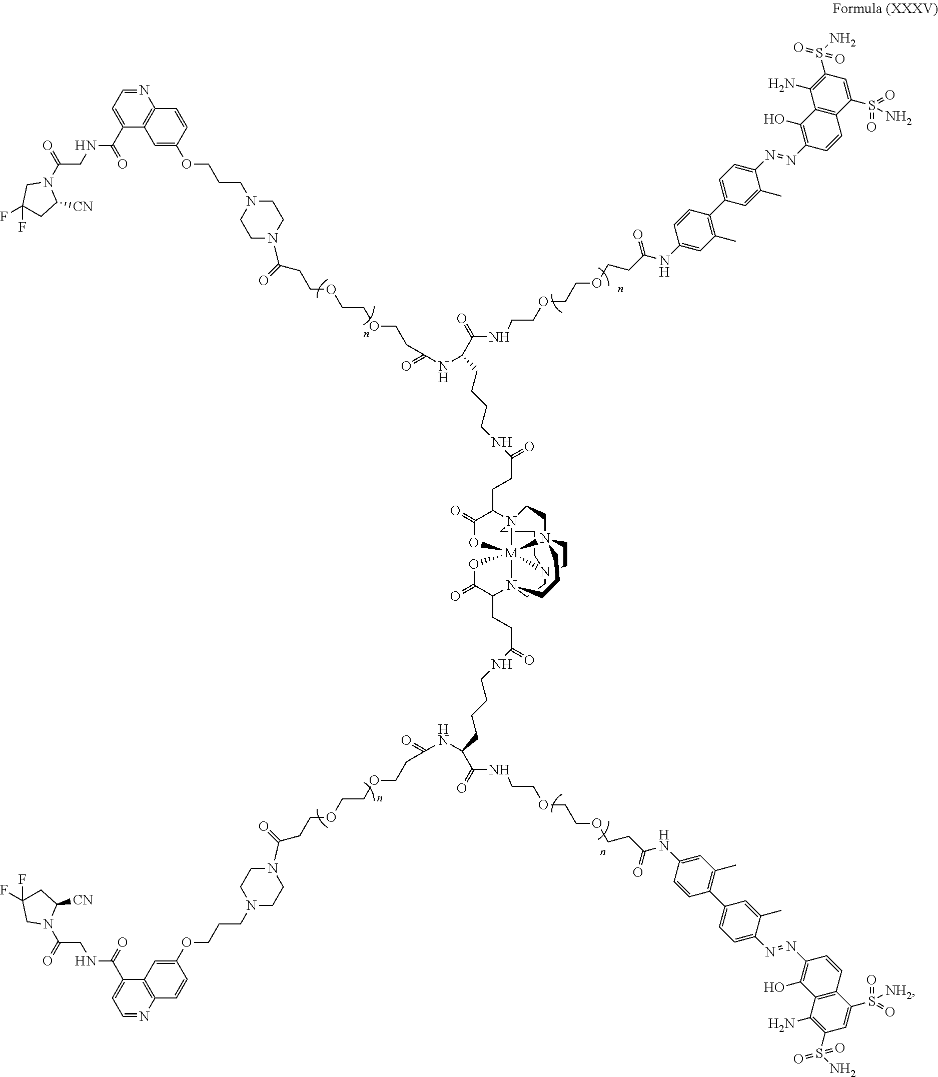

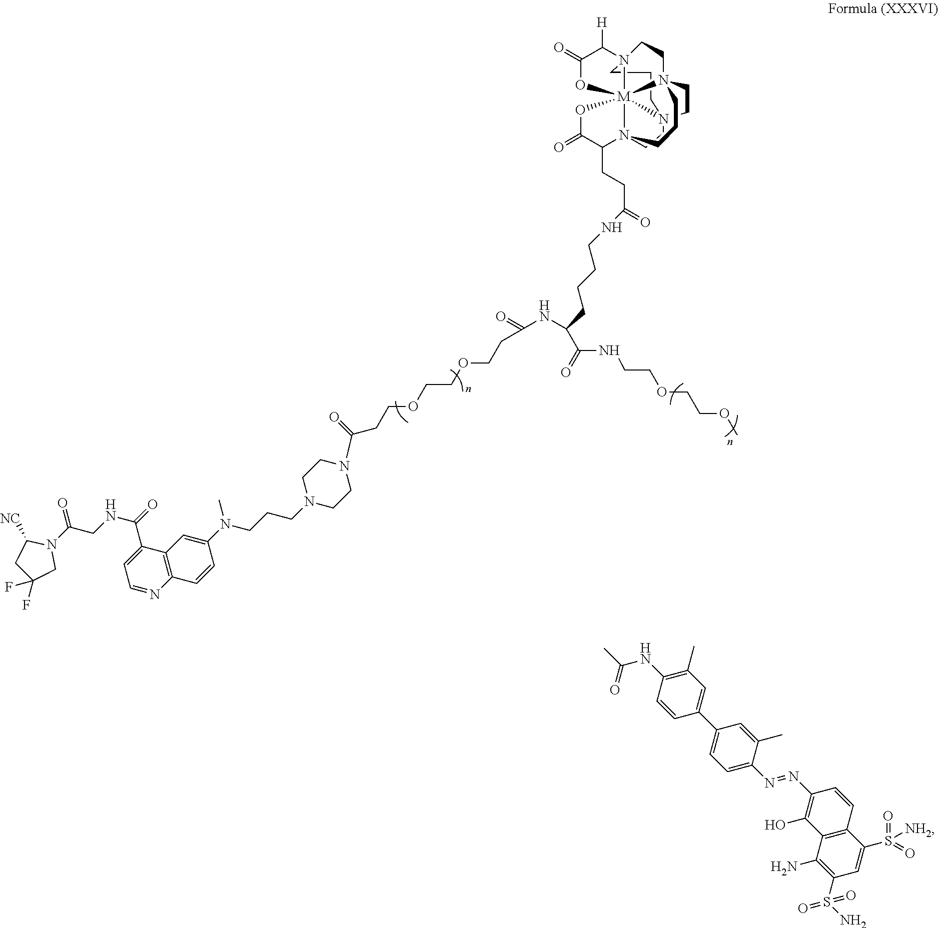

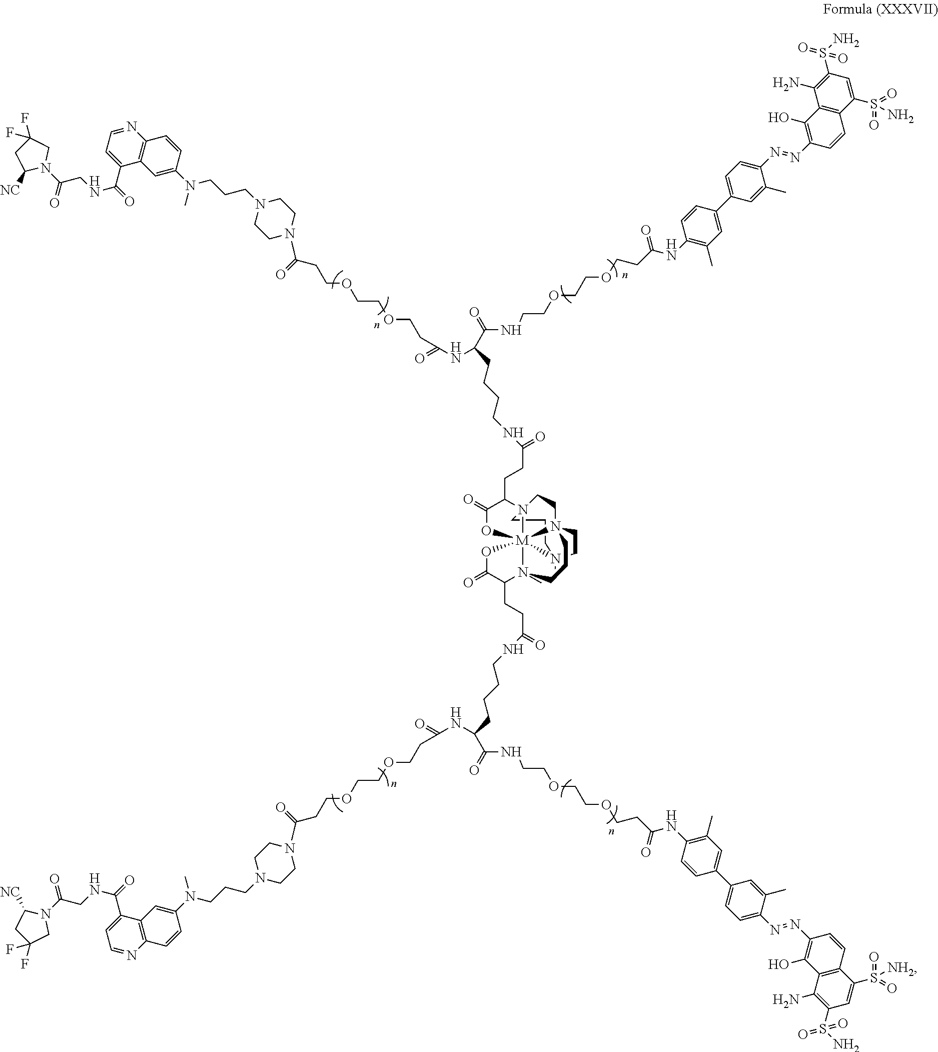

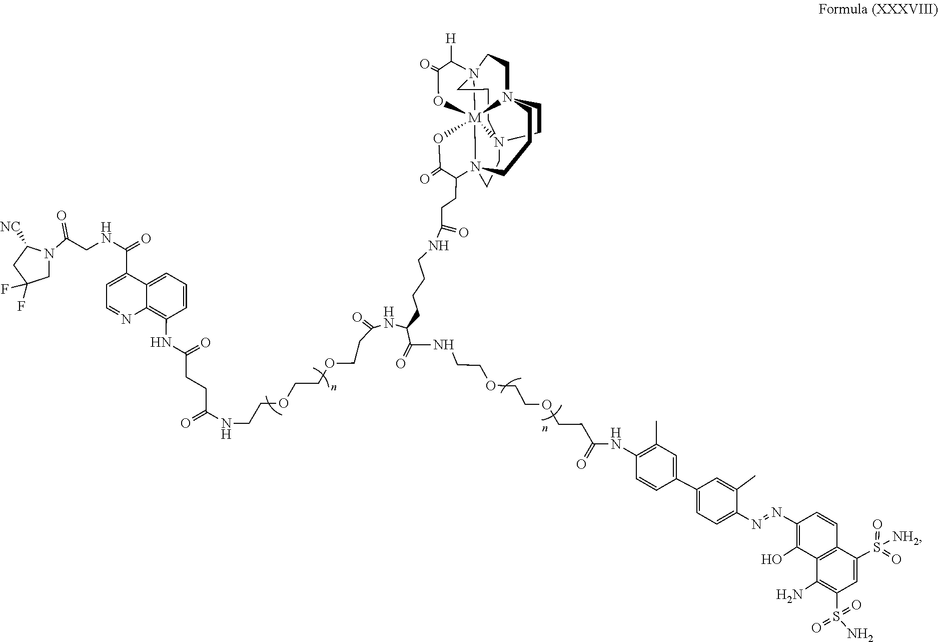

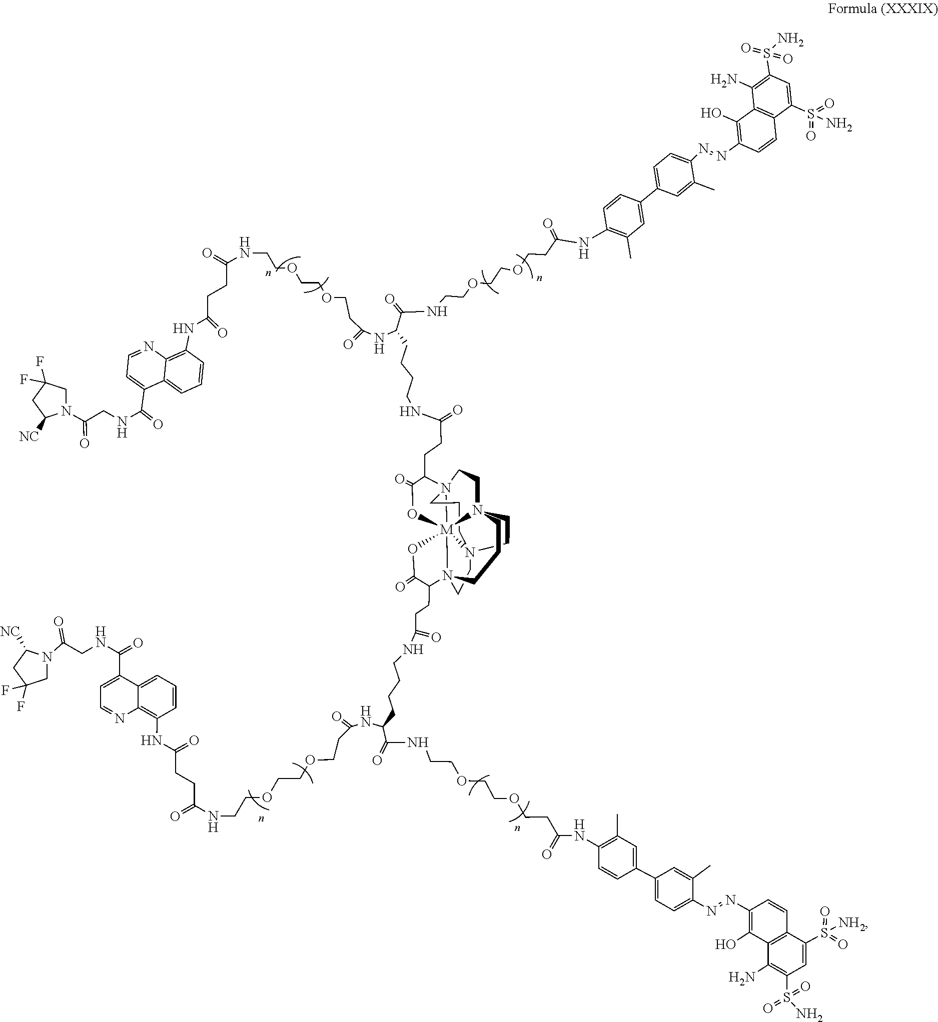

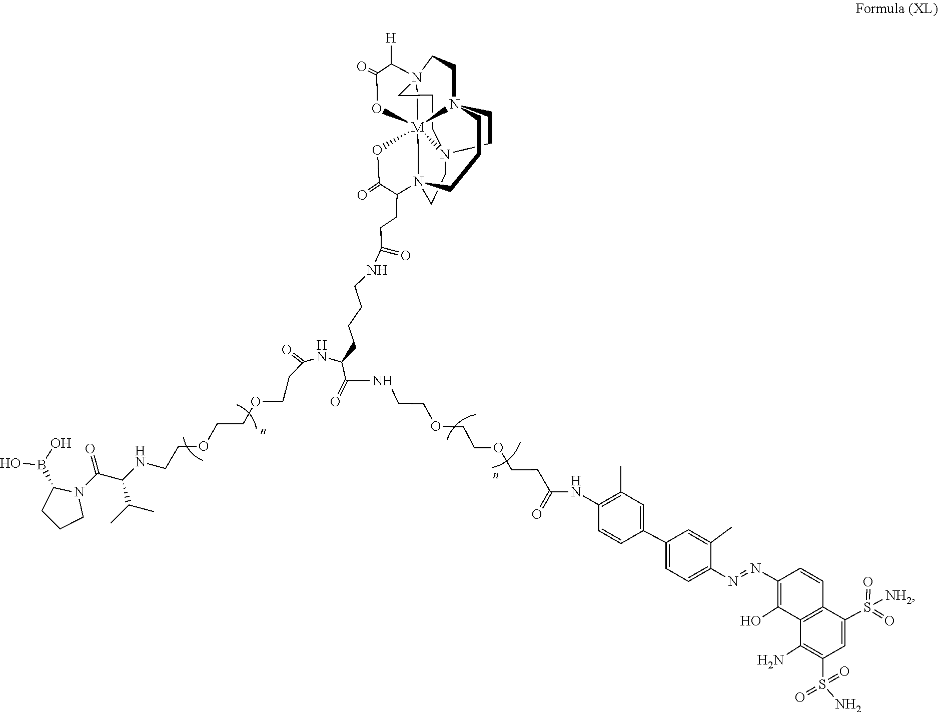

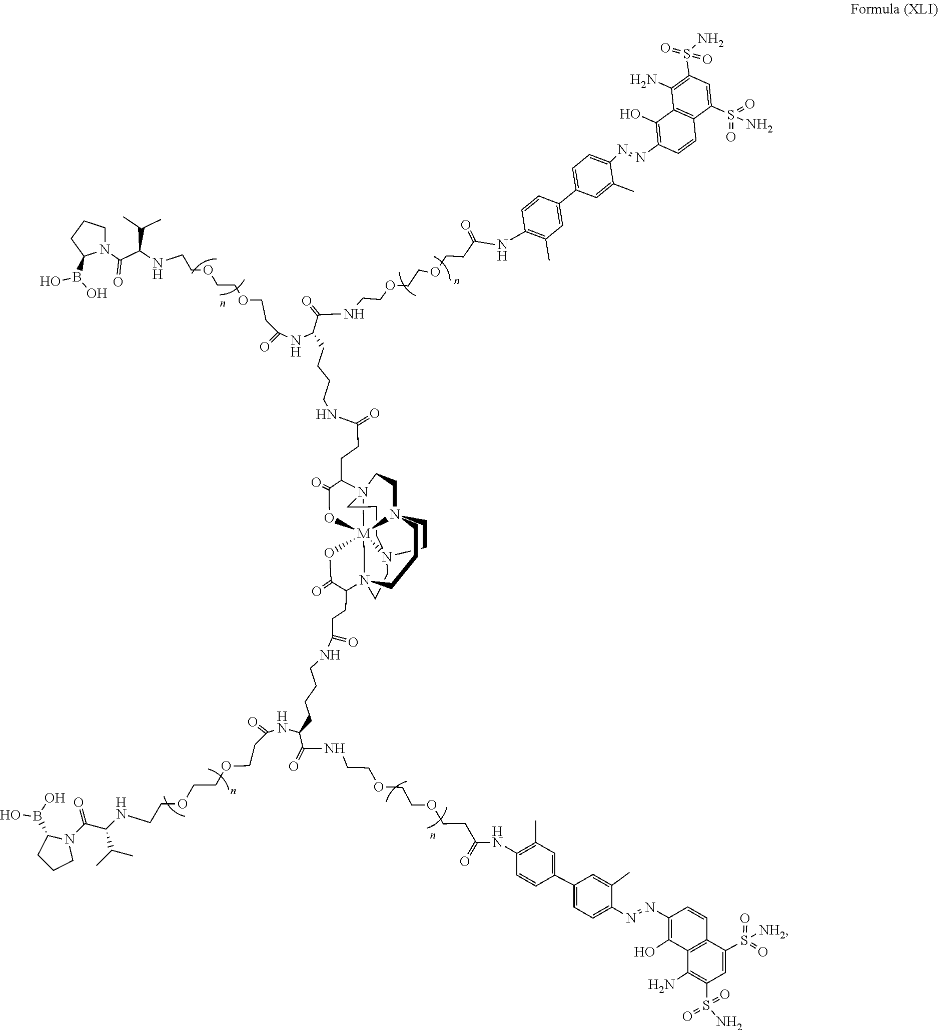

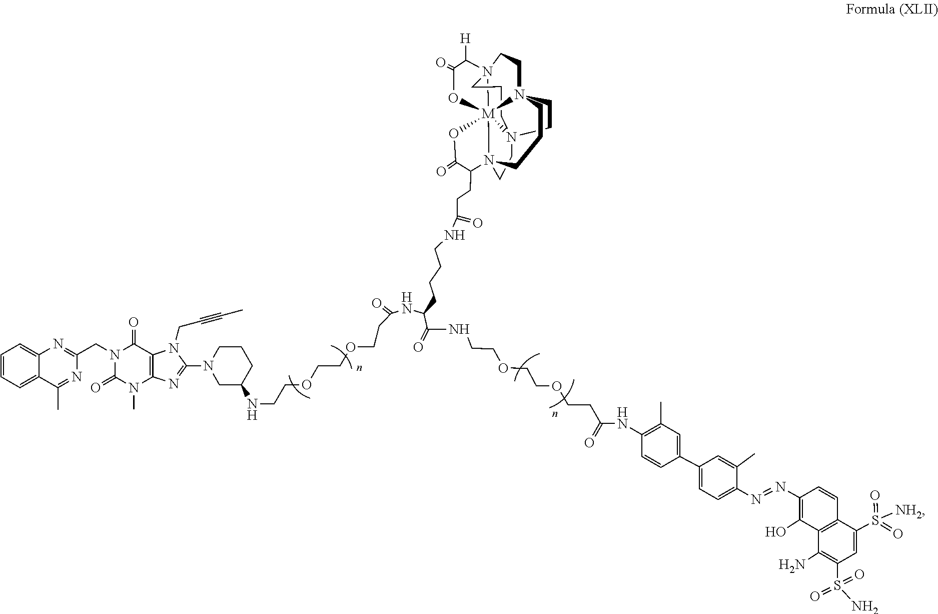

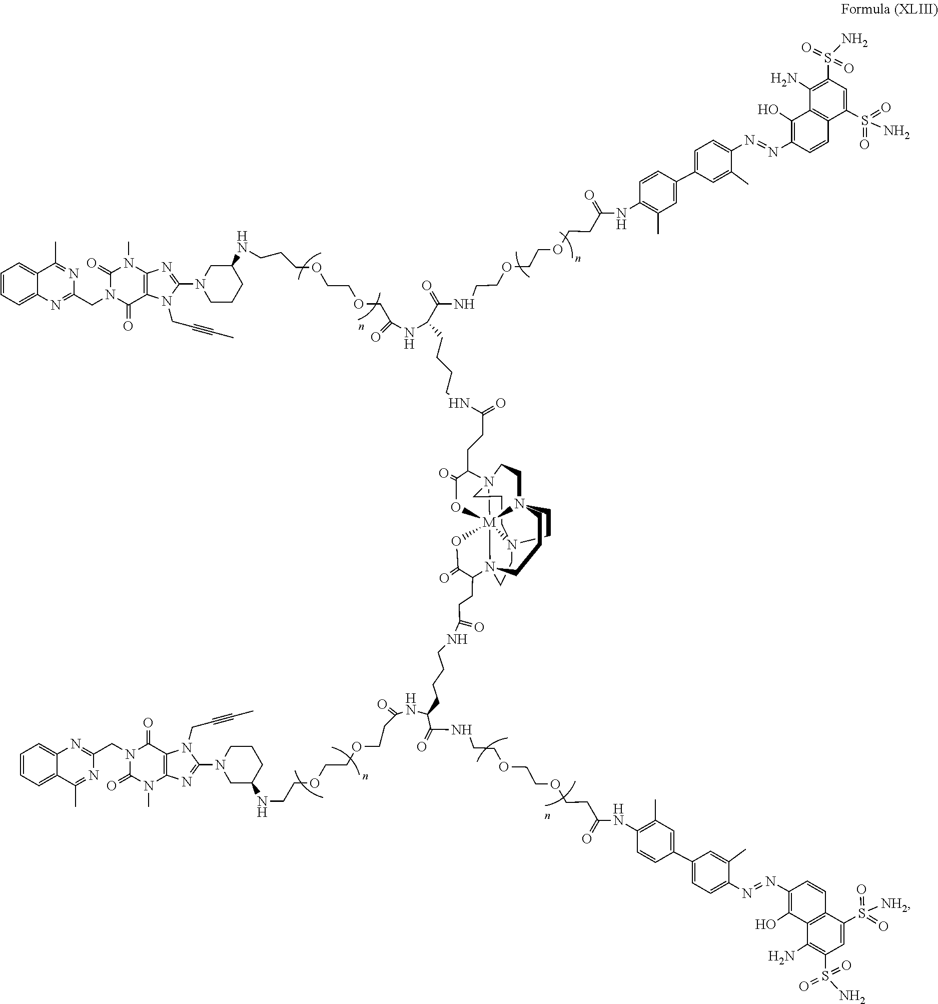

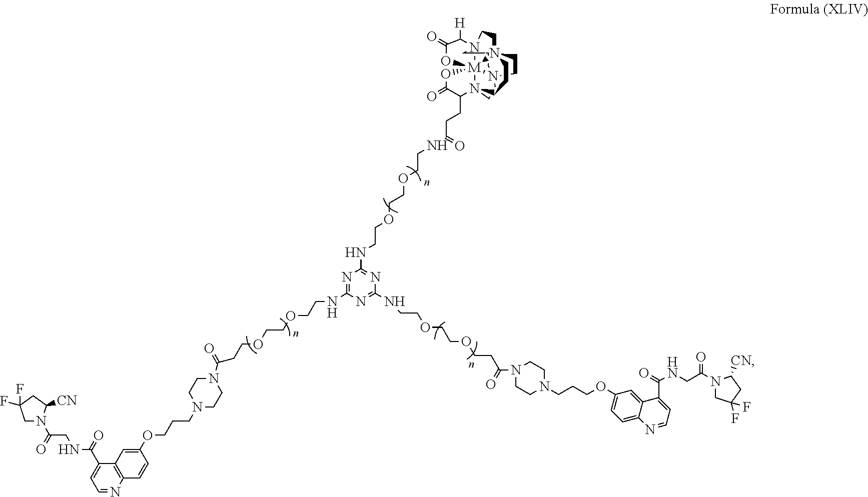

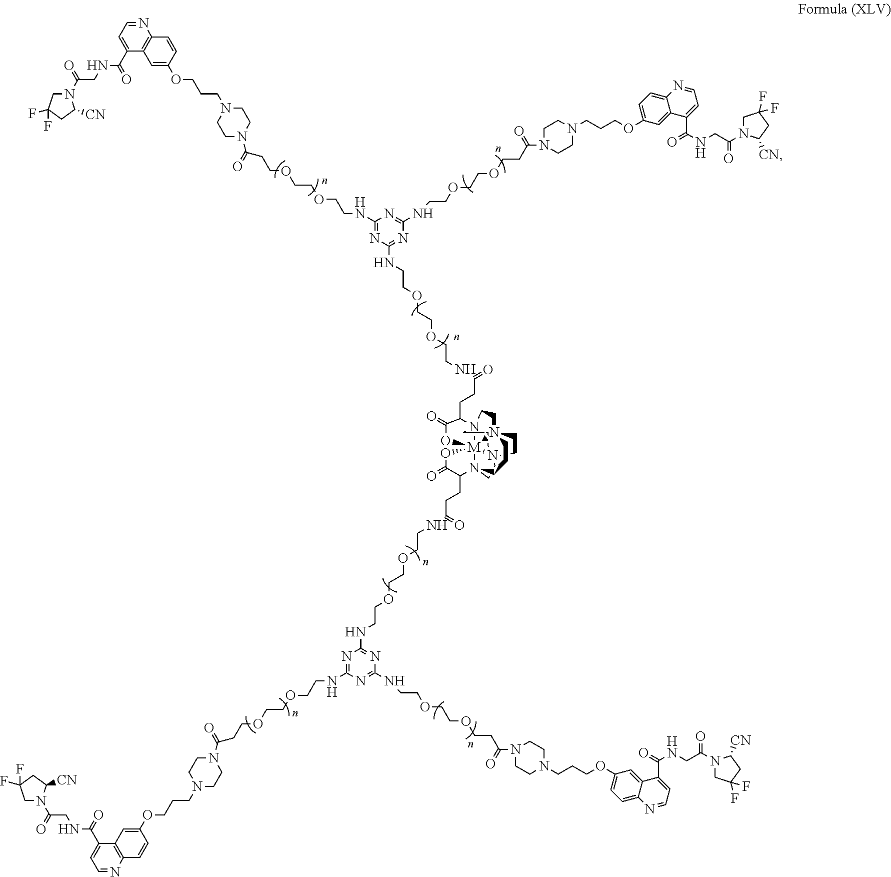

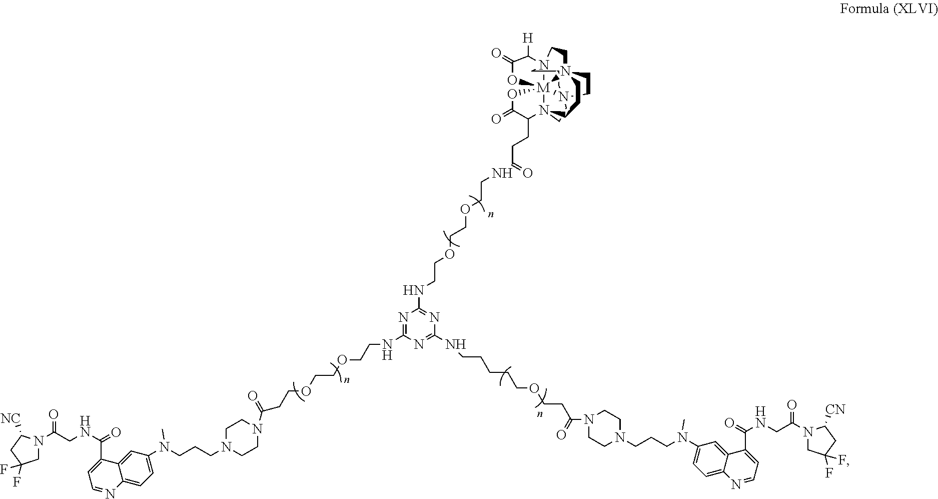

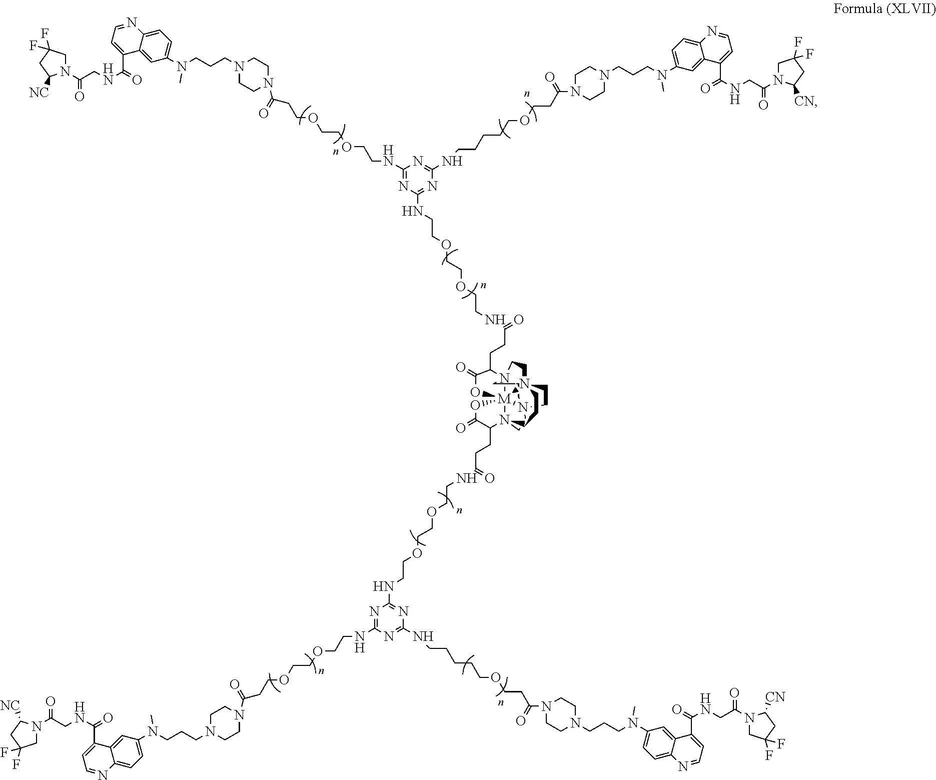

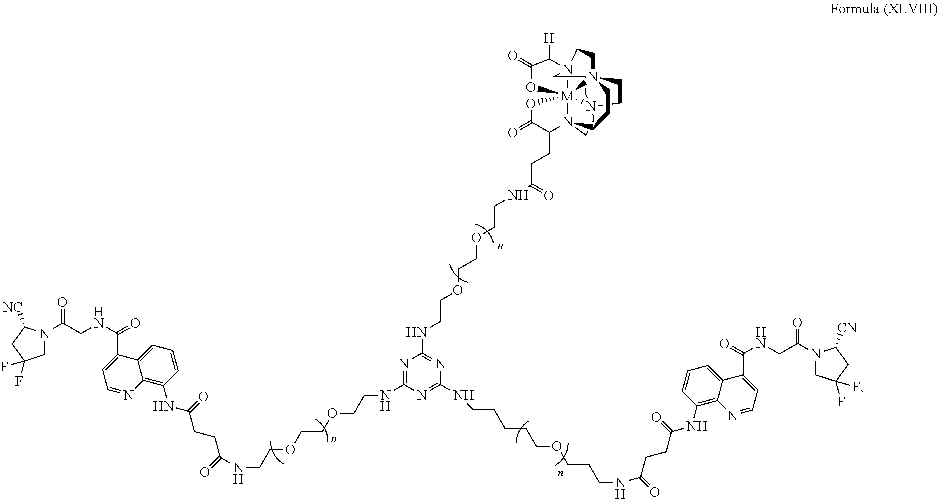

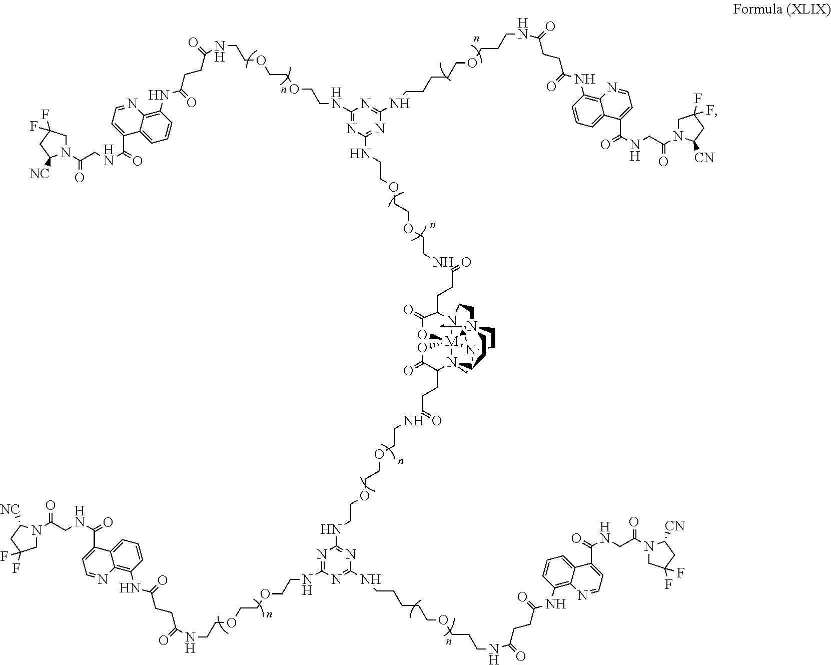

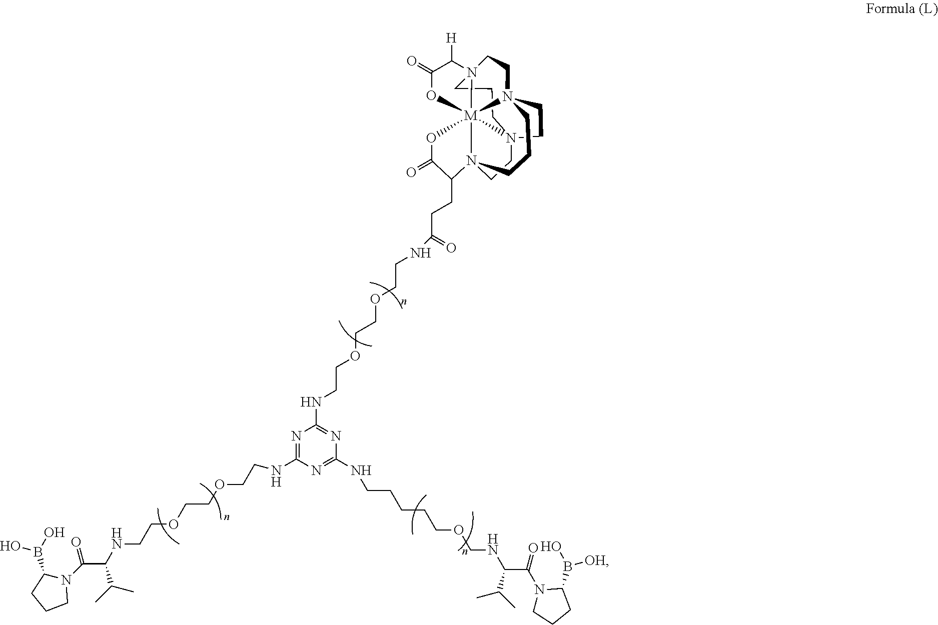

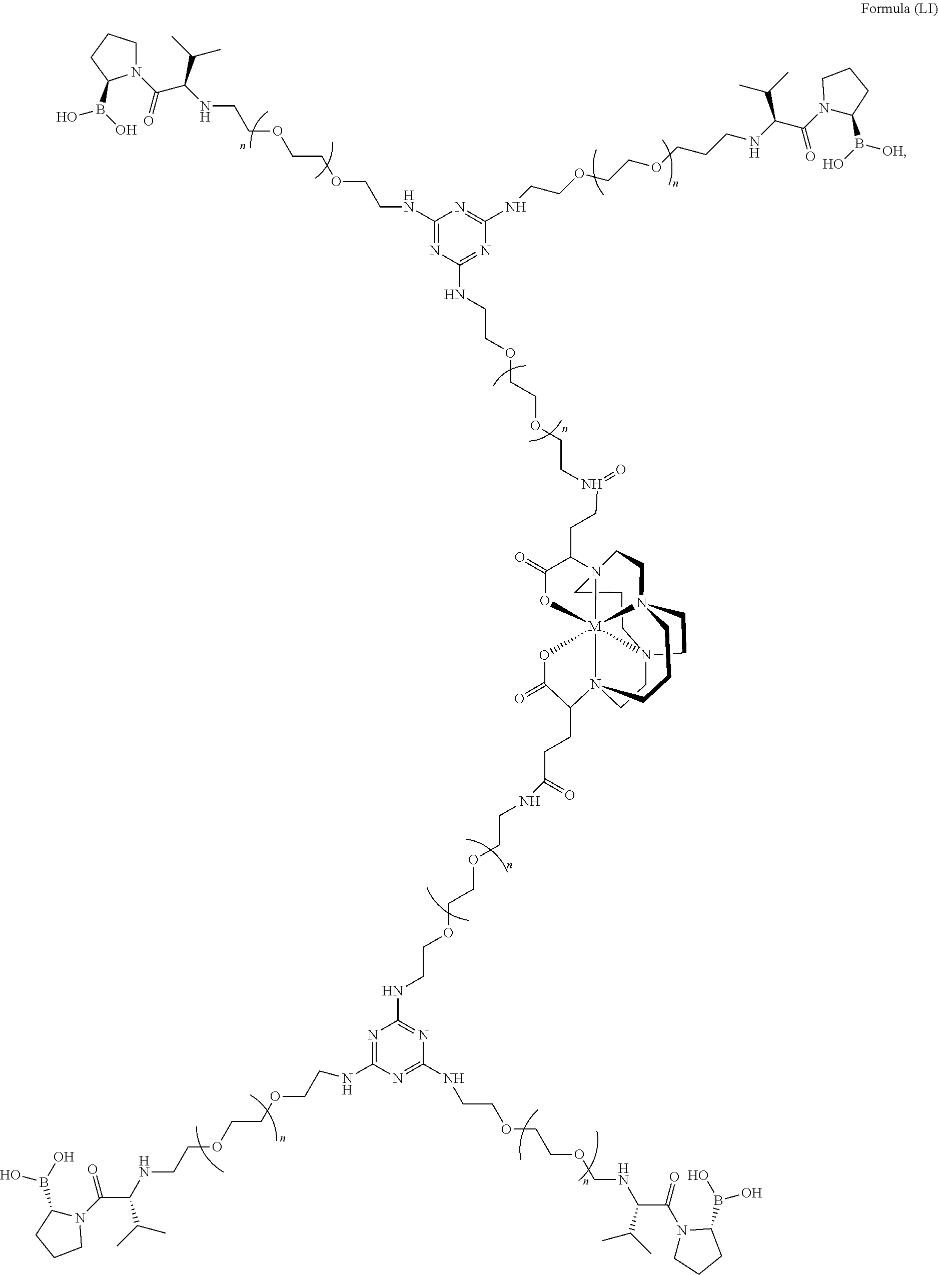

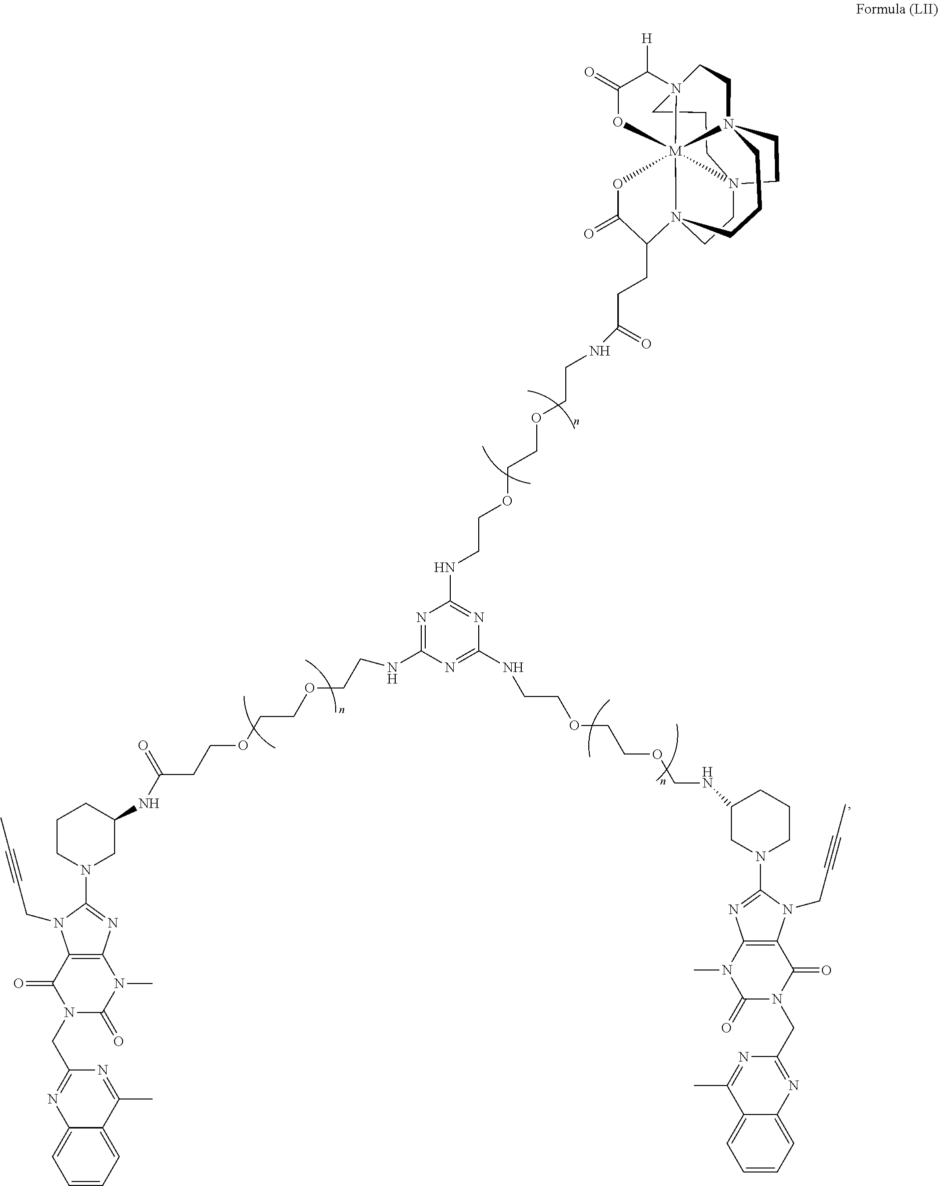

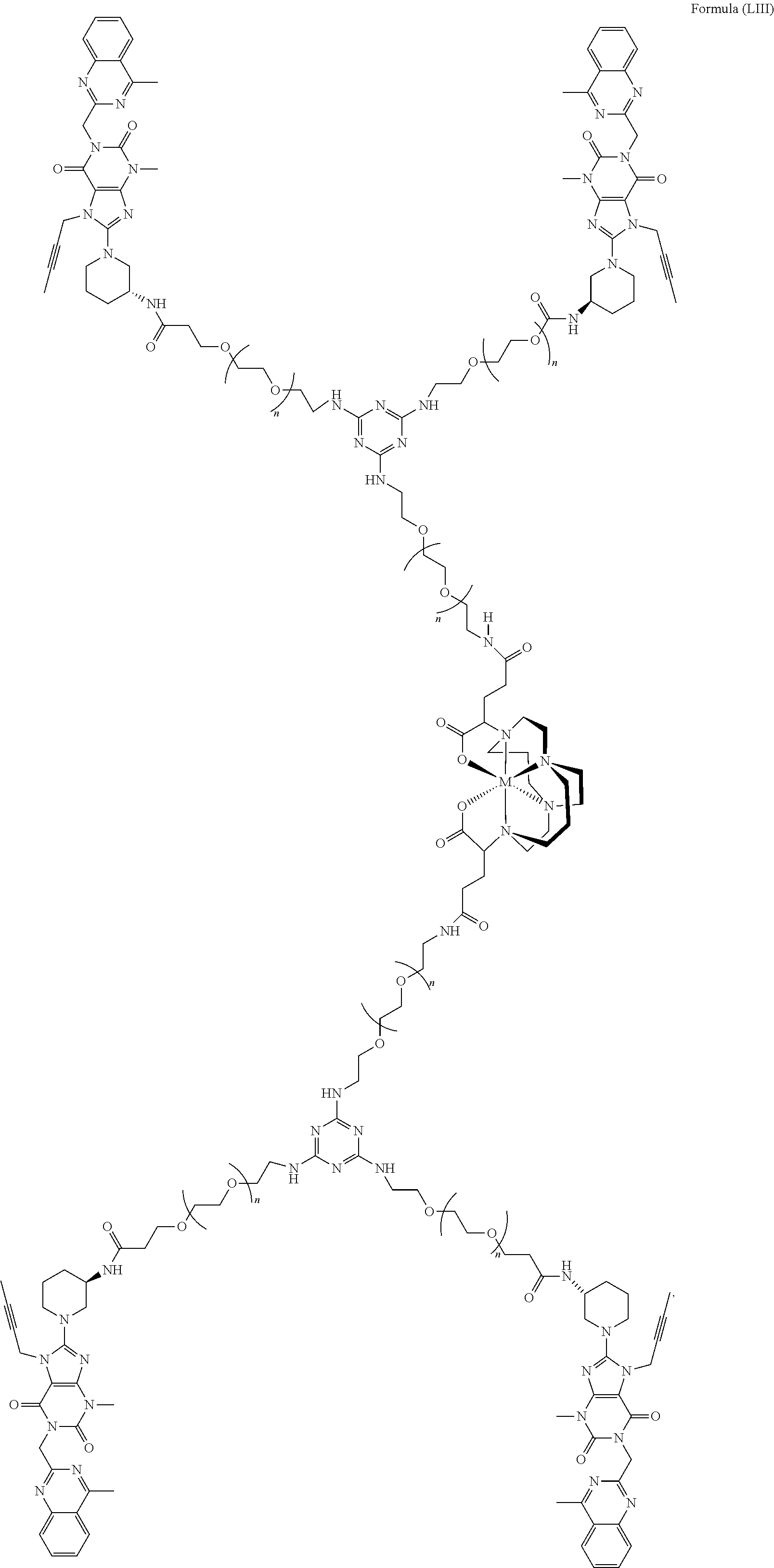

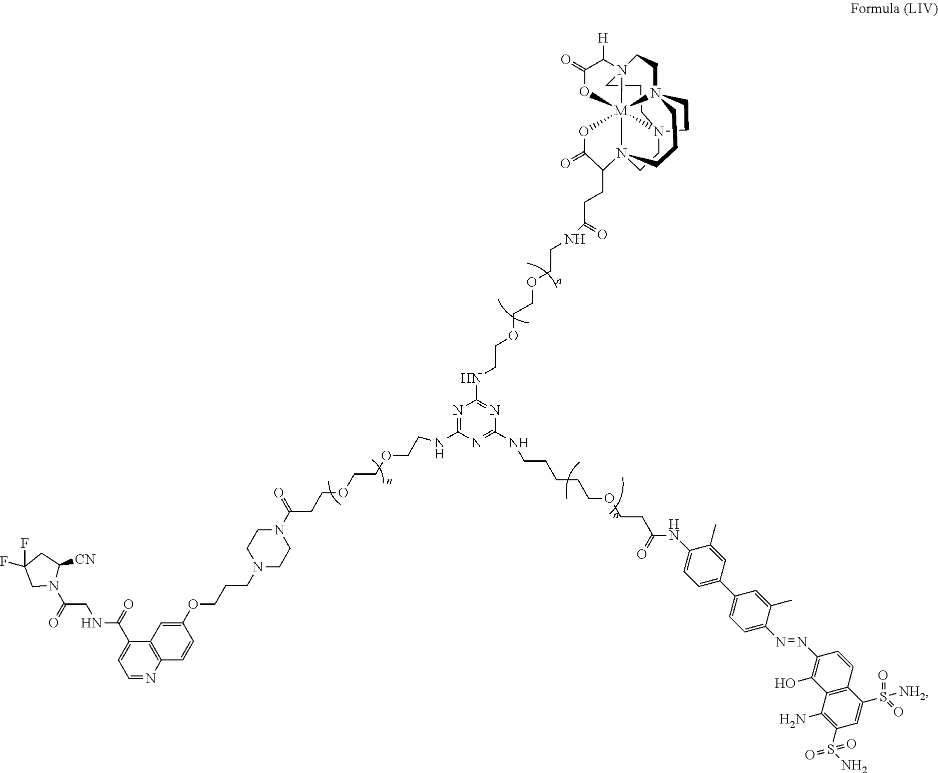

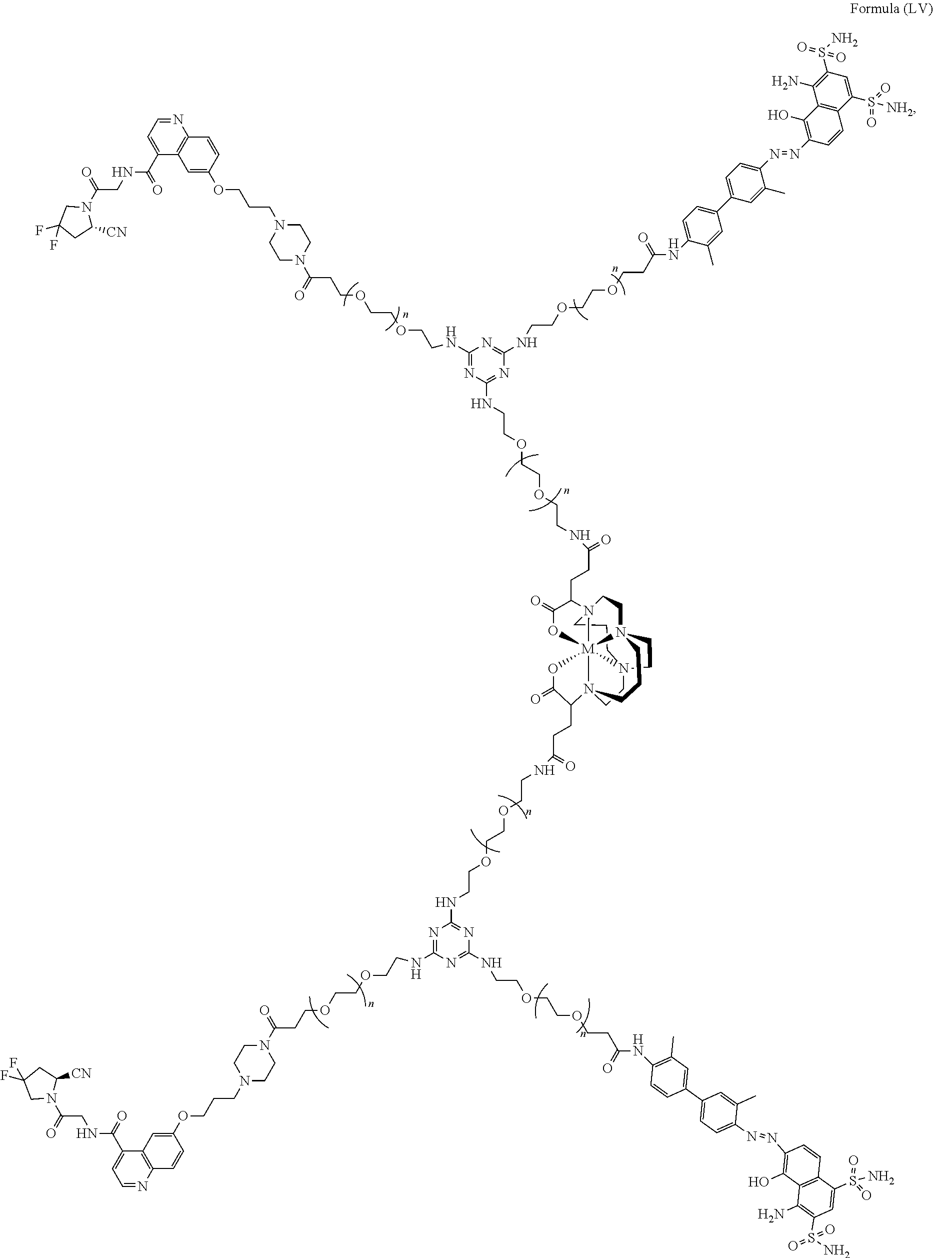

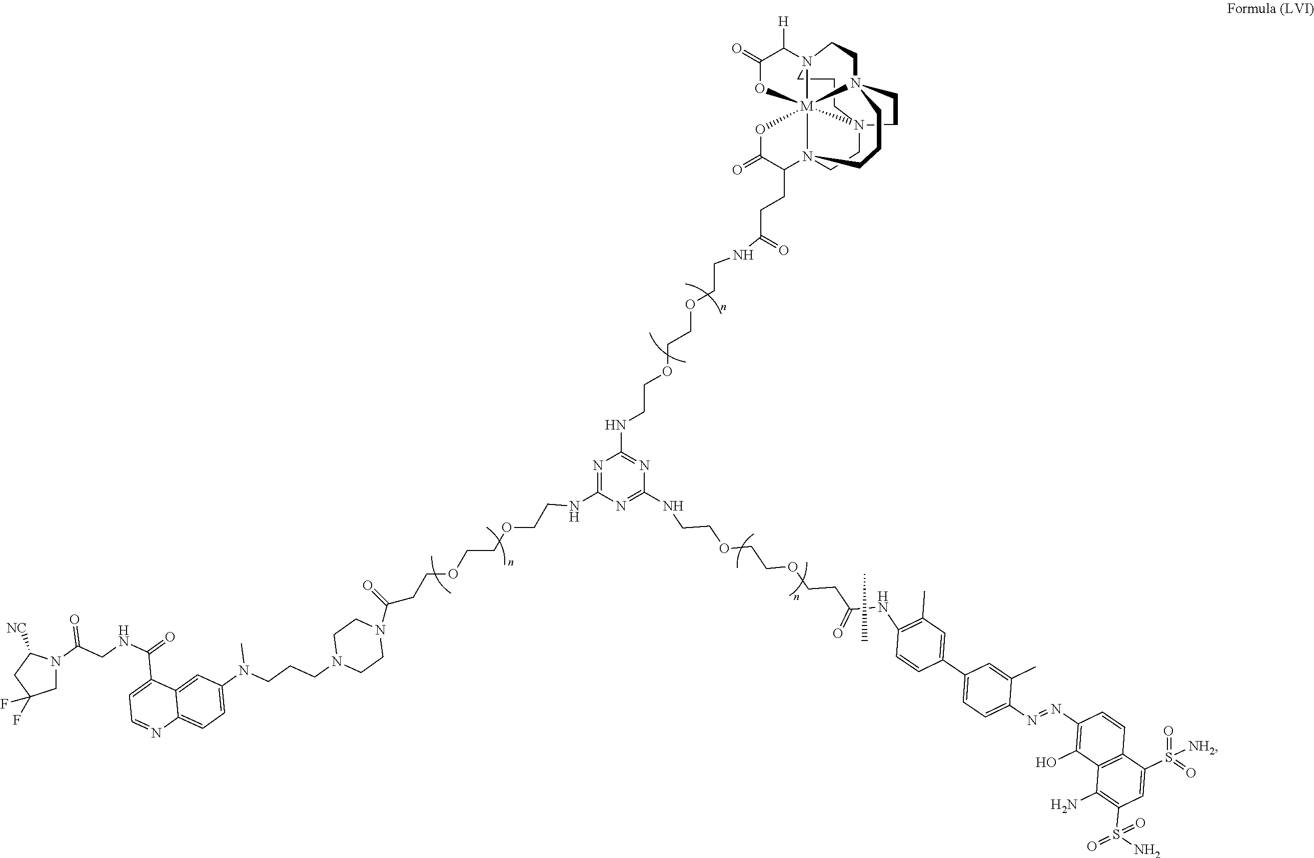

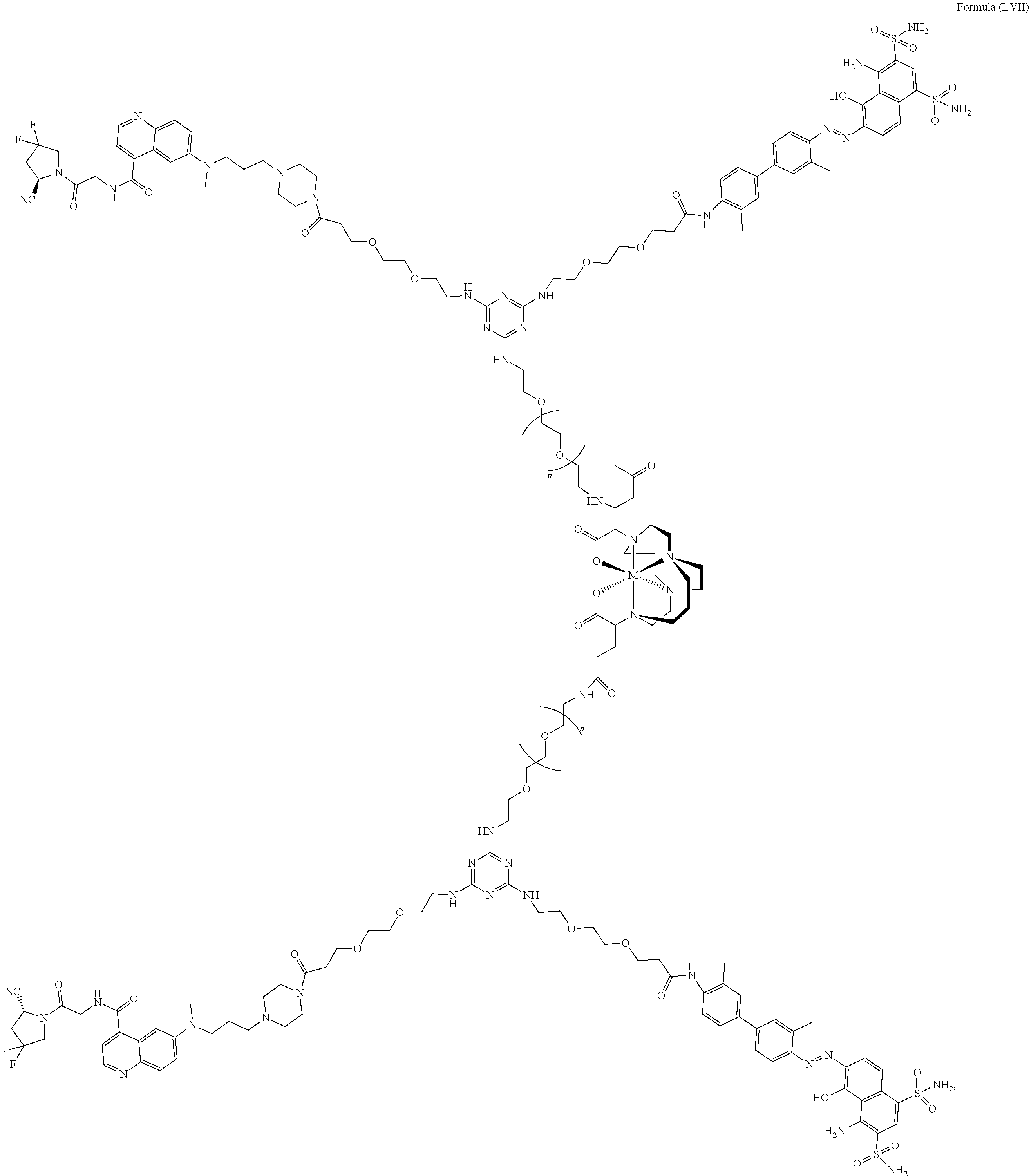

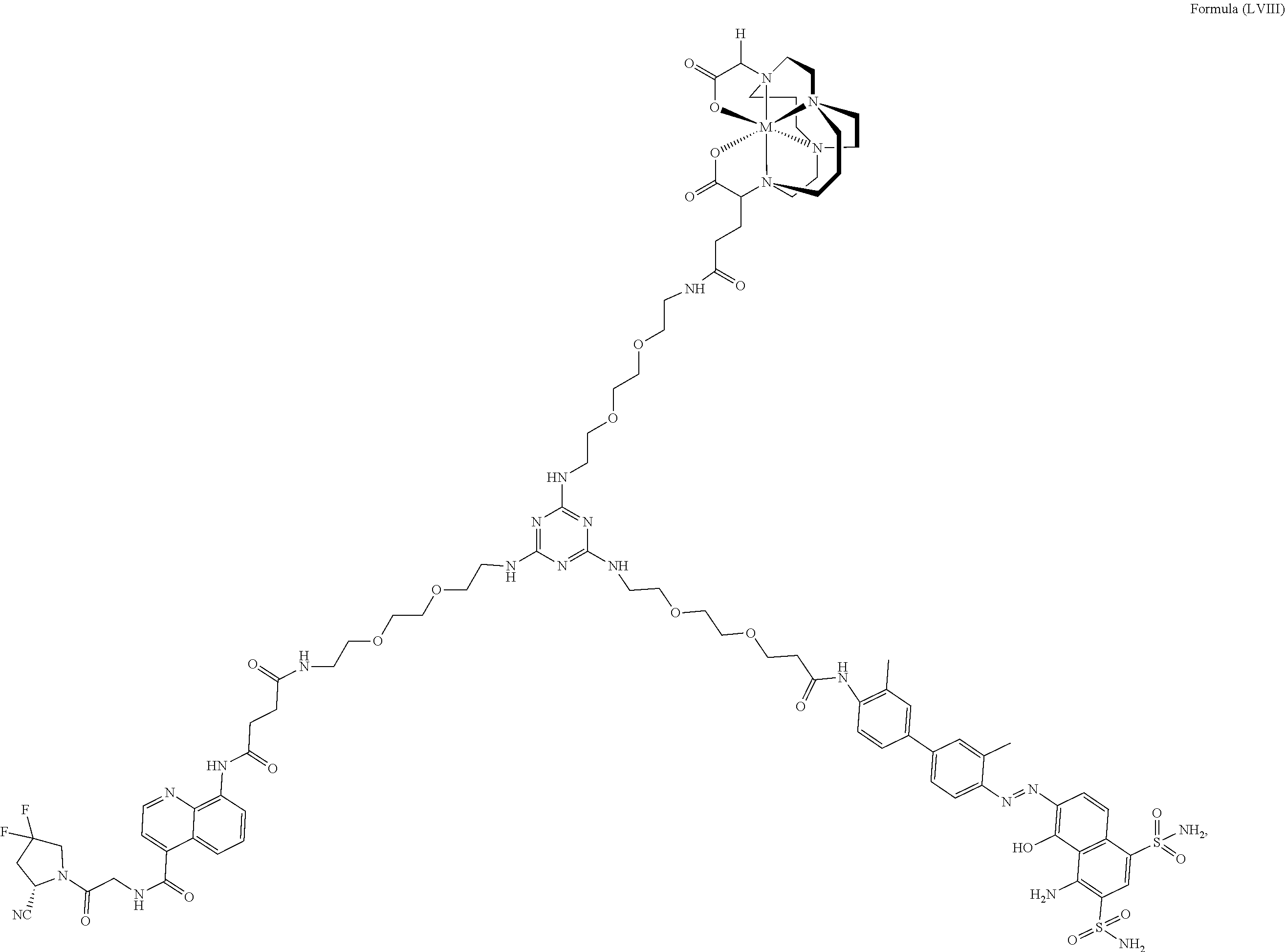

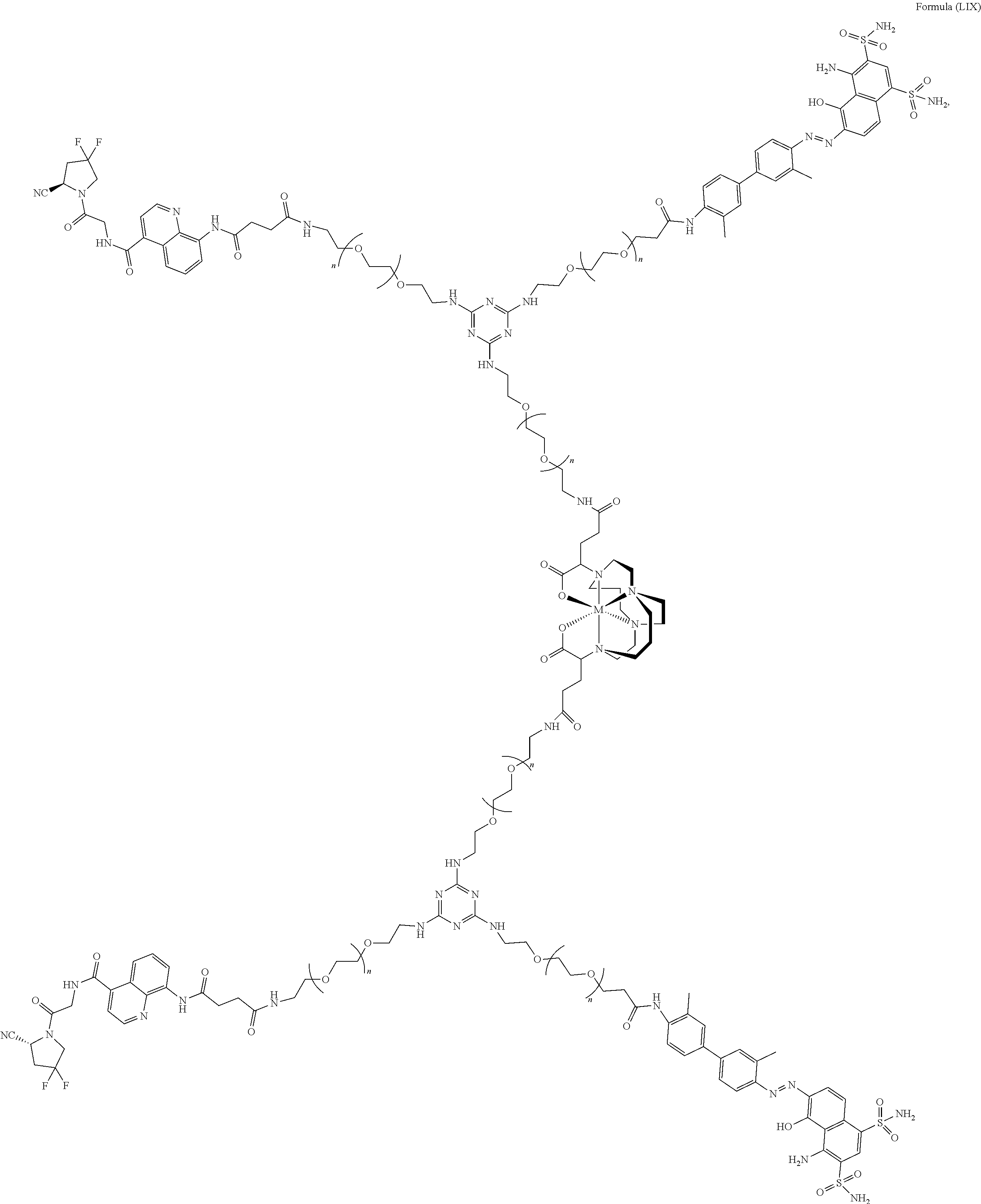

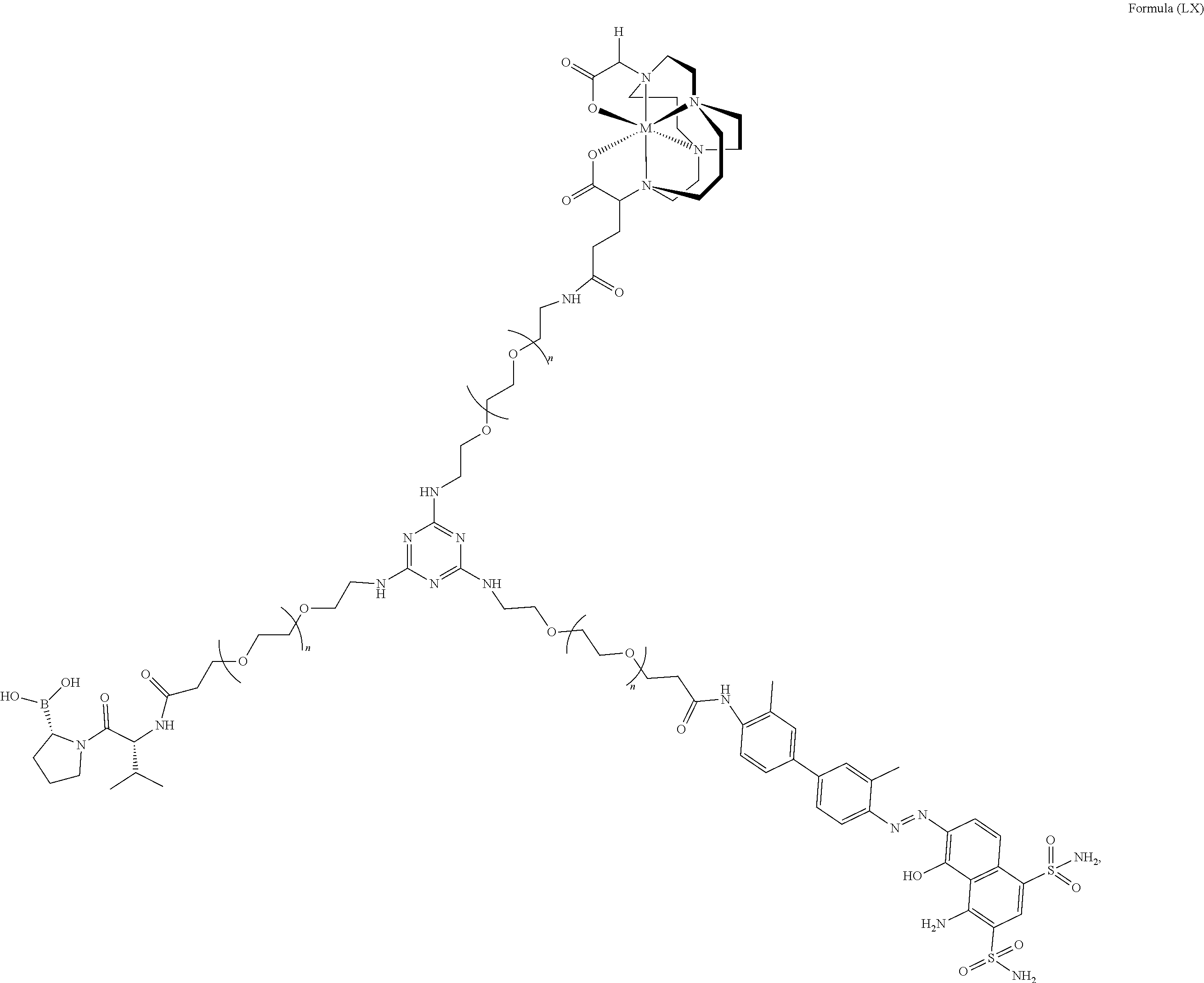

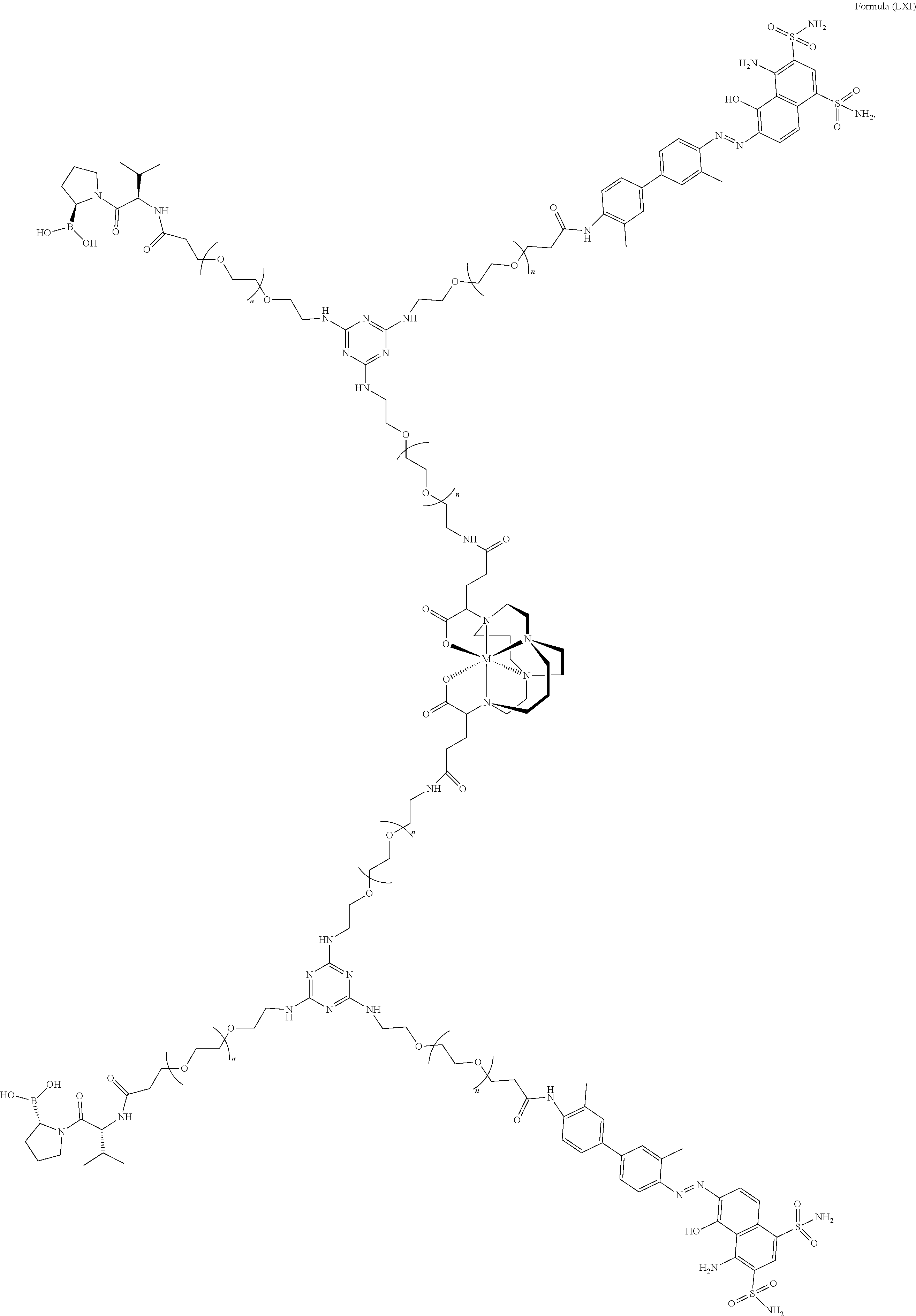

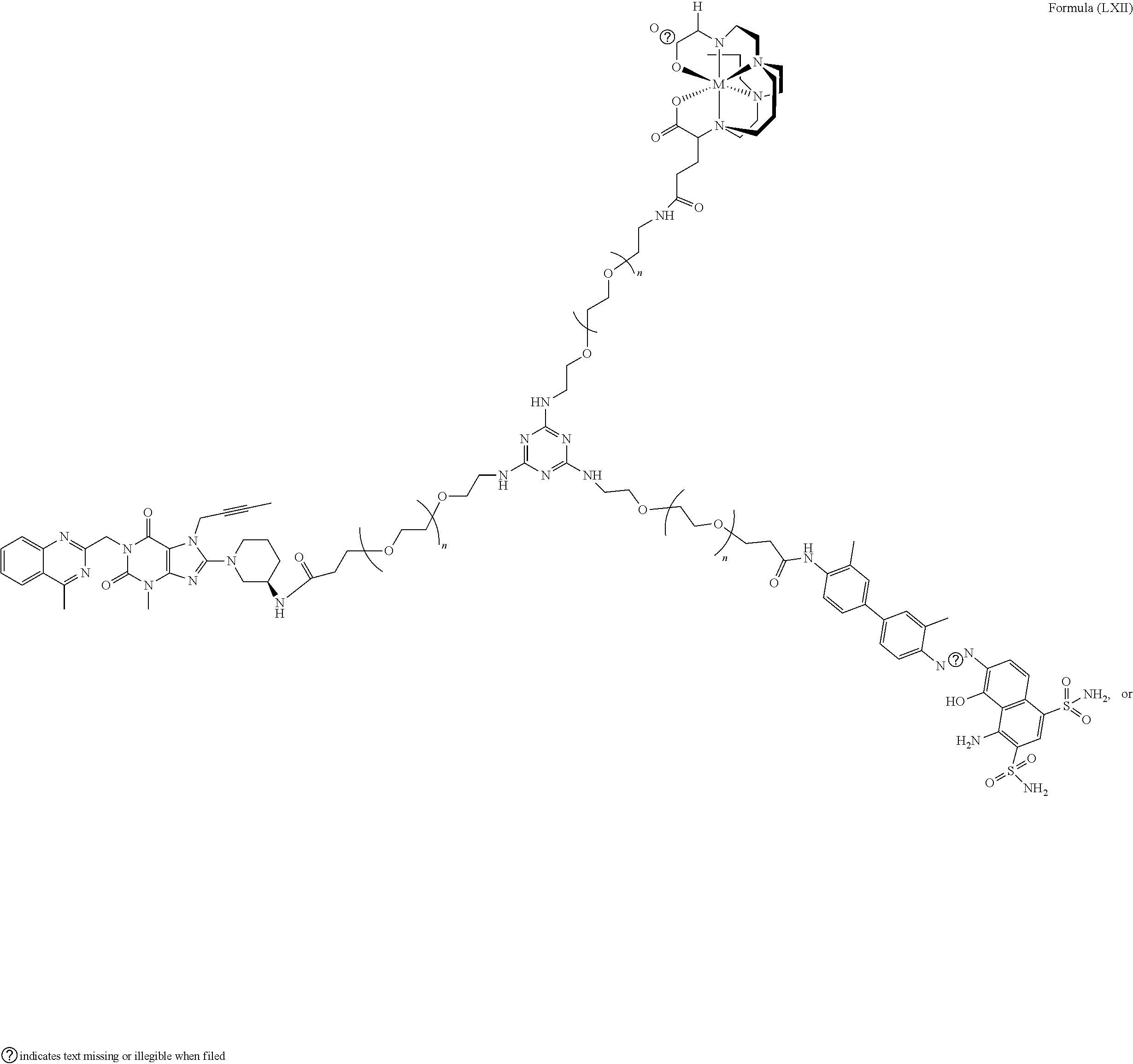

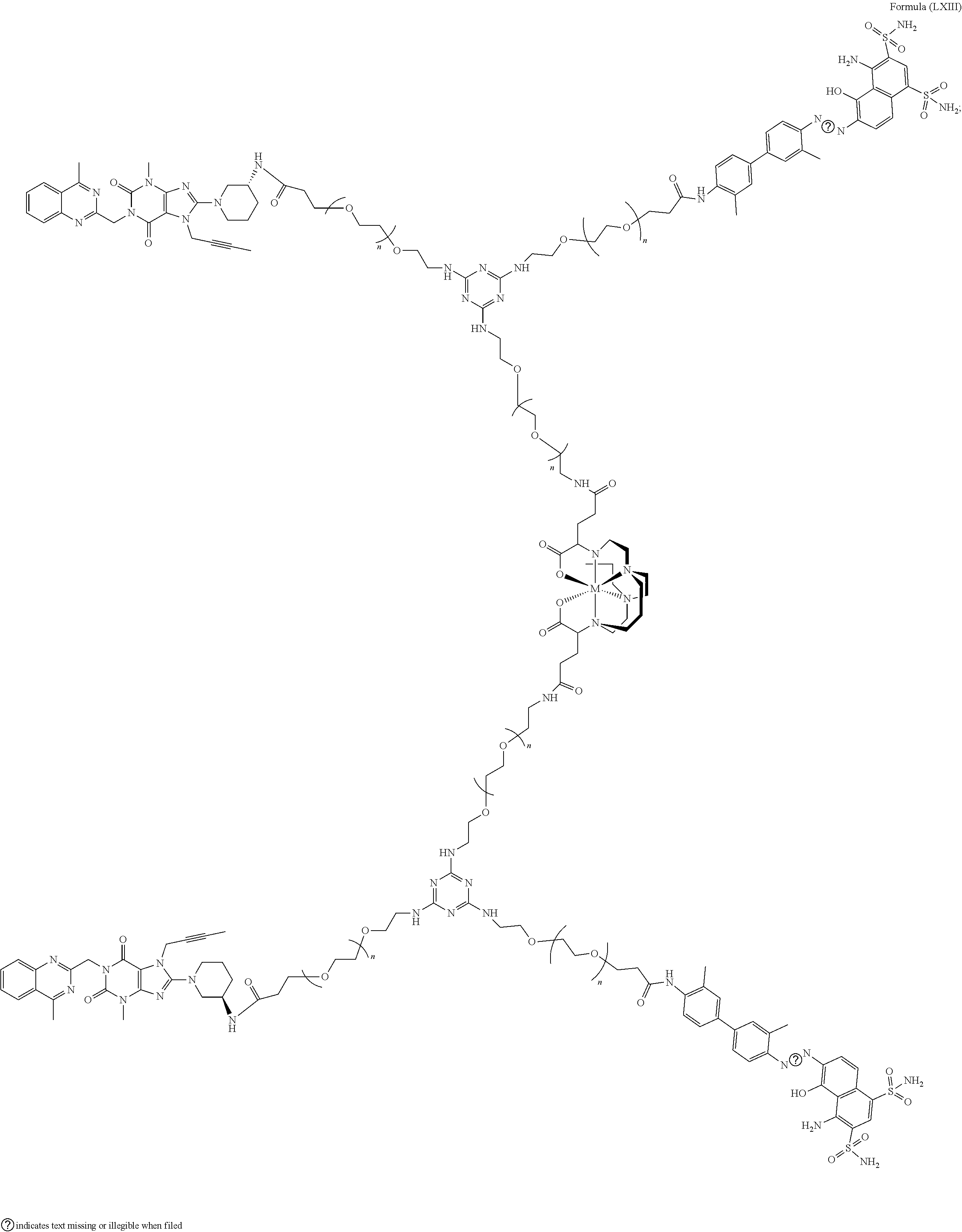

In Formula (IV) through Formula (LXIII), n is 1 to 12 ethylene glycol repeating units or 1 to 6 amino acids, and R₃ comprises —OH, —SH, or —NH₂.

In another aspect, the current disclosure also encompasses a compound of Formula (a) through Formula (h.b3), any positional isomer, any stereo isomer, or any pharmaceutically acceptable salts thereof:

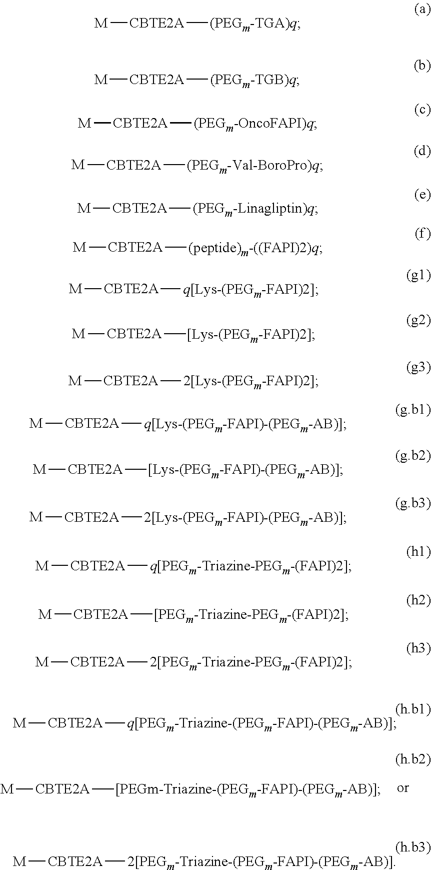

In Formula (a) through Formula (h.b3), M is a chelating copper, including but not limited to ⁶⁰Cu, ⁶¹Cu, ⁶²Cu, ⁶⁴Cu, or ⁶⁷Cu; m is zero, or any integer of 1 to 12; q is 1 or 2; PEG is polyethylene glycol; AB is an albumin binder; Lys is lysine; CBTE2A represents one of the following two moieties

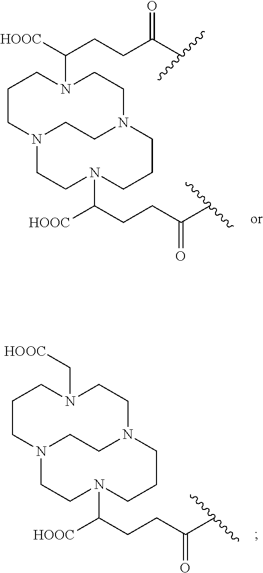 [SMILES: *C(=O)CCC(C(=O)O)N1CCCN2CCN(CCCN(C(CCC(*)=O)C(=O)O)CC2)CC1 ; *C(=O)CCC(C(=O)O)N1CCCN2CCN(CCCN(CC(=O)O)CC2)CC1]

In one aspect, FAPI is a Fibroblast Activation Protein targeting group, comprising TGA, TGB, OncoFAP, Val-BoroPro, or Linagliptin as shown below:

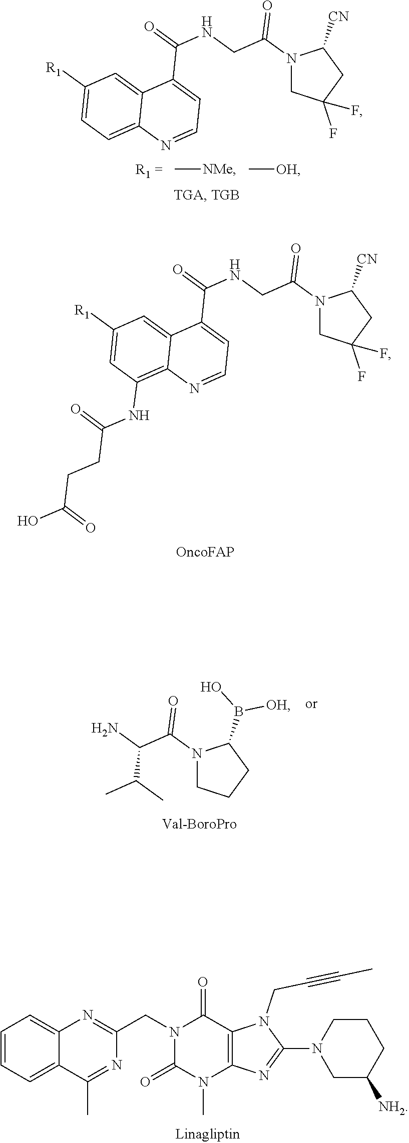

The compound of Formula (a) through Formula (h,b3) include but not limited to the below compounds:

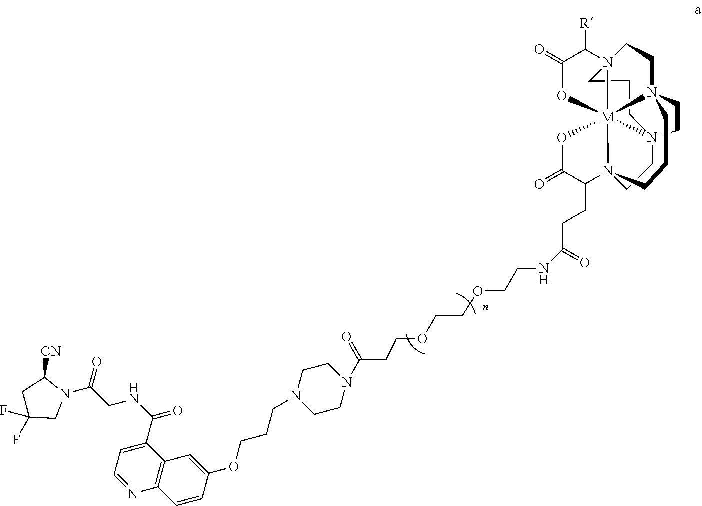

If R′ is —H, a is M-CBTE2A-PEGₘ-TGB, and if R′ is —CH₂CH₂CO-PEGₘ-TGB, a is M-CBTE2A-(PEGₘ-FAPI)₂;

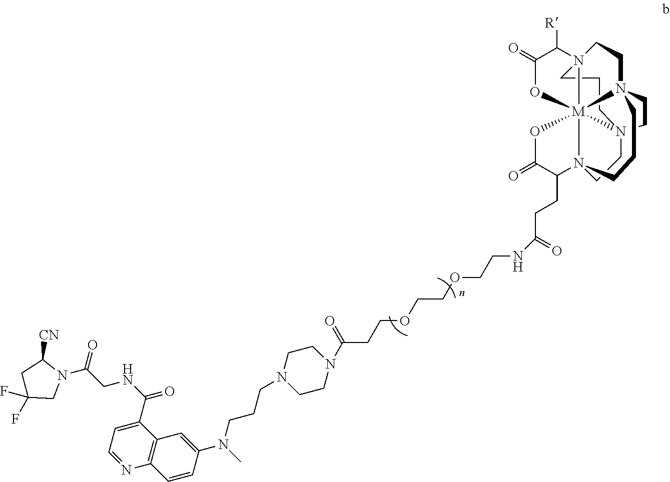

If R′ is —H, b is M-CBTE2A-PEGₘ-TGA, and if R′ is —CH₂CH₂CO-PEGₘ-TGA, b is M-CBTE2A-(PEGₘ-FAPI)₂;

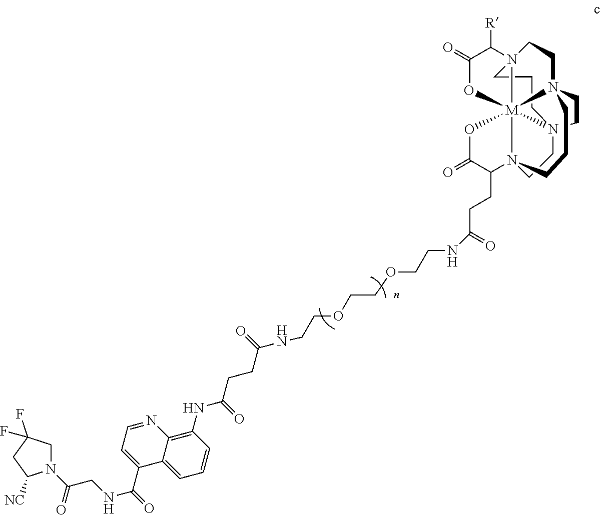

If R′ is —H, c is M-CBTE2A-PEGₘ-OncoFAP, and if R′ is —CH₂CH₂CO-PEG-OncoFAP, c is M-CBTE2A-(PEG-OncoFAP)₂;

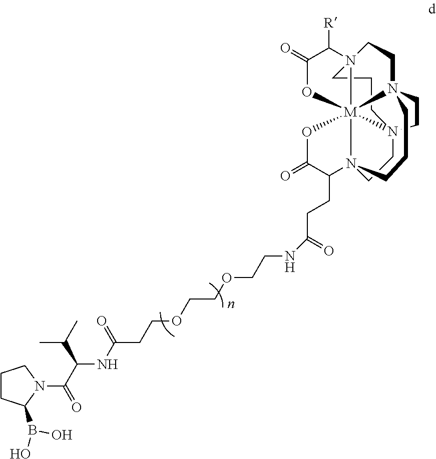

If R′ is —H, d is M-CBTE2A-PEGₘ,ValBoro-Pro, and if R′ is —CH₂CH₂CO-PEGₘ-ValBoro-Pro, d is M-CBTE2A-(PEG-ValBoro-Pro)₂.

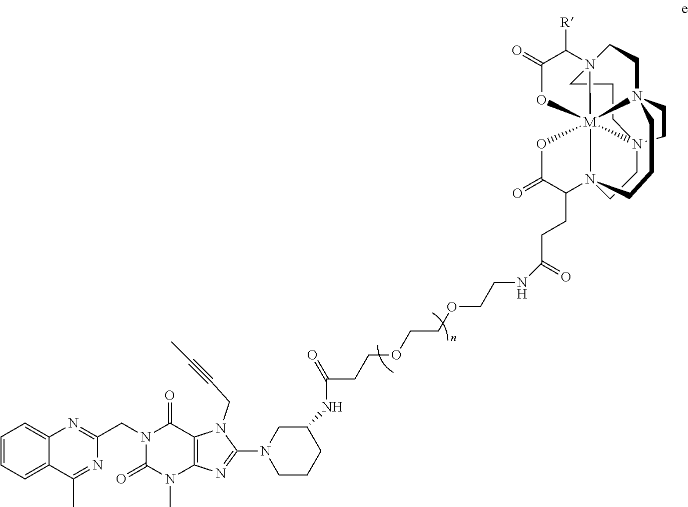

If R′ is —H, e is M-CBTE2A-PEGₘ-Linlagliptinl, and if R′ is —CH₂CH₂CO-PEGₘ,Linagliptin, e is M-CBTE2A-(PEGₘ-Linagliptin)₂;

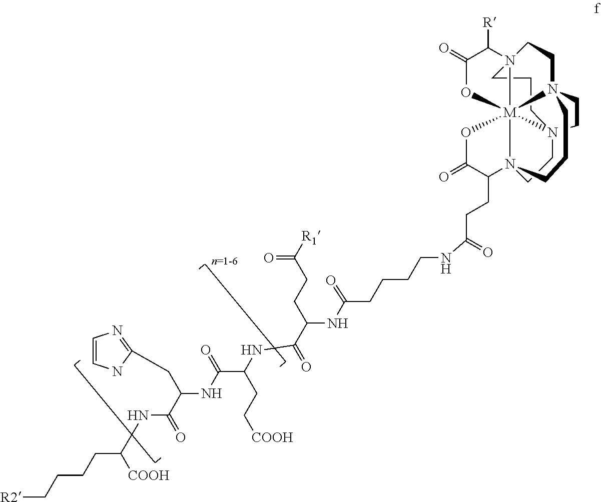

In f, if R is —H, f is M-CBTE2A-Peptide-FAPI, and if R′ is-CH₂CH₂CO—Peptide-FAPI, f is M-CBTE2A-(Peptide-FAPI)₂; R₁′ is —OH or —NH₂; and R₂′ is a chemical moiety of i, ii, iii, iv, or v:

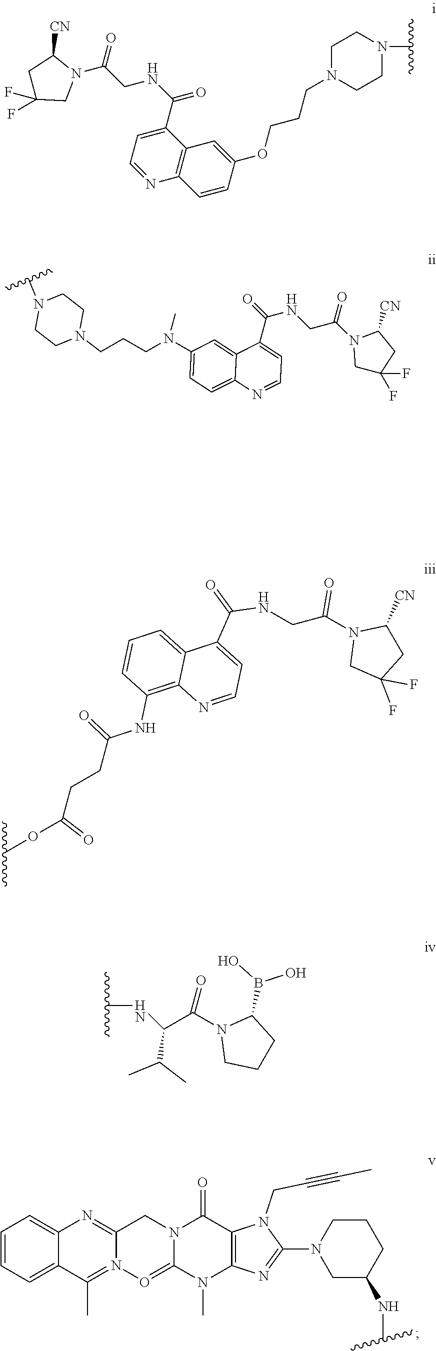

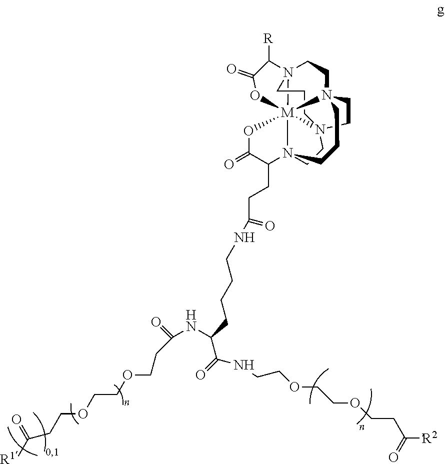

If R′ is —H, g is M-CBTE2A-[Lys-PEGₘ-(FAPI)₂], and if R′ is —CH₂CH₂CO-[Lys-PEGₘ-(FAPI)₂], g is M-CBTE2A-[Lys-PEGₘ-(FAPI)₂]; and R₁′ and R₂′ each is independently selected from the group consisting of i, ii, iiia, iv, and v:

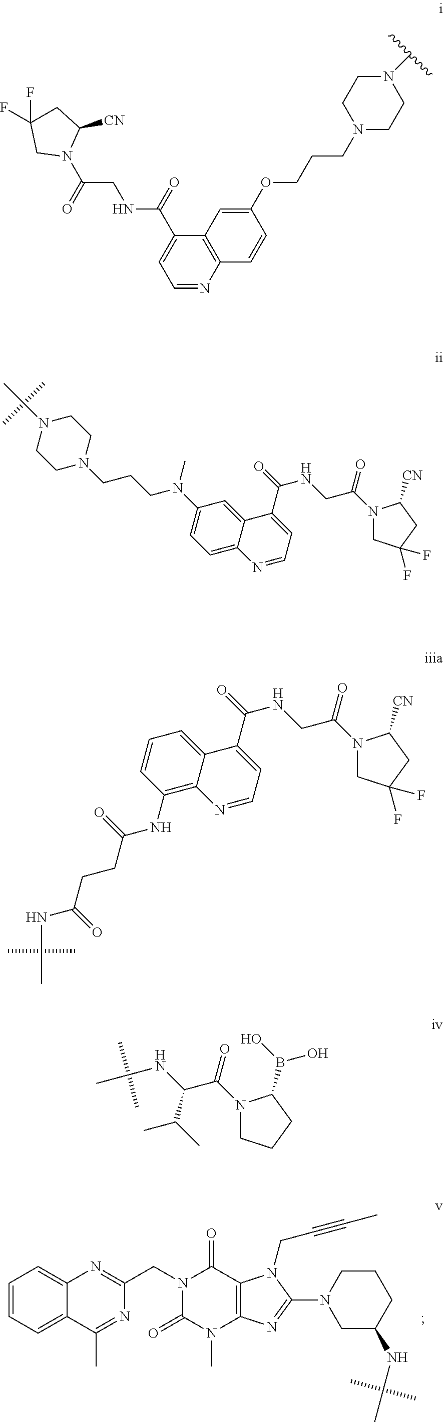

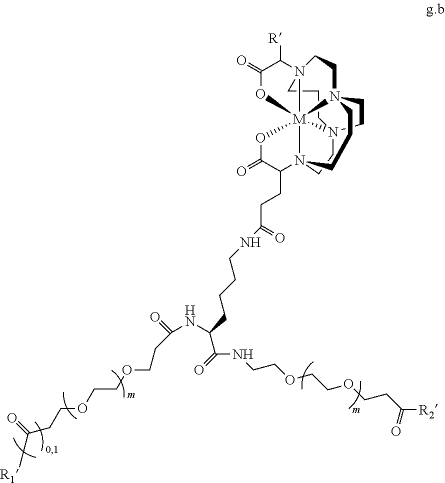

If R′ is —H, (g.b) is M-CBTE2A-[Lys-(PEGₘ-FAPI)-(PEGₘ-AB)], and if R′ is —CH₂CH₂CO-[Lys-(PEGₘ-FAPI)-(PEGₘ-AB)], (g.b) is M-CBTE2A-2[Lys-(PEGₘ-FAPI)-(PEGₘ-AB)]; and R₁′ or R₂′ each is independently selected from the group consisting of i, ii, iiia, iv, v, and vi:

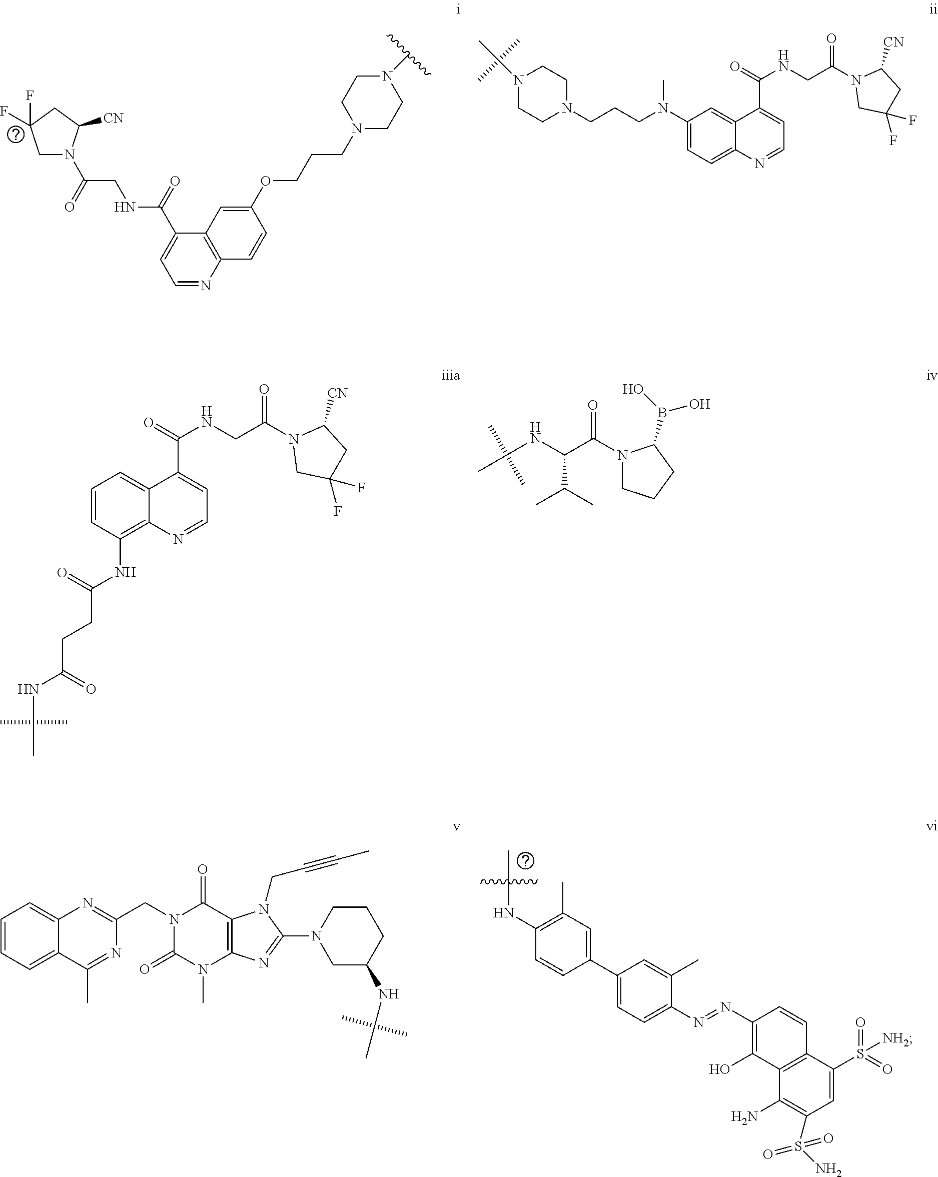

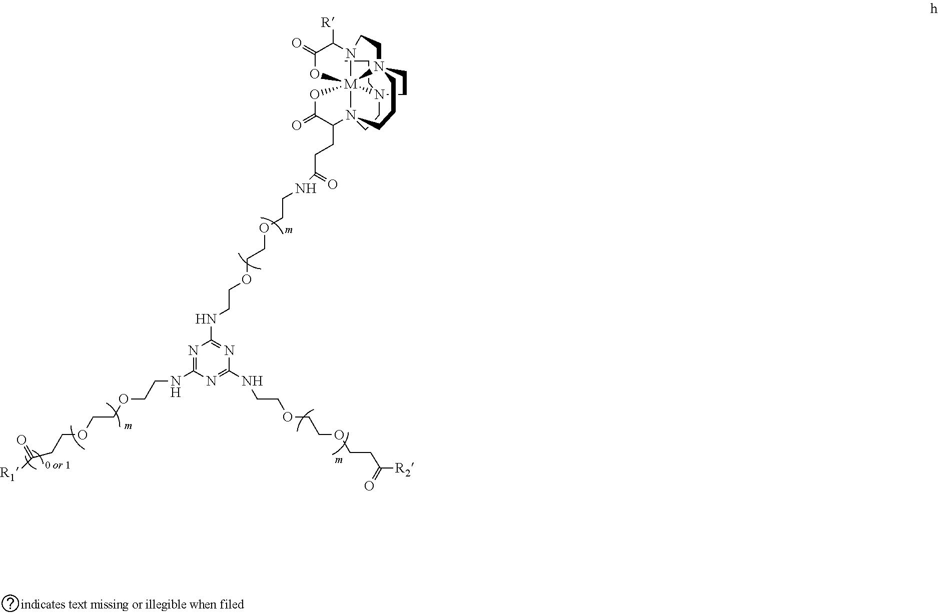

If R′ is —H, h is M-CBTE2A-[PEGₘ-Triazine-(PEGₘ-FAPI)₂], and if R′ is —CH₂CH₂CO—[PEGₘ-Triazine-(PEGₘ-FAPI)₂], h is M-CBTE2A-2[PEGₘ-Triazine-(PEGₘ-FAPI)₂]; and R₁′ and R₂′ each is independently selected from the group consisting of i, ii, iiia, iv, and v:

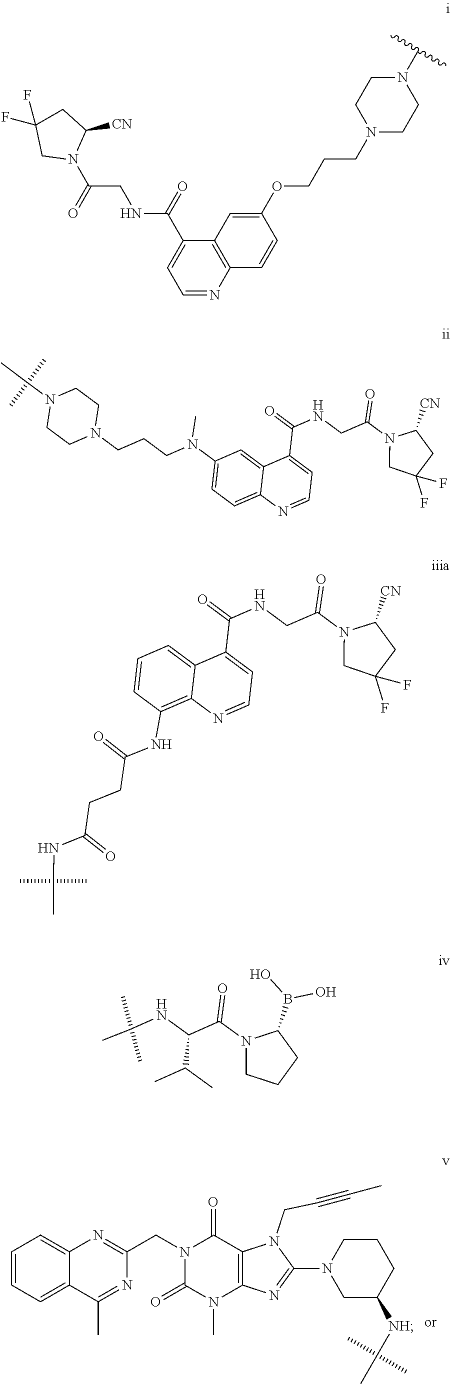

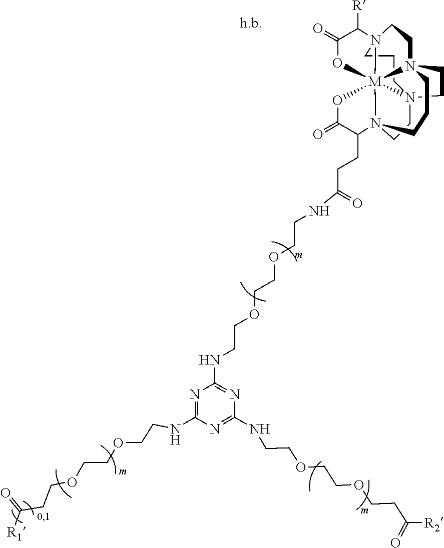

If R′ is —H, (h.b) is M-CBTE2A-[PEGₘ-Triazine-(PEGₘ-FAPI)-(PEGₘ-AB)], and if R′ is —CH₂CH₂CO-[PEGₘ-Triazine-(PEGₘ-FAPI-(PEGₘ-AB)], (h.b) is M-CBTE2A-2[PEGₘ-Triazine-(PEGₘ-FAPI)-(PEGₘ-AB)]; and R₁′ or R₂′ each is independently selected from the group consisting of i, ii, iiia, iv, v, and vi:

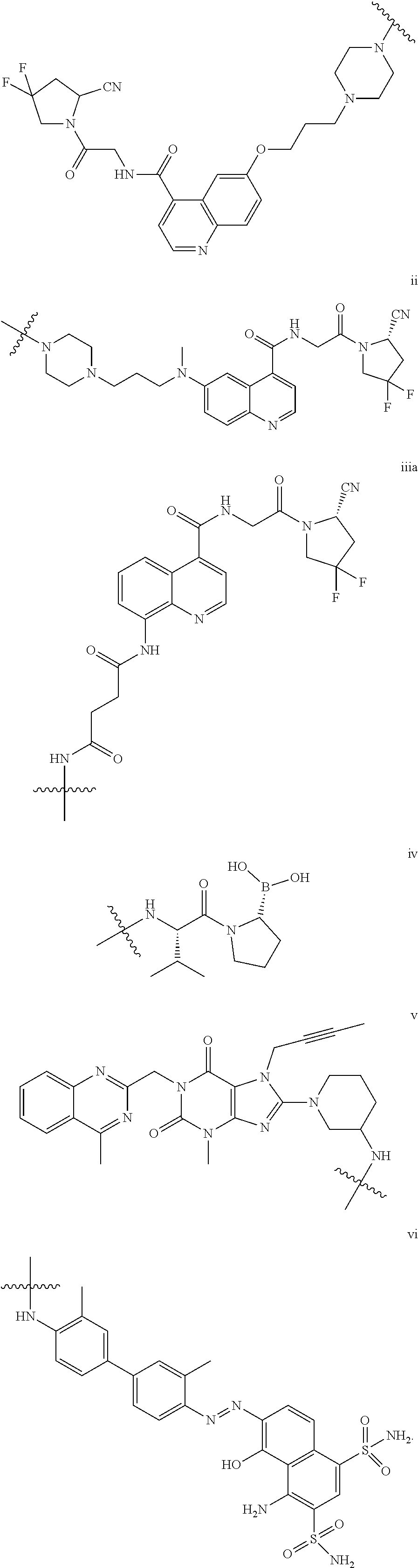

In yet another aspect, the compound comprises Formula (IIa), Formula (IIIa), or a tautomer, an enantiomer, a diastereomer, or a derivative thereof.

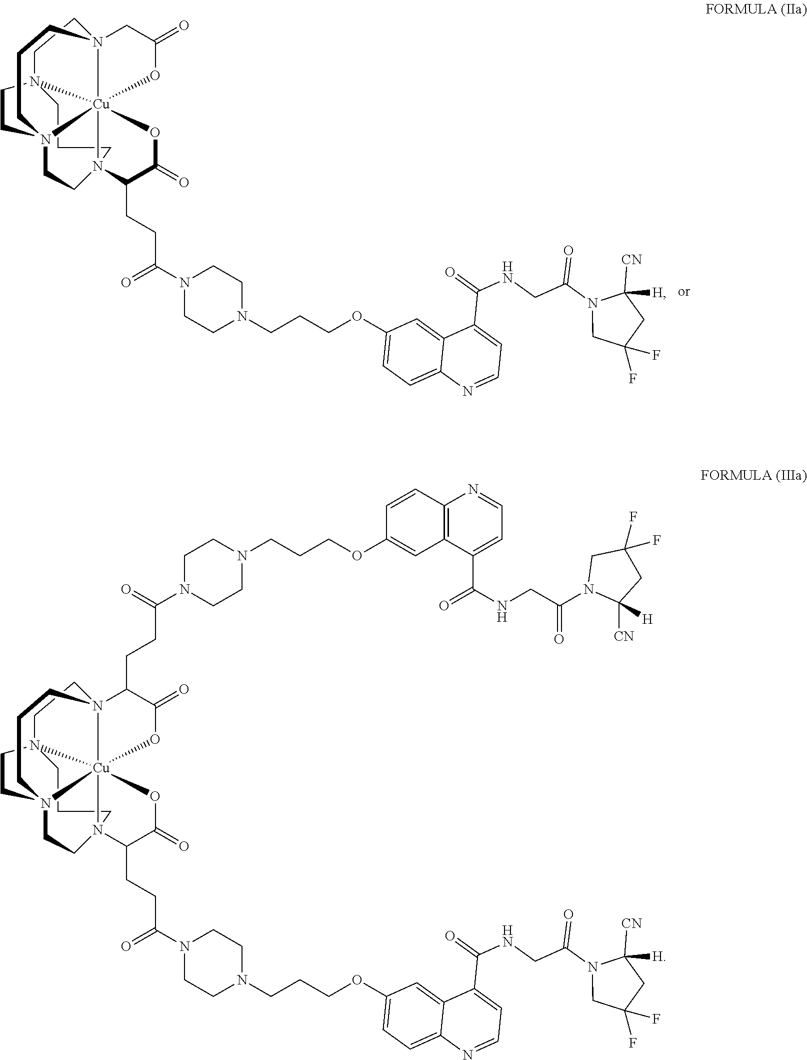

The compound comprising Formula (I), Formula (a) through Formula (h,b) may be in a free form, a positional isomer, a stereoisomer, a crystal form, or a salt. When the compound is in a positional isomer form, it is a constitutional isomer with the same carbon skeleton and have the same functional groups. They differ from each other in the position of the functional groups on the carbon chain. When the compound is in a stereo isomer form, it encompasses a tautomer, an enantiomer, a diastereomer, or a derivative. For example, it can be an optical pure form or a racemic form. When the compound is in a salt form, the salt is preferably a pharmaceutically acceptable salt. Pharmaceutically acceptable salts may include, without limitation, hydrochloride, hydrobromide, phosphate, sulfate, methanesulfonate, acetate, formate, tartaric acid, bitartrate, stearate, phthalate, hydroiodide, lactate, monohydrate, mucate, nitrate, phosphate, salicylate, phenylpropionate, isobutyrate, hypophosphite, maleic, malic, citrate, isocitrate, succinate, lactate, gluconate, glucuronate, pyruvate, oxalate, fumarate, propionate, aspartate, glutamate, benzoate, terephthalate, and the like. In other aspects, the pharmaceutically acceptable salt includes an alkaline or alkaline earth metal ion salt. In particular, sodium, potassium, or other pharmaceutically acceptable inorganic salts are used. The salt forms may be amorphous or in various polymeric forms including hydrates or solvates with alcohols or other solvents.

### Pharmaceutical Compositions Comprising the Compound of Formula (I)

Another aspect of the present disclosure provides a pharmaceutical composition comprising the compounds of any one of Formula (I)-Formula (LXIII), a tautomer, an enantiomer, a diastereomer, or a derivative thereof, and at least one pharmaceutically acceptable excipient or carrier.

A pharmaceutical composition of the disclosure comprises at least one pharmaceutically acceptable excipient. The pharmaceutically acceptable excipients are selected by those of skill in the art based upon the type of formulation. In some aspects, pharmaceutical compositions disclosed herein may comprise dual functionality as a diagnostic and a drug or a theranostic.

In some aspects, the compounds described herein may be administered parenterally.

In some aspects, pharmaceutical compositions can include pharmaceutically acceptable carriers, excipients, and/or stabilizers are nontoxic to recipients at dosages and/or concentrations used to practice the methods disclosed herein. In some aspects, pharmaceutically acceptable carriers, excipients, and/or stabilizers can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some aspects, pharmaceutical compositions described herein can further include an anti-microbial agent. In accordance with these aspects, the anti-microbial agent can, in an example, be an anti-viral, bactericidal agent, anti-fungal, or anti-bacterial agent. For example, the anti-microbial agent can be an anti-bacterial agent (antibiotic) such as doxycycline or other antibiotics such as a general antibiotic.

In certain aspects, pharmaceutical compositions herein can be formulated for parenteral administration, such as intravenous, intracerebroventricular injection, intra-cisterna magna injection, intra-parenchymal injection, or a combination thereof. In some aspects, pharmaceutical compositions herein formulated for parenteral administration can include one or more sterile liquids as pharmaceutically acceptable carriers. Non-limiting examples of sterile liquids suitable for use as pharmaceutically acceptable carriers herein can be water and oil, including those of petroleum, animal, vegetable, or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. In some aspects, pharmaceutical compositions disclosed herein can be packaged in single unit dosages or in multi-dosage forms.

In some aspects, pharmaceutical compositions herein suitable for parenteral administration can include aqueous and non-aqueous sterile injection solutions which can further contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from about 3 to about 9). The preparation of suitable parenteral formulations under sterile conditions is readily accomplished by standard pharmaceutical techniques well known to those skilled in the art.

In certain aspects, pharmaceutical compositions disclosed herein can be in unit dosage forms such as solutions or suspensions, for parenteral administration. Such parenteral composition may be a liquid composition and may be suitable for either infusion or injection. Such parenteral administration may include sterile solution for injection, sterile suspension for injection, sterile emulsions, and dispersions. Such parenteral composition may comprise a carrier or an excipient, such as one or more chelating agents, a calcium ion, isotonic agents, carriers, buffers, co-solvents, diluents, preservatives, and/or surfactants, or combinations thereof. In certain aspects, the carrier or the excipient is an aqueous carrier. And such aqueous carrier includes, but are not limited to sterile water, water for injection, sodium chloride, Ringer's injection, isotonic dextrose injection, dextrose, and lactated Ringer's injection. In some aspects, the parenteral composition comprises water for injection. In some aspect, the water for injection further comprises calcium EDTA or a calcium salt EDTA derivative, or sodium chloride in an amount such that the final solution is isotonic (e.g., 0.1%, 0.25%, 0.45% 0.65%, 0.9% sodium chloride). In some embodiments, the sodium chloride is present in an isotonic amount, such that final concentration of sodium chloride is 0.65%.

### III. PROCESSES TO PREPARE COMPOUNDS COMPRISING FORMULA (I)

In another aspect, the present disclosure also encompasses processes to prepare compounds of Formula (I)-Formula (LXIII) or a tautomer, an enantiomer, a diastereomer, or a derivative thereof or a composition. The process is exemplified in the examples. In some aspects, the current disclosure also encompasses routine modification of the processes to prepare using protocols and procedures and manufacturing methods known in the art. Below is an exemplary preparation process, comprising the steps of

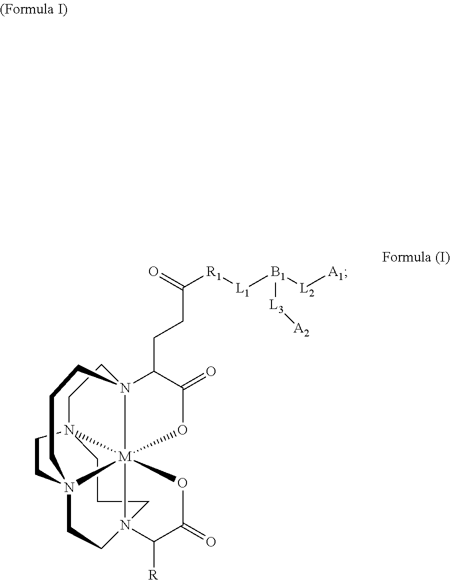

(a) contacting the compound of Formula (LXIV):

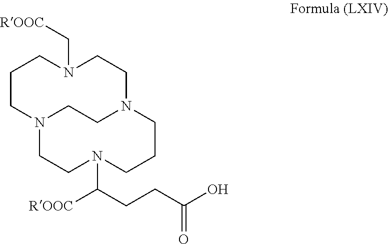

with the compound of Formula (LXV):

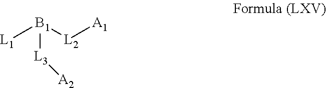

in the presence of an acyl coupling reagent and proton acceptor to form the compound of Formula (LXVI):

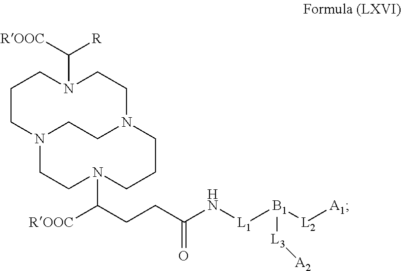

(b) contacting the compound of Formula (LXVI) with a proton acceptor or a proton donor to form the compound of Formula (LXVII).

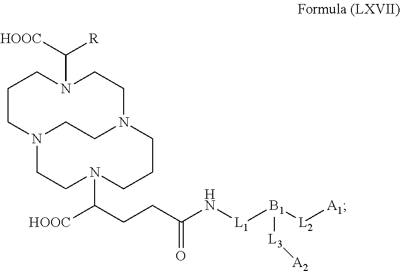

(c) contact the compound of Formula (LXVII) with a M-salt in the presence of a buffer to form the compound of Formula (I).
   In Formula (I), A₁ is a Fibroblast Activated Protein (FAP) targeting group (FAPI) or an albumin binder (AB) group, wherein the FAPI or the AB group is capable of covalent or non-covalent binding with the FAP or albumin respectively; A₂ is a Fibroblast Activated Protein (FAP) targeting group (FAPI), an albumin binder (AB) group, or absent, wherein the FAPI or the AB group is capable of covalent or non-covalent binding with the FAP or albumin respectively; R₁ is —NH, piperazine, piperidine, an alkylamine, a dicarboxylate amide, or is absent; L₁ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof; L₂ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent; L₃ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent; B₁ is a heterocyclic group, a heterocyclic alkyl, an amidoalkyl, an alkylamide, or is absent; and R is a H hydrogen or Formula (Ia)

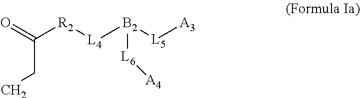

In Formula (Ia), A₃ is a Fibroblast Activated Protein (FAP) targeting group (FAPI) or an albumin binder (AB) group, wherein the FAPI or the AB group is capable of covalent or non-covalent binding with the FAP or albumin respectively; A₄ is a Fibroblast Activated Protein (FAP) targeting group (FAPI), an albumin binder (AB) group, or absent, wherein the FAP targeting group (FAPI) or the AB group is capable of covalent or non-covalent binding with the FAP or albumin respectively; R₂ is —NH, piperazine, piperidine, an alkylamine, a dicarboxylate amide, or is absent; L₄ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof; L₅ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent; L₆ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent; B₂ is a heterocyclic group, a heterocyclic alkyl, an amidoalkyl, an alkylamide, or is absent; M is ⁶⁴Cu, ⁶⁰Cu, ⁶¹Cu, ⁶²Cu or ⁶⁷Cu. In one aspect, the above method yields the compound of Formula (I) in a yield greater than 60%. In another aspect, the above method yields the compound of Formula (I) in greater than 95% radiochemical purity.

Derivatives of any of the compounds can be synthesized by chemical transformations of the compounds' functional groups using standard chemical reactions. For example, these standard chemical reactions can include, but are not limited to: polar reactions under basic conditions, polar reactions under acidic conditions, pericyclic reactions, and free radical reactions. In another example, these standard chemical reactions can include, but are not limited to: addition reactions, substitution reactions, oxidation reactions, reduction reactions, elimination reactions, hydrolysis, acylation, amidations, etherification, and esterification. Alkane functional group transformations can include, but are not limited to, free radical chlorination (hv, C₂), free radical bromination (hv, Br₂), and allylic bromination (NBS). Alkene functional group transformations can include, but are not limited to, addition of HCl, addition of HBr, addition of HI, addition of H₃O(+), chlorination (Cl₂), bromination (Br₂), iodination (I₂), chlorohydrin formation (Cl₂/H₂O), bromohydrin formation (Br₂/H₂O), ether formation (H*/ROH), oxymercuration (Hg(OAc)₂/H₂O), oxymercuration, (Hg(OAc)₂/ROH), hydroboration, epoxidation (RCO₃H), dihydroxylation (OsO₄), dihydroxylation (KMnO₄), cyclopropanation, dichlorocyclopropanation, ozonolysis (reductive workup), ozonolysis (oxidative workup), oxidative cleavage (KMnO₄), hydrogenation, rearrangements (H shift), rearrangements (alkyl shift), free radical addition of HBr, and Sharpless epoxidation. Alkyne functional group transformations can include, but are not limited to, deprotonation (acetylide formation), S_{N}2 with alkyl halides, partial reduction (Lindlar), partial, reduction (Na/NH₃), hydroboration, oxymercuration, addition of HCl, HBr, or HI, addition of HCl, HBr, or HI, hydrogenation, ozonolysis, oxidative cleavage (KMnO₄), and halogenation (Cl₂, Br₂, I₂). The substitution reaction can include, but is not limited to: alcohol formation, nitrile formation, thiol formation, ether formation, thioether formation, azides, ester formation, acetylide addition, alkanes (Gilman reagents), ammonium salt formation, alkyl chloride formation, alkyl bromide formation, alkyl iodide formation, alkyl shift, and hydride shift. Elimination reactions can include, but are not limited to: alkenes from alkyl halides, alkenes from alcohols (strong acid), alkenes from alcohols (POCl₃), alkenes from alkyl halides, E1 with rearrangement (alkyl shift), Hoffmann elimination, and alkyne formation via elimination E1 with rearrangement (hydride shift). Organometallic reactions can include, but are not limited to: Grignard formation (alkyl halides), Grignard formation (alkenyl halides), reaction of Grignards with acids, addition of Grignards to aldehydes, addition of Grignards to ketones, addition of Grignards to esters, reaction of Grignards with CO₂, addition of Grignards to nitriles, formation of organolithium reagents, formation of Gilman reagents, S_{N}2 with Gilman reagents, addition of Gilman reagents to enones, addition of Gilman to acyl halides, Heck reaction, Suzuki reaction, and Stille reaction. Reactions of epoxides can include, but are not limited to: epoxide opening (basic conditions), epoxide opening (acidic conditions), epoxide opening (diol formation), epoxide formation (from halohydrins), epoxide formation (from alkenes), and Sharpless epoxidation of alkenes. Reactions of alcohols and thiols can include, but are not limited to: deprotonation (alkoxide formation), protonation (onium ion formation), conversion to tosylates/mesylates, conversion to alkyl chlorides (SOCl₂), conversion to alkyl bromides (PBr₃), oxidation to aldehydes (PCC), oxidation to ketones (PCC+others), oxidation to carboxylic acid, (H₂CrO₄+others), protection as silyl ethers, thiol formation (S_{N}2), and thiol oxidation to disulfides. Reactions of dienes can include, but are not limited to: Diels-alder reaction, polymerization of dienes, reactions of aromatics (arenes), nitration (HNO₃/H₂SO₄), chlorination (Cl₂ plus catalyst), bromination (Br₂ plus catalyst), sulfonylation (SO₃/H₂SO₄), Friedel Crafts alkylation (R-X plus catalyst), Friedel Crafts acylation (RCOX plus catalyst), iodination (I₂/catalyst), Side chain oxidation (KMnO₄), reduction of nitro groups, reduction of aromatic ketones, Side chain bromination, nucleophilic aromatic substitution (S_{N}Ar), and aryne formation (S_{N}Ar via arynes). Reactions of aldehydes and ketones can include, but are not limited to: hydrate formation (H₂O), cyanohydrin formation (CN), reduction of aldehydes (NaBH₄), reduction of aldehydes (LiAlH₄), reduction of ketones (NaBH₄), reduction of ketones (LiAlH₄), Grignard addition to aldehydes, Grignard addition to ketones, acetal formation (ROH/H⁺), acetal hydrolysis (H₃OH⁺), imine, formation (RNH₂), Enamine formation (R₂NH), Wolff-Kishner: reduction to alkanes, Clemmensen, reduction to alkanes, oxidation to carboxylic acid (H₂CrO₄ or KMnO₄), keto-enol tautomerism, enolate formation, aldol addition reaction, alkylation of enolates, Wittig reaction (alkene formation), thioacetal formation, imine hydrolysis, oxidation to carboxylic acids (Tollens), haloform reaction, Baeyer-Villiger reaction, aldol condensation, Cannizarro reaction. Reactions of carboxylic acids can include, but are not limited to: deprotonation (carboxylate formation), formation via Grignard and CO₂, conversion to acid chloride (SOCl₂), reduction (LiAlH₄), Fischer esterification, and decarboxylation (of β-keto acids). Reactions of esters can include, but are not limited to: reduction to aldehydes (DIBAL-H), reduction to alcohols (LiAlH₄), hydrolysis to carboxylic acid (acidic), hydrolysis to carboxylic acid (basic), addition of Grignard reagents to esters, Claisen condensation, and transesterification (basic conditions). Reactions of acyl halides can include, but are not limited to: conversion to esters (ROH), conversion to carboxylic acids (H₂O), conversion to anhydrides (RCO₂), conversion to amides (RNH₂), conversion to ketones (Gilman reagents), and conversion to aldehydes (LiAlH(OtBu)₃). Reactions of α,β-unsaturated ketones (enones) can include, but are not limited to: Michael reaction (conjugate addition of enolates), conjugate addition of Gilman reagents, conjugate addition of other nucleophiles. Reactions of amines and amides can include, but are not limited to: dehydration of amides to nitriles (P₂O₅), Hofmann rearrangement, Gabriel synthesis of amines, reductive amination, formation of diazonium salts, reactions of diazonium salts, amide formation using DCC, amide formation from acid halides, and Curtius rearrangement.

### IV. METHODS OF DETECTING AND/OR TREATING CANCER

Another aspect of the present disclosure provides methods of treating and/or detecting cancer. In one aspect, the method comprises administering an effective amount of any one or more of the compounds of Formula (I)-Formula (LXIII) or a tautomer, an enantiomer, a diastereomer, or a derivative thereof or a composition comprising any one or more of the compounds of Formula (I)-Formula (LXIII) or a tautomer, an enantiomer, a diastereomer, or a derivative thereof to a subject in need thereof wherein the subject in need thereof has or is suspected of one or more cancers.

In certain aspects, methods disclosed herein may be used for detecting cancer. Thus, in some aspects, the present disclosure provides methods for detecting cancer. In some aspects, the methods disclosed herein involve a theranostic method. Theranostics as used herein is a treatment strategy that combines therapeutics with diagnostics. It associates both a diagnostic test (for example, administering a composition as disclosed herein, and imaging) that identifies patients most likely to be helped or harmed by a medication and targeted drug therapy based on the test results. In some aspects, the method disclosed herein may comprise the steps of administering a suitable amount of the compositions disclosed herein and further include an imaging step. In some aspects, the imaging technique is nuclear imaging technique like positron emission tomography (PET when M=⁶⁰Cu, ⁶¹Cu, ⁶²Cu, or ⁶⁴Cu) or single photon emission computed tomography (SPECT when M=⁶⁷Cu). In some aspects, the method comprises administering to a subject in need there of a suitable amount of a theranostic comprising the compounds disclosed herein wherein the radionuclide M as disclosed here is either ⁶⁴Cu or ⁶⁷Cu and imaging is done using a PET (⁶⁴Cu) or SPECT (⁶⁷Cu) scan.

In certain aspects, methods disclosed herein may be used for detecting, preventing, alleviating and/or treating cancer. Thus, in some aspects, the present disclosure provides methods for detecting, alleviating one or more symptoms and/or for treating cancer in a subject in need thereof by administration of any of the compounds disclosed herein, as well as a pharmaceutical composition comprising such.

In some aspects, the detection of FAP positive cancer using the methods herein may provide beneficial outcomes for patients due to for example, i) detection of smaller primary tumors and, thus the possibility of earlier diagnosis, (ii) the detection of smaller metastasis and, thus a better assessment of tumor stage, (iii) precise intra-operative guidance facilitating complete surgical removal of tumor tissue, (iv) better differentiation between inflammation and tumor tissue, (v) more precise staging of patients with tumors, (vi) better follow up of tumor lesions after antitumor therapy, (vii) the opportunity to use the molecules as theranostic agents for diagnosis and therapy. Furthermore, the molecules can be used for the diagnosis and treatment of non-malignant diseases such as chronic inflammation, atherosclerosis, fibrosis, tissue remodeling and keloid.

To perform the methods disclosed herein, an effective amount of the compounds or compositions disclosed herein may be administered to a subject who needs treatment or detection of cancer parenterally at a suitable amount as disclosed herein. Parenteral delivery may include any of intravenous injection, intravenous infusion, intradermal, intralesional, intramuscular, subcutaneous injection or depot administration. The parenteral formulation may comprise an active ingredient and a pharmaceutically acceptable carrier and/or excipient. Typical carrier or excipient includes, but are not limited to surfactants, preservatives, diluents, buffers, and co-solvents.

The compounds disclosed herein may be administered as primary therapy, or as adjunct therapy, either following local intervention (surgery, radiation, local chemotherapy) or in conjunction with at least one other chemotherapeutic agent.

Suitable subjects may include, without limit, humans, as well as companion animals such as cats, dogs, rodents, and horses; research animals such as rabbits, sheep, pigs, dogs, primates, mice, rats, and other rodents; agricultural animals such as cows, cattle, pigs, goats, sheep, horses, deer, chickens, and other fowl; zoo animals; and primates such as chimpanzees, monkeys, and gorillas. The subject can be of any age without limitation. In an aspect, the subject may be a human.

Generally, the composition will be administered in a therapeutically effective amount which includes prophylactic amounts or lower dosages for example, when combined with another agent. As used herein, “an effective amount” refers to doses of compound sufficient to provide circulating or local concentrations high enough to impart a beneficial effect on the recipient thereof. The precise amount to be administered can be determined by the skilled practitioner in view of desired dosages, side effects, and medical history of the patient. In certain aspects, a compound disclosed herein may be administered to a subject intravenously at a concentration ranging from about 0.03 mCi/kg to about 3.0 mCi/kg. For a 70-kg adult, ~2.0 mCi (for diagnosis) to 200 mCi (for radiotherapy) each injection. One injection each time; once or twice a week up to two months each course; multiple courses may be required. In some aspects, the one or more cancers comprise cancers characterized by overexpression of fibroblast activation protein (FAP) non-limiting examples of which include breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, thymus carcinoma, desmoid tumors, glioma, astrocytoma, cervix carcinoma and prostate cancer.

In some aspects, the compounds disclosed herein can also be used for detecting, preventing, alleviating and/or treating other disease conditions characterized by overexpression of fibroblast activation protein (FAP) including but not limited to (FAP) is selected from the group consisting of cancer, chronic inflammation, atherosclerosis, fibrosis, tissue remodeling and keloid disorder.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

The current disclosure is based on the surprising discovery that multivalent scaffold with FAP inhibitor appended to stable and neutral bifunctional ^{64/67}Cu-CB-TE2A complex (see FIG. 1 for a general schematic) could result in enhanced specific binding and tumor retention; often desirable in FAP-targeted theranostic application due to fast clearance of the existing quinolone-based radiotracers from the targeted sites. Furthermore, ⁶⁷Cu inclusion can make the overall radiotracer viable for endoradiotherapy and SPECT imaging, resulting in a complementary feature to diagnostic utility from ⁶⁴Cu-labeled conjugates. Collectively, the proposed conjugated labeled with copper radionuclides have potential applications in cancer theranostic development. Here in are presented in vitro and in vivo results invoking a multivalency approach with cross-bridged cyclam ligand to accommodate one or more FAP inhibitor designed to target Fibroblast Activation Protein in a renal cell carcinoma (RCC) tumorgraft line XP-185 tumor.

### Materials and Methods

Unless otherwise mentioned, all chemicals used in this study were purchased from Milipore Sigma and Alfa Aesar. All reactions were carried out under atmospheric conditions unless otherwise specified. ¹H NMRs were recorded on Bruker 400 MHz NMR spectrometer and referenced to the internal solvent signals (1.9 ppm in CD₃CN). Data are reported as follows: chemical shift (in ppm, δ), integration, multiplicity (s=singlet, d=doublet, t=triplet, q=quartet, m=multiplet, br=broad), coupling constant (in Hz). Thin layer chromatography was performed in silica gel coated aluminum plates (EMD Merck F254, 250 mm thickness). 254 nm ultraviolet light and Chemical stains were used to visualize spots. Silicycle Silicaflash P60 silica gel (mesh 230-400) was used for flash chromatography.

Preparative reversed phase HPLC was performed in an Agilent 1260 Infinity Prep HPLC system equipped with 1260 photodiode array detector and an Agilent Prep-C18 column (150×21.2 mm, 5 μm) using following conditions: linear gradients (10-70% 0.1% TFA in acetonitrile in 20 min; 20 mL/min flow rate; 254 nm UV-absorbance) of 0.1% TFA in water and 0.1% TFA in acetonitrile). Liquid Chromatography-Mass Spectrometry (LC-MS) data were recorded on Agilent 6540 Accurate-Mass Quadrupole Time-of-Flight LC/MS system equipped with 1290 UPLC. The radiolabeled compound was characterized by a Waters 600 HPLC system equipped with a Waters 2996 photodiode array detector and an in-line Shell Jr. 2000 radio detector.

Analytical radio-HPLC was carried out using Agilent 1220 Infinity II analytical HPLC system equipped with a 1220 LC diode array detector (DAD) (76337 Waldbronn, Germany), an in-line Eckert & Ziegler radio detector (Eckert & Ziegler Radiopharma, Inc. Hopkinton, MA, USA), and a C18 Atlantis® T3 column (4.6×250 mm, 5 μm) (Waters, Ireland). The following conditions were applied: linear gradients (10-50% 0.1% TFA in acetonitrile in 20 min; 20 mL/min flow rate; 254 nm UV-absorbance) of 0.1% TFA in water and 0.1% TFA in acetonitrile). The same condition was used for serum stability studies.

An aqueous buffer solution was prepared from Milli-Q water, obtained from a Millipore Gradient Milli-Q water system. CB-TE2A-(^{t}Bu)₂-COOH derivatives were synthesized according to the reported literature. FAPI was synthesized following a known procedures with slight modifications where necessary.

### Chemistry

Boc-FAPI: [(S)—N-(2-(2-cyano-4,4-difluoropyrrolidin-1-yl)-2-oxoethyl)-6-(3-(4-tert-butoxycarbonylpiperazin-1-yl)propoxy)quinoline-4-carboxamide]: A mixture of 6-(3-(4-tert-butoxycarbonylpiperazin-1-yl)-1-propoxy)quinoline-4-carboxylic acid (16.7 mg, 40.0 μmol), HBTU (17.1 mg, 45.0 μmol), DIPEA (27.5 μL, 150 μmol) and (S)-1-(2-aminoacetyl)-4,4-difluoropyrrolidine-2-carbonitrile 4-methylbenzenesulfonate (20.8 mg, 45.0 μmol) in 500 μL DMF was stirred for 16 hours at room temperature under Nitrogen balloon. After completion of the reaction, the mixture was quenched with 500 μL water and purified by semi-preparative RP-HPLC. Lyophilization of the collected fraction gave 15.0 mg (25.6 μmol; 64%) of the desired product Boc-FAPI as pale-yellow solid. LC-MS Rₜ min, 3.8 min; m/z 587.13 [M+H]⁺. Characterization data for the intermediates are as provided below:

### Characterization of Intermediates

### (S)-tert-butyl (2-(2-carbamoyl-4,4-difluoropyrrolidin-1-yl)-2-oxoethyl)carbamate (3)

A solution of 2-((tert-butoxycarbonyl)amino)acetic acid 2 (103.3 mg, 0.59 mmol) in 0.5 mL DMF was added to the mixture of 1 (102 mg, 0.54 mmol), HATU (225 mg, 0.59 mmol), DIPEA (282 μL, 1.62 mmol) in 0.5 mL DMF. The resulting mixture was stirred at room temperature for 14 hours. The crude product solution was diluted with 1.0 mL water and purified by preparative RP-HPLC to afford desired product 3 as white solid in 65% yield. LC-MS Rₜ min, 3.2 min; MS (ESI) m/z calcd for C12H19F2N3O4: 308.14; found 208.09 [M-Boc+H]⁺.

### (S)-tert-butyl (2-(2-cyano-4,4-difluoropyrrolidin-1-yl)-2-oxoethyl)carbamate (4)

Compound 3 (30.8 mg, 0.1 mmol) was dissolved in anhydrous DCM at room temperature. Pyridine (8 μL, 0.1 mmol) and TFAA (14 μL, 0.1 mmol) were added successively. After stirring the solution for two hours, another portion of Pyridine and TFAA was added. The solution was further stirred for 4 hours. The solvent was removed under reduced pressure and the crude product was dissolved in 1 mL acetonitrile. The crude product was purified by preparative RP-HPLC to afford desired product 4 as white solid in 80% yield. LC-MS Rₜ min, 4.1 min; MS (ESI) m/z calcd for C12H17F2N303: 290.13; found 190.08 [M-Boc+H]⁺.

### 6-hydroxyquinoline-4-carboxylic acid (6)

6-methoxyquinoline-4-carboxylic acid (5) (101.6 mg, 0.5 mmol) was heated in 2.5 mL 48% HBr solution at 120° C. for 16 hours. Yellow crystals were formed when the solution was cooled to room temperature. The solid crystals were collected through filtration and were thoroughly washed with acetone followed by diethyl ether which afforded 99% of the desired product 6.

### 6-(3-chloropropoxy)quinoline-4-carboxylic acid (7)

Compound 6 (37.8 mg, 0.2 mmol) was mixed with a solution of Cesium carbonate (325 mg, 1 mmol) and 1-bromo-3-chloropropane (126 mg, 0.8 mmol) in 1.0 mL DMSO. The resulting mixture was stirred at 60° C. for 14 hours. After completion of the reaction, the solution was cooled to room temperature and then diluted with 1.0 mL water followed by 1.0 mL acetonitrile. The mixture was added with 0.5 mL 6 M NaOH and stirred for 2 hours at room temperature. The undissolved solids were filtered out and the crude product in the filtrate was purified with preparative RP-HPLC to afford desired product as pale-yellow solid in 35% yield. LC-MS Rₜ min, 3.0 min; MS (ESI) m/z calcd for C13H12ClNO3: 266.05; found 266.06 [M+2]⁺.

### 6-(3-(4-(tert-butoxycarbonyl)piperazin-1-yl)propoxy)quinoline-4-carboxylic acid (8)

Compound 7 (26.5 mg, 0.1 mmol), sodium iodide (100 mg, 0.66 mmol) and tert-butyl piperazine-1-carboxylate (100 mg, 0.53 mmol) were dissolved in 0.5 mL DMF. The reaction mixture was stirred at 60° C. for 14 hours. The mixture was cooled to room temperature and was diluted with 0.5 mL water and 0.5 mL acetonitrile. The crude product was purified by preparative RP-HPLC to afford desired product 8 as white solid in 58% yield. LC-MS Rₜ min, 2.7 min; MS (ESI) m/z calcd for C22H29N3O5: 416.20; found 416.22 [M+H]⁺.

CB-TE2A-(^{t}Bu)₂-(FAPI): Boc-FAPI (7.0 mg, 12.0 μmol) was first treated with excess para-toluenesulfonic acid monohydrate (19.2 mg, 99.6 μmol) in acetonitrile solvent at 40° C. for 3 hours. After the Boc-deprotection step was completed as confirmed by LCMS analysis, the residual solvent was removed under reduced pressure in rotary evaporator. The crude FAPI was directly used for the next reaction and added to the mixture of CB-TE2A-(^{t}Bu)₂-(COOH) (6.0 mg, 10.0 μmol), HBTU (3.75 mg, 10.0 μmol) and DIPEA (9.0 μL, 50 μmol) in 500 μL DMF. The resulting mixture was then stirred at room temperature for 24 hours under N₂ balloon. After removing DMF under reduced pressure, the crude product was reconstituted in acetonitrile and purified by semi-preparative RP-HPLC. Lyophilization of the collected fraction gave 4.0 mg (4.0 μmol; 40%) of the desired conjugate product CB-TE2A-(^{t}Bu)₂-(FAPI) as white solid. LC-MS Rₜ min, 3.5 min; MS (ESI) m/z calcd for C₅₁H₇₆F₂N₁₀O₈: 995.58; found 995.45 [M+H]⁺.

CB-TE2A-(FAPI): CB-TE2A-(^{t}Bu)₂-(FAPI) (3.3 mg, 3.3 μmol) was reacted with TFA at room temperature for 3 hours. After evaporation of the solvent, the residue was purified by semi-preparative RP-HPLC. Lyophilization of the collected fraction gave 2.7 mg (3.0 μmol; 93%) of the desired conjugate product CB-TE2A-(FAPI) as white solid. LC-MS Rₜ min, 2.6 min; MS (ESI) m/z calcd for C₄₃H₆₀F₂N₁₀O: 883.46; found 883.49 [M+H]⁺.

CB-TE2A-(^{t}Bu)₂-(FAPI)₂: Boc-FAPI (16.4 mg, 28.0 μmol) obtained in previous step was first treated with excess p-toluenesulphonic acid monohydrate (19.2 mg, 99.6 μmol) in acetonitrile solvent at 40° C. for 3 hours. After the Boc-deprotection step was confirmed with LCMS analysis, the residual solvent was removed under reduced pressure in rotary evaporator. The crude FAPI was directly used for the next reaction and added to the mixture containing CB-TE2A-(^{t}Bu)₂-(COOH) (4.8 mg, 8.0 μmol), HBTU (6 mg, 16.0 μmol) and DIPEA (14.6 μL, 80 μmol) in 500 μL DMF. The resulting mixture was then stirred at room temperature for 24 hours under N₂ balloon. After removing DMF under reduced pressure, the crude product was reconstituted in acetonitrile and purified by semi-preparative RP-HPLC. Lyophilization of the collected fraction gave 3.0 mg (2.0 μmol; 25%) of the desired conjugate product CB-TE2A-(^{t}Bu)₂-(FAPI)₂ as pale-yellow solid. LC-MS Rₜ min, 3.6 min; MS (ESI) m/z calcd for C₇₂H₁₀₆F₄N₁₆O₁₂: 1535.81; found 1535.79 [M+H]⁺.

CB-TE2A-(FAPI)₂: CB-TE2A-(^{t}Bu)₂-(FAPI)₂ (2.2 mg, 1.5 μmol) was reacted with TFA at room temperature for 3 hours. After evaporation of the solvent, the residue was purified by semi-preparative RP-HPLC. Lyophilization of the collected fraction gave 1.6 mg (1.1 μmol; 75%) of the desired conjugate product CB-TE2A-(FAPI)₂ as white solid. LC-MS Rₜ min, 3.4 min; MS (ESI) m/z calcd for C₇₀H₉₀F₄N₁₆O₁₂: 1423.69; found 1423.51 [M+H]⁺.

⁶⁴Cu radiolabeling of CB-TE2A FAPI conjugate: 3-4 mCi of ⁶⁴CuCl₂ in 0.1 M HCl was added to a 1.5 mL vial containing 2 μg of CB-TE2A FAPI conjugate in 200 μL of 0.4 M NH₄OAc (pH=6.5). The resulting mixture was acidified to pH 6.0 by adding 30 μL of 0.1 M HCl. The vial was shaken and incubated at 75° C. for 30 minutes. After quenching the reaction with 5 μL of 5 mM diethylenetriaminepentaacetic acid (DTPA) and incubating for 5 min, the mixture was passed through a Sep-Pak C-18 plus cartridge preconditioned with 5 mL ethanol and 5 mL water. The cartridge was then rinsed two more times with 5 mL water, followed by elution of the ⁶⁴Cu-labeled product using pure ethanol. Radiochemical purity of the corresponding product was analyzed by radio-HPLC.

Serum Stability study

100 μL of human serum in microcentrifugation vials was added with an aliquot (ca. 50 μCi) of ⁶⁴Cu-CB-TE2A-(FAPI) or ⁶⁴Cu-CB-TE2A-(FAPI)₂. The vials were then incubated at 37° C. in a water bath at different time point (1, 4 and 24 h). Following precipitation of the serum proteins with 420 μL of ethanol, the vials were centrifuged for 5 min at a speed of 14,000 rpm. The transchelated ⁶⁴Cu into serum proteins from ⁶⁴Cu-CB-TE2A-(FAPI) or ⁶⁴Cu-CB-TE2A-(FAPI)₂ complex was analyzed using radio-HPLC.

### Western Blot Assay

XP-185 tumor was cultured in the RPMI 1640 or DMEM medium containing 10% FBS at 37° C. in 5% CO₂. Western blotting and histopathological staining were performed according to our previously published protocol described in the Supplemental Material of the article titled “Theranostic Small-Molecule Prodrug Conjugate for Neuroendocrine Prostate Cancer” and published in Pharmaceutics. 2023 February; 15(2):481, authored by Gonzalez P, Debnath S, Chen Y A, Hernandez E, Jha P, Dakanali M, Hsieh J T, and Sun X. A. The entire article is incorporated by reference herewith.

### Small Animal PET/CT Imaging

Small animal PET/CT imaging experiments were acquired using a MEDISO PET/CT Multimodality System in XP-185 tumor-bearing SCID mice. Purified ⁶⁴Cu-CBTE2A-(FAPI) and ⁶⁴Cu-CBTE2A-(FAPI) 2 were diluted to the concentration of 4-5 MBq which was injected into the mice (n=4) intravenously using tail vein injection. 2% isoflurane anesthesia was used during all dynamic and static PET imaging procedures. Unlabeled FAPI at a concentration of 10 μg/kg was co-injected with ⁶⁴Cu-CBTE2A-(FAPI)₂ to perform blocking experiment.

### Example 1: Synthesis of Monovalent and Divalent Cross-Bridged Cyclam-FAPI Conjugate

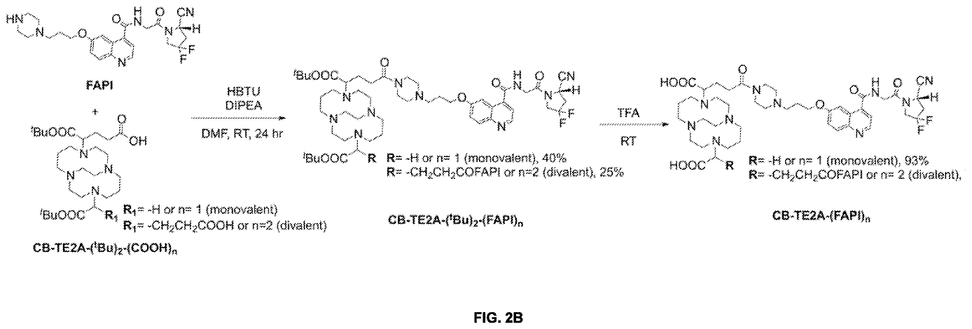

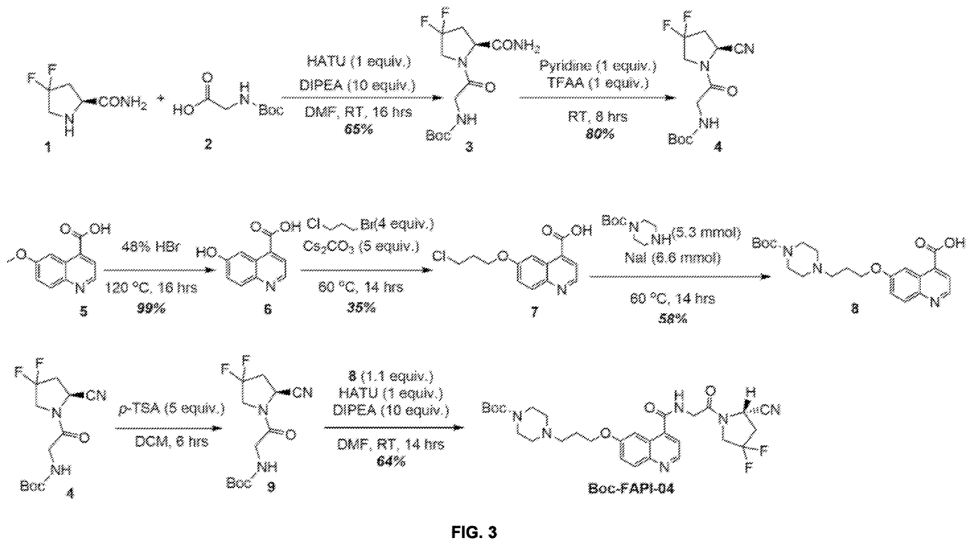

The desired monovalent and divalent cross-bridged cyclam-FAPI conjugates were prepared using a synthetic scheme presented in FIG. 2B. Prior to the conjugation, bicyclic CB-TE2A-(^{t}Bu)₂-COOH derivatives were synthesized. In brief, stoichiometry-controlled alkylation of CB-Cyclam gave mono-(n=1) and di-(n=2) benzylated CB-TE2A-(^{t}Bu)₂-(COOBn)ₙ which was followed by Palladium catalyzed hydrogenolysis reaction to obtain the corresponding acid products nearly in quantitative yields, CB-TE2A(tBu)2-(COOH)ₙ (n=1 or 2) in high yields. Similarly, FAPI ligand was synthesized following known procedures reported in literature. A HATU promoted coupling reaction between Quinoline-4-carboxylic acid derivative and Pyrrolidine-Glycine conjugate resulted the desired Boc-protected Boc-FAPI in 64% yield (FIG. 3). Boc-group removal was achieved with excess of p-TSA (para-toluenesulfonic acid) in acetonitrile solvent at heated condition, yielding FAPI in crude form which was subsequently subjected to the next coupling reaction with CB-TE2A-(^{t}Bu)₂-COOH derivative without further purification. CB-TE2A-(^{t}Bu)₂-COOH or CB-TE2A-(^{t}Bu)₂-(COOH)₂ and free amine (—NH₂) group of FAPI (1.2 equiv. for monovalent and 3.5 equiv. for divalent) underwent amide bond formation reaction at room temperature in the presence of HBTU to give desired ^{t}Bu-protected conjugate products in moderate yields of 40% and 25% for monovalent CB-TE2A-(^{t}Bu)₂-FAPI and divalent CB-TE2A-(^{t}Bu)₂-(FAPI)₂ conjugate, respectively (FIG. 2B). Finally, two ^{t}Bu- groups were removed under trifluoroacetic acid (TFA) condition to obtain the final and desired conjugates CB-TE2A-(FAPI)₂ and CB-TE2A-(FAPI) (FIG. 2A) in good yields of 75% and 93% respectively. One such FAPI is (S)—N-(2-(2-cyano-4,4-difluoropyrrolidin-1-yl)-2-oxoethyl)-6-(3-(piperazin-1-yl)propoxy)quinoline-4-carboxamide (FAPI04), which was prepared according to a published procedure. Coupling of CB-TE2A(tBu)₂-(COOH)ₙ (n=1 or n=2) with FAPI04 provided CB-TE2A-(tBu)₂-FAPI04 and CB-TE2A-(tBu)2-(FAPI04)₂, which were deprotected by neat trifluoroacetic acid (TFA) to afford CB-TE2A-(FAPI04) and CB-TE2A-(FAPI04)₂ in yields of 93% and 75%, respectively. Their labelling with 64Cu resulted in high radiochemical yields (>60%) and >98% radiochemical purity. In vitro cell binding potency of the radioconjugates were compared in RENCA-FAP (FAP^{high}) and RENCA (FAP^{low}) cells. Small animal PET/CT imaging was performed using a MEDISO PET/CT scanner (nanoScan® PET/CT) in SCID mice bearing XP-185 (FAP^{high}, a clear cell renal cell carcinoma (ccRCC) tumorgraft line) and BALB/c mice bearing RENCA-FAP (FAP^{high}) xenografts.

### Example 2: Western Blotting, Radiolabeling and In-Vitro Serum Stability

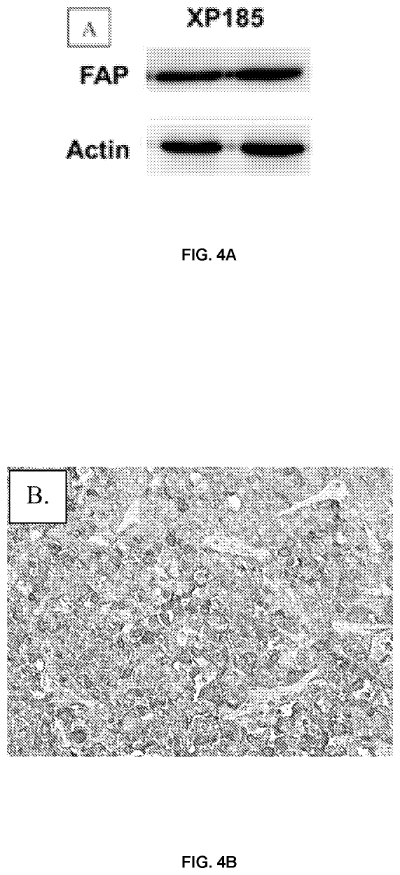

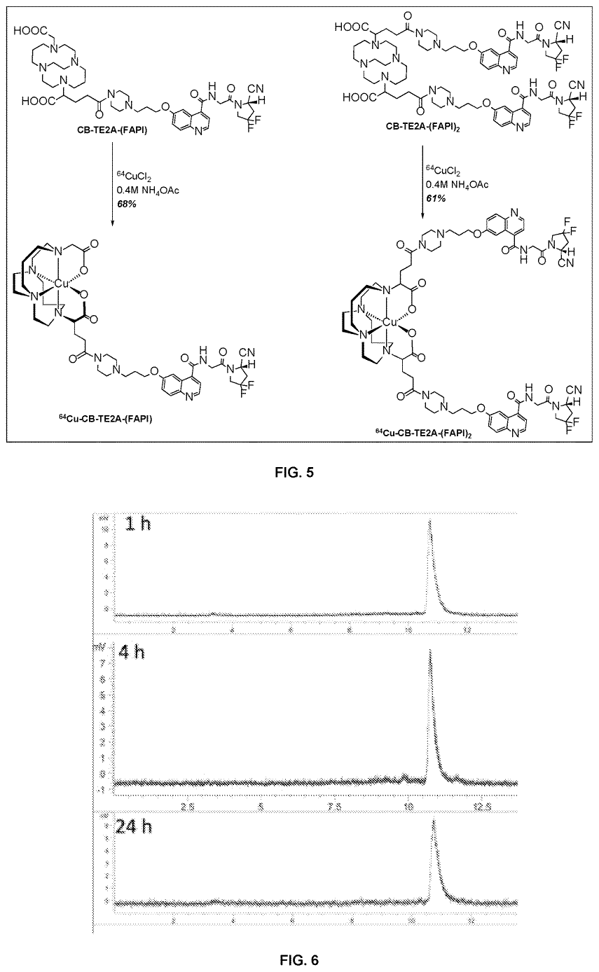

FAP expression in XP-185 tumor xenograft was confirmed with western blot and immunohistochemistry (FIGS. 4A and 4B). Radiolabeling reactions of [⁶⁴Cu]Cu²⁺ (3-4 mCi) with monovalent or divalent conjugates (5 μg) were performed in NH₄OAc buffer (pH 6.5) at 75° C. for 30 minutes. The crude radiolabeled conjugates were purified with preconditioned C18 Sep-Pak light cartridge. Non-decay radiochemical yields and radiochemical purity for both conjugates were over 60% (FIG. 5) and 97% respectively.

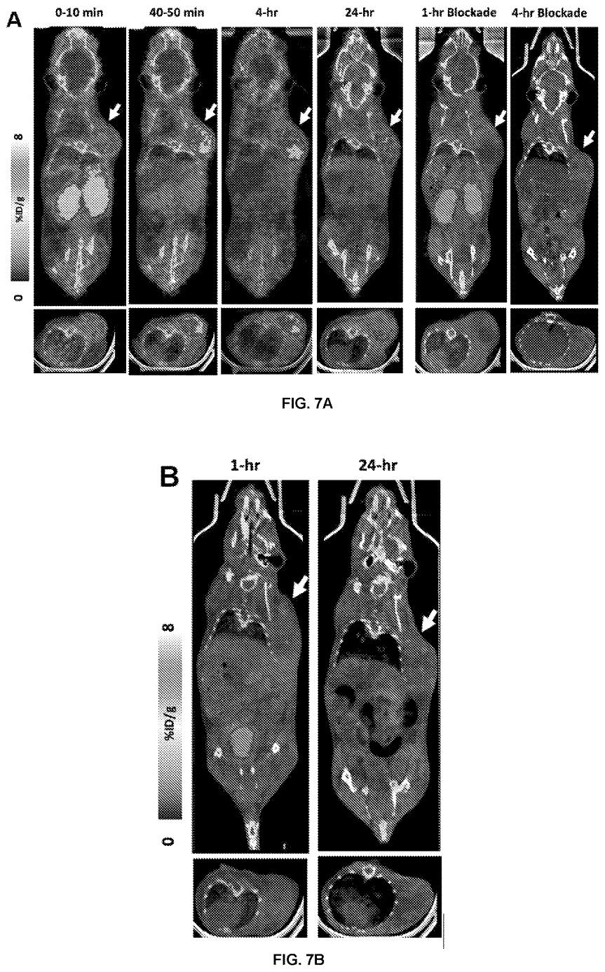

In vitro stability of divalent conjugate was accessed using radio-HPLC analysis of γ traces obtained from the purified sample after incubation of a mixture of 50 μCi of the conjugate and 100 μL of human serum at 37° C. for three time points of 1-hour, 4-hour, and 24-hour (n=3 for each time point). The results showed no leaching of free ⁶⁴Cu²⁺ and/or decomposition of the divalent tracer even until 24 hours (FIG. 6).

### Example 3: In Vivo PET/CT Imaging

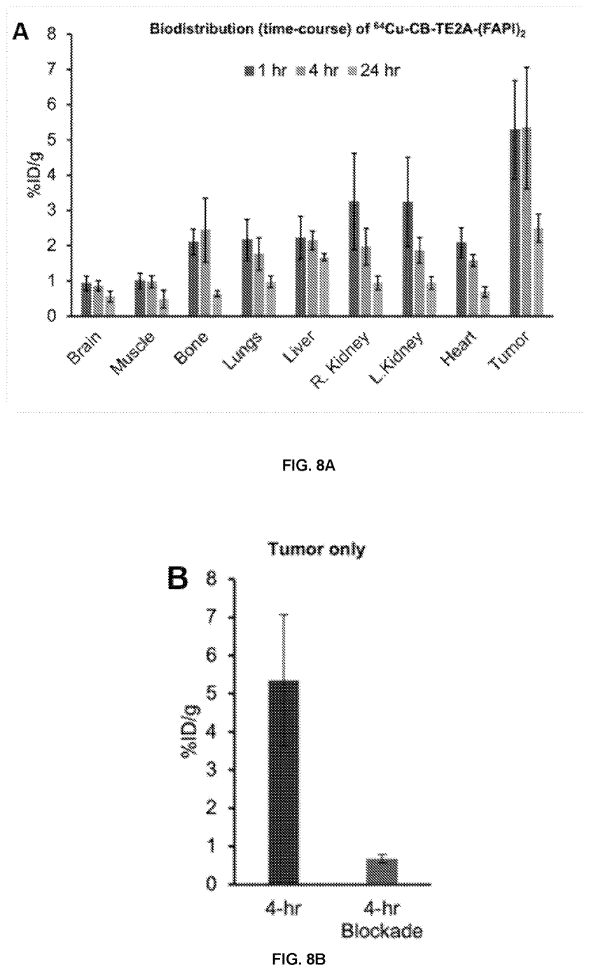

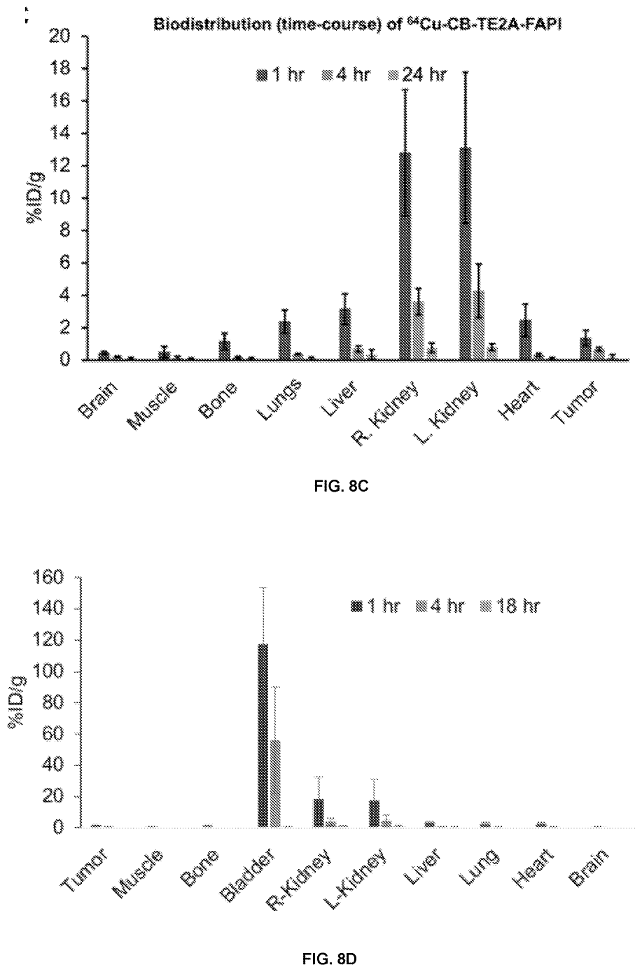

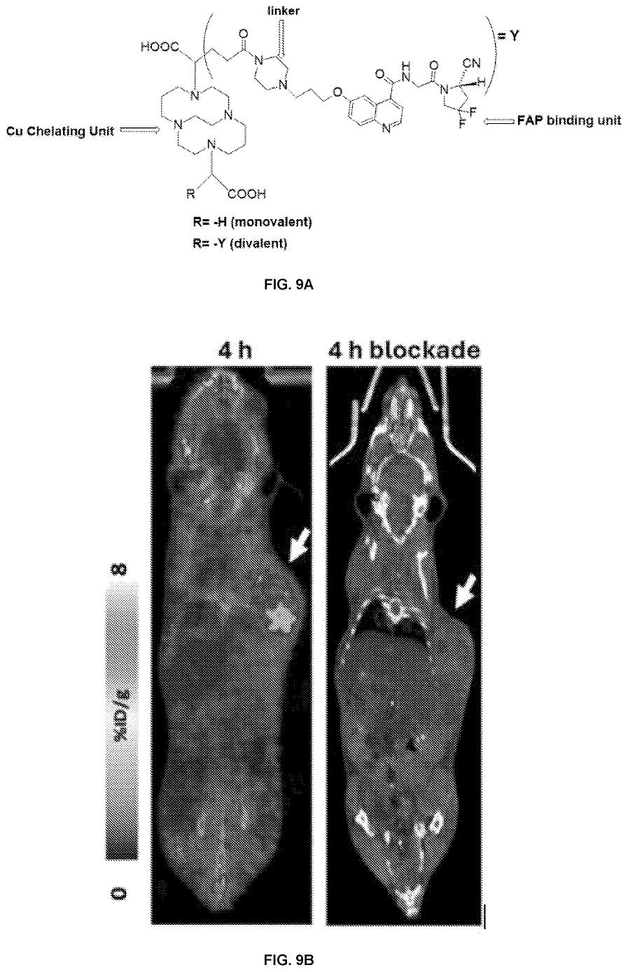

The in vivo PET/CT images were obtained in Mediso PET/CT Multimodality System. Imaging data analysis was performed by comparing biodistribution of divalent tracer ⁶⁴Cu-CB-TE2A-(FAPI)₂ and monovalent tracer ⁶⁴Cu-CB-TE2A-(FAPI) in XP-185 xenograft in right flank of severe combined immunodeficiency (SCID) mice (as shown with white arrow in FIGS. 7A and 7B). Following tail vein injection of radiotracer, images were acquired at three time points: 1-hour dynamic scan, 4-hr static scan, and 24-hour static scan. A comparative biodistribution results show that the tumor uptake of the divalent radiotracer is at least 3 times higher than the monovalent conjugate throughout the selected time points, with highest tumor uptake recorded at 1-hour (5.3±1.4) and 4-hour time point (5.3±1.7) for divalent tracer, and 1-hr time point (1.4±0.5) in the case of monovalent tracer (Table 1). Interestingly, divalent tracer had a higher uptake in tumor lesions compared to selected normal organs except Bladder even until 24 hours after injection. Although both tracers started retaining in the tumor site within 10 minutes of injection, the divalent tracer persisted until 24 hours as shown in left four images in FIG. 7A. Monovalent tracer, on the other hand, distributed in bladder, kidneys, liver, heart, and lungs more than in the tumor lesions and is mostly cleared out from normal organs including tumor site after 1-hour post injection (FIG. 8A-C).

TABLE 1
Tumor uptake of monovalent and divalent FAPI conjugate as measured
from PET imaging quantitation and post-PET biodistribution.
Data are presented as % IDg⁻¹ ± standard deviation (n = 4)

| | | | | 4-hr |
|---|---|---|---|---|
| | 1-hr PET | 4-hr PET | 24-hr PET | Blockade |
| [⁶⁴Cu]-CB-TE2A- | 5.3 ± 1.4 | 5.3 ± 1.7 | 2.5 ± 0.4 | 0.67 ± 0.12 |
| (FAPI)₂ | | | | |
| [⁶⁴Cu]-CB-TE2A- | 1.4 ± 0.5 | 0.67 ± 0.1 | 0.16 ± 0.01 | |
| FAPI | | | | |

Another study to assess binding specificity of the divalent conjugate was conducted by co-injecting excess (100-fold) of FAP specific ligand FAPI. As seen in two right images in the left panel of FIG. 7A and bar diagram in FIGS. 8B and 8D, tumor uptake was significantly reduced in the presence of FAPI (0.67±0.12% ID/g).

### Example 4: Results and Discussion

Fibroblast Activation Protein (FAP)-targeted theranostic radiopharmaceuticals have shown promising potential for cancer diagnosis and treatment due to the ubiquitous presence of FAP within tumor microenvironment of solid cancer malignancies. With judicious choice of FAP binding units and linkers appended to optimal chelators, the design and development of radiotheranostic copper radiopharmaceuticals has generated a considerable interest in the field, given that there exists an ideal theranostic pair of copper radionuclides: ⁶⁴Cu (t_{1/2}=12.7 h; 17.4% β+; Eβ+max=653 keV) and ⁶⁷Cu (t_{1/2}=2.58 d; 100% β-; Eβ-max=562 keV). FAP-targeted small molecule-based copper radiopharmaceuticals have emerged to be popular radiotheranostic candidates in the field of nuclear medicine. As a result, multiple preclinical and clinical studies have been undertaken to test their ability to diagnose and treat cancer as well as other non-cancerous diseases. Nevertheless, the development is limited to conjugates derived from DOTA or DOTAGA or NOTA chelated with ⁶⁸Ga and ¹⁷⁷Lu radioisotopes for the respective diagnostic and therapeutic application. While these metal ions share some common physical and chemical features, the differential coordination chemistry can pose a challenging approach to design the precursors that are suitable to both metal ions. Therefore, an ideal theranostic radionuclide pair like^{64/67}Cu with same coordination chemistry can be a suitable alternative.

The choice of cross-bridged cyclam ligand presented in this disclosure is inspired by its optimal copper ion binding properties, particularly when ionizable carboxymethyl pendant arms are available for complete coordination to theranostic copper radioisotopes. For example, 1,4,8,11-tetraaza-bicyclo[6.6.2]hexadecane-4,11-diacetic acid (CB-TE2A), the chelator that forms one of the most stable complexes with ⁶⁴Cu has yet to play a role in the reported efforts. The procedure, data and results present the design, synthesis, and comparative evaluation of monovalent and divalent FAP-targeting conjugates constructed from bifunctional chelator scaffold (BFS) CB-TE2A. Its favorable in vivo pharmacokinetics while also offering multivalency features for enhanced tumor retention was of particular interest. Indeed, a prime focus in recently developed FAP-targeted radiotracers is tuning efficacy for tumor retention through ‘polyvalent effect’ or ‘albumin hitchhiking’ approach. Therefore, cross-bridged cyclam derived FAPI conjugate would be a valid option for the same.

The present disclosure reflects a proof-of-concept study focused on developing novel copper-based radiotheranostic that targets FAP in CAF in solid tumor. The XP-185, an RCC PDX model, was chosen for its ability to represent an ideal CAF environment. Our work initiated with a simple and practical two step synthetic approach to prepare both monovalent and divalent conjugate precursors. A late-stage amide bond coupling reaction and TFA promoted ^{t}Bu- group deprotection afforded both the desired precursors. Of note, the initial attempt in coupling reaction between NHS activated CB-TE2A-(^{t}Bu)₂-CONHS derivative and amine group of FAPI was unsuccessful and resulted in the desired conjugated products in only trace yields. It is also worth noting that during the synthesis of divalent conjugate CB-TE2A-(FAPI)₂, TFA promoted hydration of cyanide (—CN) group to amide (—CONH₂) was formed as a major product when the reaction was continued for several hours (>12 hours). Therefore, reaction time was maintained below a 3-hour to avoid as much overreaction of desired nitrile product to the amide byproduct as possible. Surprisingly, monovalent conjugate CB-TE2A-(FAPI) was obtained selectively under TFA reaction even after an 8-hour reaction time. Following successful synthesis of the precursors, the radiolabeling scope was evaluated with in-house cyclotron produced ⁶⁴Cu, which was highly efficient and consistent. Furthermore, serum binding results demonstrated a robustness of the radiotracer under the in vitro condition.

Increased tumor distribution and improved in vivo pharmacokinetics of divalent tracer [⁶⁴Cu-CB-TE2A-(FAPI)₂] compared to monovalent tracer [⁶⁴Cu-CB-TE2A-(FAPI)] in XP-185 xenograft in plausible, given the enhanced local concentration of FAPI in the former. In other words, local concentration of FAPI can increase when one FAPI moiety binds while the other remains intact in the proximity of FAP sites. However, whether both FAPI moieties bind to FAP site simultaneously remains a subject of detailed analysis and is out of the scope of this work. Nevertheless, the outcomes corroborate our and others previous observation regarding efficacy of multivalent scaffolds design in tumor targeting for PET/SPECT imaging related application. Similarly, a high kidney uptake and rapid clearance of both radiotracers from liver can be attributed to their hydrophilicity property (FIGS. 8A and 8C). Interestingly, kidney uptake for monovalent tracer is higher than divalent tracer at 1-hr p.i. (FIG. 8C), further indicating the increased hydrophilicity likely due to one less quinoline ring in the former. In fact, log P value measured for the monovalent tracer was more negative than the divalent tracer (−1.6 vs −1.2). Furthermore, the strong inhibition in tracer uptake in tumor region caused by the co-injection of excess of FAP specific ligand FAP strongly suggests FAP mediated uptake of the radiotracer (FIG. 8B). A 1-hour blockade experiment depicting high uptake in kidneys and bladder (FIG. 8D) could possibly be the result of elimination route of the radiotracer via these organs and not the FAP mediated uptake of the radiotracer.

Overall, divalent tracer ⁶⁴Cu-CB-TE2A-(FAPI)₂ characterized by FAP specific, prolonged tumor retention and improved tumor uptake as well as fast clearance from non-target organs is a promising candidate for radiotheranostic application with ⁶⁷Cu isotope. Specifically, for FAPI04, the in vitro total cell uptake vs. nonspecific uptake ratios of monovalent and divalent tracers were found to be ~17.38 and ~4.77 for RENCA-FAP (FAP^{high}) and ~2.19 and ~2.09 for RENCA (FAP^{low}) cells, respectively. In addition, a blockade of 1 mM of cold FAPI04 ligand resulted in a ~14-fold and ~4-fold reduction of FAP-binding of the monovalent and divalent tracer for RENCA-FAP (FAP^{high}) cells. The monovalent tracer showed a rapid cell internalization compared to the divalent tracer at the initial time frame (30-60 min) for RENCA-FAP (FAP^{high}), while the divalent tracer internalized noticeably more between 90-120 min. The percentage of injected dose per gram (% ID/g) of [⁶⁴Cu]Cu-CB-TE2A-(FAPI04)₂ was significantly higher in RENCA-FAPhigh tumor (6.25±1.87% ID/g, 4 h p.i.) than in the muscle (0.33±0.13% ID/g, 4 h p.i.) out to 24 h p.i. (FIG. 1). In addition, the divalent radiotracer showed 3 times higher tumor uptake than the monovalent conjugate in mice bearing XP-185 tumorgraft during the 4 h PET scan. Further, co-injection of 100-fold cold FAPI04 resulted in a substantial reduction in tumor uptake of the divalent tracer (0.67±0.12% ID/g 4 h p.i.), indicative of the FAP-targeting specificity of the agent. These data suggests that the BFS-based multivalent strategy can be used for FAP-targeted radiotheranostic agent development. Particularly, the enhanced uptake and prolonged retention of the divalent agent is a desired feature for effective radionuclide therapy.

### Example 5. Additional FAP-Targeted Radiopharmaceuticals

More FAP-targeted small molecule-based radiopharmaceuticals are to be synthesized and tested. Such radiopharmaceuticals include, but not limited to:

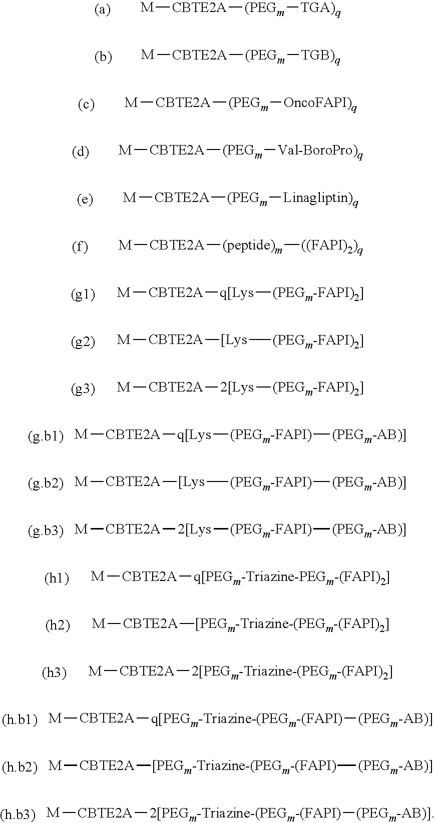

Here, M denotes a chelating copper, including but not limited to, ⁶⁰Cu, ⁶¹Cu, ⁶²Cu, ⁶⁴Cu, or ⁶⁷Cu; m is zero, or any integer of 1 to 12; and q is 1 or 2; PEG is polyethylene glycol; AB is an albumin binder; Lys is lysine; CBTE2A represents one of the following two moieties:

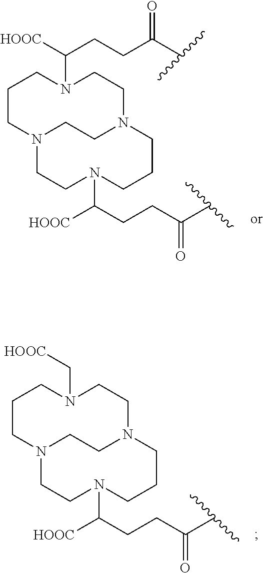 [SMILES: *C(=O)CCC(C(=O)O)N1CCCN2CCN(CCCN(C(CCC(*)=O)C(=O)O)CC2)CC1 ; *C(=O)CCC(C(=O)O)N1CCCN2CCN(CCCN(CC(=O)O)CC2)CC1]

FAPI is a Fibroblast Activation Protein targeting group, including but not limited to, TGA, TGB, OncoFAP, Val-BoroPro, and Linagliptin shown below:

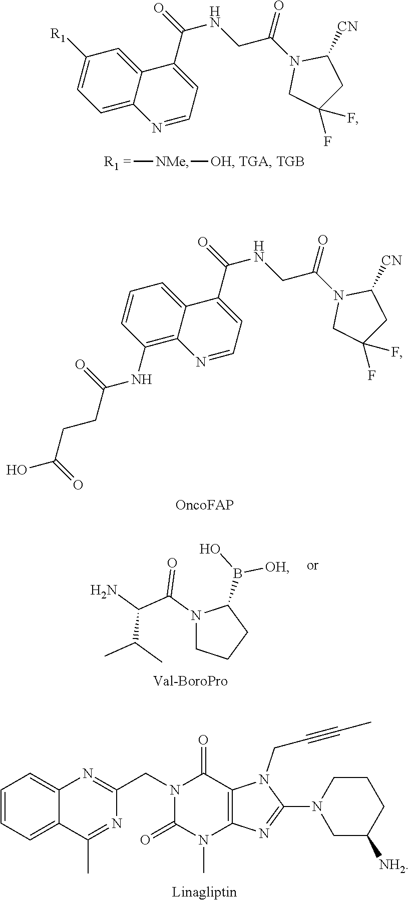

Further, some exemplary structures of Formula (a) through Formula (h.b3) are shown below. They are by no means limiting, but merely an illustration of the radiopharmaceuticals contemplated by the present disclosure. All structures expressly include any positional isomers, stereo isomers, and/or pharmaceutically acceptable salts.

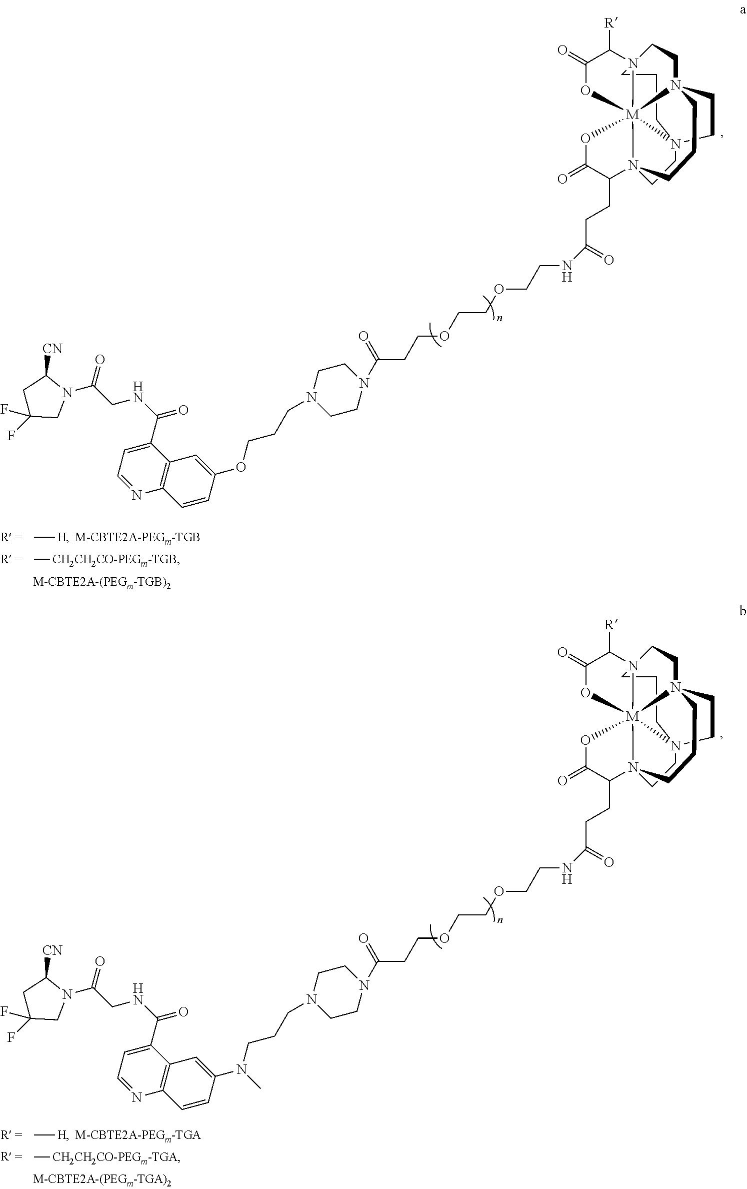

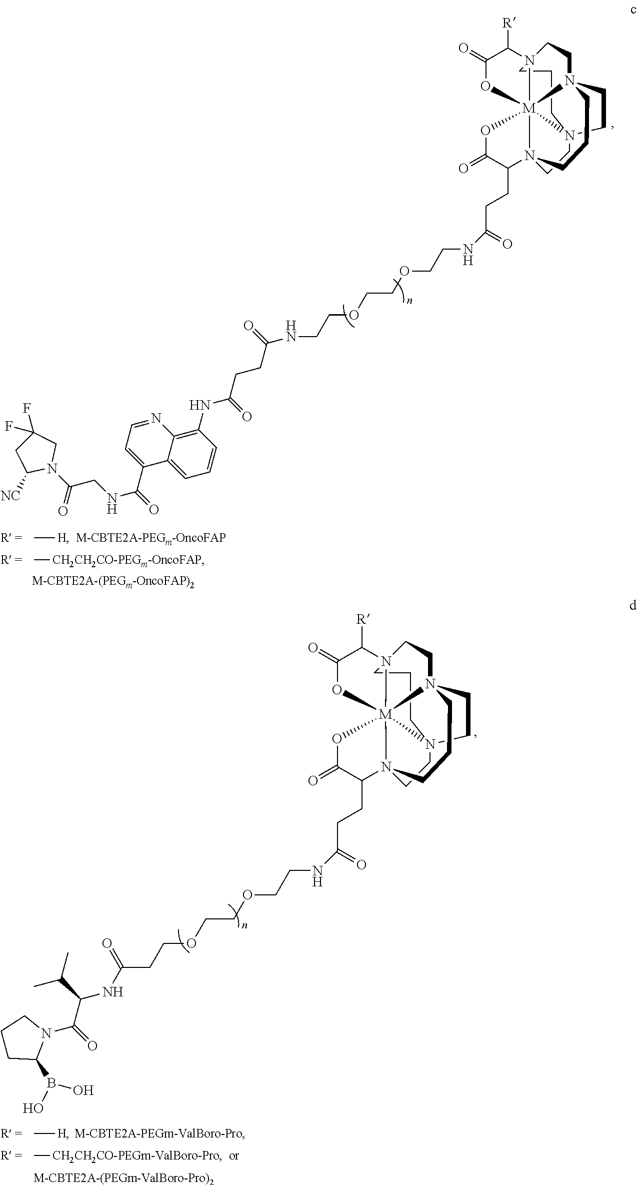

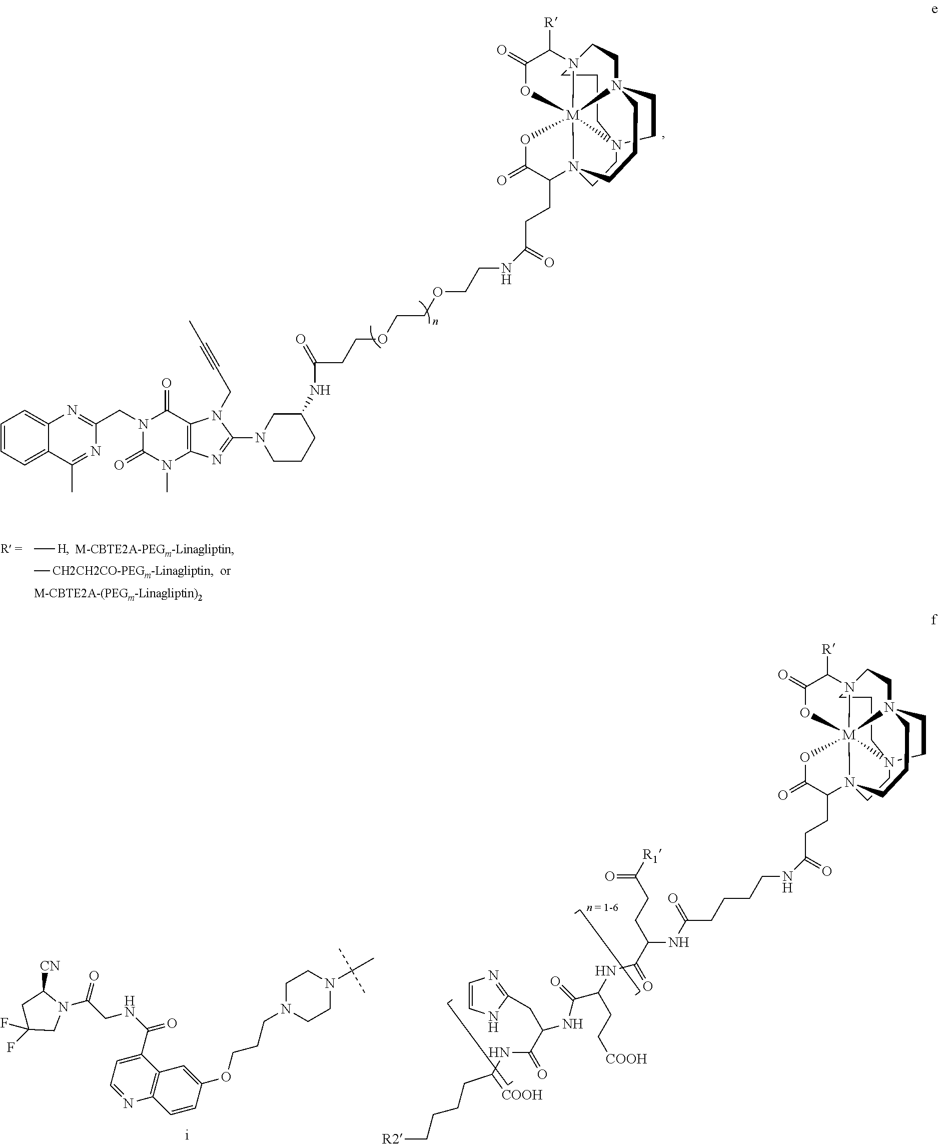

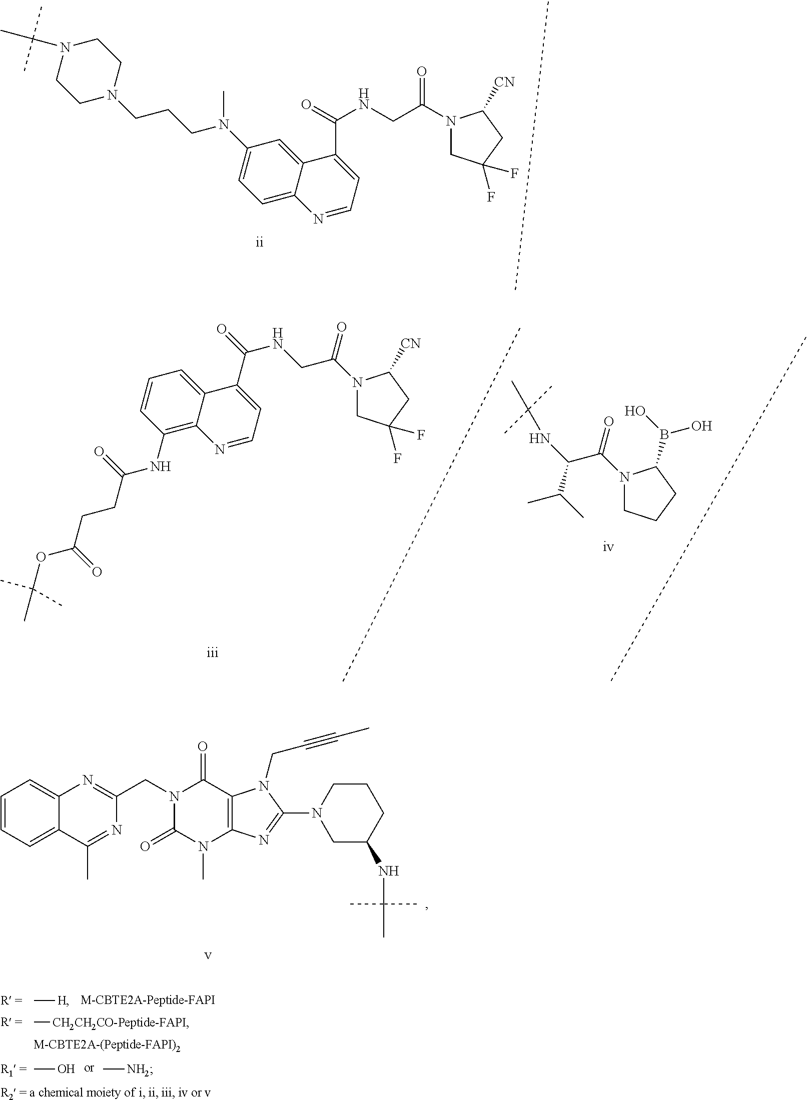

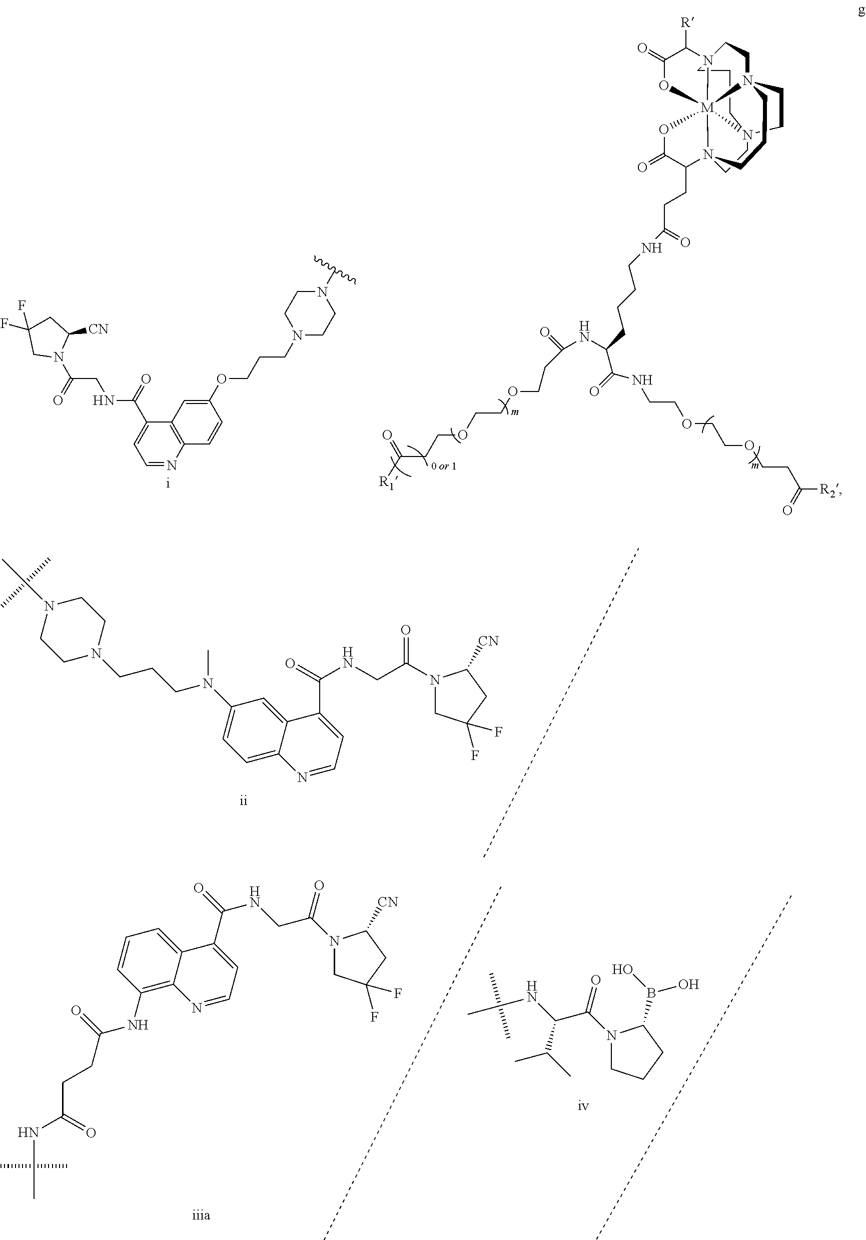

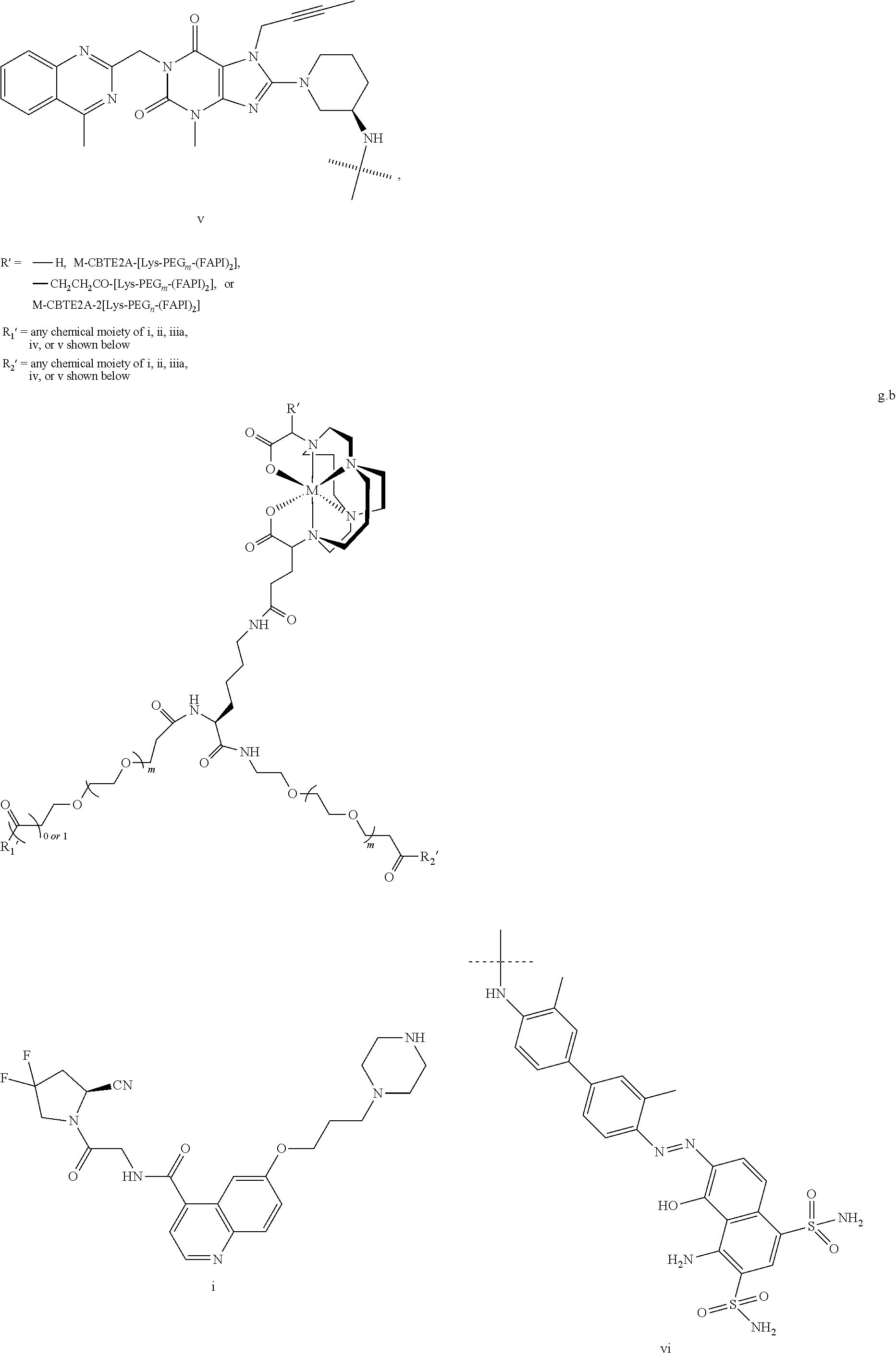

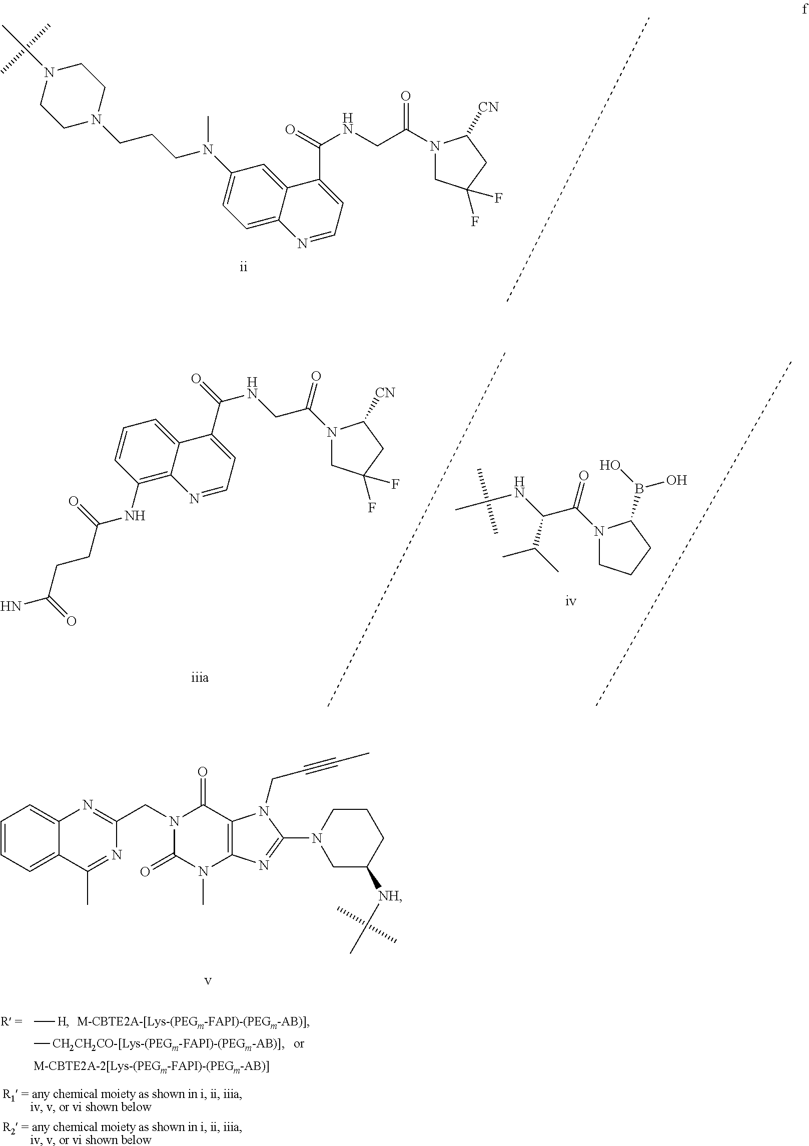

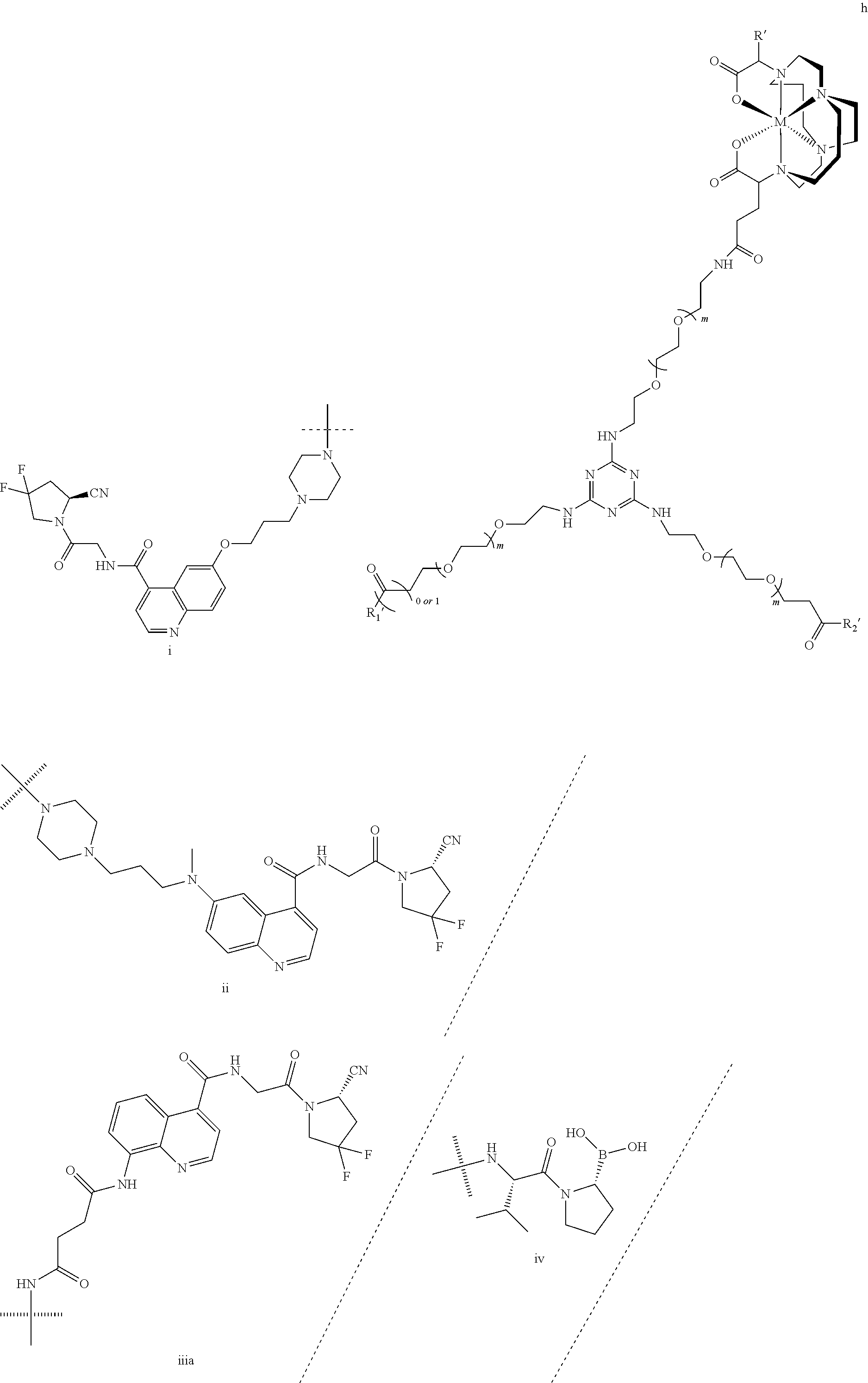

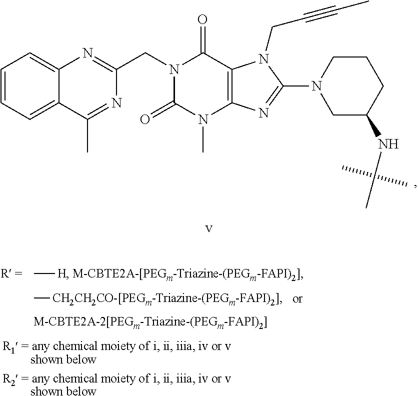

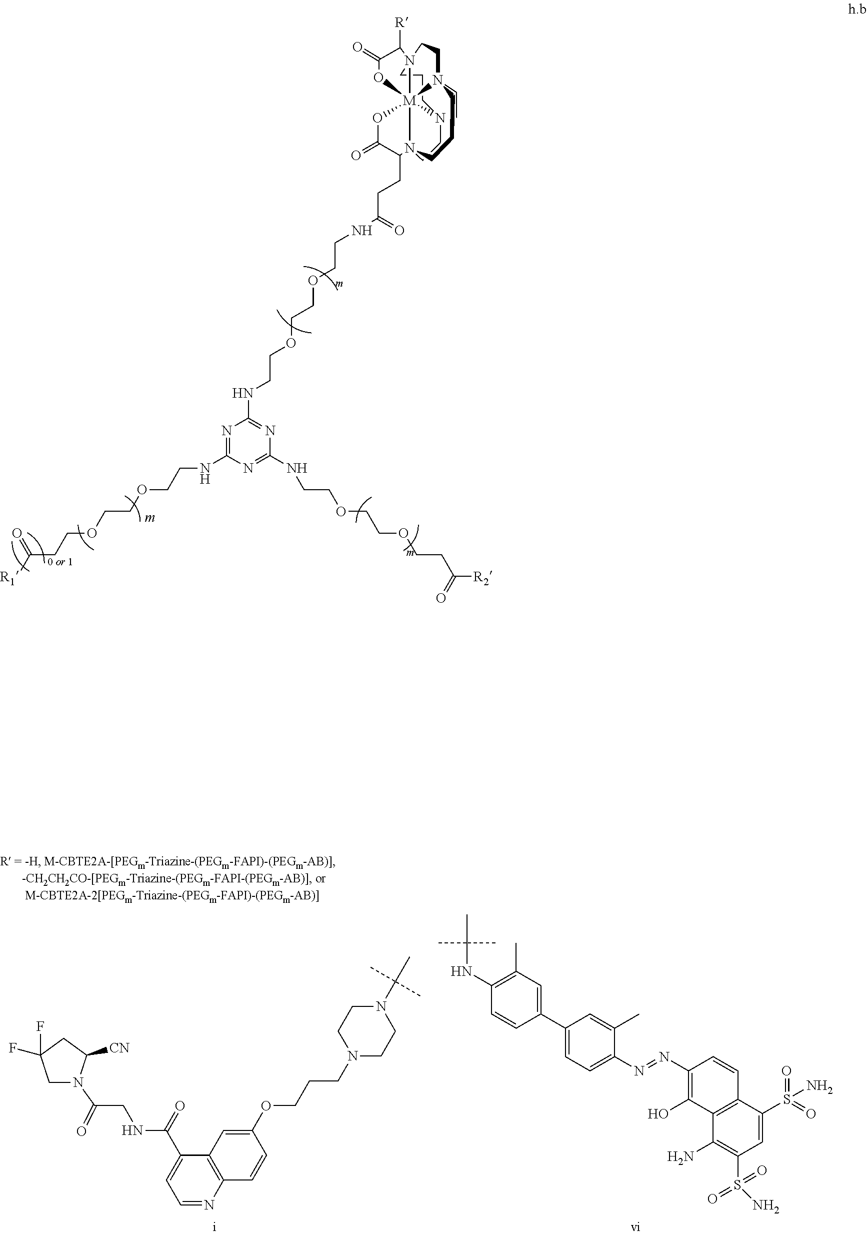

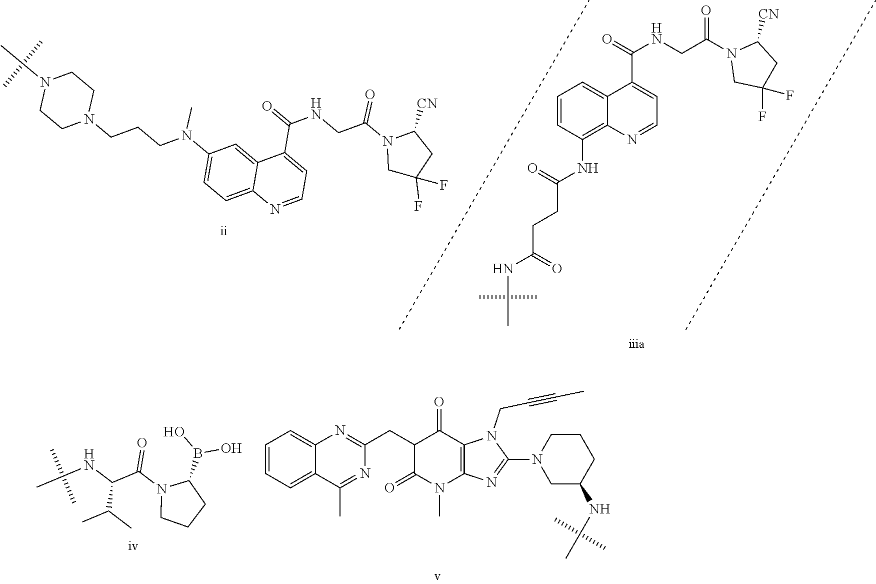

What is claimed is:

1. A compound of Formula (I), any positional isomer, any stereo isomer, or any pharmaceutically acceptable salt thereof: 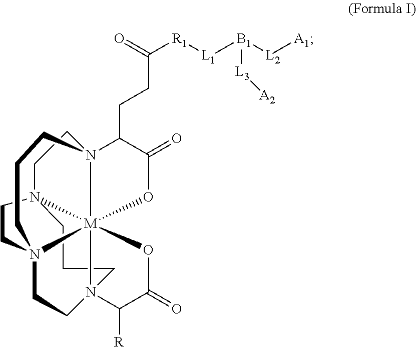
wherein A₁ is a Fibroblast Activated Protein (FAP) targeting group (FAPI) or an albumin binder (AB) group, wherein the FAPI or the AB group is capable of covalent or non-covalent binding with the FAP or albumin respectively;
A₂ is a Fibroblast Activated Protein (FAP) targeting group (FAPI), an albumin binder (AB) group, or absent, wherein the FAPI or the AB group is capable of covalent or non-covalent binding with the FAP or albumin respectively;
R₁ is —NH, piperazine, piperidine, an alkylamine, a dicarboxylate amide, or is absent;
L₁ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof;
L₂ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent;
L₃ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent;
B₁ is a heterocyclic group, a heterocyclic alkyl, an amidoalkyl, an alkylamide, or is absent;
R is hydrogen or Formula (Ia) 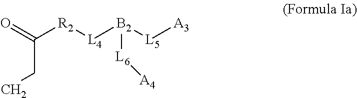
wherein,
A₃ is a Fibroblast Activated Protein (FAP) targeting group (FAPI) or an albumin binder (AB) group, wherein the FAPI or the AB group is capable of covalent or non-covalent binding with the FAP or albumin respectively;
A₄ is a Fibroblast Activated Protein (FAP) targeting group (FAPI) or an albumin binder (AB) group, or is absent, wherein the FAPI or the AB group is capable of covalent or non-covalent binding with the FAP or albumin respectively;
R₂ is —NH, piperazine, piperidine, an alkylamine, a dicarboxylate amide, or is absent;
L₄ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof;
L₅ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent;
L₆ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent;
B₂ is a heterocyclic group, a heterocyclic alkyl, an amidoalkyl, an alkylamide, or is absent;
M is ⁶⁴Cu, ⁶⁰Cu, ⁶¹Cu, ⁶²Cu or ⁶⁷Cu.

2. The compound of claim 1, wherein the FAPI comprises the following:
targeting group A (TGA): 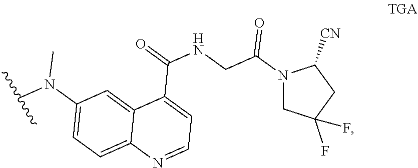 [SMILES: *N(C)c1ccc2nccc(C(=O)NCC(=O)N3CC(F)(F)C[C@H]3C#N)c2c1]
targeting group B (TGB): 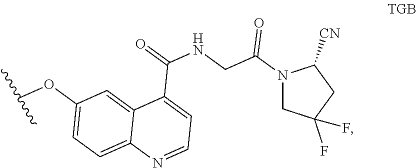 [SMILES: *Oc1ccc2nccc(C(=O)NCC(=O)N3CC(F)(F)C[C@H]3C#N)c2c1]
targeting group C (TGC): 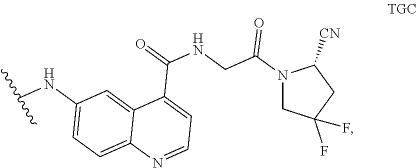 [SMILES: *Nc1ccc2nccc(C(=O)NCC(=O)N3CC(F)(F)C[C@H]3C#N)c2c1]
targeting group D (TGD): 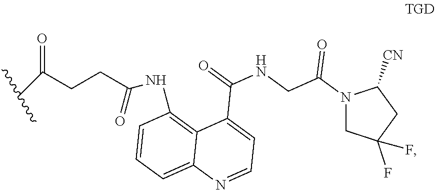 [SMILES: *C(=O)CCC(=O)Nc1cccc2nccc(C(=O)NCC(=O)N3CC(F)(F)C[C@H]3C#N)c12]
targeting group E (TGE): 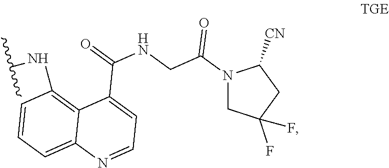 [SMILES: *Nc1cccc2nccc(C(=O)NCC(=O)N3CC(F)(F)C[C@H]3C#N)c12]
targeting group F (TGF): 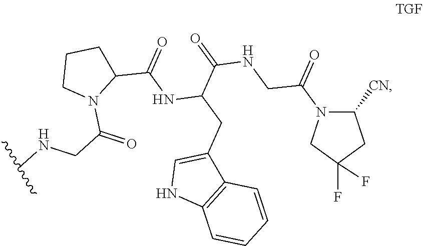 [SMILES: *NCC(=O)N1CCCC1C(=O)NC(Cc1c[nH]c2ccccc12)C(=O)NCC(=O)N1CC(F)(F)C[C@H]1C#N]
targeting group G (TGG): 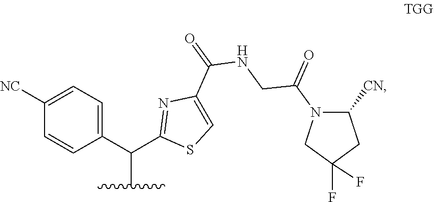 [SMILES: *C(c1ccc(C#N)cc1)c1nc(C(=O)NCC(=O)N2CC(F)(F)C[C@H]2C#N)cs1]
targeting group H (TGH): 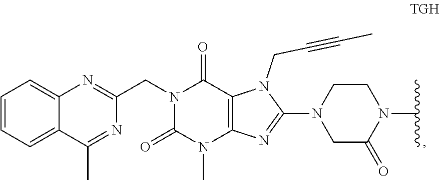 [SMILES: *N1CCN(c2nc3c(c(=O)n(Cc4nc(C)c5ccccc5n4)c(=O)n3C)n2CC#CC)CC1=O]
targeting group I (TGI): 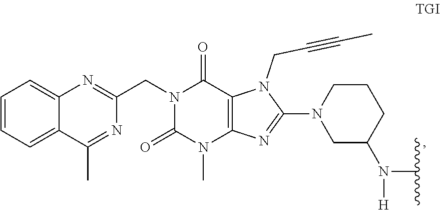 [SMILES: *NC1CCCN(c2nc3c(c(=O)n(Cc4nc(C)c5ccccc5n4)c(=O)n3C)n2CC#CC)C1]
targeting group J (TGJ):

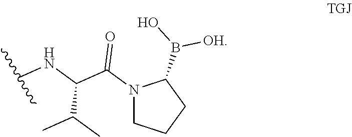 [SMILES: *N[C@H](C(=O)N1CCC[C@H]1B(O)O)C(C)C]

3. The compound of claim 1, wherein the FAPI or the AB group is a tautomer, an enantiomer, a diastereomer, or a derivative thereof.

4. The compound any one of the claims 1-3, wherein the polyethylene glycol (PEG) and the functionalized polyethylene glycol (functionalized PEG) linker comprise 1 to 12 ethylene glycol repeating units; and wherein the functionalized PEG linker comprises one or more functional groups selected from the group consisting of amine, amide, carbonyl, carboxylate, heterocyclic, amidoalkylcarbonyl, amidoalkylcarboxylate, alkylamine, alkylamide, alkylcarbony, alkylcarboxylate, heterocyclic alkyl, and any combination thereof.

5. The compound of any one of the claims 1-4, wherein the peptidomimetic or peptide linker comprises 1 to 10 amino acid residues.

6. The compound of claim 5, wherein the peptidomimetic or peptide linker comprises a lysine residue.

7. The compound of any one of the claims 1-6, wherein the AB group comprises:

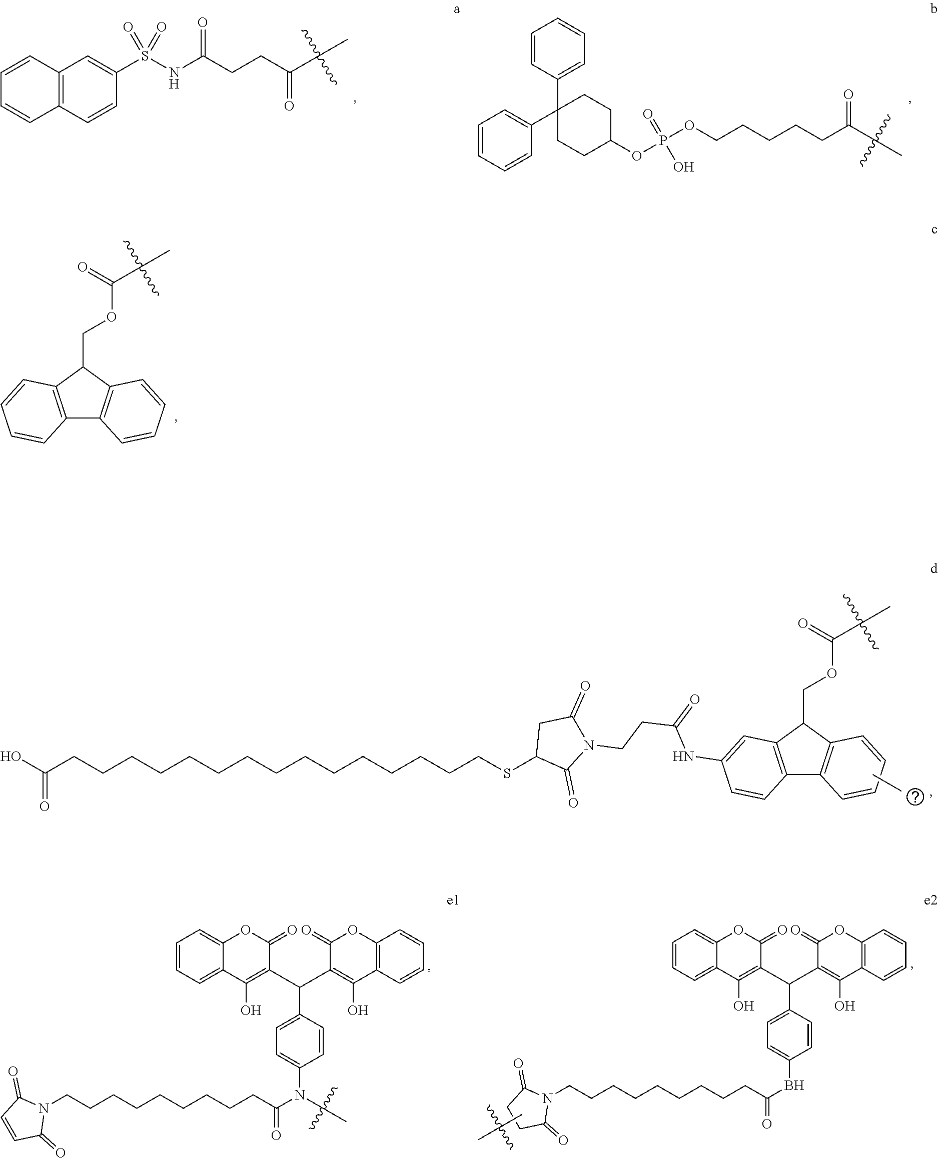

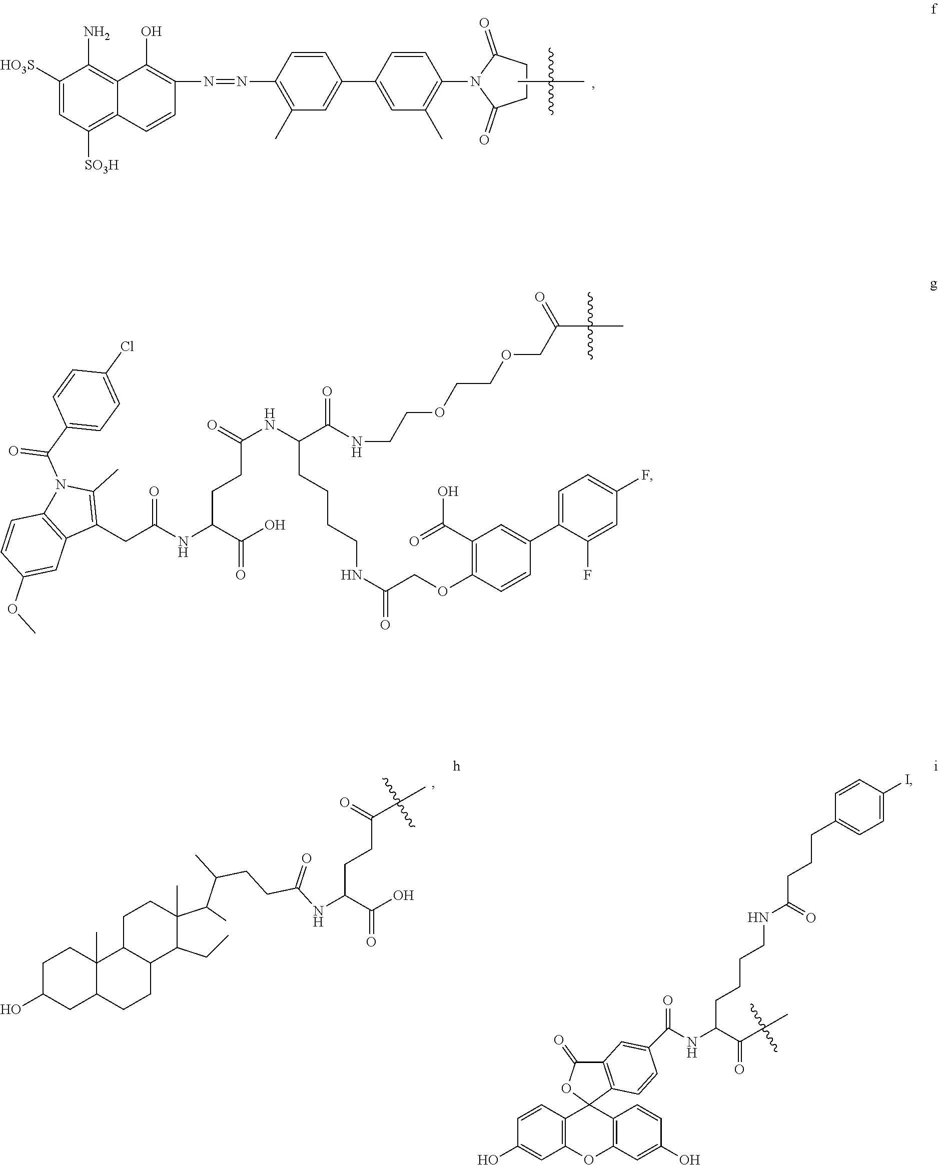

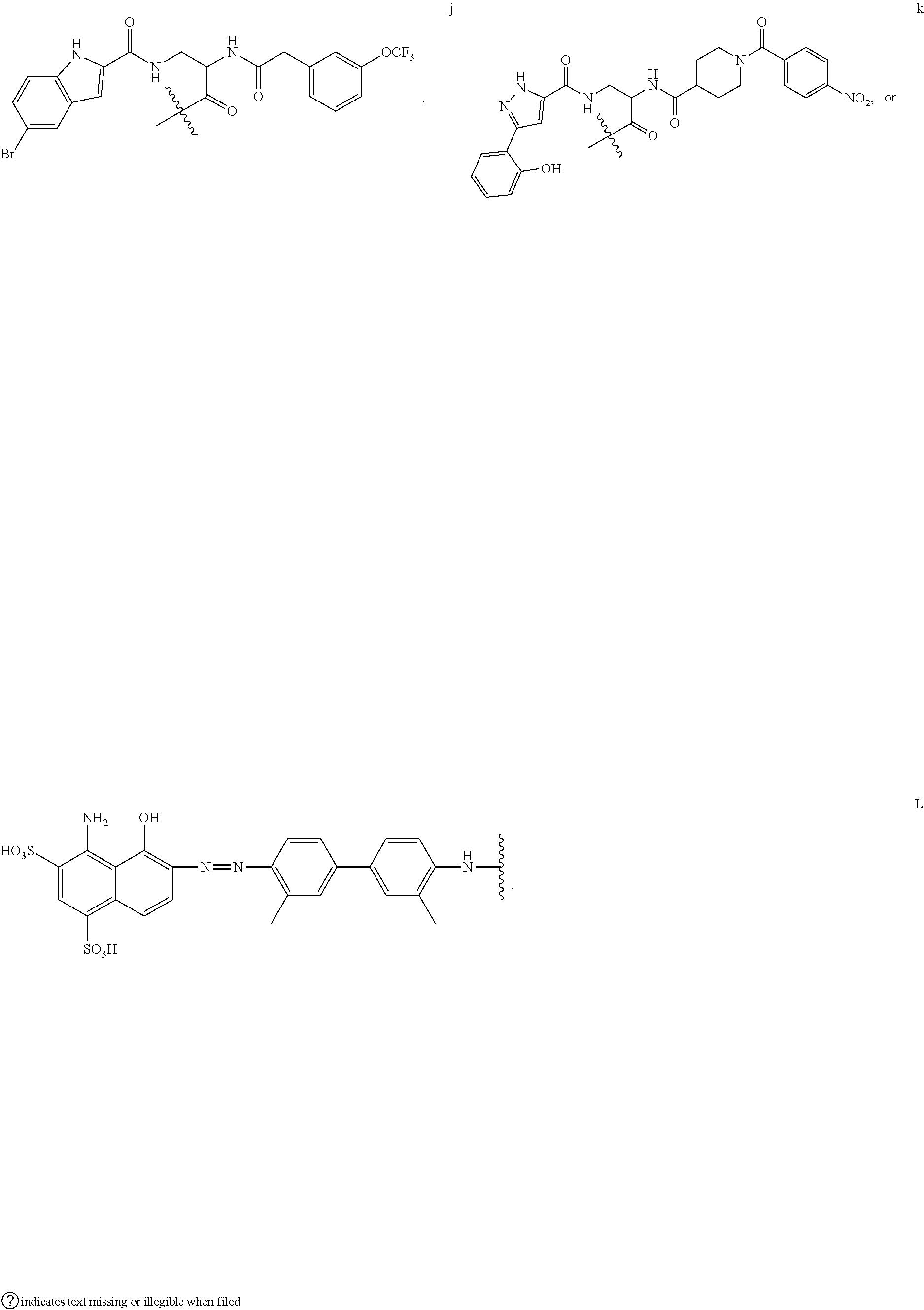 [SMILES: *C(=O)C(CNC(=O)c1cc2cc(Br)ccc2[nH]1)NC(=O)Cc1cccc(OC(F)(F)F)c1 ; *C(=O)C(CNC(=O)c1cc(-c2ccccc2O)n[nH]1)NC(=O)C1CCN(C(=O)c2ccc([N+](=O)[O-])cc2)CC1 ; *Nc1ccc(-c2ccc(N=Nc3ccc4c(S(=O)(=O)O)cc(S(=O)(=O)O)c(N)c4c3O)c(C)c2)cc1C]

8. The compound of any one of claims 1-7, wherein the heterocyclic group of B₁ and/or B₂ is selected from a triazine, a triazine amine, or a triazine aminoalkyl.

9. The compound of any one of the claims 1-8, wherein the compound of Formula (I) is capable of targeting to a Fibroblast Activated Protein (FAP).

10. The compound of any one of the claims 1-9, wherein the compound is Formula (II):

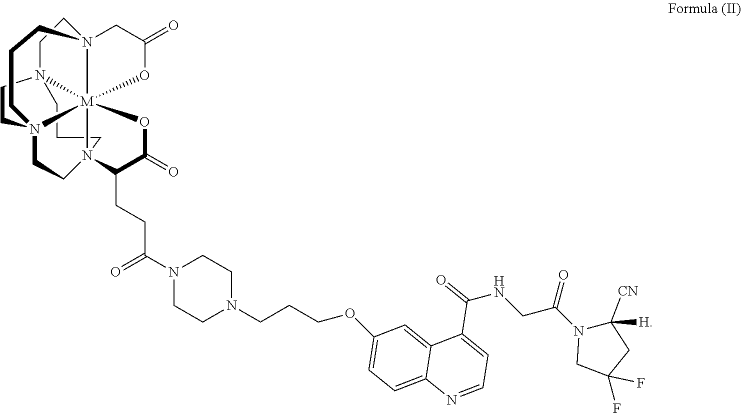

11. The compound of any one of the claims 1-9, wherein the compound is Formula (III):

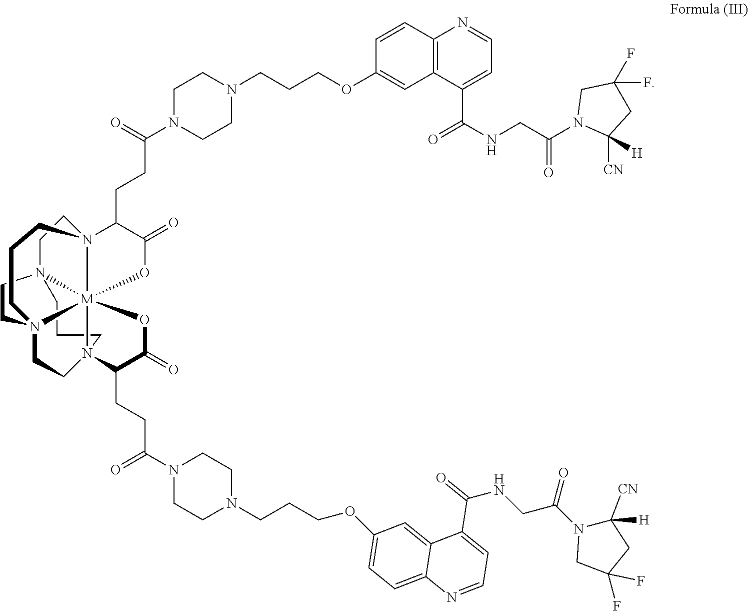

12. The compound of any one of the claims 1-9, wherein the compound is selected from: 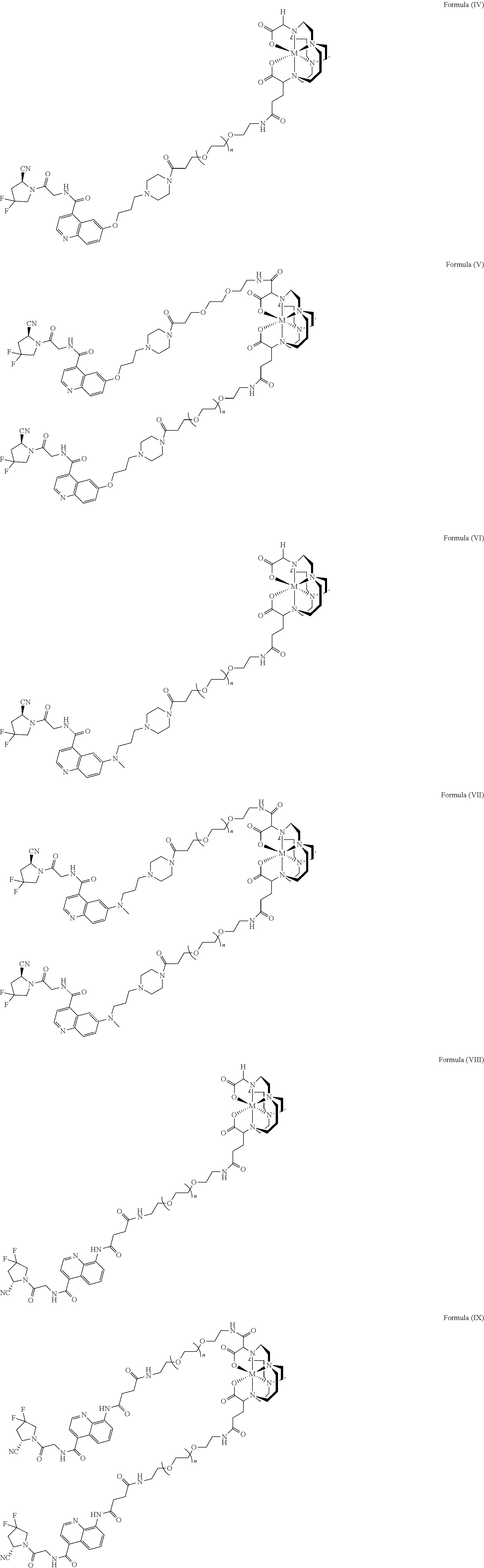 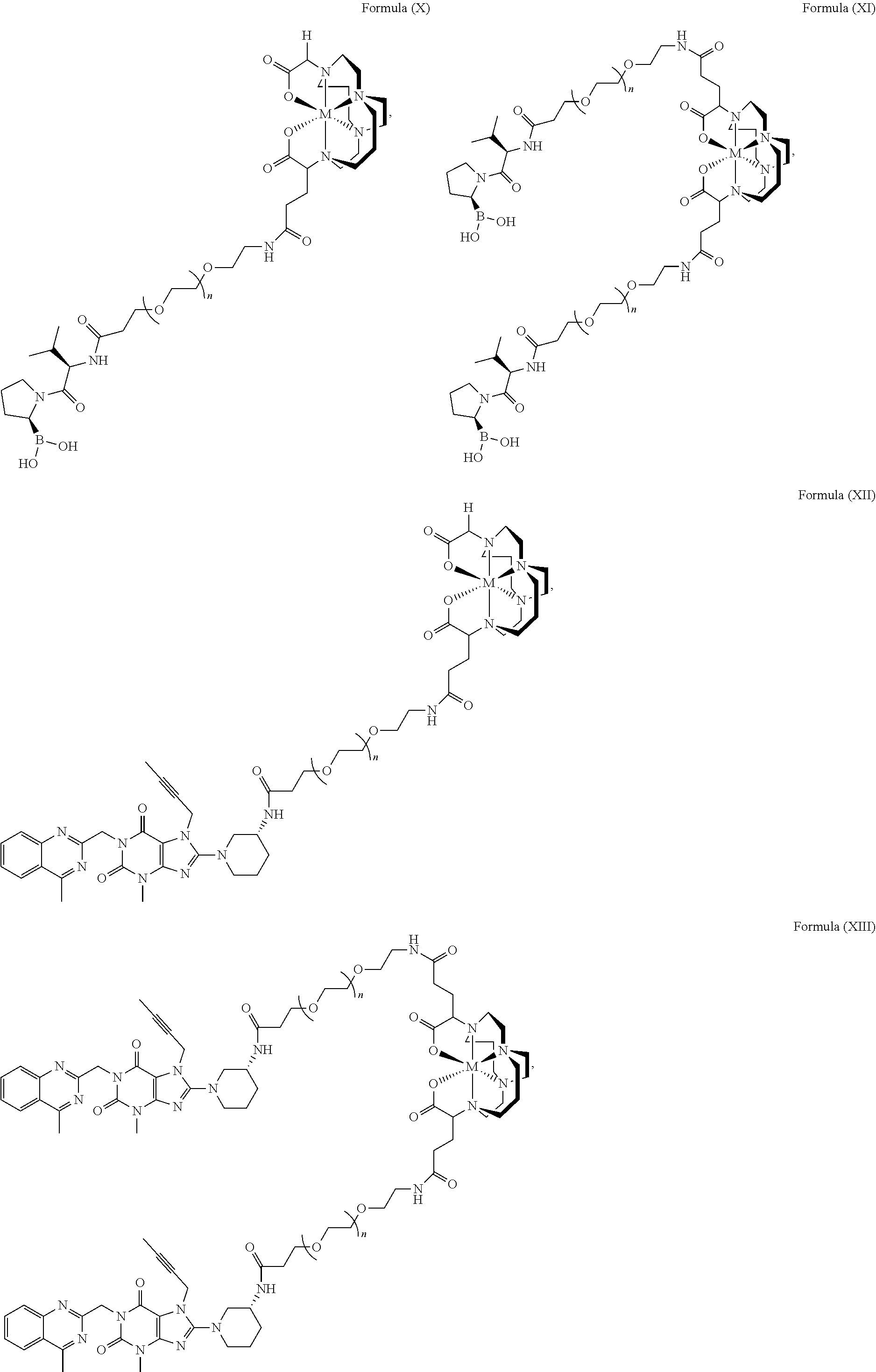 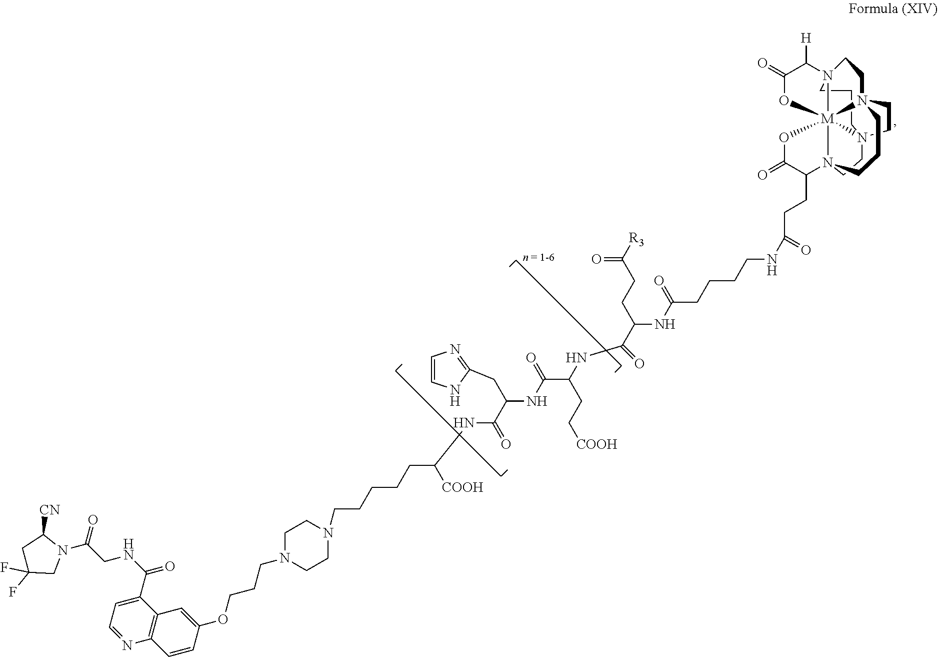 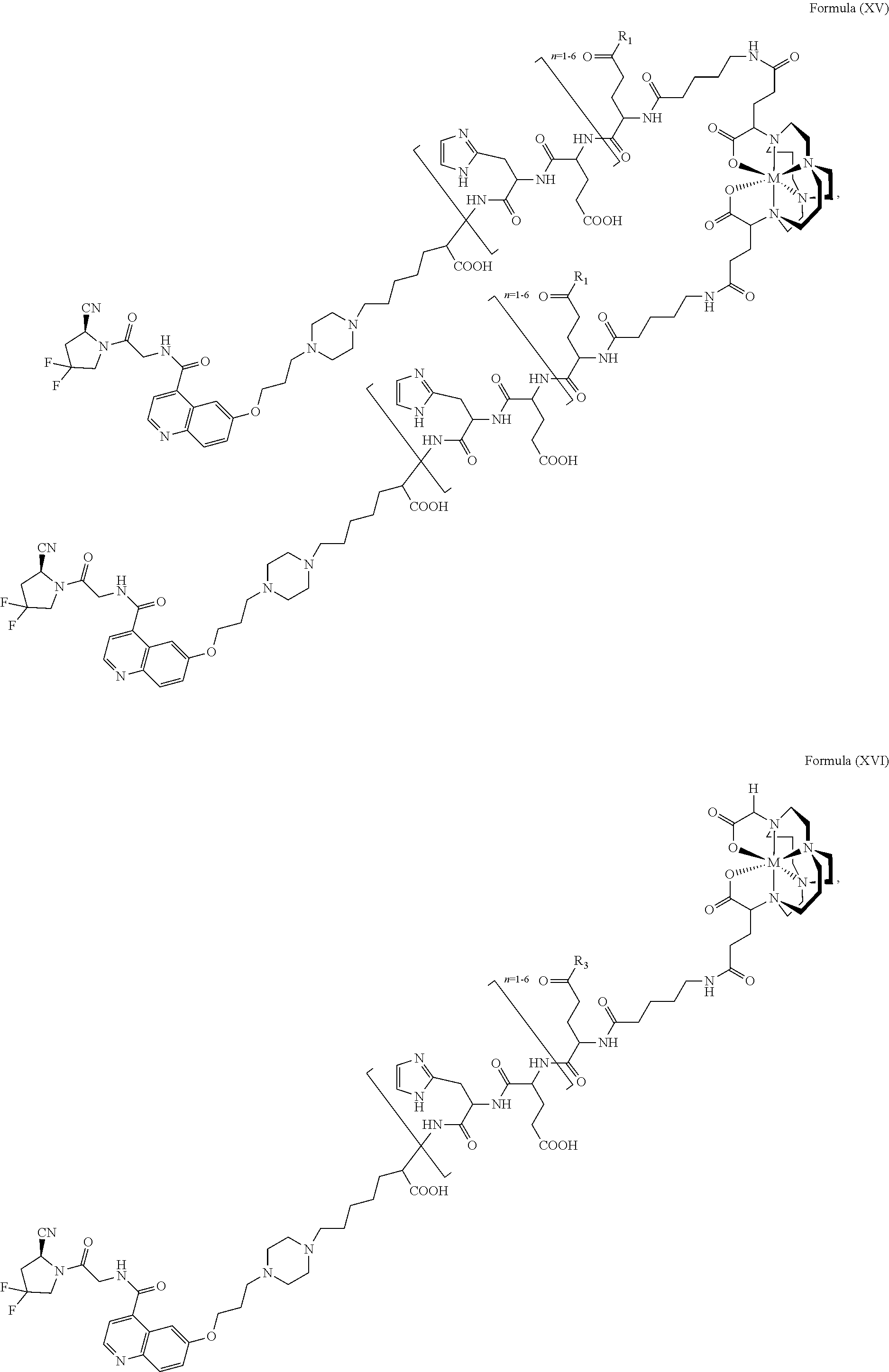 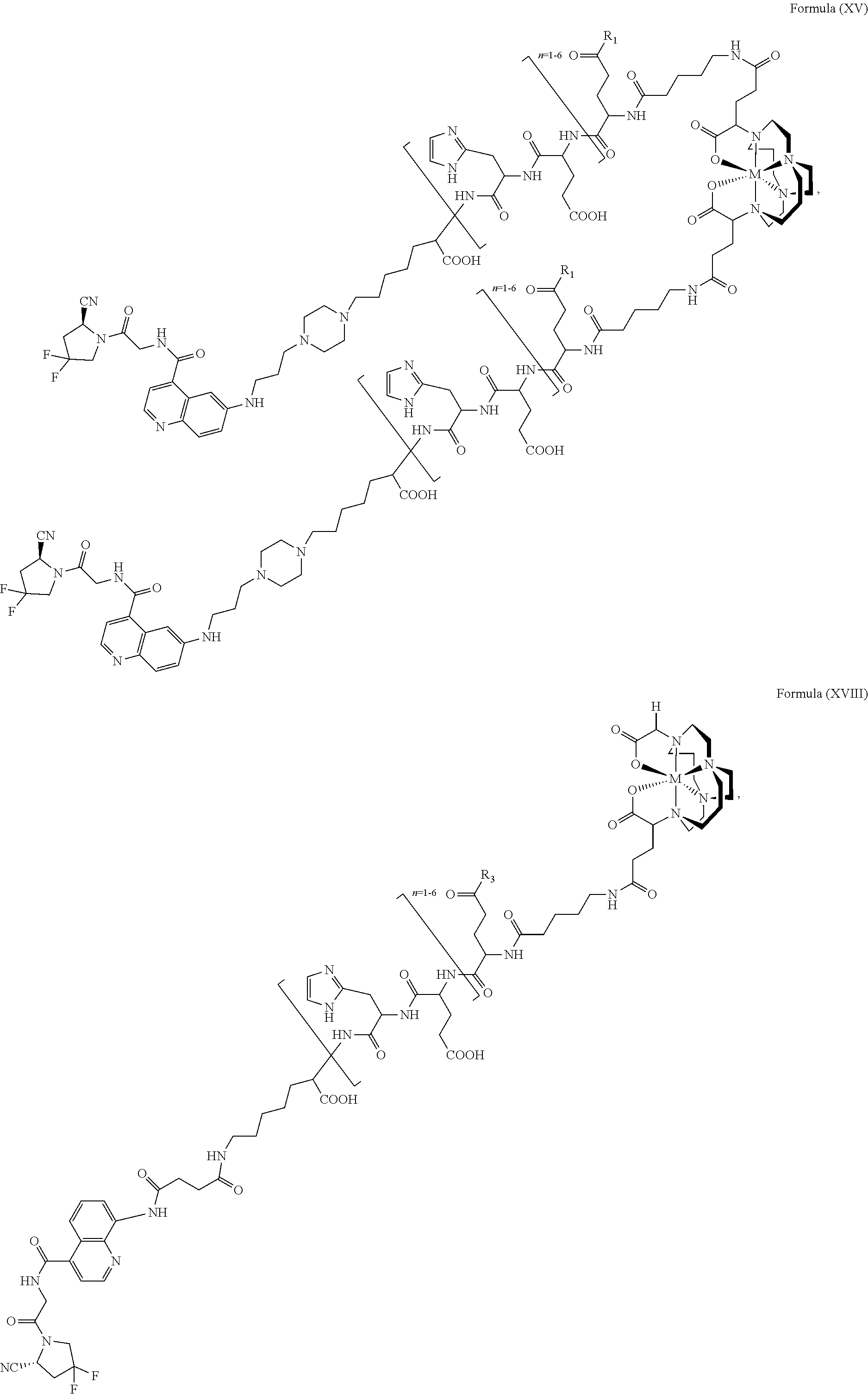 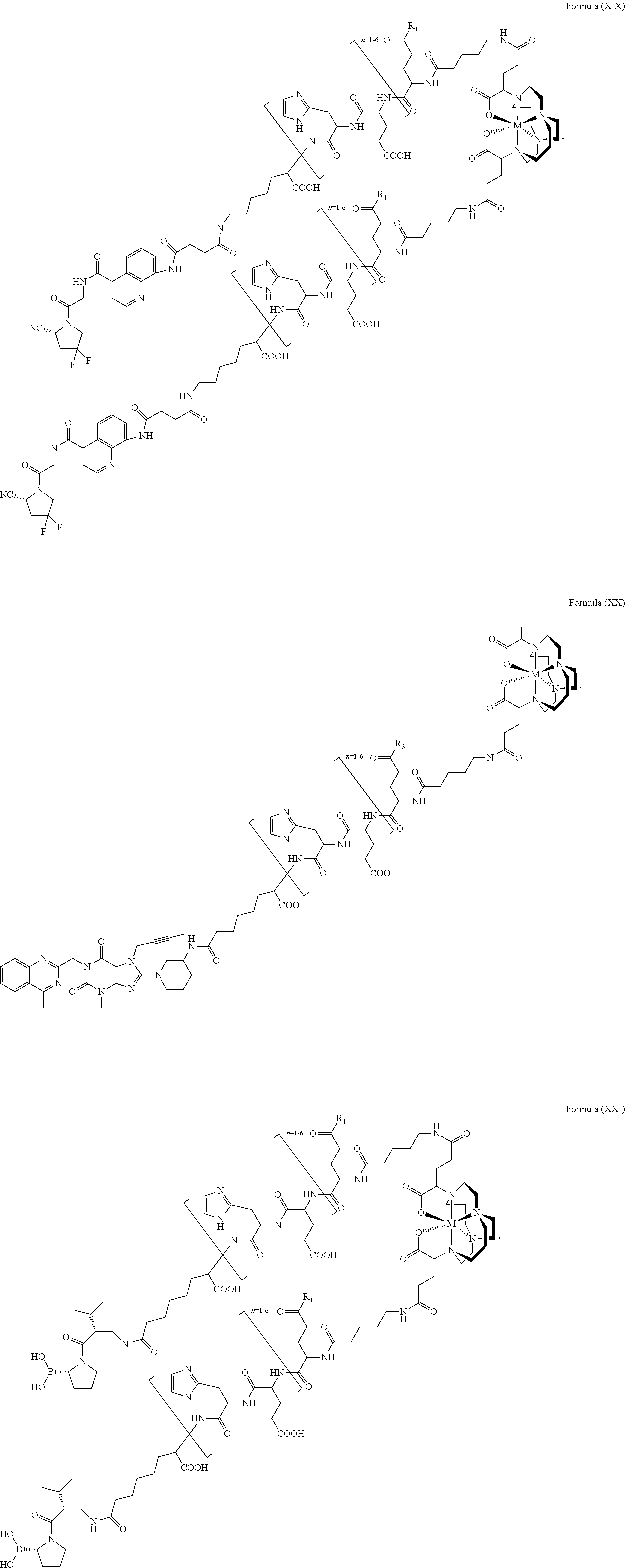 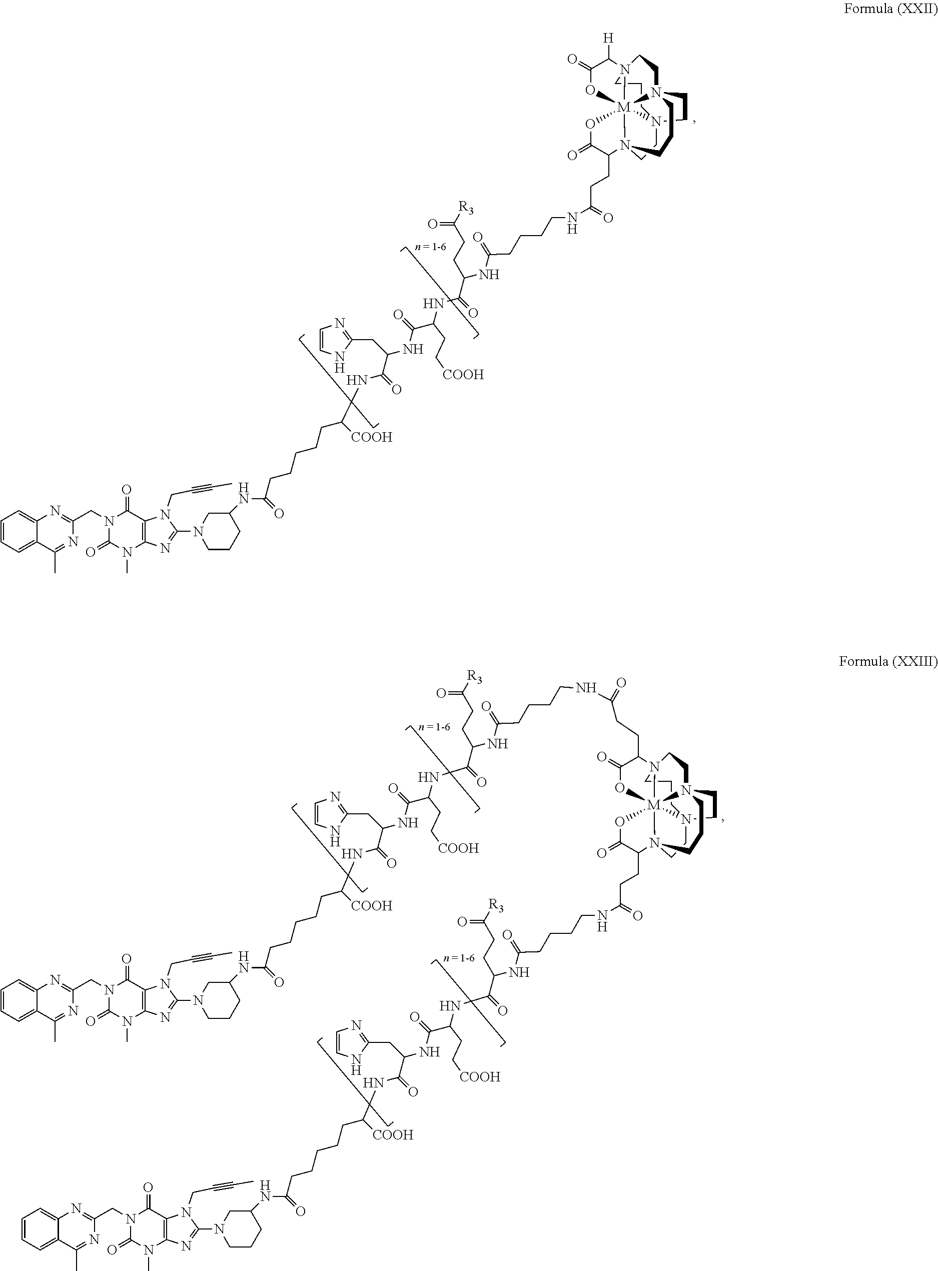 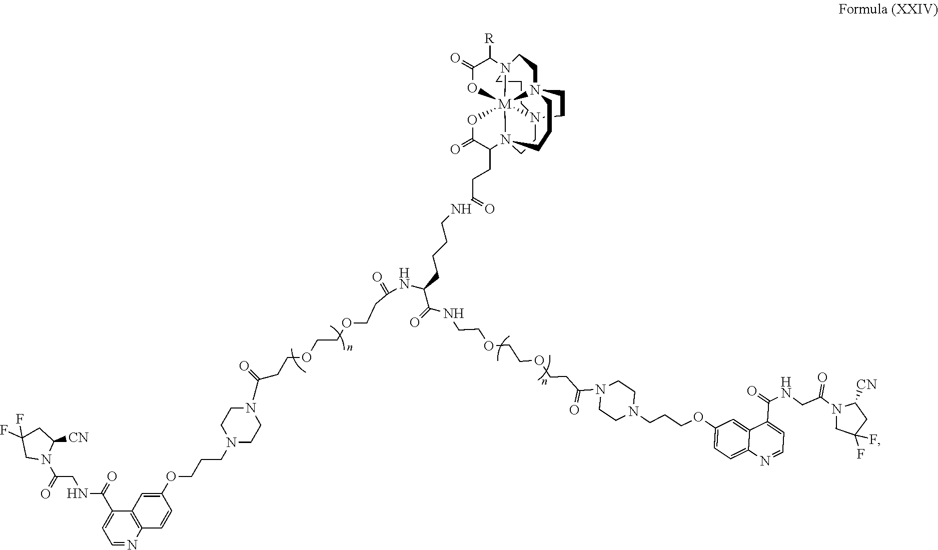 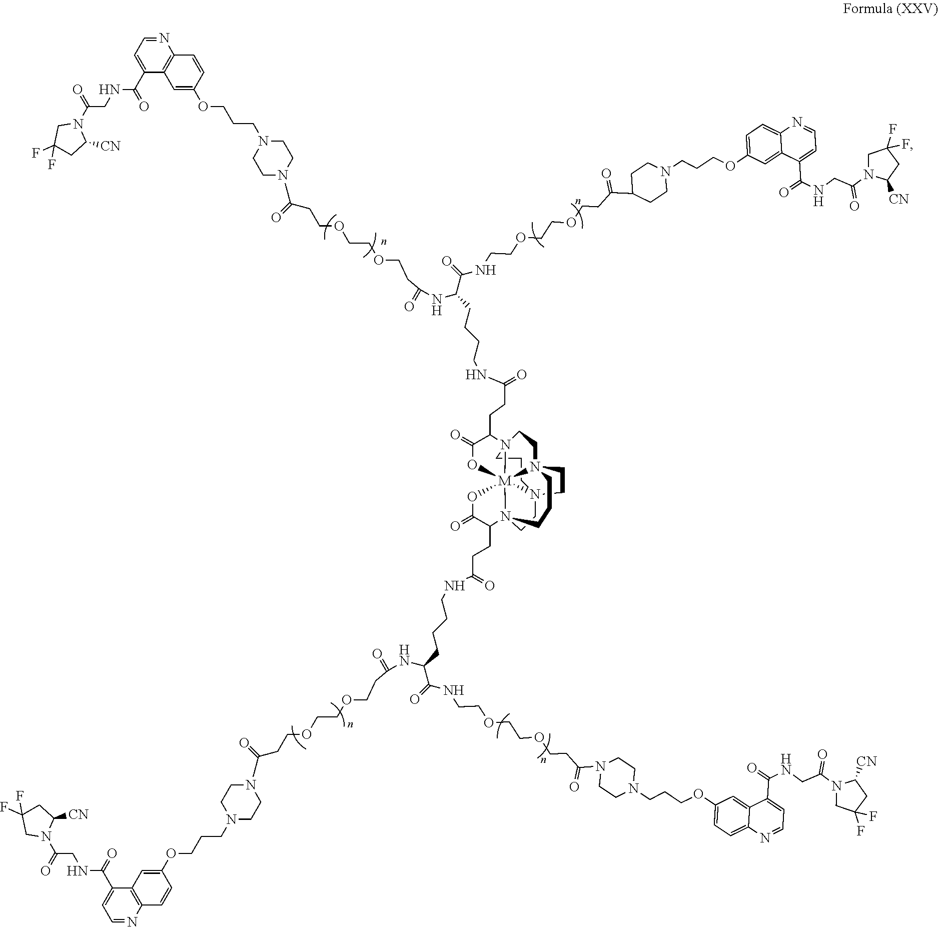 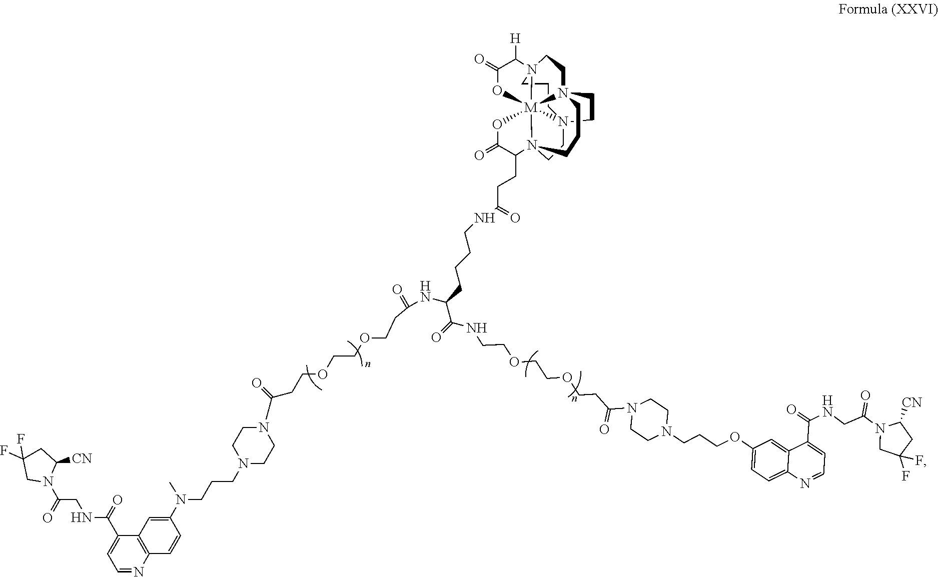 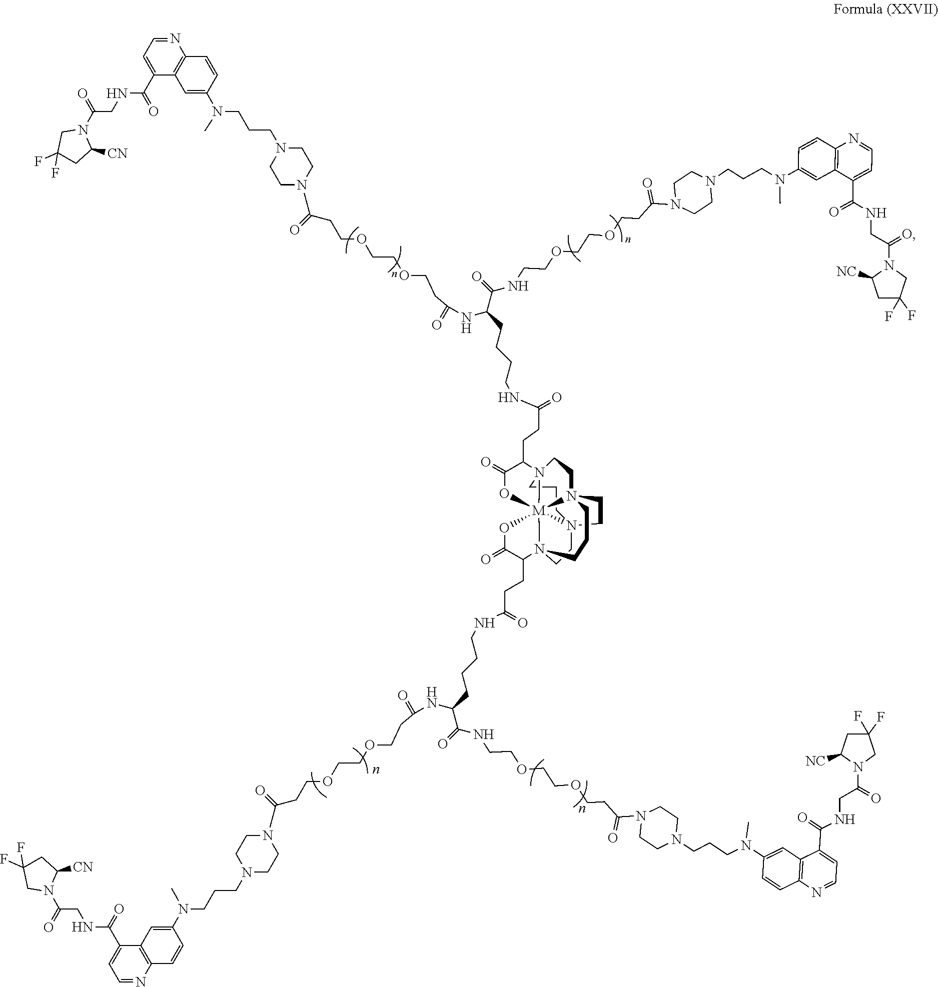 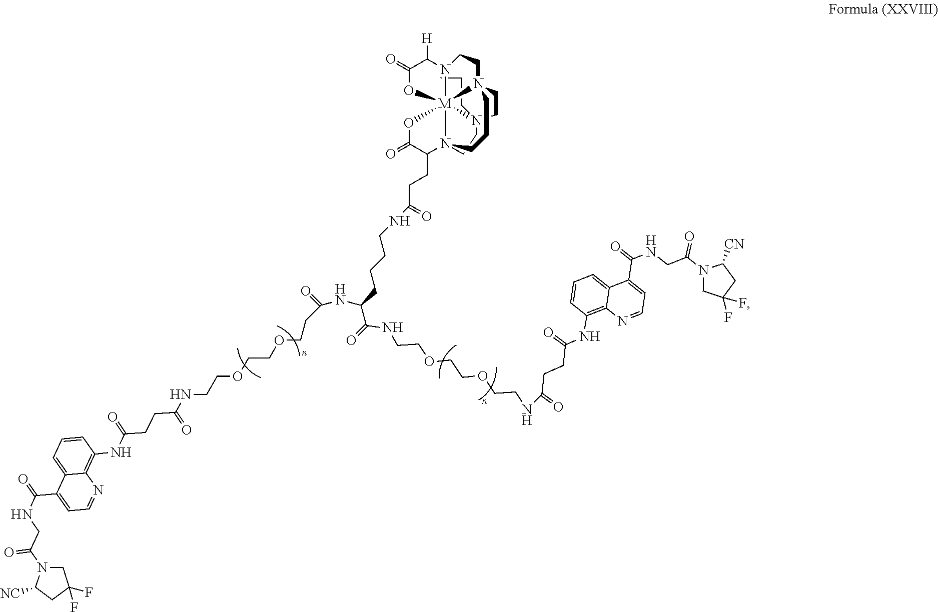 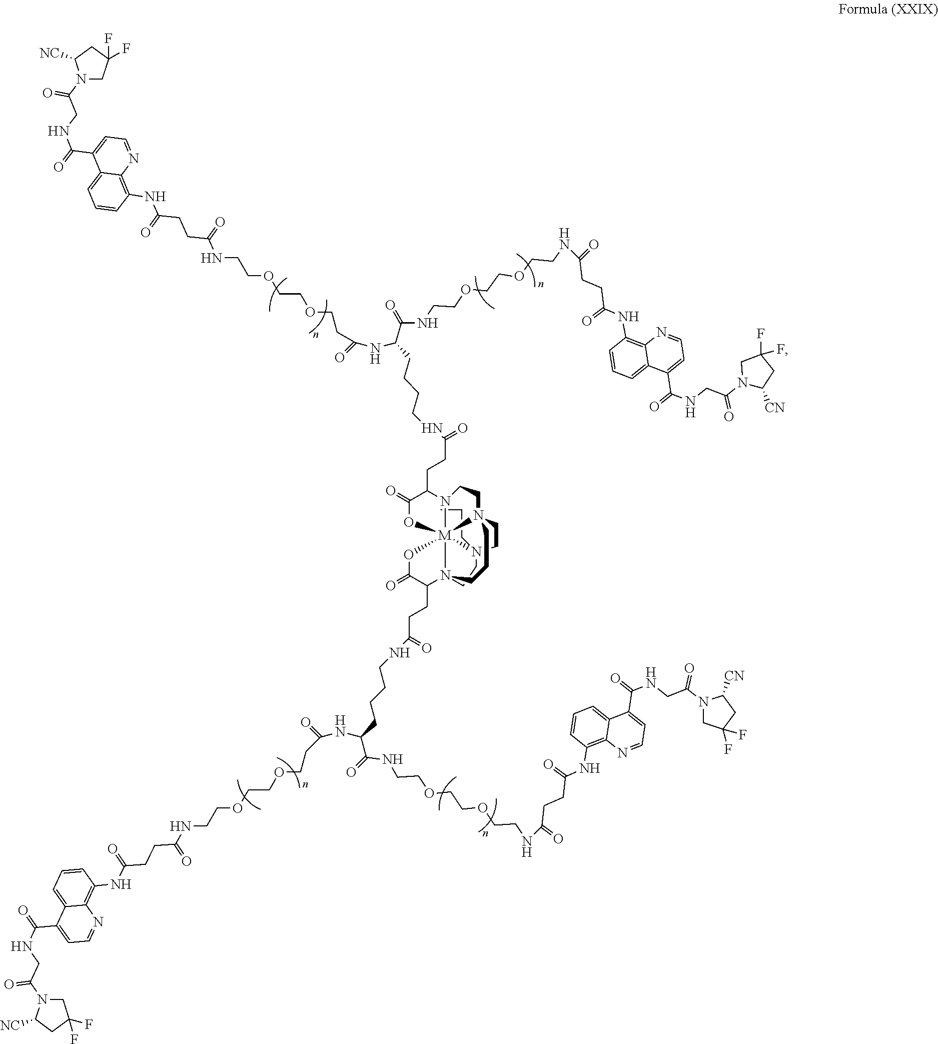 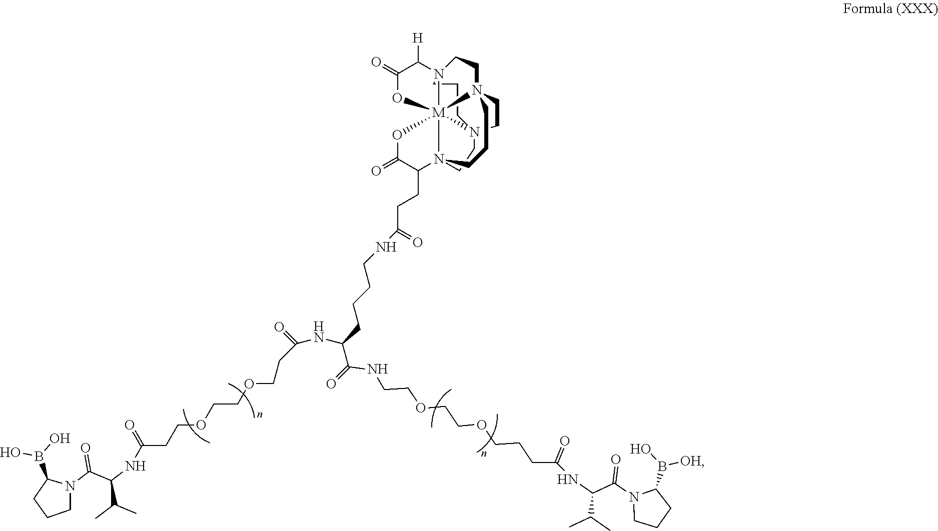 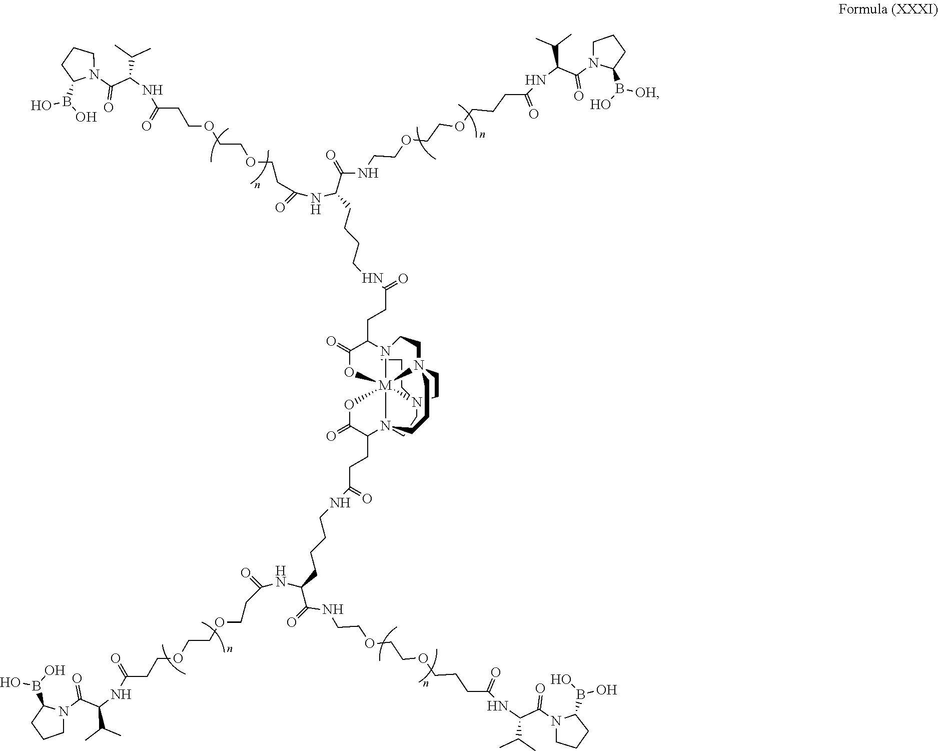 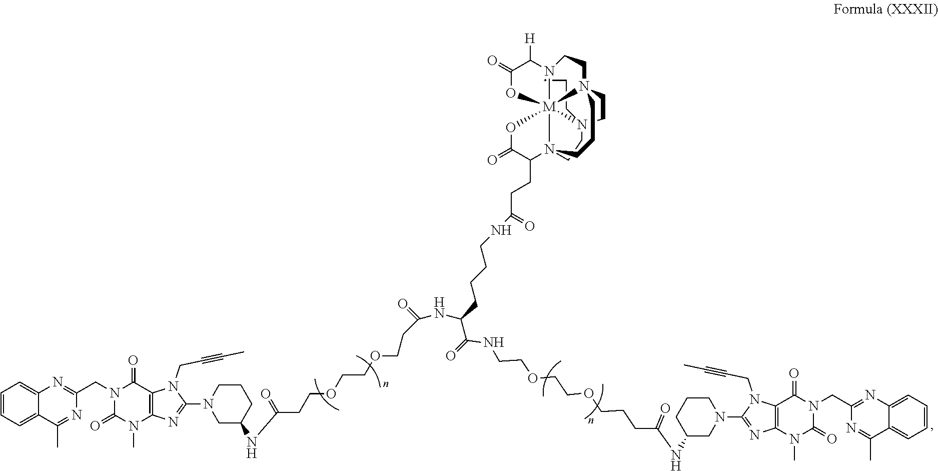 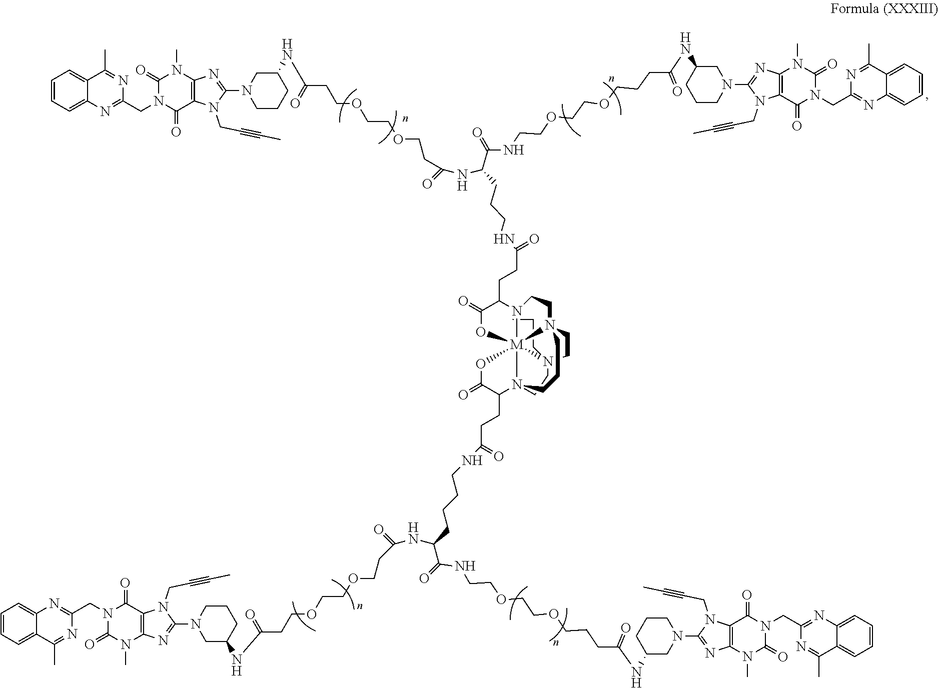 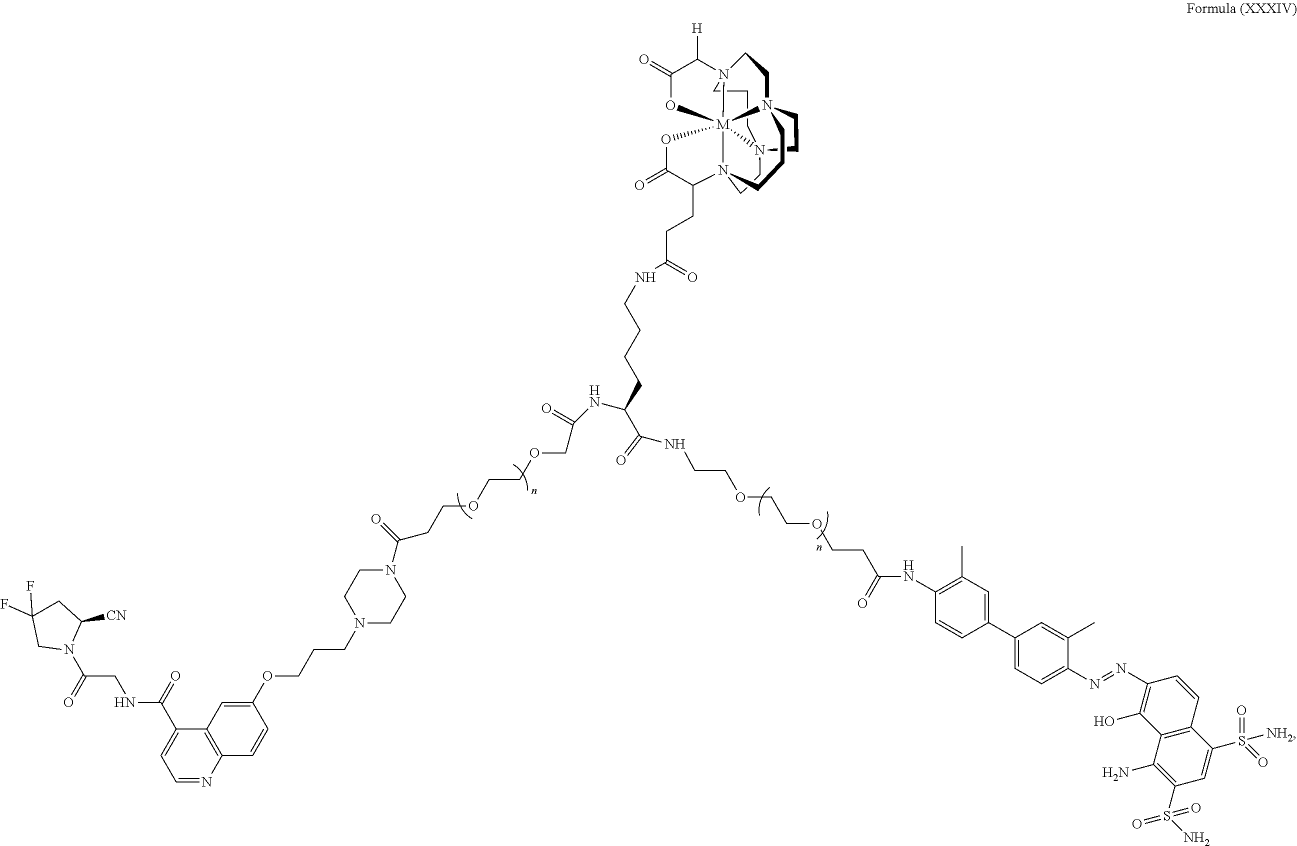 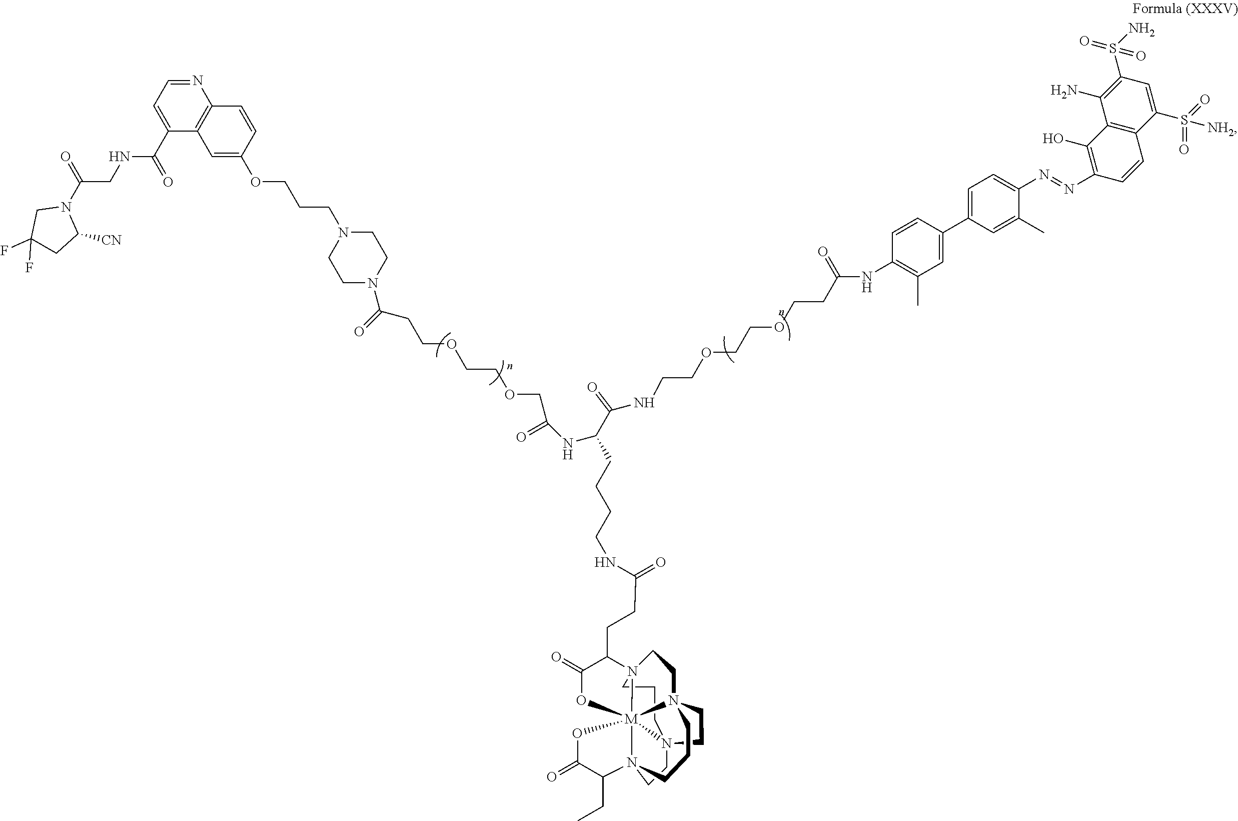 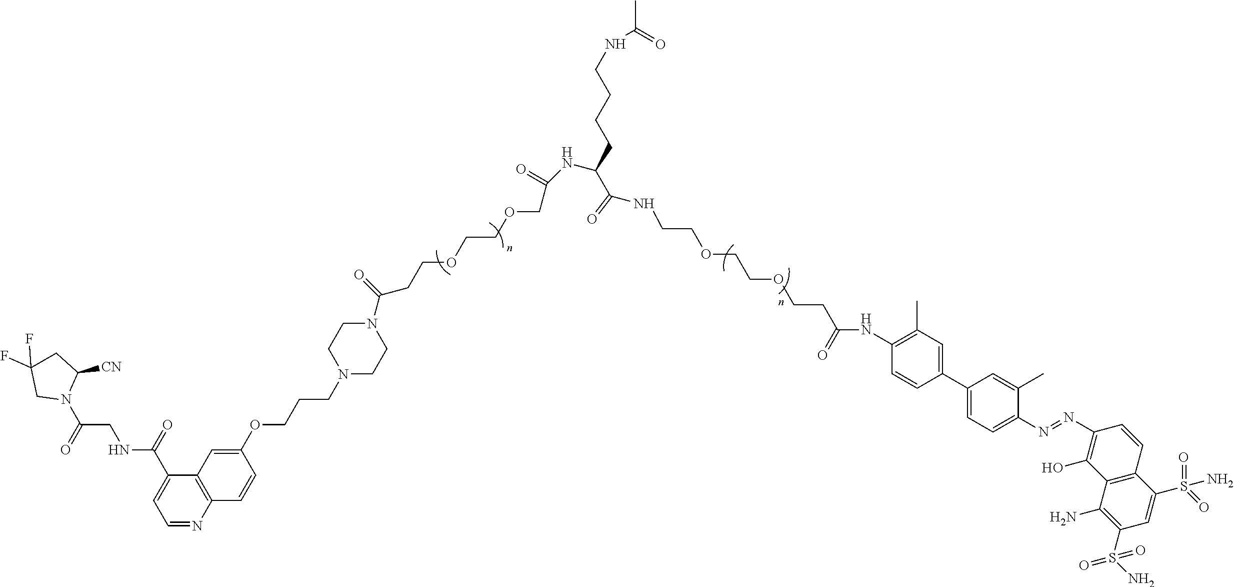 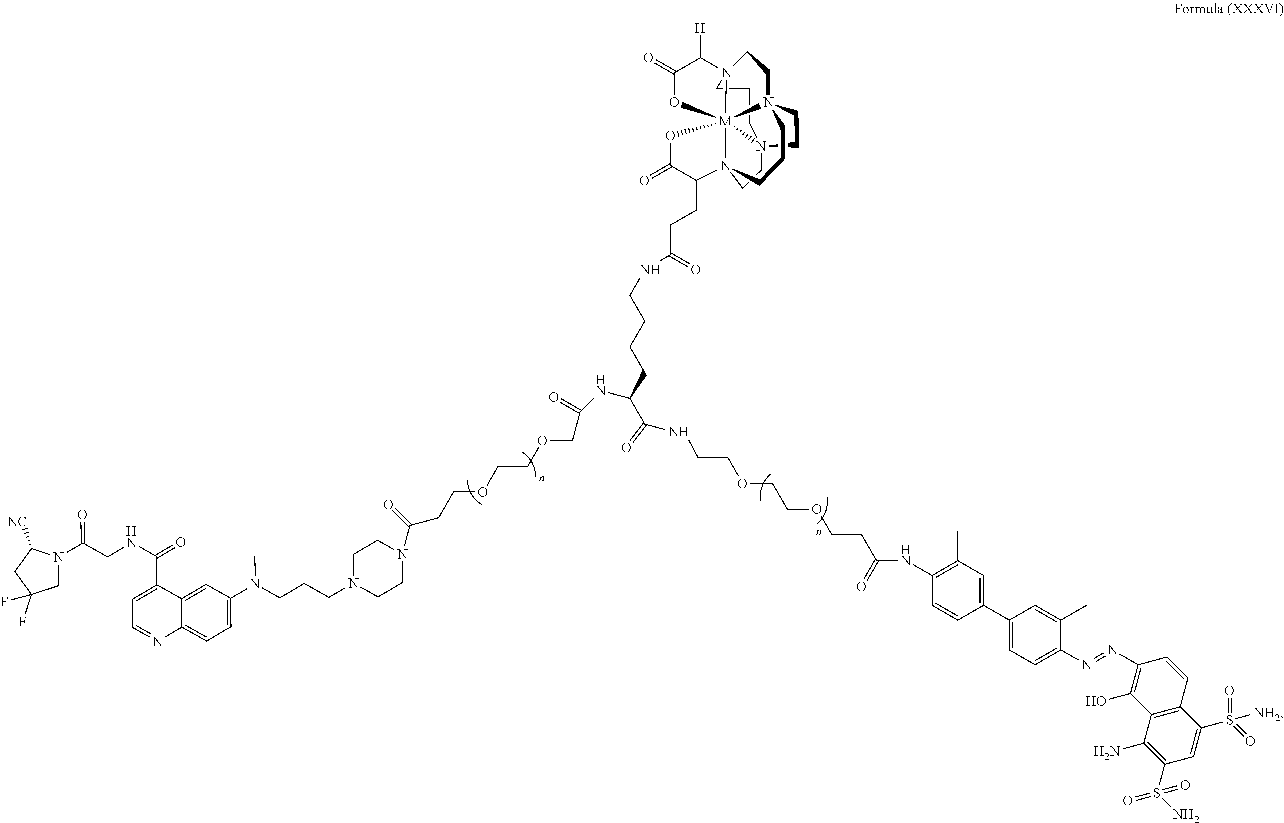 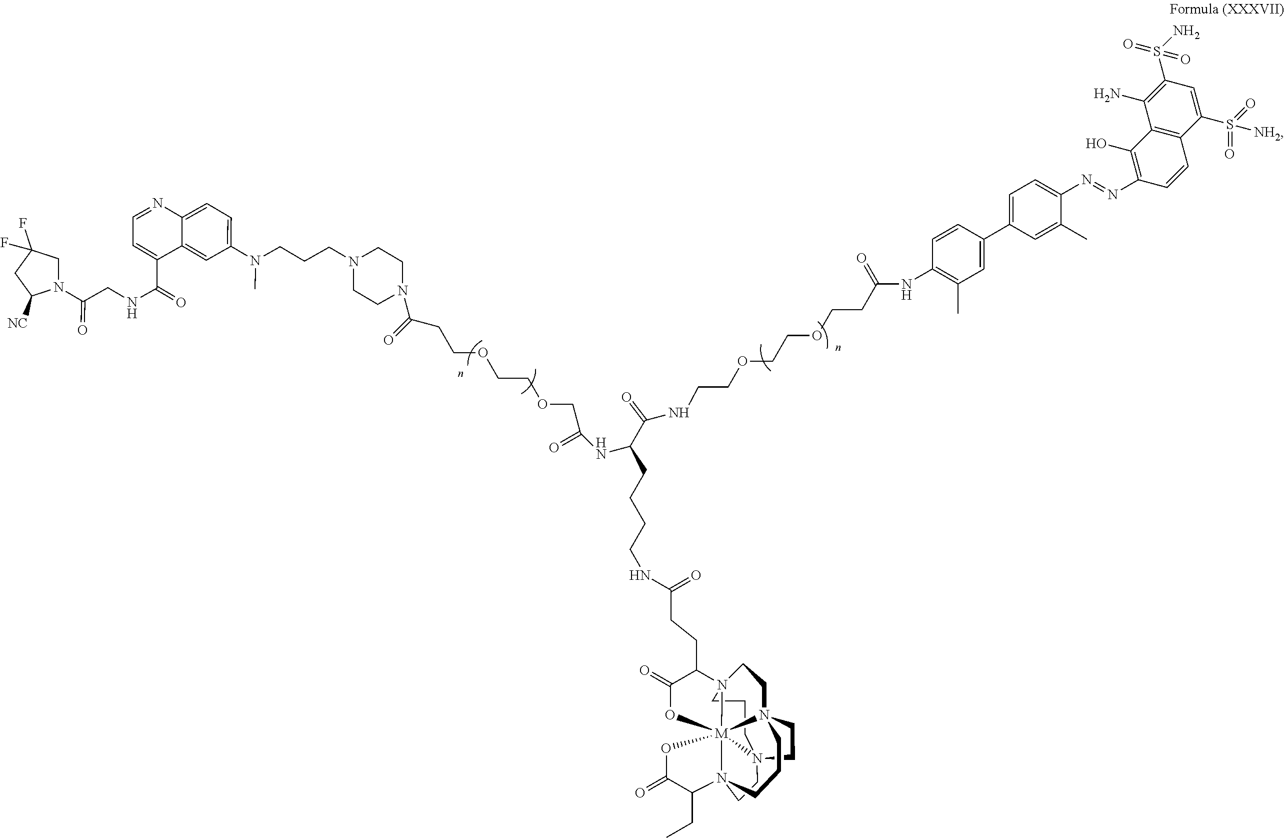 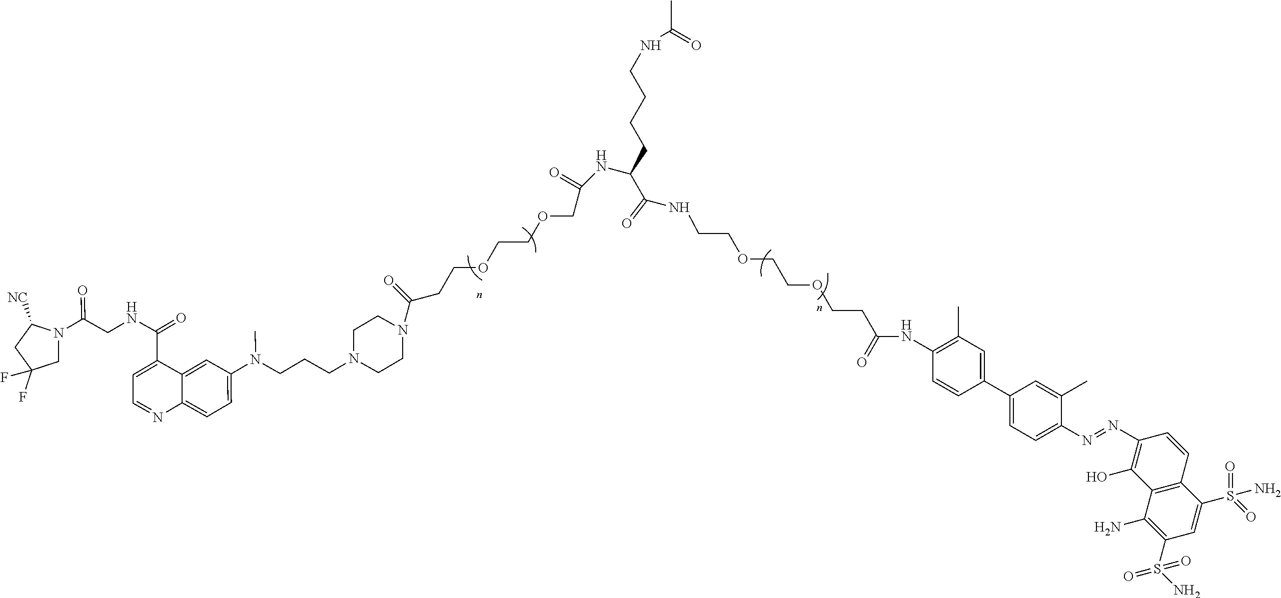 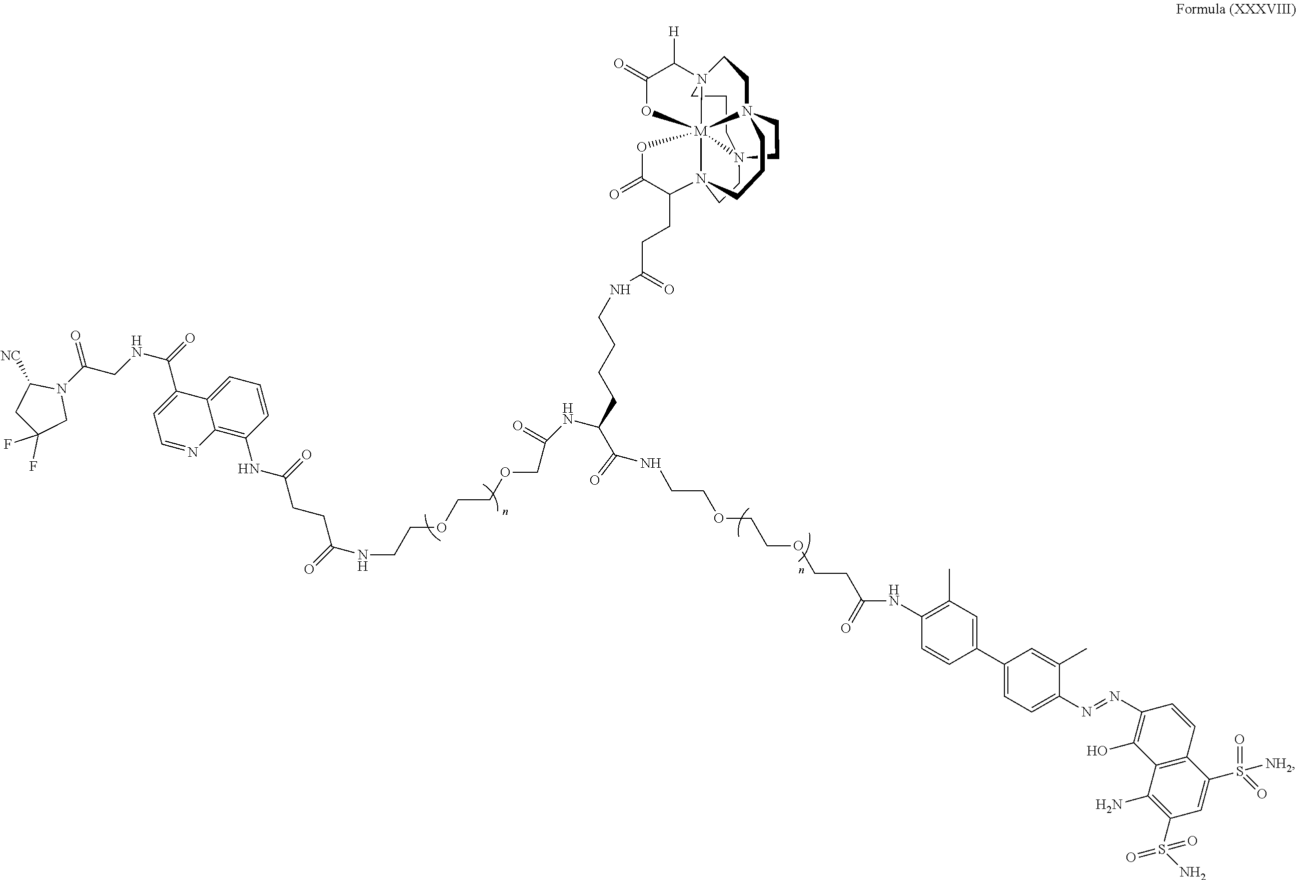 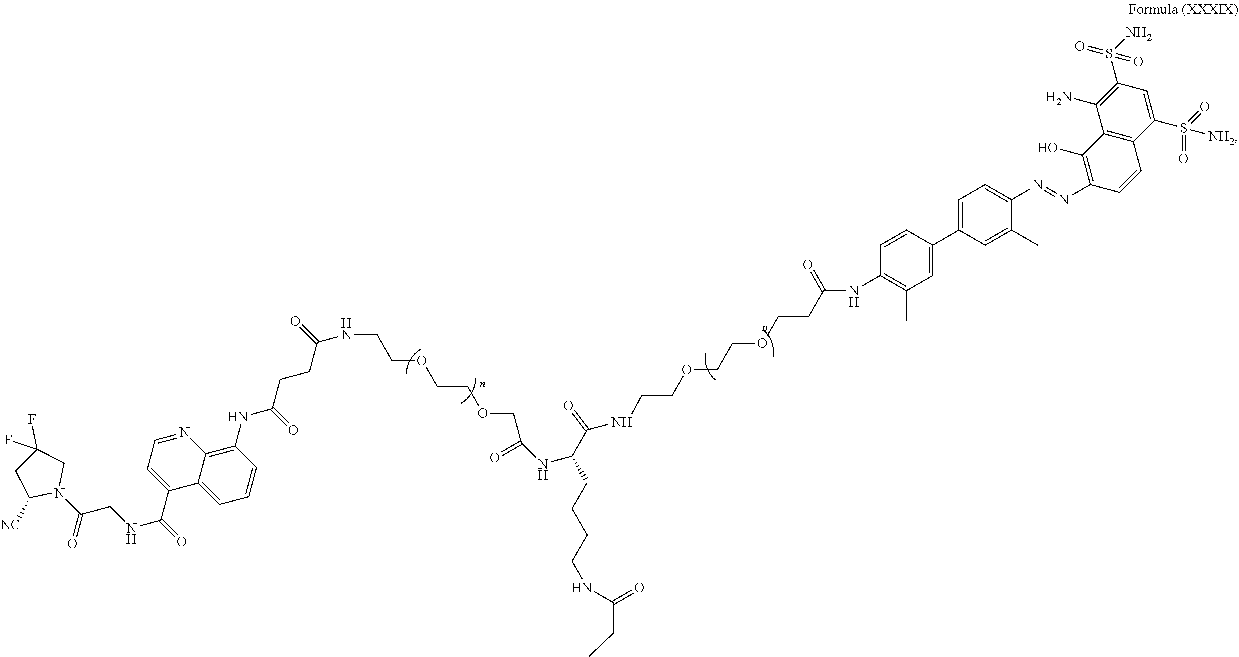 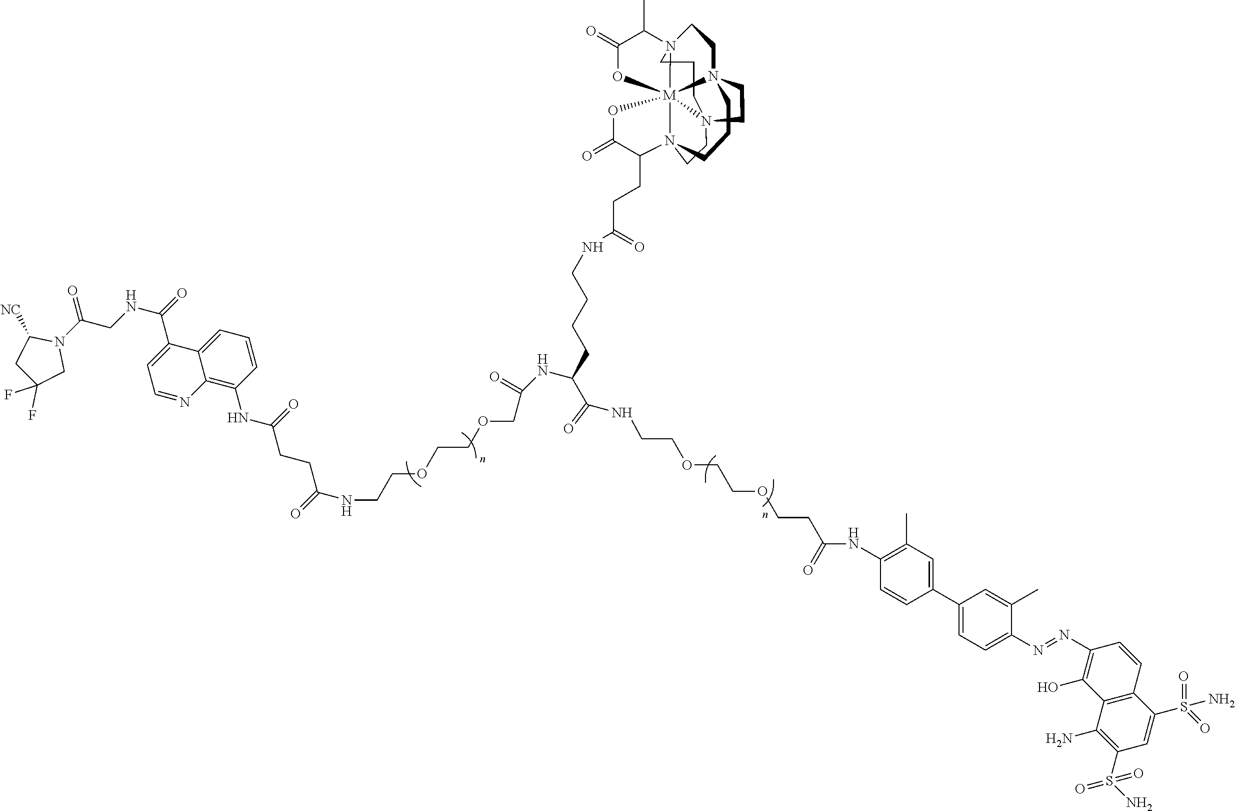 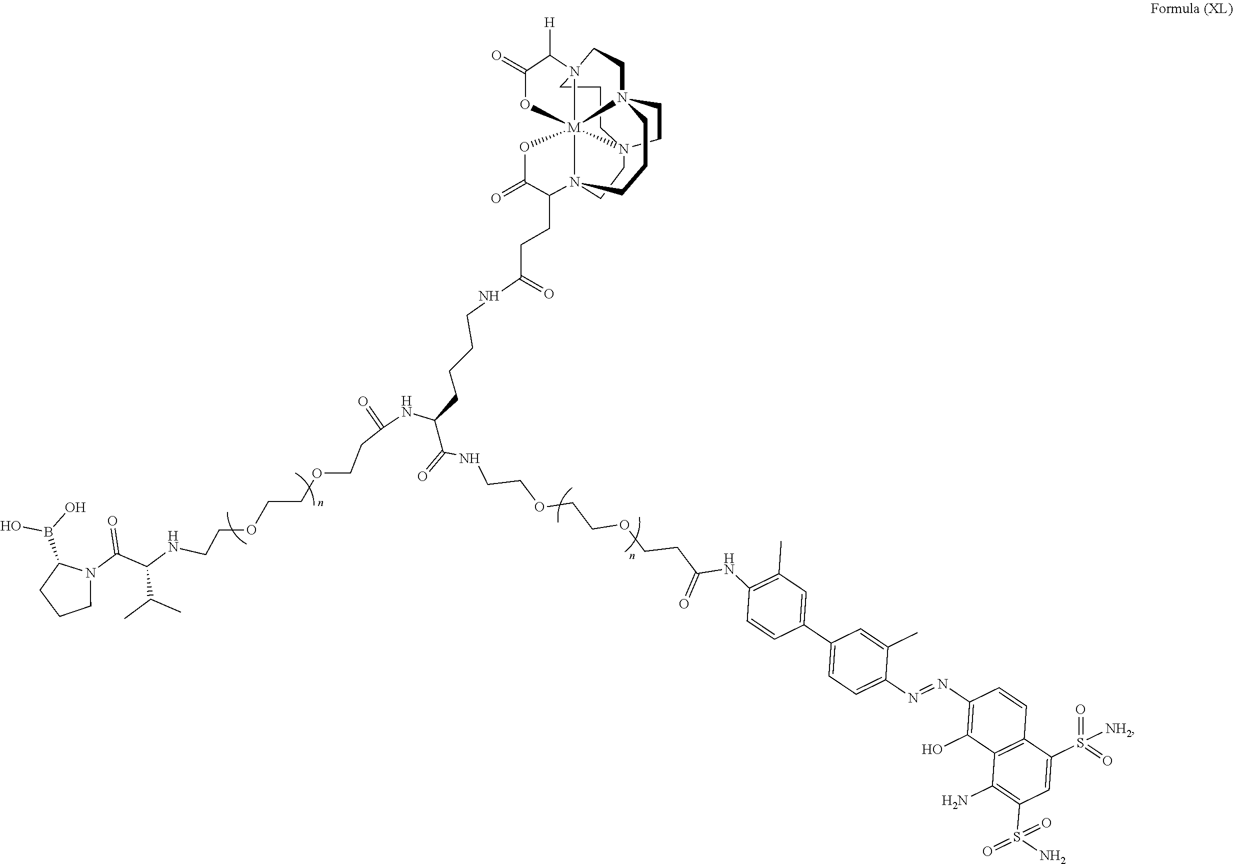 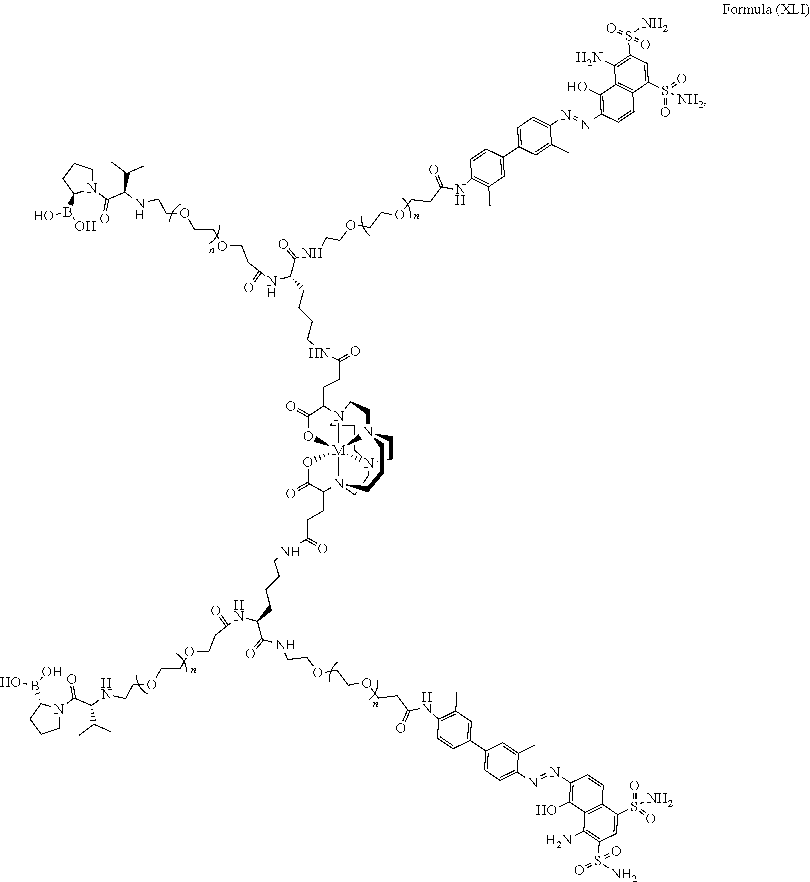 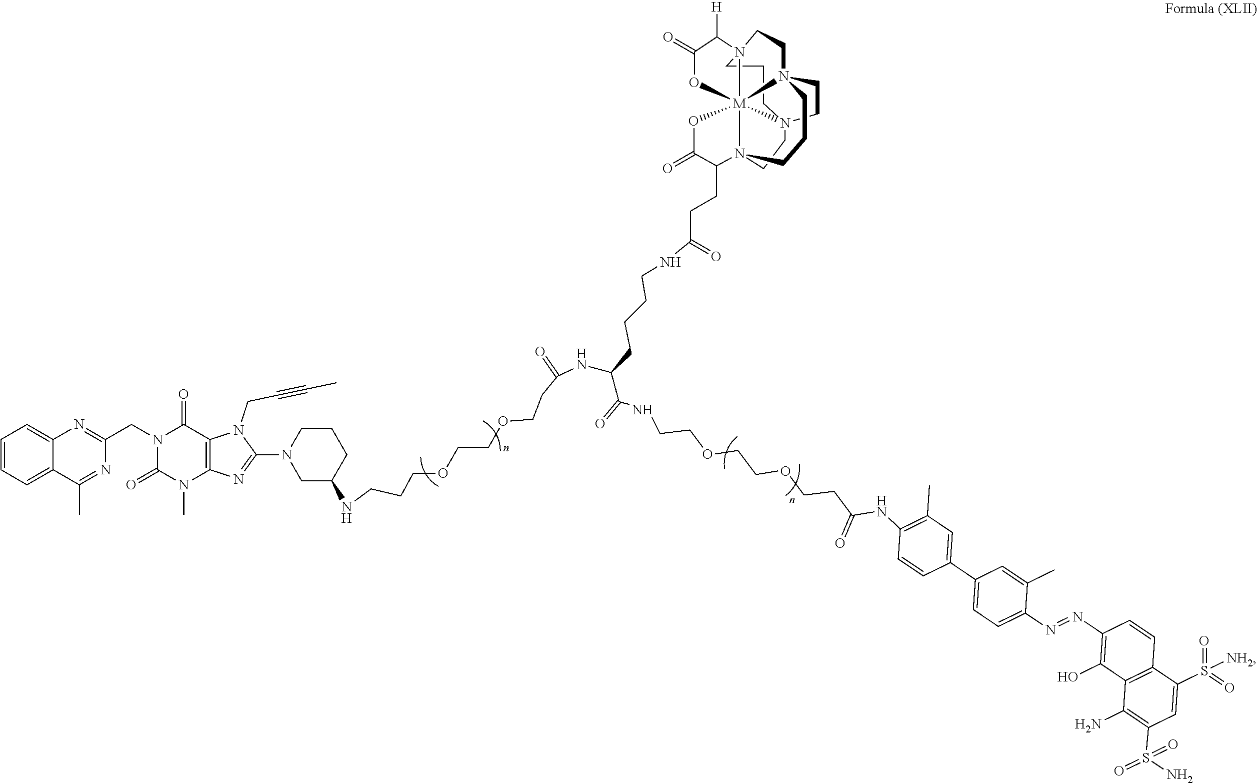 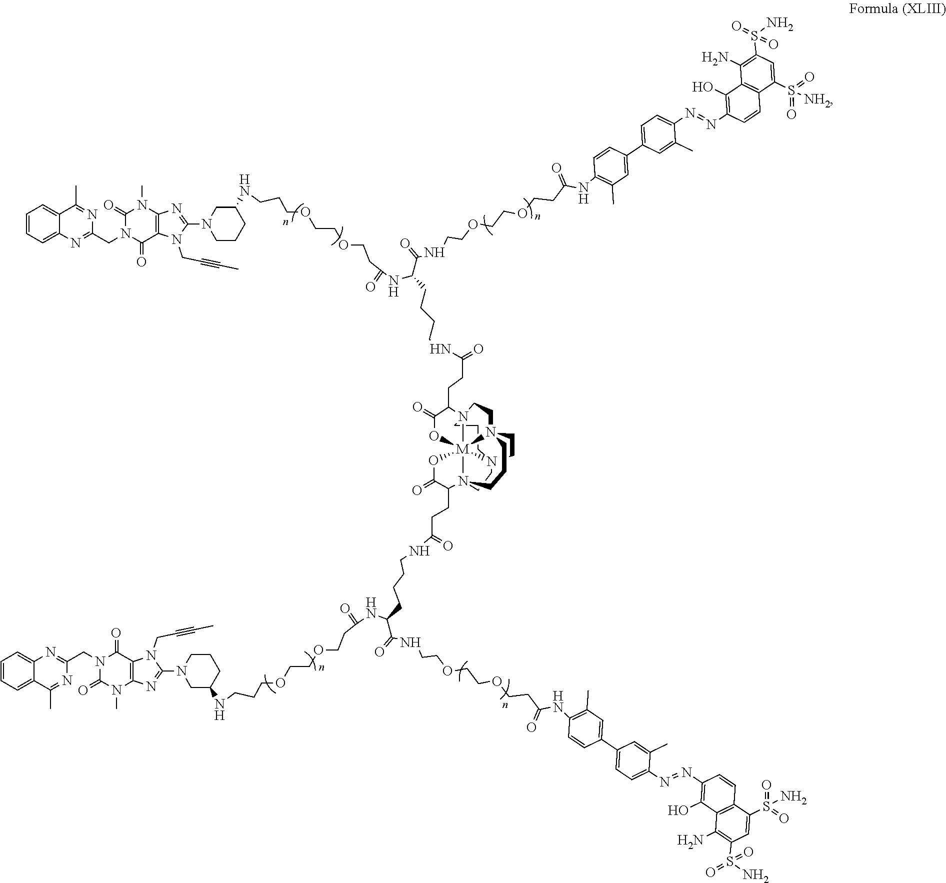 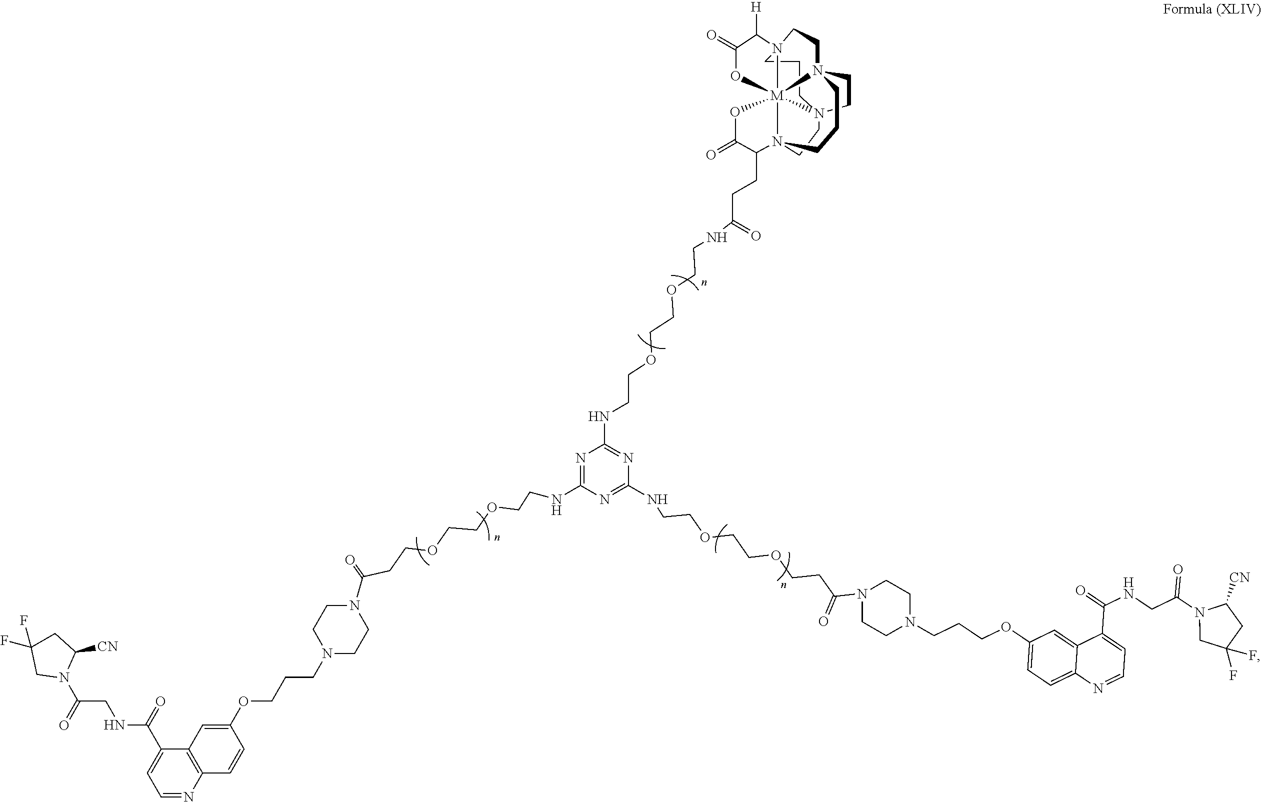 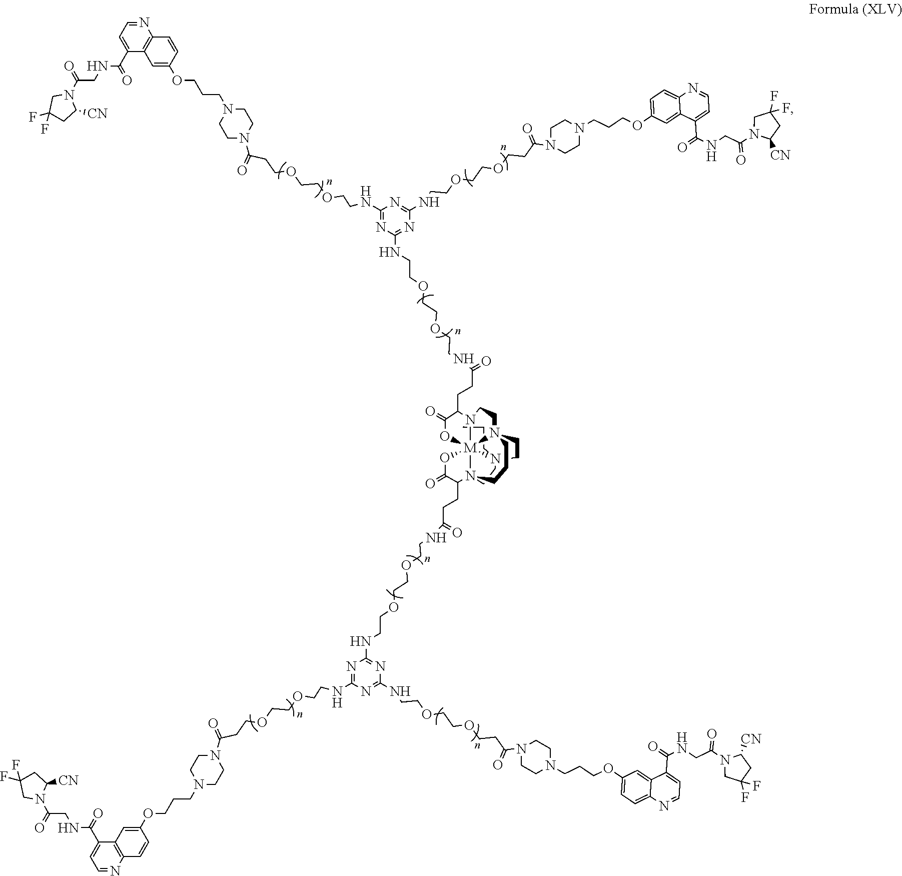 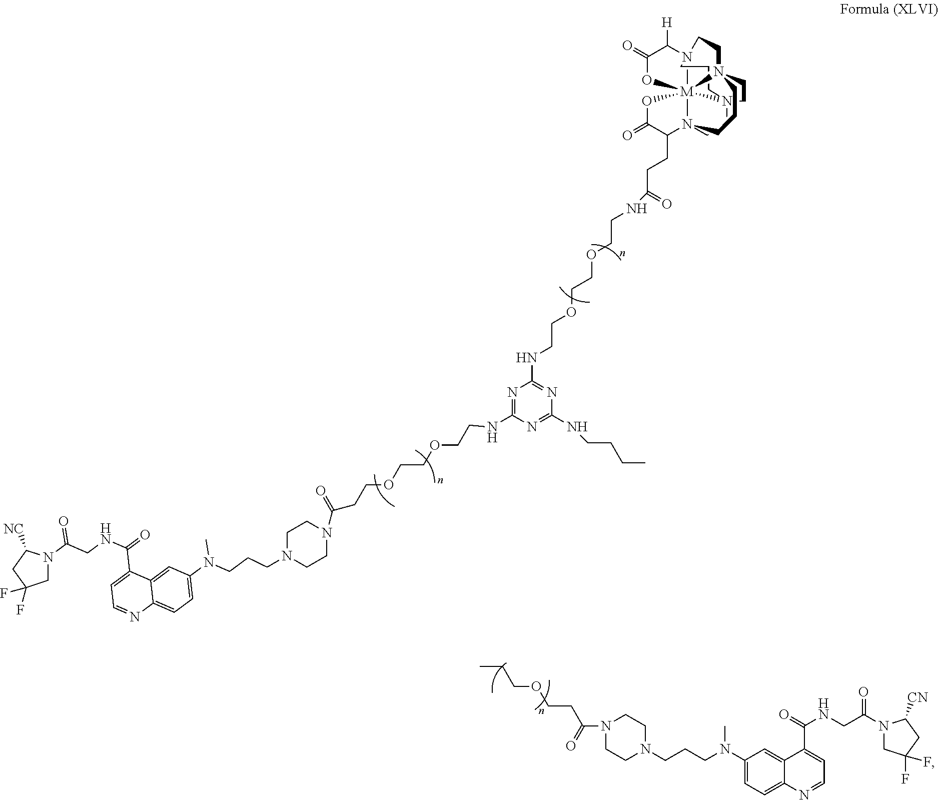 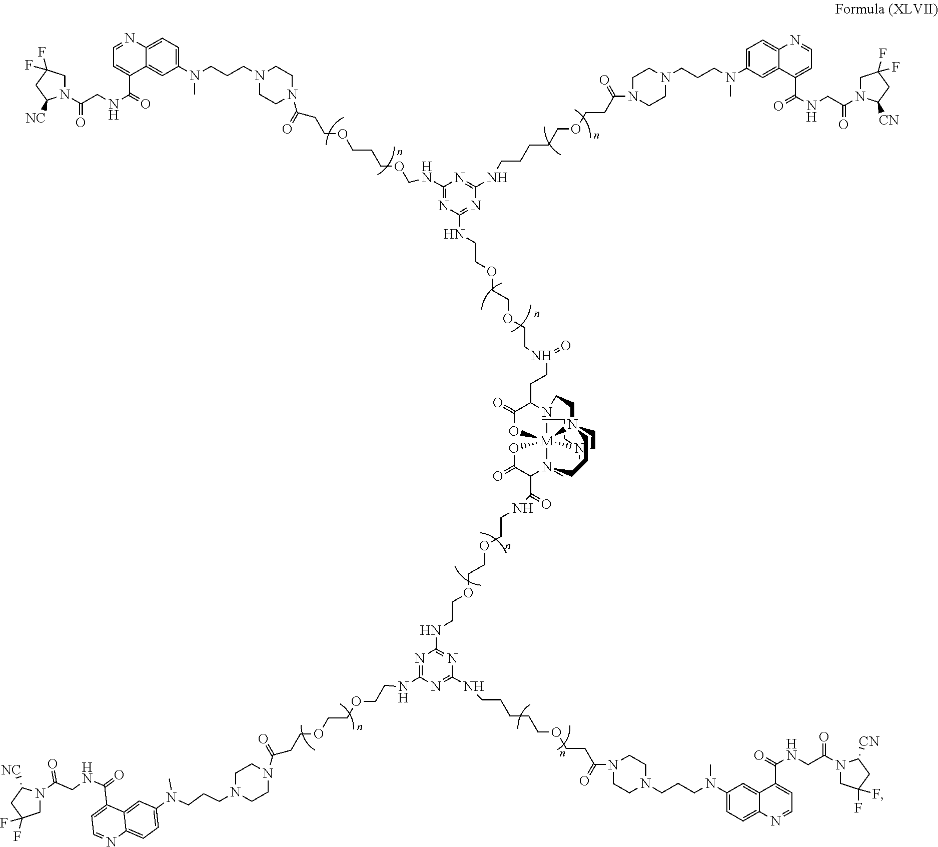 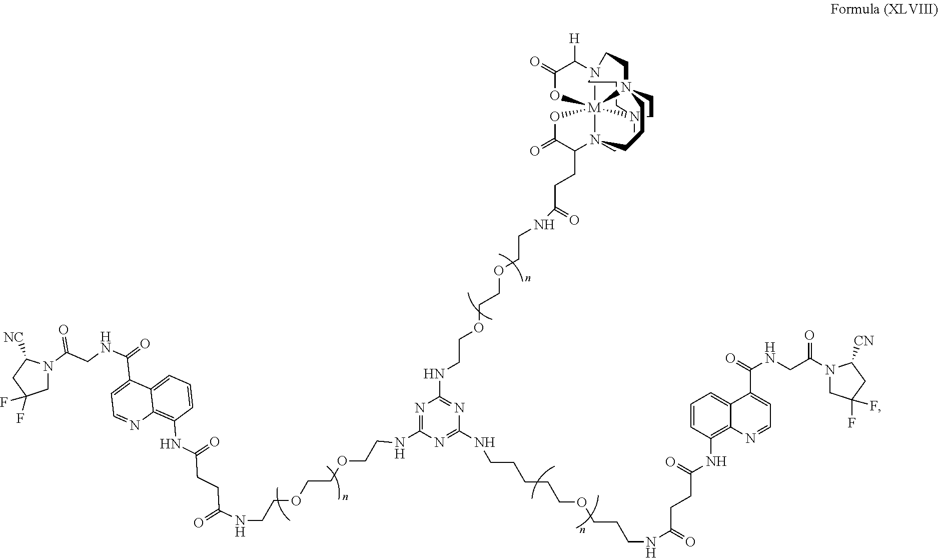 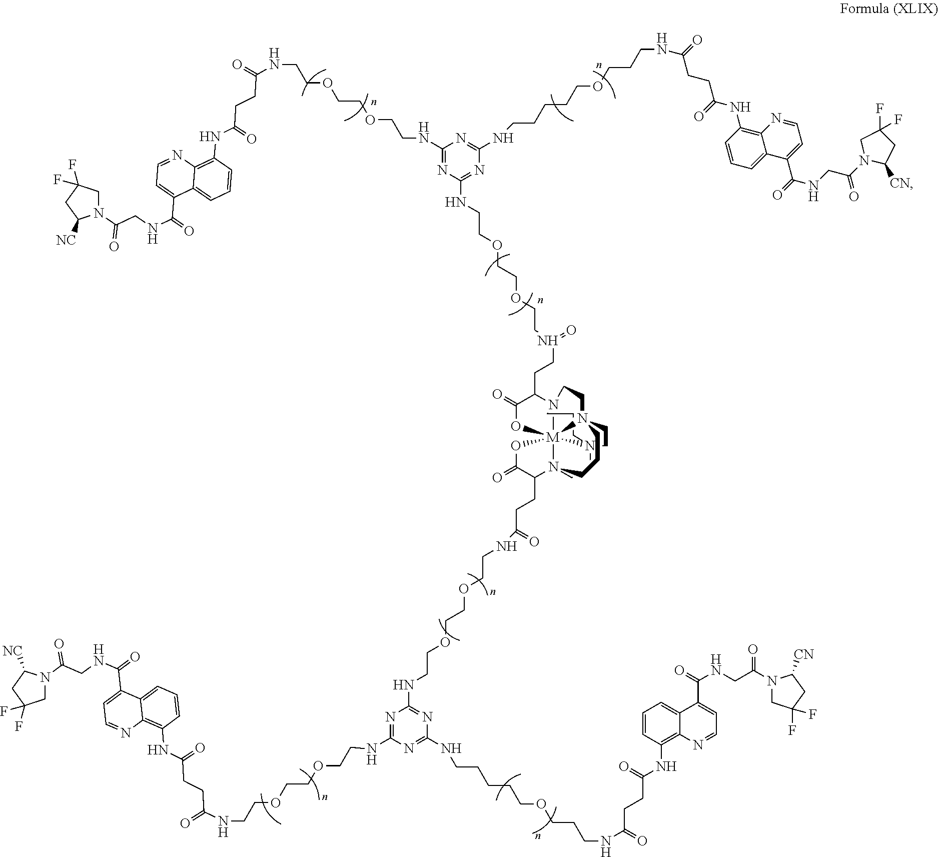 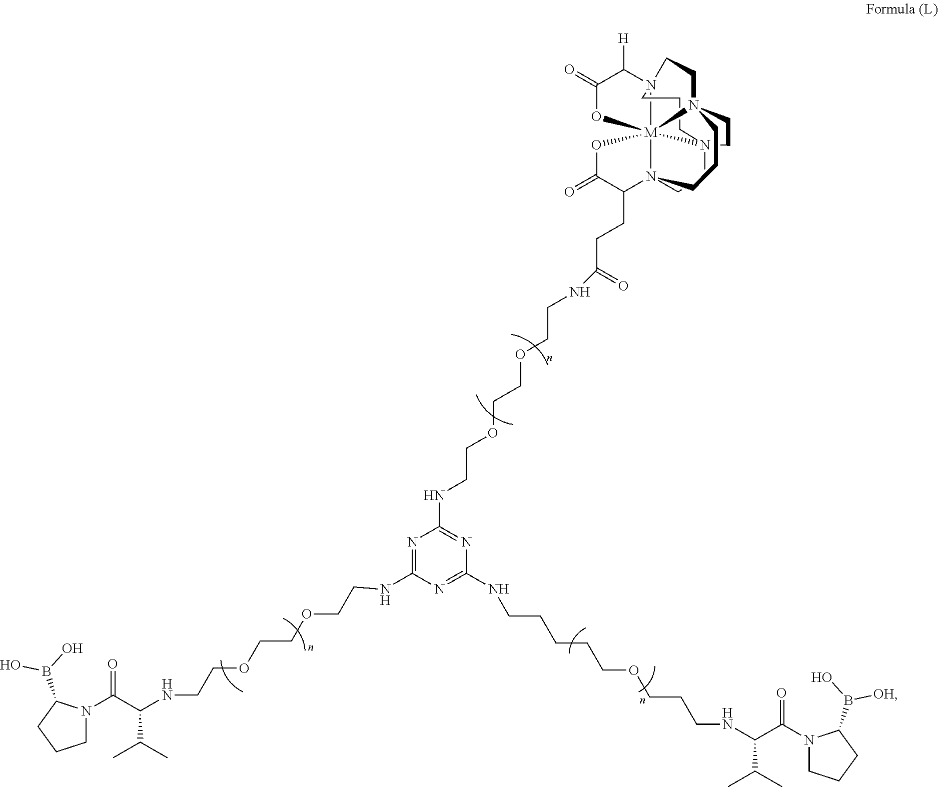 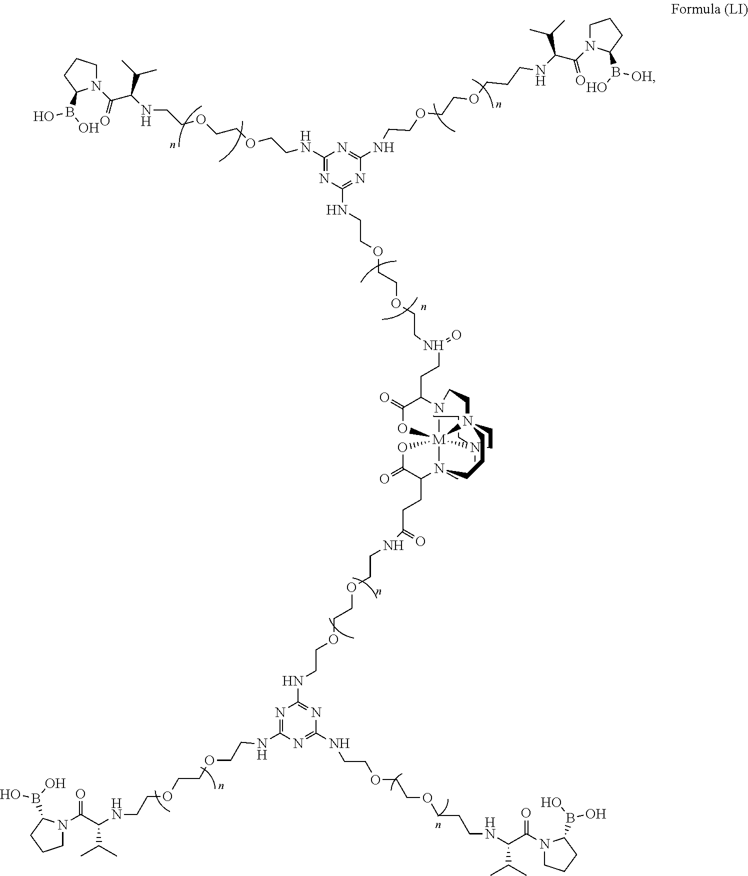 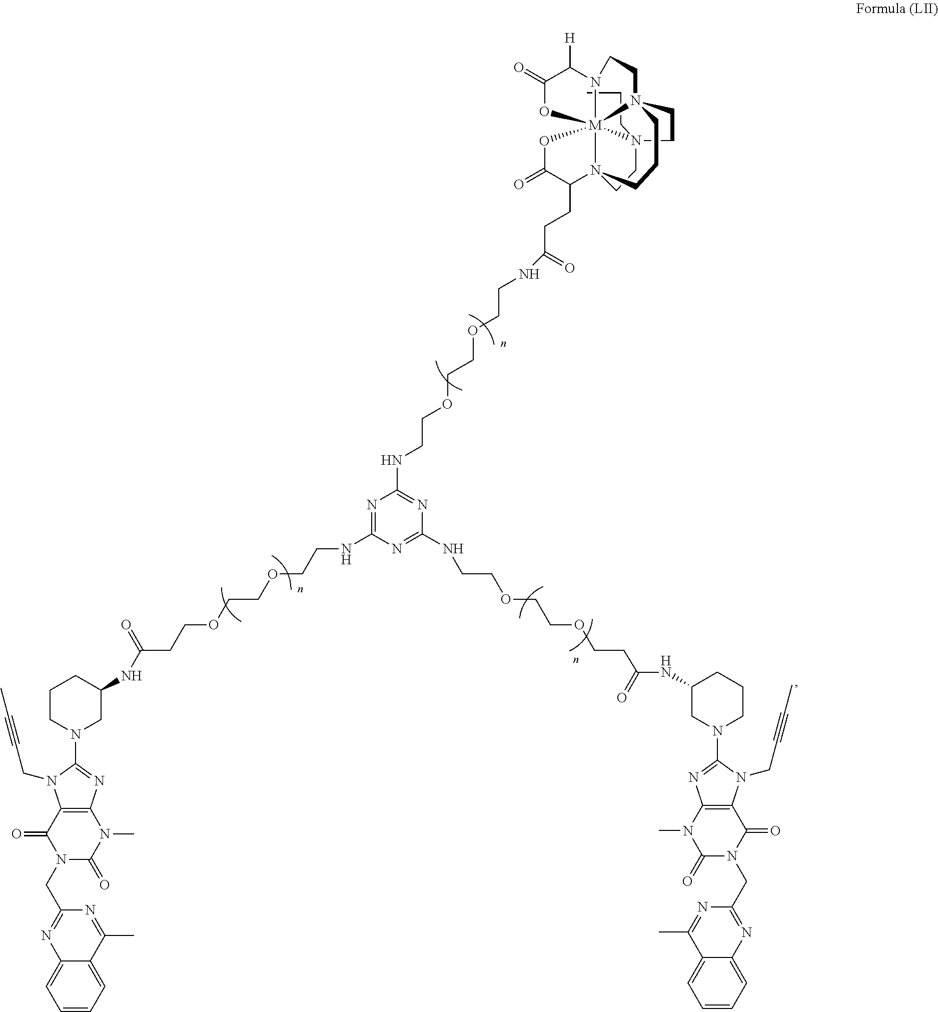 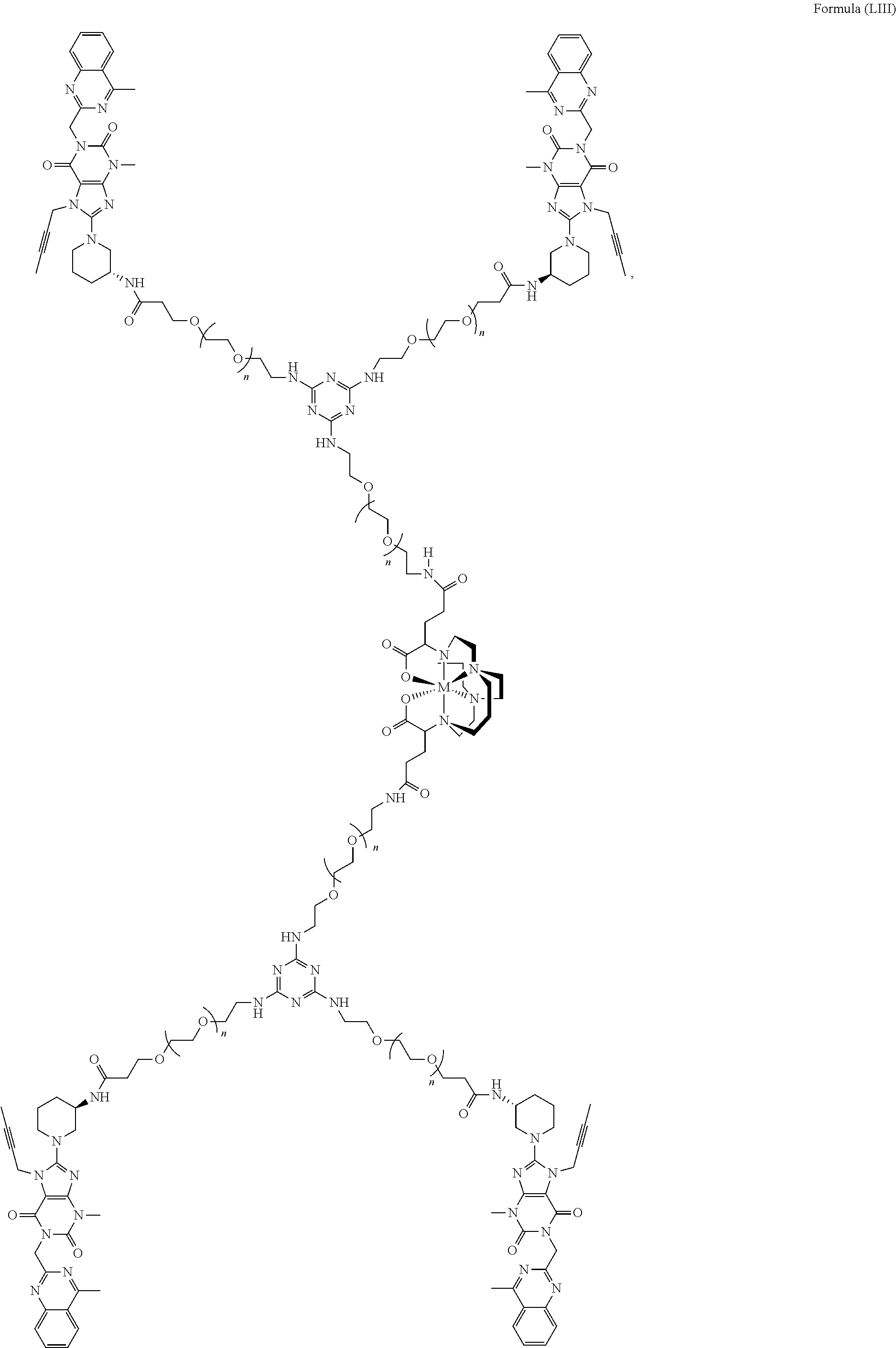 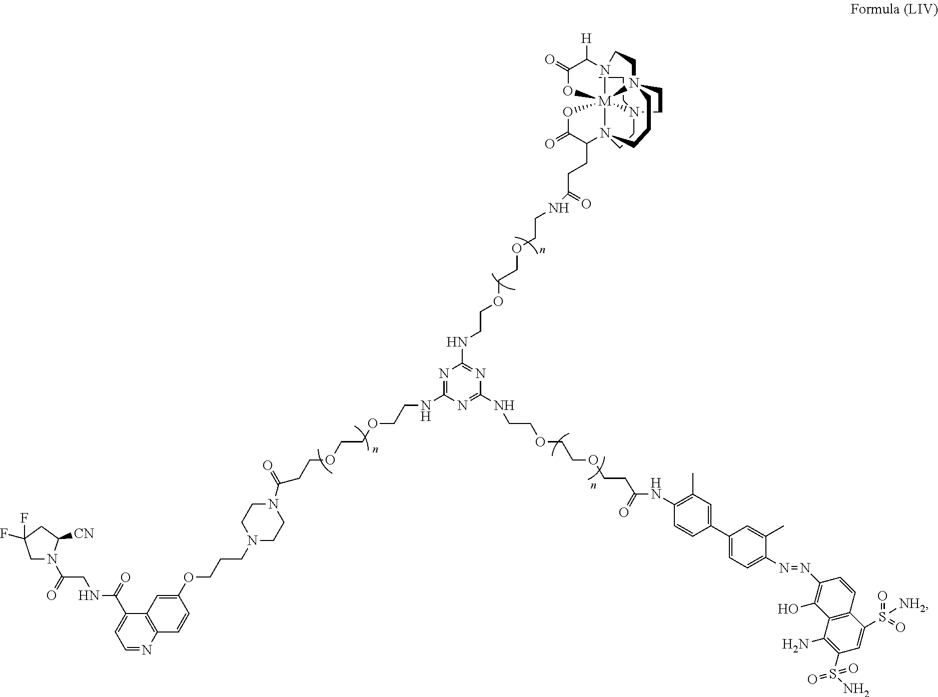 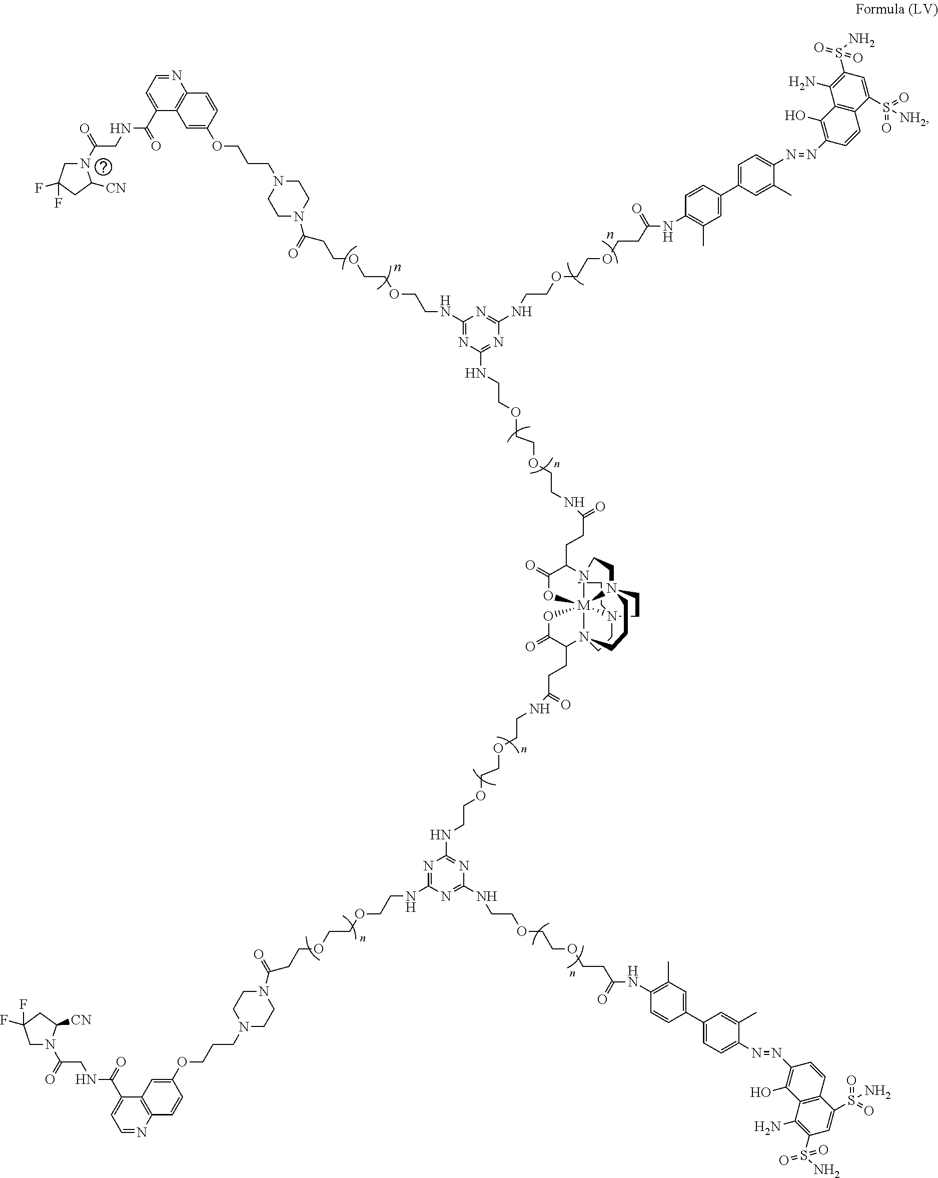 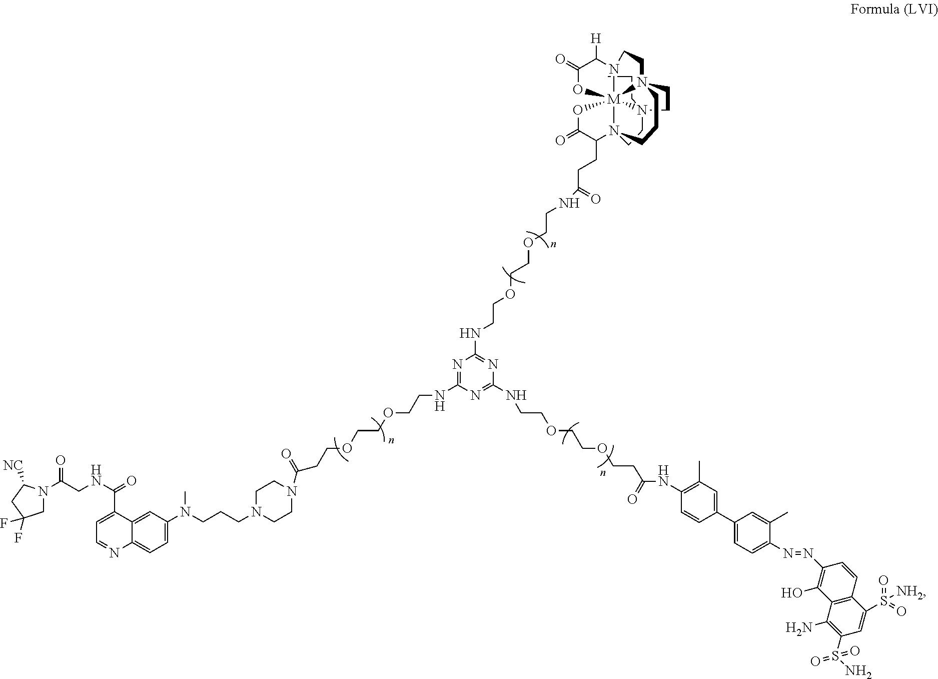 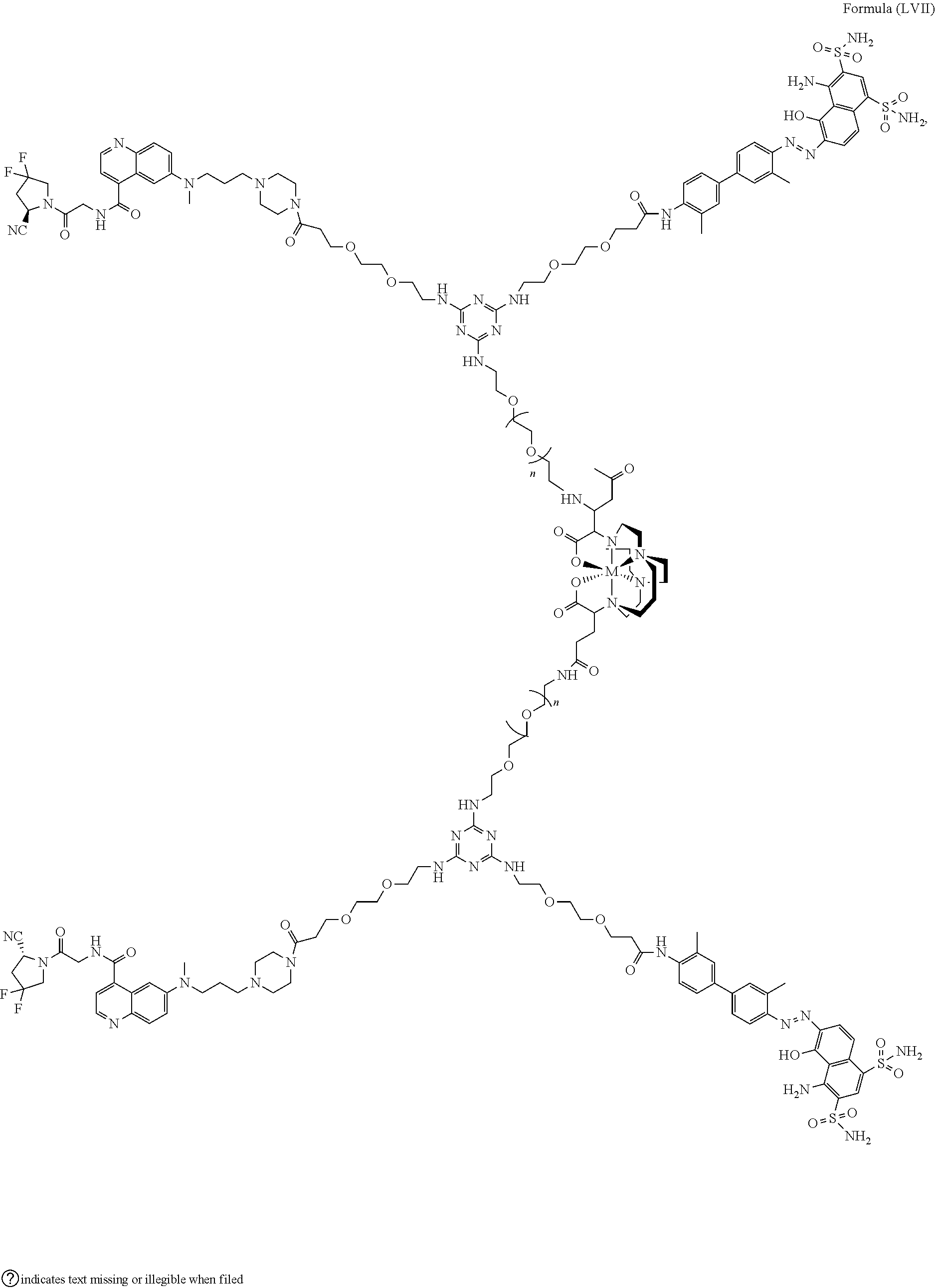 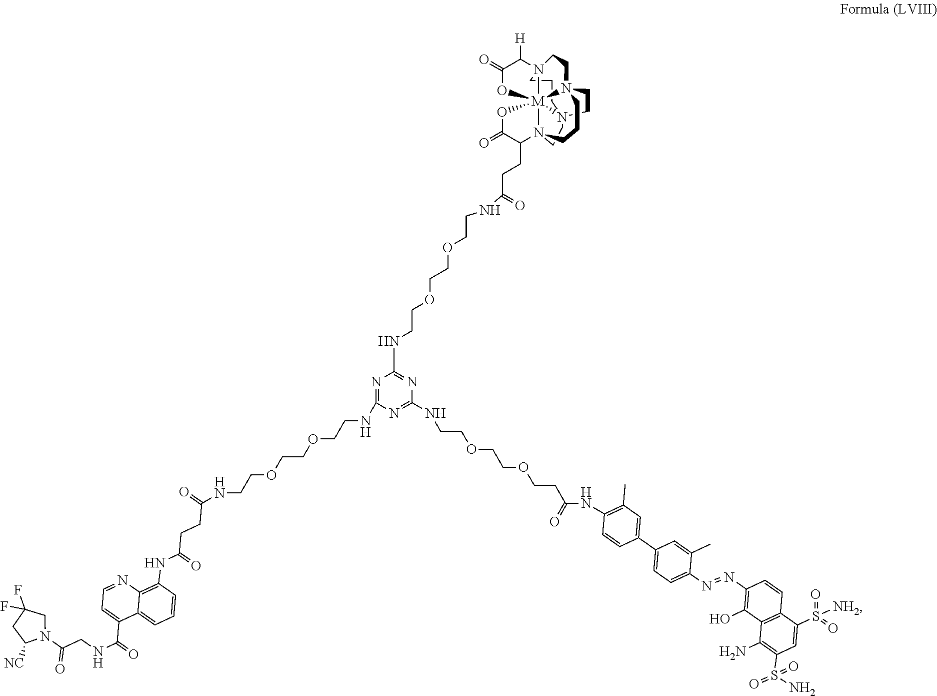 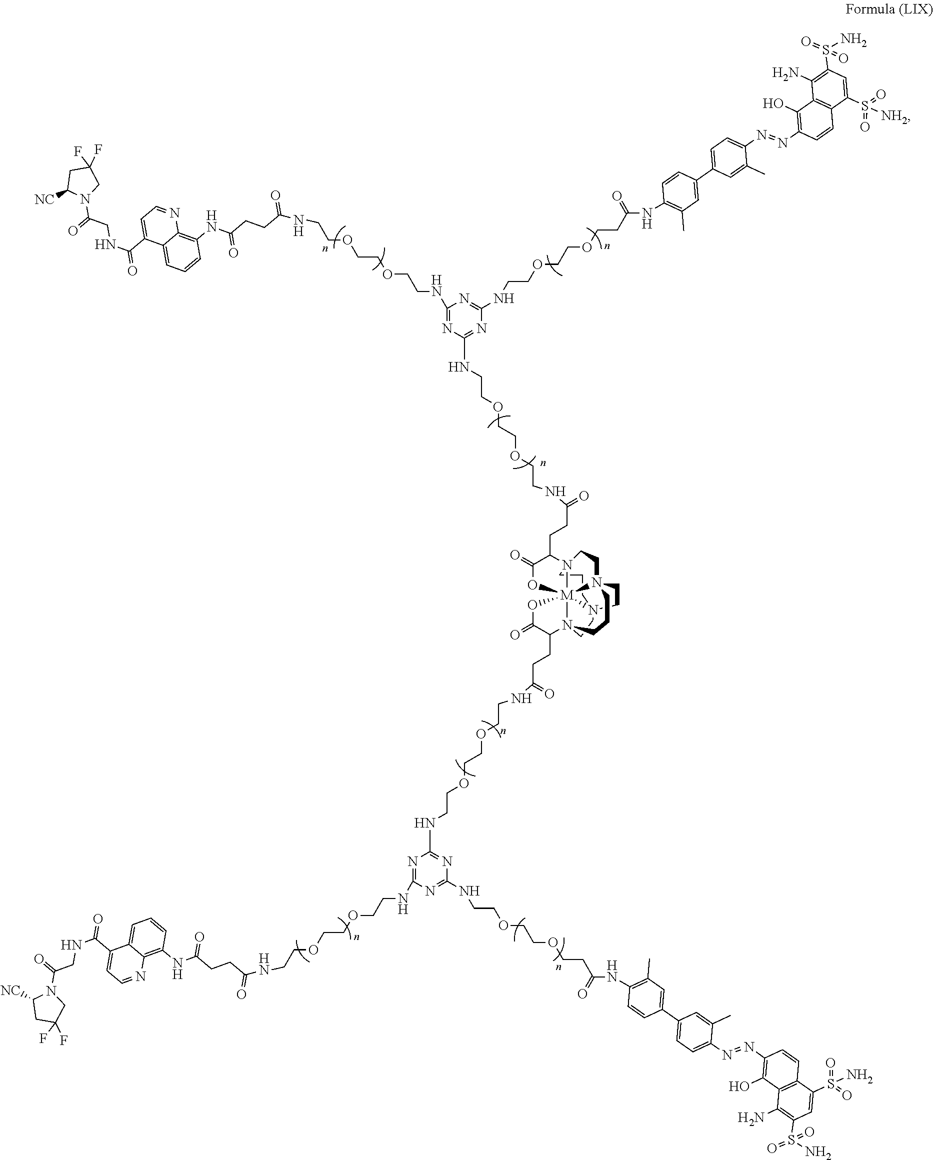 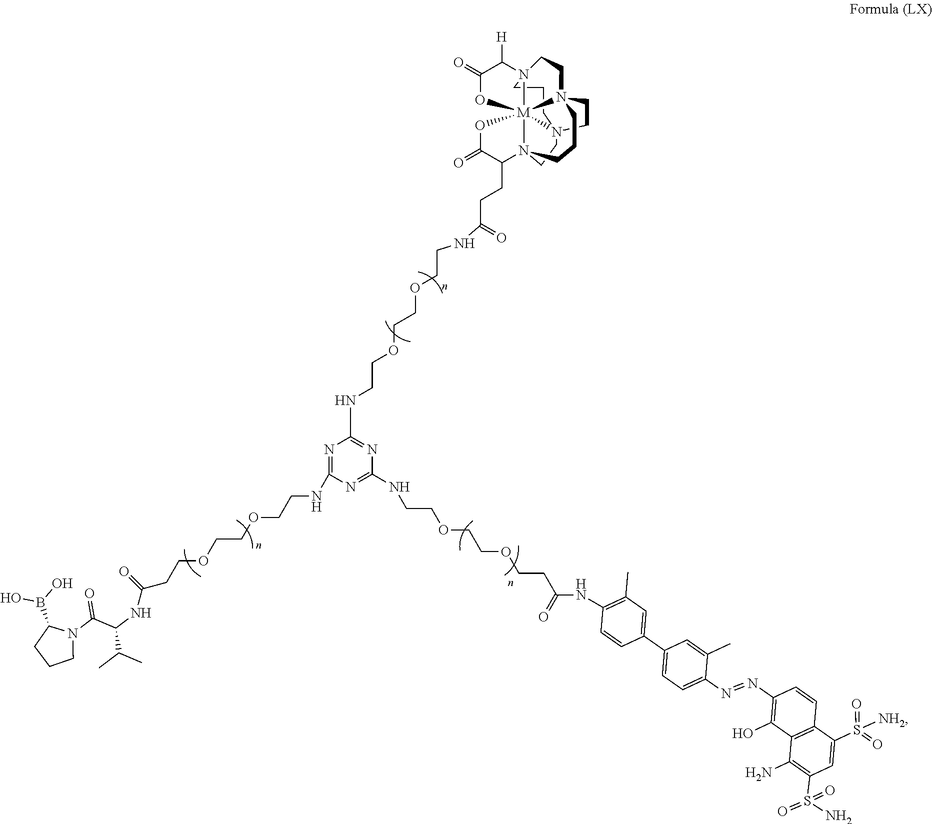 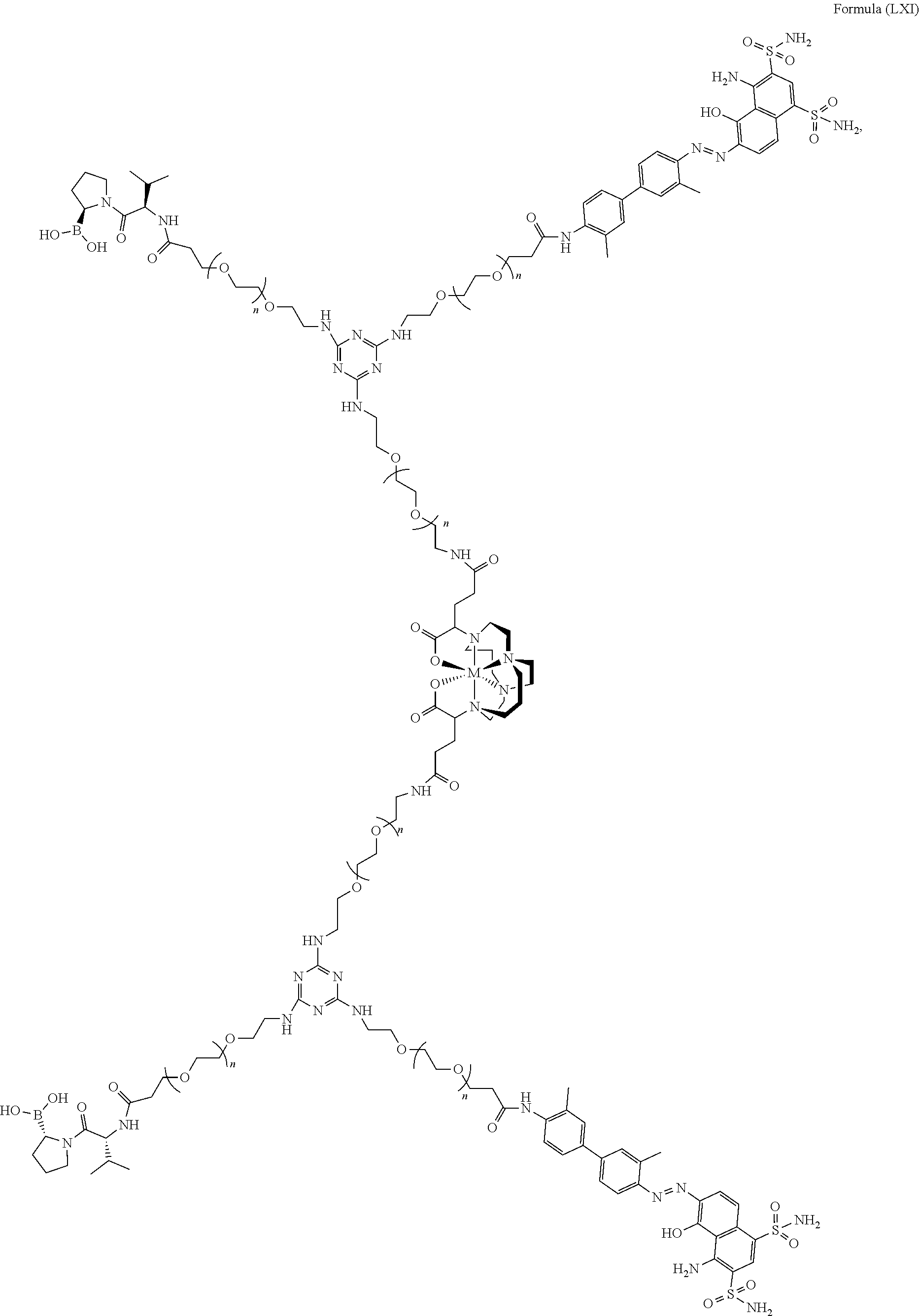 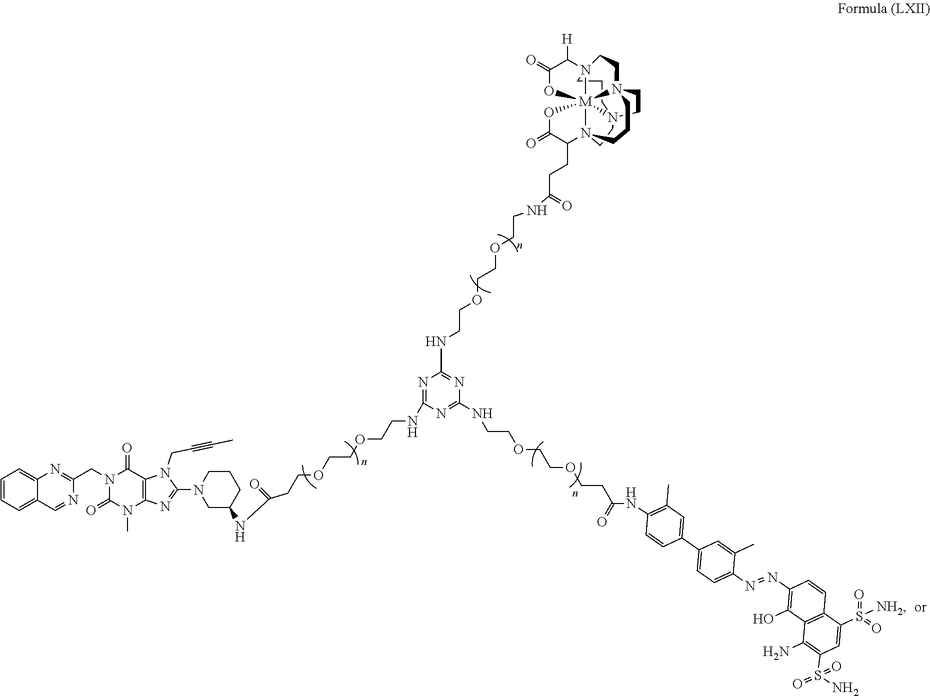 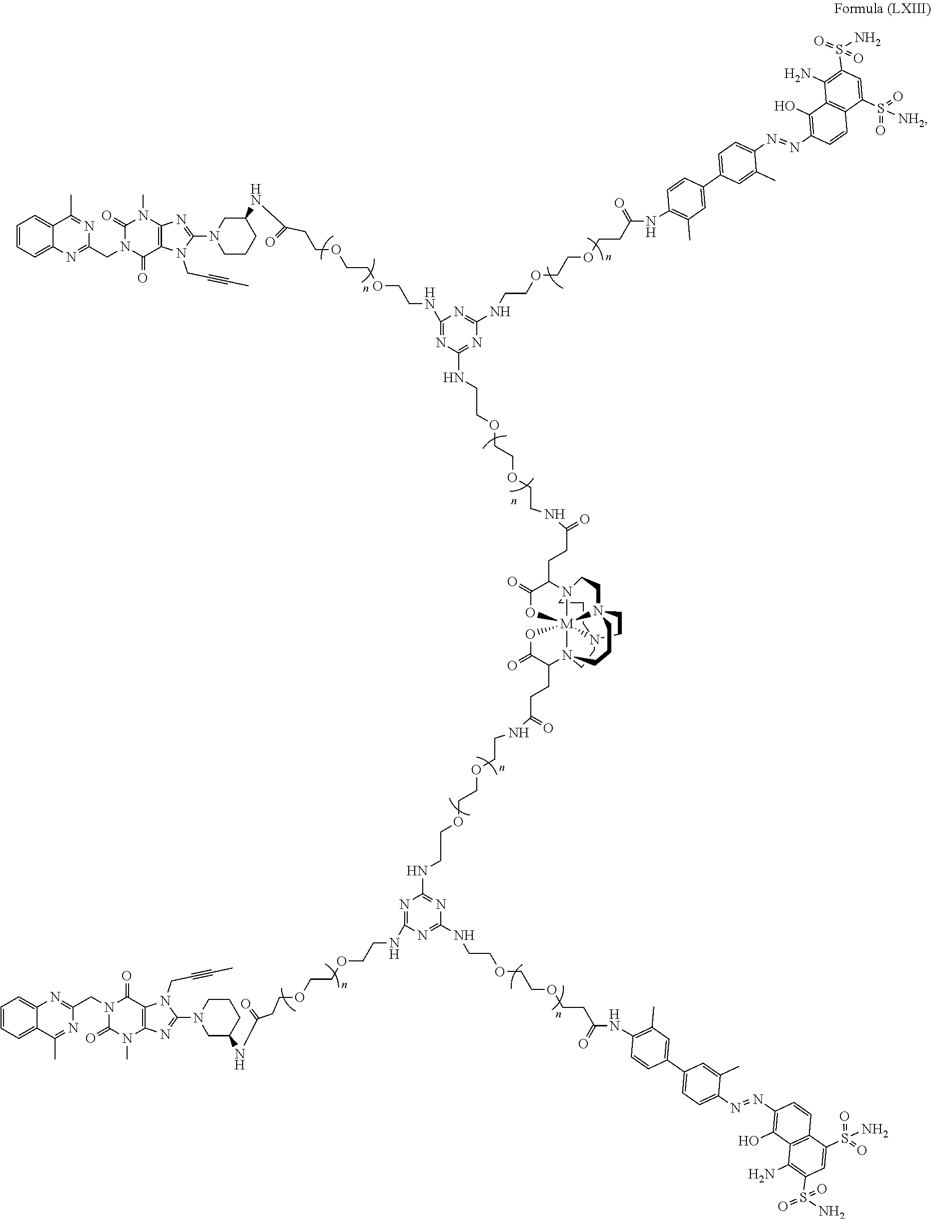
wherein n is 1 to 12 ethylene glycol repeating units or 1 to 6 amino acids; and
wherein R₃ comprises —OH, —SH, or —NH₂.

13. A compound of Formula (a) through Formula (h.b3), any positional isomer, any stereo isomer, or any pharmaceutically acceptable salts thereof: 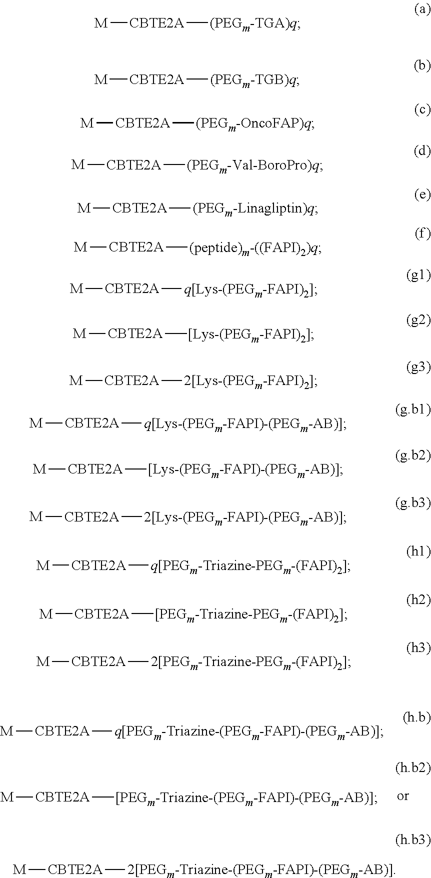
wherein
M is a chelating copper comprising ⁶⁰Cu, ⁶¹Cu, ⁶²Cu, ⁶⁴Cu, or ⁶⁷Cu;
m is zero, or any integer of 1 to 12;
q is 1 or 2;
PEG is polyethylene glycol;
AB is an albumin binder;
Lys is lysine;
CBTE2A represents one of the following two moieties 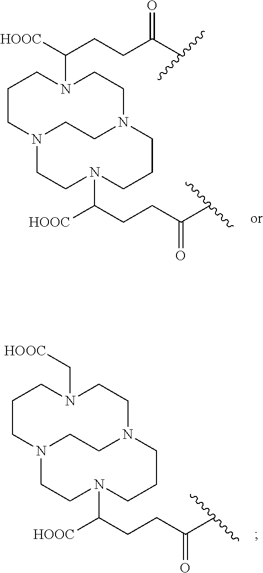 [SMILES: *C(=O)CCC(C(=O)O)N1CCCN2CCN(CCCN(C(CCC(*)=O)C(=O)O)CC2)CC1 ; *C(=O)CCC(C(=O)O)N1CCCN2CCN(CCCN(CC(=O)O)CC2)CC1]
FAPI is a Fibroblast Activation Protein targeting group, comprising TGA, TGB, OncoFAP, Val-BoroPro, or Linagliptin:

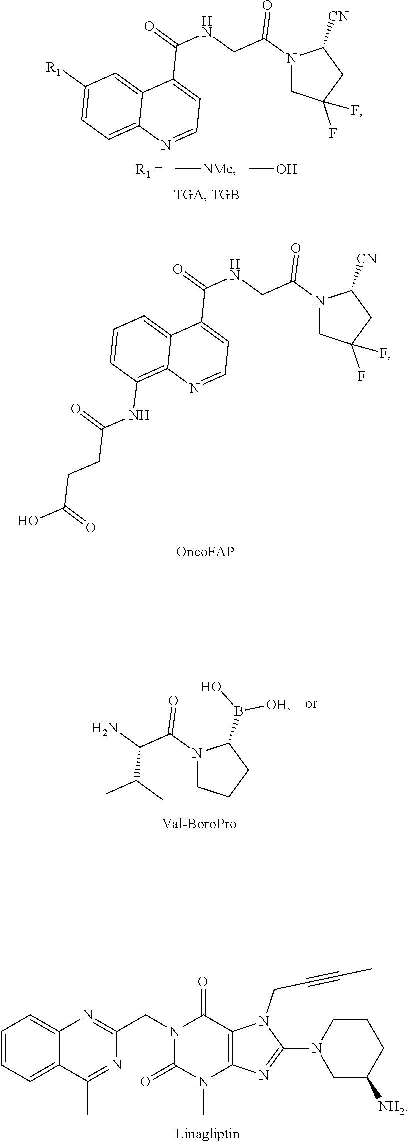

14. The compound of claim 13, wherein the compound comprises Formula (a), Formula (b), Formula (c), Formula (d), Formula (e), Formula (f), Formula (g), Formula (g.b), Formula (h), or Formula (h.b): 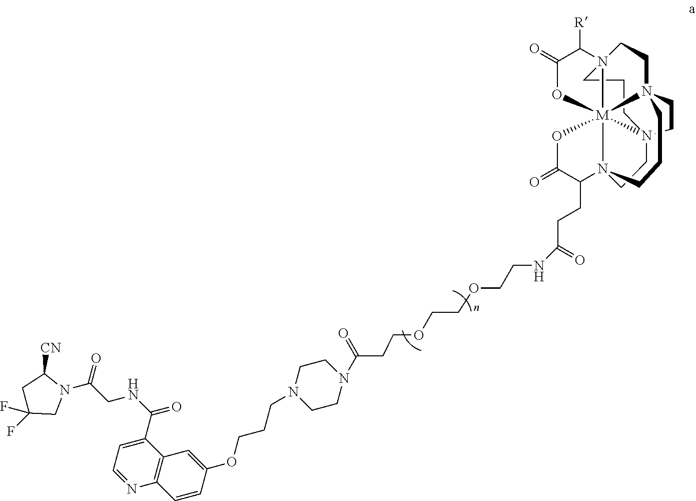
wherein if R′ is —H, a is M-CBTE2A-PEGₘ-TGB, and if R′ is —CH₂CH₂CO-PEGₘ-TGB, ais M-CBTE2A-(PEGₘ-TG B)₂; 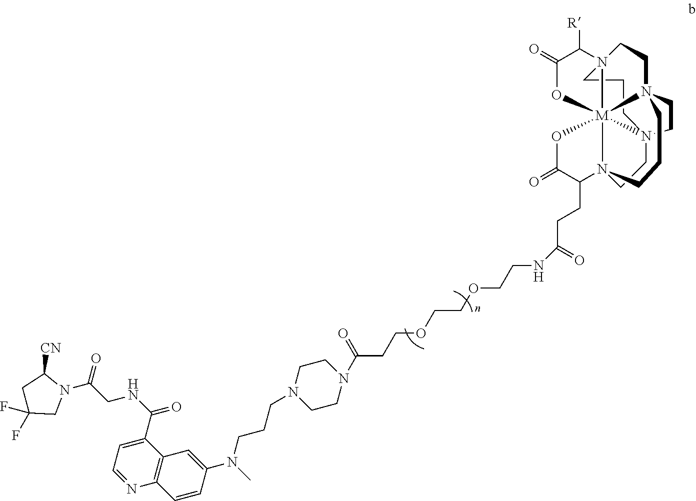
wherein if R′ is —H, b is M-CBTE2A-PEGₘ-TGA, and if R′ is —CH₂CH₂CO-PEGₘ-TGA, b is M-CBTE2A-(PEGₘ-TGA)₂; 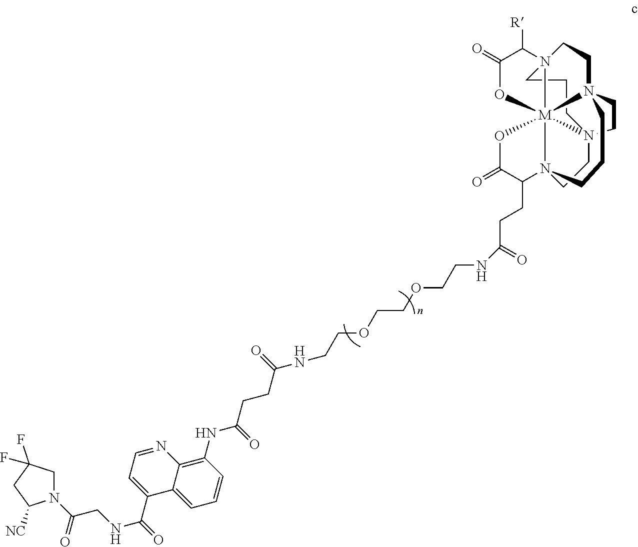
wherein if R′ is —H, c is M-CBTE2A-PEGₘ-OncoFAP, and if R′ is —CH₂CH₂CO-PEGₘ-OncoFAP, c is M-CBTE2A-(PEGₘ-OncoFAP)₂; 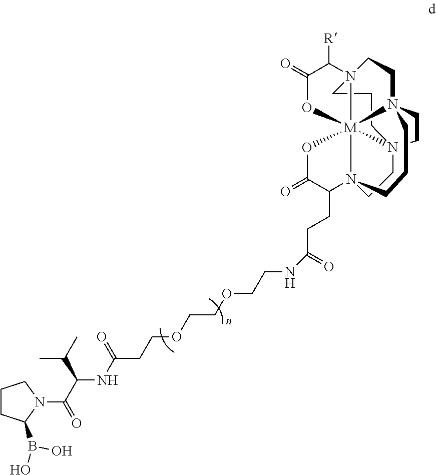
wherein if R′ is —H, d is M-CBTE2A-PEGₘ-ValBoro-Pro, and if R′ is —CH₂CH₂CO-PEGₘ-ValBoro-Pro, d is M-CBTE2A-(PEGₘ-ValBoro-Pro)₂; 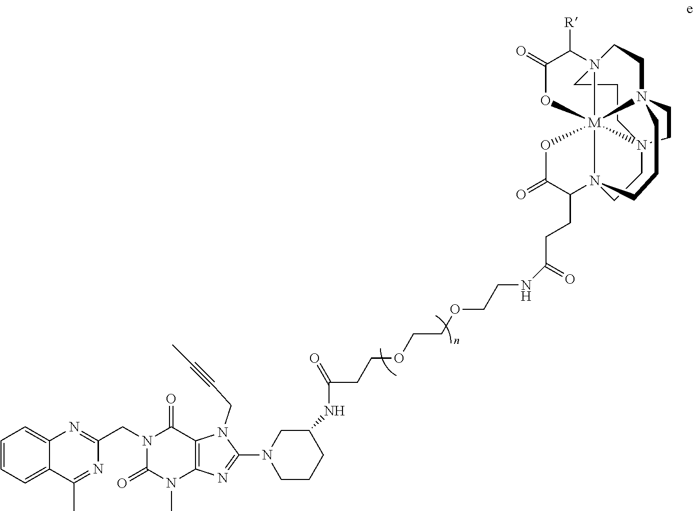
wherein if R′ is —H, e is M-CBTE2A-PEGₘ-Linagliptin, and if R′ is —CH₂CH₂CO-PEGₘ, Linagliptin, e is M-CBTE2A-(PEGₘ-Linagliptin)₂; 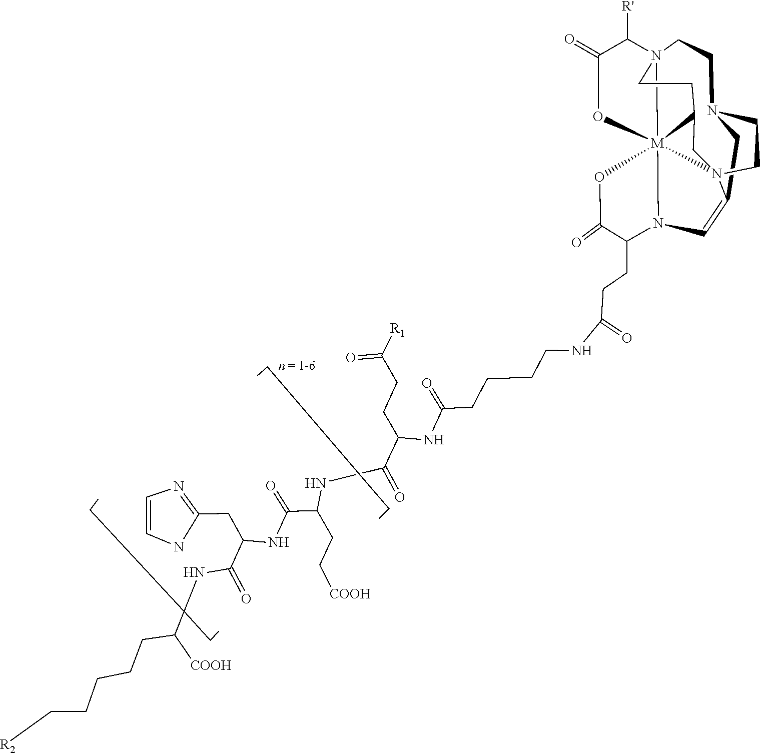
wherein
if R′ is —H, f is M-CBTE2A-Peptide-FAPI, and if R′ is —CH₂CH₂CO-Peptide-FAPI, f is M-CBTE2A-(Peptide-FAPI)₂;
R₁′ is —OH or —NH₂;
R₂′ is a chemical moiety of i, ii, iii, iv, or v: 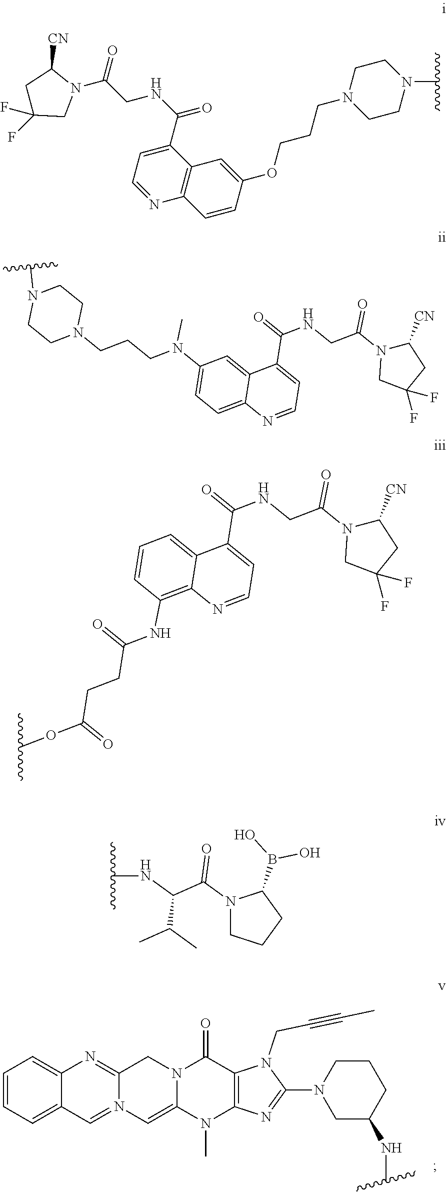 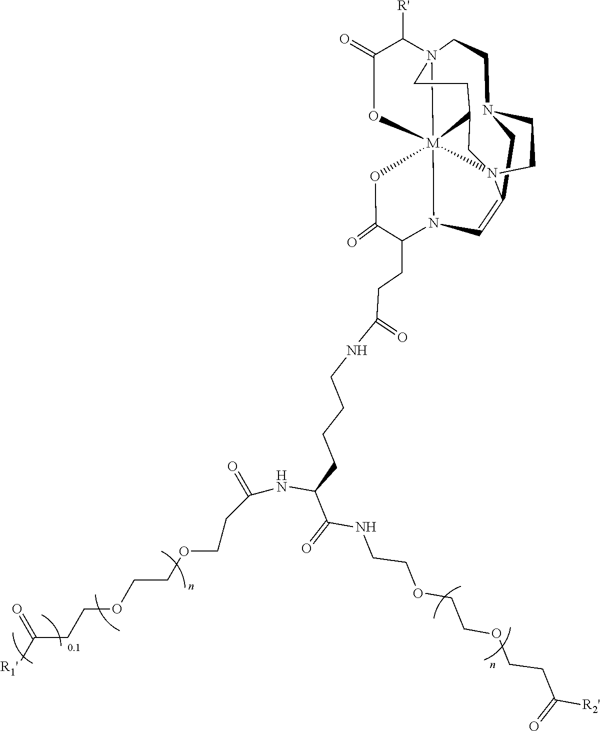
wherein
if R′ is —H, g is M-CBTE2A-[Lys-PEGₘ-(FAPI)₂], and if R′ is —CH₂CH₂CO-[Lys-PEGₘ-(FAPI)₂], g is M-CBTE2A-[Lys-PEGₘ-(FAPI)₂], and
R₁′ and R₂′ each is independently selected from the group consisting of i, ii, iiia, iv, and v. 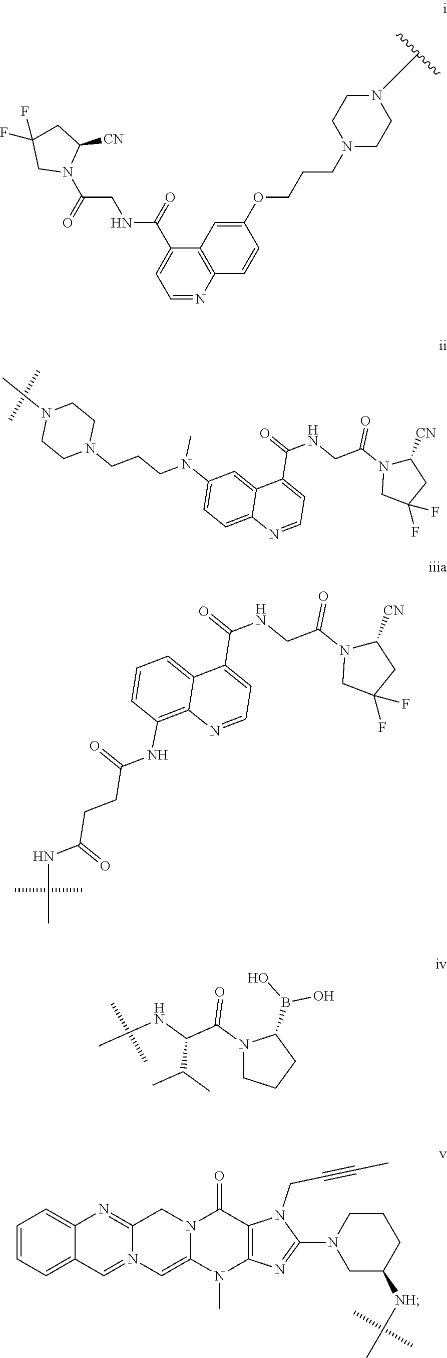 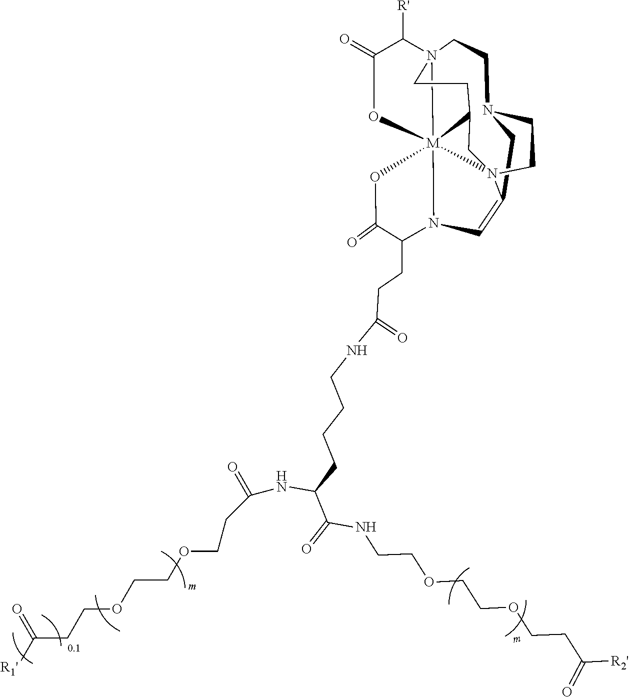
wherein
if R′ is —H, (g.b) is M-CBTE2A-[Lys-(PEGₘ-FAPI)-(PEGₘ-AB)], and if R′ is —CH₂CH₂CO-[Lys-(PEGₘ-FAPI)-(PEGₘ-AB)], (g.b) is M-CBTE2A-2[Lys-(PEGₘ-FAPI)-(PEGₘ-AB)], and
R₁′ or R₂′ each is independently selected from the group consisting of i, ii, iiia, iv, v, and vi: 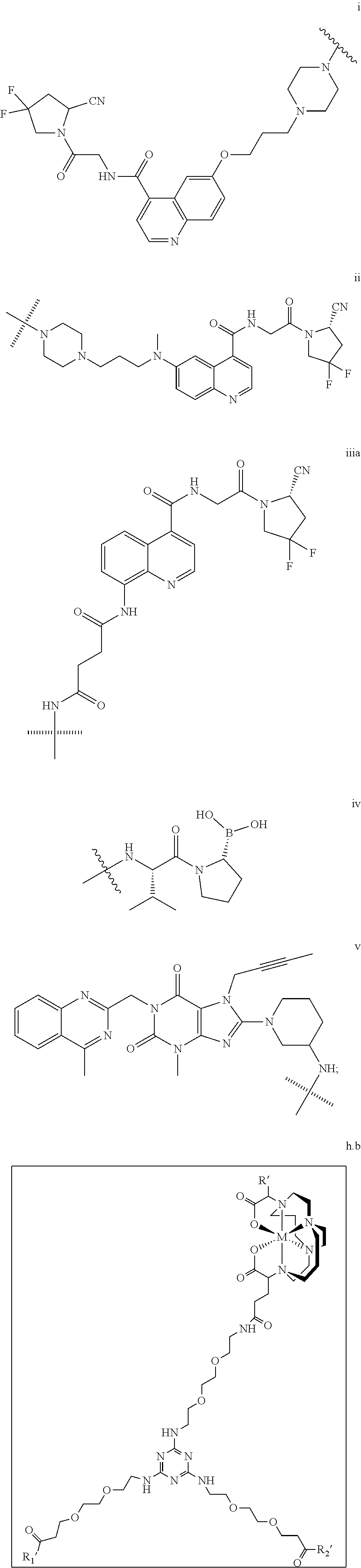
wherein if R′ is —H, h is M-CBTE2A-[PEGₘ-Triazine-(PEGₘ-FAPI)₂], and if R′ is —CH₂CH₂CO—[PEGₘ-Triazine-(PEGₘ-FAPI)₂], h is M-CBTE2A-2[PEGₘ-Triazine-(PEGₘ-FAPI)₂]; and
R₁′ and R₂′ each is independently selected from the group consisting of i, ii, iiia, iv, and v. 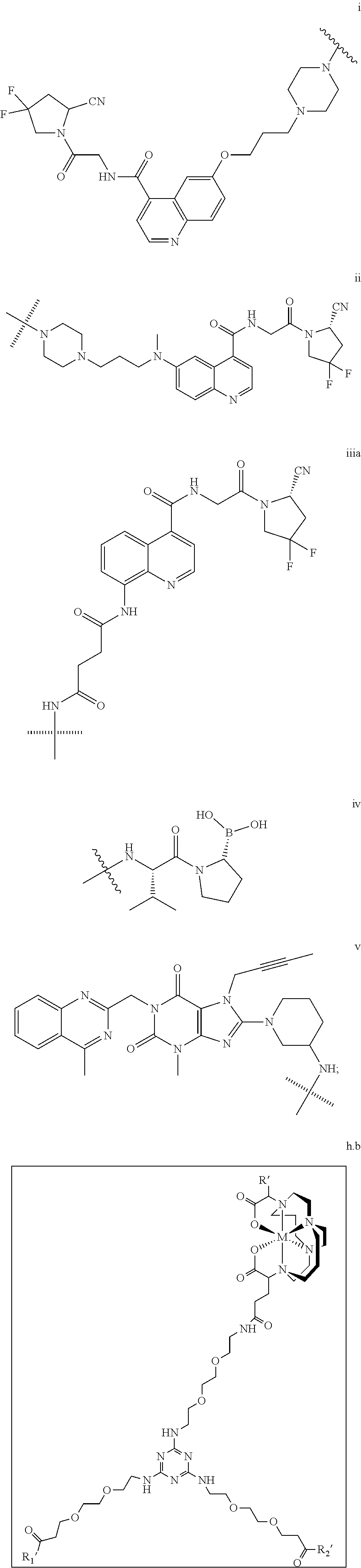
wherein if R′ is —H, (h.b) is M-CBTE2A-[PEGₘ-Triazine-(PEGₘ-FAPI)-(PEGₘ-AB)], and if R′ is —CH₂CH₂CO-[PEGₘ-Triazine-(PEGₘ-FAPI-(PEGₘ-AB)], (h.b) is M-CBTE2A-2[PEGₘ-Triazine-(PEGₘ-FAPI)-(PEGₘ-AB)];
and
R₁′ or R₂′ each is independently selected from the group consisting of i, ii, iiia, iv, v, and vi:

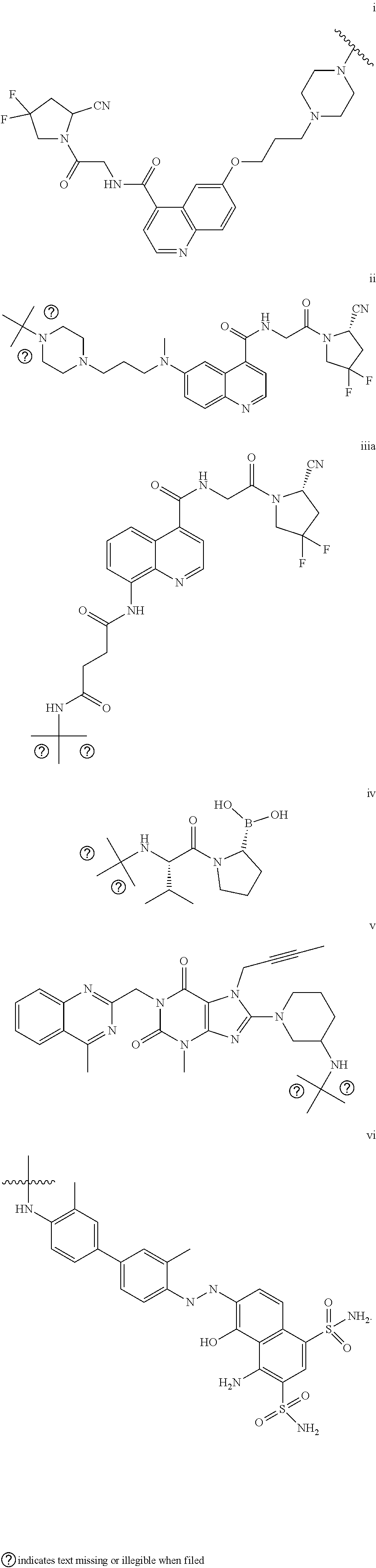

15. The compound of claims 13-14, wherein the compound comprises Formula (IIa) or Formula (IIIa).

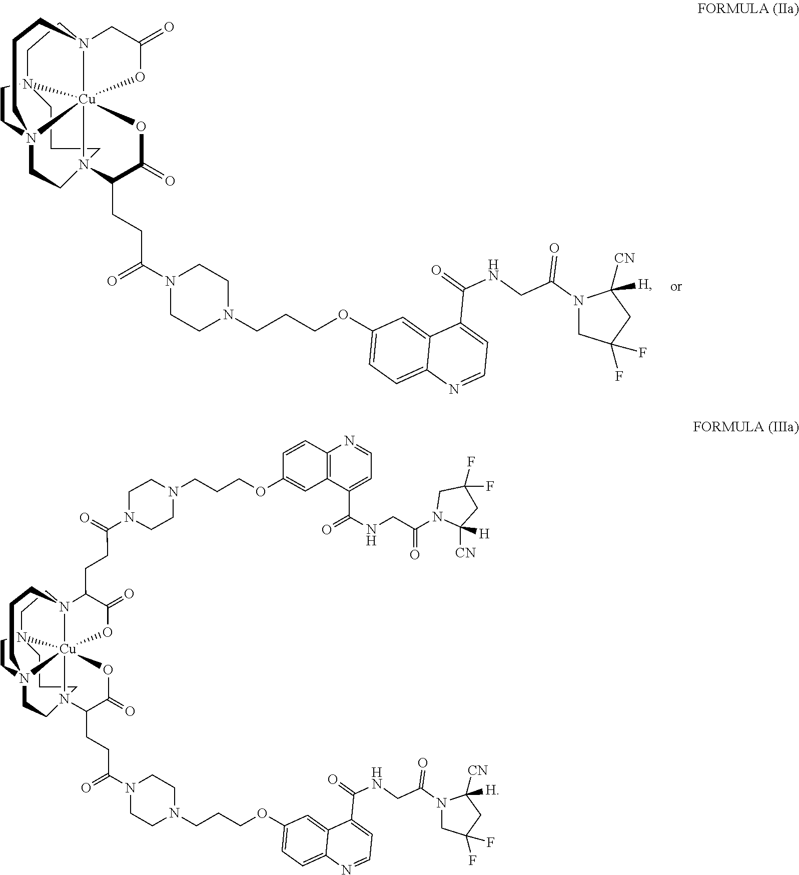

16. A pharmaceutical composition comprising the compound of any one of claims 1-15 and at least one pharmaceutically acceptable excipient or carrier.

17. A method of preparing the compound of Formula (I): 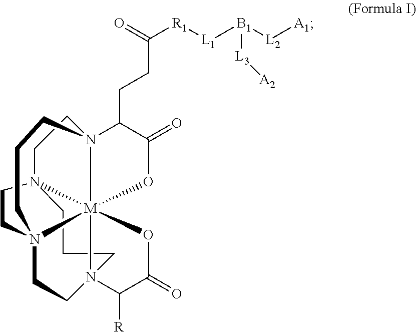
the method comprising the steps of:
(a) contacting the compound of Formula (LXIV): 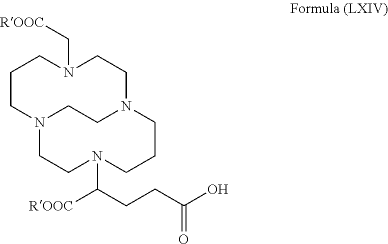
with the compound of Formula (LXV): 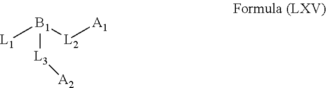
in the presence of an acyl coupling reagent and proton acceptor to produce the compound of Formula (LXVI): 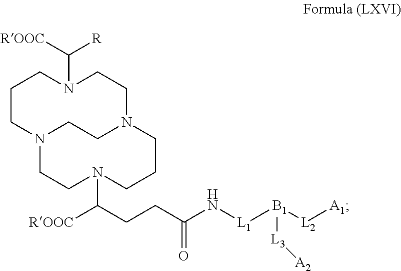
(b) contacting the produced compound of Formula (LXVI) with a proton acceptor or a proton donor to produce the compound of Formula (LXVII). 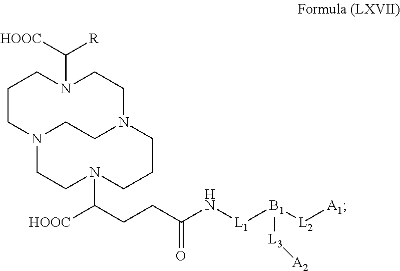
and
(c) contact the produced compound of Formula (LXVII) with a M-salt in the presence of a buffer to produce the compound of Formula (I);
wherein in Formula (I)
A₁ is a Fibroblast Activated Protein (FAP) targeting group (FAPI) or an albumin binder (AB) group, wherein the FAPI or the AB group is capable of covalent or non-covalent binding with the FAP or albumin respectively;
A₂ is a Fibroblast Activated Protein (FAP) targeting group (FAPI), an albumin binder (AB) group, or absent, wherein the FAPI or the AB group is capable of covalent or non-covalent binding with the FAP or albumin respectively;
R₁ is —NH, piperazine, piperidine, an alkylamine, a dicarboxylate amide, or is absent;
L₁ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof;
L₂ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent;
L₃ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent;
B₁ is a heterocyclic group, a heterocyclic alkyl, an amidoalkyl, an alkylamide, or is absent;
R is hydrogen or Formula (Ia) 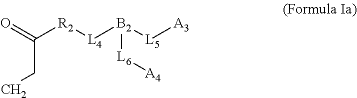
wherein,
A₃ is a Fibroblast Activated Protein (FAP) targeting group (FAPI) or an albumin binder (AB) group, wherein the FAPI or the AB group is capable of covalent or non-covalent binding with the FAP or albumin respectively;
A₄ is a Fibroblast Activated Protein (FAP) targeting group (FAPI), an albumin binder (AB) group, or absent, wherein the FAPI or the AB group is capable of covalent or non-covalent binding with the FAP or albumin respectively;
R₂ is —NH, piperazine, piperidine, an alkylamine, a dicarboxylate amide, or is absent;
L₄ is a linker comprising an alkyl, an alkylamine, an aminocarboxylate, a peptide, a peptide alkyl, a peptidomimetic, a peptidomimetic alkyl, a polyethylene glycol (PEG), a PEG alkyl, a functionalized PEG, a triazine-PEG, or any combination thereof;
L₅ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alkyl, a peptide, a PEG, a functionalized polyethylene glycol (PEG), any combination thereof, or is absent;
L₆ is a linker comprising an alkyl, an alkylamine, an aminocarboxylate, an alkylcarbonyl, an alkylamide, an alkylamidoalkylcarbonyl, an alkyl PEG carbonyl, a PEG alkylcarbonyl, a PEG alklyl, a peptide, a PEG, a functionalized polyethylene glycol (PEG), any combination thereof, or is absent;
B₂ is a heterocyclic group, a heterocyclic alkyl, an amidoalkyl, an alkylamide, or is absent;
M is ⁶⁴Cu, ⁶⁰Cu, ⁶¹Cu, ⁶²Cu or ⁶⁷Cu.

18. The method of claim 17, wherein the method yields the compound of Formula (I) in a yield greater than 60%.

19. The method of claims 17-18, wherein the method yields the compound of Formula (I) in greater than 95% radiochemical purity.

20. A method of treating and/or diagnosing of a disease characterized by overexpression of fibroblast activation protein (FAP), the method comprising administering an effective amount of a compound according to any one of claims 1-15 or a pharmaceutical composition according to claim 16 to a subject in need thereof.

21. The method of claim 20, wherein the method is a method of treating a disease and M is ⁶⁷Cu.

22. The method of claim 20, wherein the method is a method of diagnosing a disease and M is ⁶⁴Cu, ⁶⁰Cu, ⁶¹Cu, ⁶²Cu or ⁶⁷Cu.

23. The method of claims 21-22, wherein disease characterized by overexpression of fibroblast activation protein (FAP) selected from cancer, chronic inflammation, atherosclerosis, fibrosis, tissue remodeling and keloid disorder, breast cancer, renal cell carcinoma, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.

24. The method of any one of claims 20-23, wherein the diagnosis and/or treatment of renal cell carcinoma.

25. The method of claim 20, further comprising imaging evaluation of the subject.

26. The method of claim 25, wherein the imaging evaluation comprises one or more of a positron emission tomography (PET), or single photon emission computer tomography (SPECT).

27. The method of any one of the claims 20-26, further comprising administering additional treatments including but not restricted to chemotherapy, radiation therapy and/or drugs before, simultaneously or after the pharmaceutical composition.

28. The method of any one of the claims 20-27, wherein the subject is a human.

29. The method of any one of claims 20-28, wherein the subject is diagnosed with or is suspected of having a disease characterized by overexpression of fibroblast activation protein (FAP).

30. The method of claim 29, wherein the subject is diagnosed with or suspected of having any one or more of a cancer, chronic inflammation, atherosclerosis, fibrosis, tissue remodeling and keloid disorder, breast cancer, renal cell carcinoma, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.

31. Use of the compound of any one of claims 1-15 as a radiopharmaceutical active or a radiotheranostic active.

32. Use of the compound of any one of claims 1-15 in the manufacture of a medicament for radiopharmaceutical therapy or radiotheranostic therapy.
